(19) United States
(12) Patent Application Publication
(10) Pub. No.: US 2025/0368634 A1
(43) Pub. Date: Dec. 4, 2025

(54) PHENYL AMIDE COMPOUNDS AND METHODS OF USE

(71) Applicant: SCHRÖDINGER, INC., New York, NY (US)

(72) Inventors: Jeremy GREENWOOD; Evelyne HOUANG, New York, NY (US); Zef KONST, New York, NY (US); Abba LEFFLER, New York, NY (US); Adam LEVINSON, New York, NY (US); Andrew PLACZEK, New York, NY (US); Anatoly RUVINSKY, New York, NY (US)

(73) Assignee: SCHRÖDINGER, INC., New York, NY (US)

(21) Appl. No.: 18/874,779

(22) PCT Filed: Jun. 13, 2023
(86) PCT No.: PCT/US2023/068371

§ 371 Date: Dec. 13, 2024

Related U.S. Application Data
(60) Provisional application No. 63/352,053, filed on Jun. 14, 2022.

(51) Int. Cl. C07D 413/12; A61K 31/443; A61K 31/4439; A61K 31/496; A61K 31/519; A61K 31/5375; C07D 211/58; C07D 265/30; C07D 401/12; C07D 405/12; C07D 487/04; C07D 487/10; C07D 513/10

(52) U.S. Cl. CPC C07D 413/12; A61K 31/443; A61K 31/4439; A61K 31/496; A61K 31/519; A61K 31/5375; C07D 211/58; C07D 265/30; C07D 401/12; C07D 405/12; C07D 487/04; C07D 487/10; C07D 513/10

(57) ABSTRACT

The present application relates to compounds of Formula (I), as defined herein, and pharmaceutically acceptable salts thereof. The present application also describes pharmaceutical composition comprising a compound of Formula (I), and pharmaceutically acceptable salts thereof, and methods of using the compounds and compositions for inhibiting certain protein-protein interactions, and for treating cancer.

### TECHNICAL FIELD

This present application relates to phenyl amide compounds that are useful for treating proliferative disorders such as cancer.

### BACKGROUND

Cancer is characterized by aberrant cell growth and proliferation. Ras proteins are critical components of signaling networks responsible for controlling cellular proliferation, differentiation, and survival. See, e.g., Fernandes-Medarde and Santos, Genes Cancer, Vol. 2, No. 3, pp. 344-358 (2011). Ras is a GTPase that acts as a molecular switch between an active GTP-bound state and an inactive GDP-bound state. GTP-bound Ras can activate several downstream signaling pathways involved in cell cycle progression, survival, and apoptosis.

Guanine nucleotide exchange factors (GEFs), such as SOS1, are required to activate Ras by facilitating the exchange of GDP (inactive Ras) for GTP (active Ras). SOS1 is itself activated by Ras via an allosteric interaction, which strongly activates the GEF function of SOS1, thus creating a positive feedback loop between SOS1 and Ras. See, e.g., Bandaru, et al., Cold Spring Harb. Perspect Med., Vol. 9, No. 2, a031534 (2019). Mutations in Ras occur in many human cancers, but currently no drug targeting Ras proteins has been approved. See Hillig, et al., Proc. Nat. Acad. Sci., Vol. 117, No. 7, pp. 2551-2560 (2019). Thus, there remains a need for novel therapeutics to disrupt Ras signaling.

### SUMMARY

It has now been found that certain phenyl amide compounds are inhibitors of SOS1 activity, and are useful for treating various diseases and disorders, such as cancers.

Accordingly, provided herein is a compound of the Formula (I):

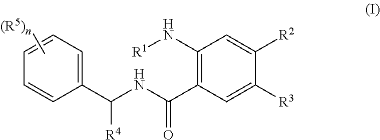

or a pharmaceutically acceptable salt thereof, wherein R¹, R², R³, R⁴, R⁵ and “n” are as defined herein.

Also provided herein is a pharmaceutical composition comprising a compound of Formula (I), or a pharmaceutically acceptable salt thereof, and at least one pharmaceutically acceptable excipient.

Also provided herein is a method of inhibiting mammalian cell proliferation, in vitro or in vivo, comprising contacting a cell with an effective amount of a compound of Formula (I) or a pharmaceutically acceptable salt thereof, or a pharmaceutical composition thereof as defined herein.

Also provided herein is a method of treating cancer in a subject in need of such treatment, comprising administering to the subject an effective amount of a compound of Formula (I), or a pharmaceutically acceptable salt thereof, or a pharmaceutical composition thereof as defined herein.

Also provided herein is a method of treating a SOS1-associated cancer in a subject in need of such treatment, comprising administering to the subject an effective amount of a compound of Formula (I), or a pharmaceutically acceptable salt thereof, or a pharmaceutical composition thereof as defined herein.

Also provided herein is a method of treating a Ras pathway-associated disease or disorder in a subject, comprising administering to a subject identified or diagnosed as having a Ras pathway-associated disease or disorder an effective amount of a compound of Formula (I), or a pharmaceutically acceptable salt thereof, or a pharmaceutical composition thereof as defined herein, to the subject.

Also provided herein is a method of treating a Ras pathway-associated cancer in a subject, comprising administering to a subject identified or diagnosed as having a Ras pathway-associated cancer an effective amount of a compound of Formula (I), or a pharmaceutically acceptable salt thereof, or a pharmaceutical composition thereof as defined herein, to the subject.

Also provided herein is a method of treating a Ras-associated disease or disorder in a subject, comprising administering to a subject identified or diagnosed as having a Ras-associated disease or disorder an effective amount of a compound of Formula (I), or a pharmaceutically acceptable salt thereof, or a pharmaceutical composition thereof as defined herein, to the subject.

Also provided herein is a method of treating a Ras-associated cancer in a subject, comprising administering to a subject identified or diagnosed as having a Ras-associated cancer an effective amount of a compound of Formula (I), or a pharmaceutically acceptable salt thereof, or a pharmaceutical composition thereof as defined herein, to the subject.

Also provided herein is a method of treating a SOS1-associated cancer in a subject, comprising administering to a subject identified or diagnosed as having a SOS1-associated cancer an effective amount of a compound of Formula (I), or a pharmaceutically acceptable salt thereof, or a pharmaceutical composition thereof as defined herein, to the subject.

Also provided herein is a method for treating cancer in a subject in need thereof, comprising:
(a) determining that the cancer is associated with a dysregulation of a Ras pathway gene, a Ras pathway protein, or expression or activity or level of any of the same; and
(b) administering to the subject an effective amount of a compound of Formula (I), or a pharmaceutically acceptable salt thereof, or a pharmaceutical composition thereof as defined herein, to the subject.

Also provided herein is a method for treating cancer in a subject in need thereof, comprising administering to the subject an effective amount of a compound of Formula (I), or a pharmaceutically acceptable salt thereof, or a pharmaceutical composition thereof as defined herein to a subject determined to have a cancer is associated with a dysregulation of a Ras pathway gene, a Ras pathway protein, or expression or activity or level of any of the same.

Also provided herein is a method for treating cancer in a subject in need thereof, comprising:
(a) determining that the cancer is associated with a dysregulation of a Ras gene, a Ras protein, or expression or activity or level of any of the same; and
(b) administering to the subject an effective amount of a compound of Formula (I), or a pharmaceutically acceptable salt thereof, or a pharmaceutical composition thereof as defined herein, to the subject.

Also provided herein is a method for treating cancer in a subject in need thereof, comprising administering to the subject an effective amount of a compound of Formula (I), or a pharmaceutically acceptable salt thereof, or a pharmaceutical composition thereof as defined herein to a subject determined to have a cancer is associated with a dysregulation of a Ras gene, a Ras protein, or expression or activity or level of any of the same.

Also provided herein is a method for treating cancer in a subject in need thereof, comprising administering to the subject an effective amount of a compound of Formula (I), or a pharmaceutically acceptable salt thereof, or a pharmaceutical composition thereof as defined herein to a subject determined to have a cancer is associated with a dysregulation of a SOS1 gene, a SOS1 protein, or expression or activity or level of any of the same.

Also provided herein is a method for treating cancer in a subject in need thereof, comprising:
(a) determining that the cancer is associated with a dysregulation of a SOS1 gene, a SOS1 protein, or expression or activity or level of any of the same; and
(b) administering to the subject an effective amount of a compound of Formula (I), or a pharmaceutically acceptable salt thereof, or a pharmaceutical composition thereof as defined herein, to the subject.

Also provided herein is a method for inhibiting mammalian cell proliferation, comprising contacting the mammalian cell with a compound of Formula (I), or a pharmaceutically acceptable salt thereof.

Also provided herein is a method for inhibiting Ras pathway activity in a mammalian cell, comprising contacting the mammalian cell with a compound of Formula (I), or a pharmaceutically acceptable salt thereof.

Also provided herein is a method for inhibiting SOS1 activity in a mammalian cell, comprising contacting the mammalian cell with a compound of Formula (I), or a pharmaceutically acceptable salt thereof.

Also provided herein is a method for inhibiting Ras activity in a mammalian cell, comprising contacting the mammalian cell with a compound of Formula (I), or a pharmaceutically acceptable salt thereof.

Also provided herein is a method for inhibiting a SOS1-Ras protein-protein interaction in a mammalian cell, comprising contacting the mammalian cell with a compound of Formula (I), or a pharmaceutically acceptable salt thereof.

Also provided herein is a method for inhibiting metastasis in a subject having a particular cancer in need of such treatment, comprising administering to the subject an effective amount of a compound of Formula (I), or a pharmaceutically acceptable salt thereof, or a pharmaceutical composition comprising a compound of Formula (I), or a pharmaceutically acceptable salt thereof.

Also provided herein is a compound of Formula (I), or a pharmaceutically acceptable salt thereof, or a pharmaceutical composition thereof as defined herein for use in the treatment of cancer.

Also provided herein is a compound of Formula (I), or a pharmaceutically acceptable salt thereof, or a pharmaceutical composition thereof as defined herein for use in the treatment of a Ras pathway-associated disease or disorder.

Also provided herein is a compound of Formula (I), or a pharmaceutically acceptable salt thereof, or a pharmaceutical composition thereof as defined herein for use in the treatment of a Ras pathway-associated cancer.

Also provided herein is a compound of Formula (I), or a pharmaceutically acceptable salt thereof or a pharmaceutical composition thereof as defined herein for use in the treatment of cancer and/or inhibiting metastasis associated with a particular cancer.

Also provided herein is a compound of Formula (I), or a pharmaceutically acceptable salt thereof for use in the inhibition of a SOS1-Ras protein-protein interaction in a mammalian cell.

Also provided herein is a compound of Formula (I), or a pharmaceutically acceptable salt thereof, defined herein in the manufacture of a medicament for the inhibition of a SOS1-Ras protein-protein interaction in a mammalian cell.

Also provided herein is a compound of Formula (I), or a pharmaceutically acceptable salt thereof, as defined herein, in the manufacture of a medicament for the treatment of a Ras pathway-associated disease or disorder.

Also provided herein is a compound of Formula (I), or a pharmaceutically acceptable salt thereof, as defined herein, in the manufacture of a medicament for the treatment of a Ras pathway-associated cancer.

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

Other features and advantages of the invention will be apparent from the following detailed description and figures, and from the claims.

### DETAILED DESCRIPTION

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the invention, and the naming of the compounds does not exclude any tautomer. An example of a tautomeric forms includes the following example:

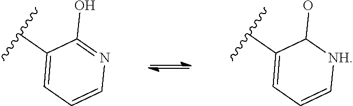 [SMILES: *c1cccnc1O ; *C1=CC=CNC1O]

It will be appreciated that certain compounds provided herein may contain one or more centers of asymmetry and may therefore be prepared and isolated in a mixture of isomers such as a racemic mixture, or in an enantiomerically pure form. An example of an enantiomeric form includes the following example:

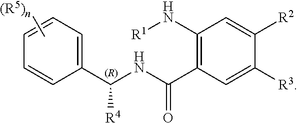

The term “halo” refers to one of the halogens, group 17 of the periodic table. In particular the term refers to fluorine, chlorine, bromine and iodine. Preferably, the term refers to fluorine or chlorine.

The term “C₁₋₃ alkyl” refers to a linear or branched saturated hydrocarbon chain containing 1, 2, or 3 carbon atoms, for example, methyl, ethyl, n-propyl.

The term “C₁₋₆ alkyl” refers to a linear or branched saturated hydrocarbon chain containing 1, 2, 3, 4, 5 or 6 carbon atoms, for example, methyl, ethyl, n-propyl, iso-propyl, n-butyl, sec-butyl, tert-butyl, n-pentyl and n-hexyl.

The term “C₁₋₆ alkylene” refers to a straight or branched divalent hydrocarbon (alkyl) chain linking the rest of the molecule to a radical group, consisting solely of carbon and hydrogen, respectively, containing 1, 2, 3, 4, 5 or 6 carbon atoms. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain.

The term “C₁₋₁₂ alkyl” refers to a linear or branched saturated hydrocarbon chain containing 1, 2, 3, 4, 5, 6, 7, 8, 9, 10, 11, or 12 carbon atoms, for example, methyl, ethyl, n-propyl, iso-propyl, n-butyl, sec-butyl, tert-butyl, n-pentyl, n-hexyl, n-heptyl, n-octyl, n-nonyl, n-decyl, n-undecyl, and n-dodecyl.

The term “alkoxy” as used herein, alone or in combination, refers to an alkyl ether radical, wherein the term alkyl is as defined above. Alkoxy groups may have the general formula: alkyl-O—. As for alkyl group, alkoxy groups can have any suitable number of carbon atoms, such as C₁-C₃. Alkoxy groups include, for example, methoxy, ethoxy, propoxy, isopropoxy, butoxy, 2-butoxy, isobutoxy, secbutoxy, tertbutoxy, pentoxy, hexoxy, and the like. The alkoxy groups can be further optionally substituted as defined herein.

The term “C₁₋₆ haloalkyl” refers to a C₁₋₆ alkyl group, as defined herein, substituted with at least one halogen atom independently chosen at each occurrence, for example fluorine, chlorine, bromine, and iodine. The halogen atom(s) may be present at any position on the alkyl group. For example, C₁₋₆ haloalkyl may refer to chloromethyl, fluoromethyl, diflouoromethyl, trifluoromethyl, chloroethyl e.g., 1-chloroethyl and 2-chloroethyl, trichloroethyl e.g., 1,2,2-trichloroethyl, 2,2,2-trichloroethyl, fluoroethyl e.g. 1-fluoromethyl and 2-fluoroethyl, difluoroethyl e.g. 1,1-difluoroethyl, 2,2-difluoroethyl, 1,2-difluoroethyl, trifluoroethyl e.g. 1,2,2-trifluoroethyl and 2,2,2-trifluoroethyl, chloropropyl, trichloropropyl, fluoropropyl, trifluoropropyl. Similarly, a “C₁₋₃ haloalkyl” refers to a C₁₋₃ alkyl group, as defined herein, substituted with at least one halogen atom independently chosen at each occurrence (e.g., fluorine, chlorine, bromine, and iodine) at any position on the alkyl group.

As used herein, the term “amine” refers to a —NR′R″ group in cases where the amine is an end group, as defined below, and is used herein to describe a —NR′— group in cases where the amine is a linking group.

The phrase “end group” describes a group (a substituent) that is attached to another moiety in the compound via one atom thereof.

The phrase “linking group” describes a group (a substituent) that is attached to another moiety in the compound via two or more atoms thereof.

The amine group can therefore be a primary amine, where both R′ and R″ are hydrogen, a secondary amine, where R′ is hydrogen and R″ is a C₁₋₆ alkyl, or a tertiary amine, where each of R′ and R″ is independently C₁₋₆ alkyl.

As used herein, the term “heteroaryl” refers to a 5 to 10-membered mono- or bicyclic group wherein at least one ring in the system is aromatic; and wherein one or more carbon atoms in at least one ring in the system is/are replaced with a heteroatom independently selected from N, O, and S. Non-limiting examples of heteroaryl groups include furanyl, furazanyl, thiofuranyl, benzothiophenyl, phthalazinyl, pyrrolyl, oxazolyl, benzoxazolyl, 1,2,3-oxadiazolyl, 1,2,4-oxadiazole, thiazolyl, 1,2,3-thiadiazolyl, 1,2,4-thiadiazolyl, benzothiazolyl, imidazolyl, benzimidazolyl, indolyl, indazole, pyrazolyl, benzopyrazolyl, isoxazolyl, benzoisoxazole, isothiazolyl, triazolyl, benzotriazolyl, thiadiazolyl, tetrazolyl, pyridinyl, pyridazinyl, pyrimidinyl, pyrazinyl, purinyl, pteridinyl, quinolinyl, isoquinolinyl, quinazolinyl, quinoxalinyl, cinnolinyl, triazinyl, 2,3-dihydrobenzofuranyl, and 5,6,7,8-tetrahydroimidazo[1,5]pyridinyl. In some embodiments, the heteroaryl has from one to three heteroatoms independently selected from N, O, and S.

As used herein, the term “cycloalkyl” refers to a saturated or partially unsaturated mono- or bicyclic carbon group having 3 to 10 carbon atoms, such as C₃-C₁₀ cycloalkyl groups and C₃-C₆ cycloalkyl groups. Bicyclic cycloalkyl groups include fused, spiro, and bridged ring systems. Non-limiting examples of cycloalkyl groups include phenyl, 2,3-dihydro-1H-indene, cyclopropyl, cyclobutyl, cyclopentyl, cyclohexyl, spiro[2.3]hexyl, spiro[3.3]heptanyl, and bicyclo[1.1.1]pentyl, bicyclo[2.2.1]heptyl, and spiro[2.5]octyl.

The term “heterocyclyl” refers to a saturated or partially unsaturated hydrocarbon monocyclic or bicyclic ring system, having 3 to 10 ring atoms, that is not aromatic, having at least one heteroatom within the ring selected from N, O and S. Bicyclic heterocyclyl groups include fused, spiro, and bridged ring systems. The heterocyclyl group may be denoted as, for example, a “5 to 10-membered heterocyclyl group,” which is a ring system containing 5, 6, 7, 8, 9 or 10 atoms at least one being a heteroatom. Similarly, a “5- to 8-membered heterocyclyl” is a ring system containing 5, 6, 7, or 8 atoms at least one being a heteroatom. Heterocyclyl groups can, for example, have 1, 2, 3, or more, heteroatoms. In some embodiments, a heterocyclyl group has one or two independently selected heteroatoms. A heterocycle may further contain one or more carbonyl or thiocarbonyl functionalities, so as to make the definition include oxo-systems and thio-systems such as lactams, lactones, cyclic imides, cyclic thioimides and cyclic carbamates. The heterocyclyl group may be bonded to the rest of the molecule through any carbon atom or through a heteroatom such as nitrogen. Exemplary heterocyclyl groups include, but are not limited to azepanyl, 1,3-dioxolane, 1,4-dioxolanyl, maleimidyl, succinimidyl, dioxopiperazinyl, hydantoinyl, imidazolinyl, imidazolidinyl, isoxazolinyl, isoxazolidinyl, oxazolinyl, oxazolidinyl, oxazolidinonyl, thiazolinyl, thiazolidinyl, morpholinyl, oxiranyl, piperidinyl N-oxide, piperidinyl, piperazinyl, pyrrolidinyl, pyrrolidonyl, pyrrolidionyl, 4-piperidonyl, pyrazolinyl, pyrazolidinyl, 2-oxopyrrolidinyl, tetrahydropyranyl, quinuclidineyl, 4H-pyranyl, azetidinyl, oxetanyl, octahydrocyclopenta[c]pyrrole, 2-azaspiro[3.3]heptanyl, 3-oxabicyclo[3.1.0]hexanyl, 3-azabicyclo[3.1.0]hexanyl, 3-azabicyclo[3.1.1]heptanyl, 4-azaspiro[2.5]octanyl, 6-azaspiro[3.5]nonanyl, 2,6-diazaspiro[3.3]heptanyl, 7-azabicyclo[2.2.1]heptanyl, 2-azabicyclo[2.2.1]heptanyl, 2,5-diazabicyclo[2.2.2]octanyl, 2,5-diazabicyclo[2.2.1]heptanyl, 2-oxabicyclo[2.1.1]hexanyl, 3-azabicyclo[3.2.1]octanyl, hexahydro-1H-cyclopenta[c]pyrrolyl, 3-oxa-9-azabicyclo[3.3.1]nonanyl, and hexahydro-1H-pyrrolizinyl.

The term “oxo,” as used herein, means an oxygen that is double bonded to a carbon atom. Where a substituent is oxo, a person having ordinary skill in the art will recognize that the oxo is attached through a double bond in accordance with the normal rules of chemical valency.

The term “cyano” as used herein, alone or in combination, refers to—CN.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

The term “optionally substituted” means the anteceding group or groups may be substituted or unsubstituted.

The compounds of Formula (I) include pharmaceutically acceptable salts thereof. In addition, the compounds of Formula (I) also include other salts of such compounds which are not necessarily pharmaceutically acceptable salts, and which may be useful as intermediates for preparing and/or purifying compounds of Formula (I) and/or for separating enantiomers of compounds of Formula (I).

It will further be appreciated that the compounds of Formula (I) or their salts may be isolated in the form of solvates, and accordingly that any such solvate is included within the scope of the present invention. For example, compounds of Formula (I) and salts thereof can exist in unsolvated as well as solvated forms with pharmaceutically acceptable solvents such as water, ethanol, and the like.

In some embodiments, the compounds of Formula (I) include the compounds of Examples 1-81 and stereoisomers and pharmaceutically acceptable salts and solvates thereof. In some embodiments, the compounds of Examples 1-81 are in the free base form. In some embodiments, the compounds of Examples 1-81 are in the form of a pharmaceutically acceptable salt.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some embodiments, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris (hydroxymethyl)methylamine, and salts with amino acids such as arginine, lysine, and the like, or by other methods previously determined. The pharmacologically acceptable salts not specifically limited as far as it can be used in medicaments. Examples of a salt that the compounds described herein with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, for example addition salts with mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present invention.

The ability of test compounds to act as inhibitors of the SOS1-Ras (e.g., KRas (e.g., KRas G12C)) interaction may be demonstrated by the biological assays described herein. IC₅₀ values for inhibiting the SOS1-Ras interaction are shown in Table A.

In some embodiments, the compounds provided herein exhibit brain and/or central nervous system (CNS) penetrance. Such compounds are capable of crossing the blood brain barrier and inhibiting SOS1 activity in the brain and/or other CNS structures. In some embodiments, the compounds provided herein are capable of crossing the blood brain barrier in an effective amount. For example, treatment of a subject with cancer (e.g., a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) such as a Ras pathway-associated brain or CNS cancer) can include administration (e.g., oral administration) of the compound to the subject. In some such embodiments, the compounds provided herein are useful for treating a primary brain tumor or metastatic brain tumor. For example, a Ras pathway-associated primary brain tumor or metastatic brain tumor.

Compounds of Formula (I), or a pharmaceutically acceptable salt thereof, are useful for treating diseases and disorders which can be treated with a SOS1 inhibitor, such as a Ras pathway-associated disease or disorder (e.g., a SOS1-associated disease or disorder, a Ras-associated disease or disorder (e.g., a KRas-associated disease or disorder, a HRas-associated disease or disorder, and/or a NRas-associated disease or disorder), an EGFR-associated disease or disorder, an ErbB2-associated disease or disorder, an ErbB3-associated disease or disorder, an ErbB4-associated disease or disorder, a NF1-associated disease or disorder, a PDGFR-A-associated disease or disorder, a PDGFR-B-associated disease or disorder, a FGFR1-associated disease or disorder, FGFR2-associated disease or disorder, FGFR3-associated disease or disorder, a IGF1 R-associated disease or disorder, a INSR-associated disease or disorder, a ALK-associated disease or disorder, a ROS-associated disease or disorder, a TrkA-associated disease or disorder, a TrkB-associated disease or disorder, a TrkC-associated disease or disorder, a RET-associated disease or disorder, a c-MET-associated disease or disorder, a VEGFR1-associated disease or disorder, a VEGFR2-associated disease or disorder, a VEGFR3-associated disease or disorder, an AXL-associated disease or disorder, a SHP2-associated disease or disorder, a RAF-associated disease or disorder (e.g., a BRAF-associated disease or disorder), a PI3K-associated disease or disorder, an AKT-associated disease or disorder, an mTOR-associated disease or disorder, a MEK-associated disease or disorder, an ERK-associated disease or disorder, or a combination thereof), including hematological cancers, solid tumors, Neurofibromatosis type 1 (NF1), Noonan Syndrome (NS), LEOPARD syndrome, Capillary Malformation-Arteriovenous Malformation Syndrome (CM-AVM), Costello Syndrome (CS), Cardio-Facio-Cutaneous Syndrome (CFC), Legius Syndrome, and Hereditary gingival fibromatosis.

Compounds of Formula (I) or a pharmaceutically acceptable salt thereof are useful for treating diseases and disorders which can be treated with a SOS1 inhibitor, such as a Ras pathway-associated cancer, including hematological cancers and solid tumors.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the term “subject” refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

In some embodiments, the subject has been identified or diagnosed as having a cancer with a dysregulation of a Ras pathway gene (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), a Ras pathway protein (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or expression or activity, or level of any of the same (a Ras pathway-associated cancer) (e.g., as determined using a regulatory agency-approved, e.g., FDA-approved, assay or kit). In some embodiments, the subject has a tumor that is positive for a dysregulation of a Ras pathway gene, a Ras pathway protein, or expression or activity, or level of any of the same (e.g., as determined using a regulatory agency-approved assay or kit). The subject can be a subject with a tumor(s) that is positive for a dysregulation of a Ras pathway gene, a Ras pathway protein, or expression or activity, or level of any of the same (e.g., identified as positive using a regulatory agency-approved, e.g., FDA-approved, assay or kit). The subject can be a subject whose tumors have a dysregulation of a Ras pathway gene, a Ras pathway protein, or expression or activity, or a level of the same (e.g., where the tumor is identified as such using a regulatory agency-approved, e.g., FDA-approved, kit or assay). In some embodiments, the subject is suspected of having a Ras pathway-associated cancer. In some embodiments, the subject has a clinical record indicating that the subject has a tumor that has a dysregulation of a Ras pathway gene, a Ras pathway protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a Ras pathway gene, a Ras pathway protein, or expression or activity, or level of any of the same (a Ras pathway-associated cancer).

In some embodiments, the subject has been identified or diagnosed as having a cancer with a dysregulation of a Ras gene, a Ras protein, or expression or activity, or level of any of the same (a Ras-associated cancer) (e.g., as determined using a regulatory agency-approved, e.g., FDA-approved, assay or kit). In some embodiments, the subject has a tumor that is positive for a dysregulation of a Ras gene, a Ras protein, or expression or activity, or level of any of the same (e.g., as determined using a regulatory agency-approved assay or kit). The subject can be a subject with a tumor(s) that is positive for a dysregulation of a Ras gene, a Ras protein, or expression or activity, or level of any of the same (e.g., identified as positive using a regulatory agency-approved, e.g., FDA-approved, assay or kit). The subject can be a subject whose tumors have a dysregulation of a Ras gene, a Ras protein, or expression or activity, or a level of the same (e.g., where the tumor is identified as such using a regulatory agency-approved, e.g., FDA-approved, kit or assay). In some embodiments, the subject is suspected of having a Ras-associated cancer. In some embodiments, the subject has a clinical record indicating that the subject has a tumor that has a dysregulation of a Ras gene, a Ras protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a Ras gene, a Ras protein, or expression or activity, or level of any of the same (a Ras-associated cancer).

In some embodiments, the subject has been identified or diagnosed as having a cancer with a dysregulation of a KRas gene, a KRas protein, or expression or activity, or level of any of the same (a KRas-associated cancer) (e.g., as determined using a regulatory agency-approved, e.g., FDA-approved, assay or kit). In some embodiments, the subject has a tumor that is positive for a dysregulation of a KRas gene, a KRas protein, or expression or activity, or level of any of the same (e.g., as determined using a regulatory agency-approved assay or kit). The subject can be a subject with a tumor(s) that is positive for a dysregulation of a KRas gene, a KRas protein, or expression or activity, or level of any of the same (e.g., identified as positive using a regulatory agency-approved, e.g., FDA-approved, assay or kit). The subject can be a subject whose tumors have a dysregulation of a KRas gene, a KRas protein, or expression or activity, or a level of the same (e.g., where the tumor is identified as such using a regulatory agency-approved, e.g., FDA-approved, kit or assay). In some embodiments, the subject is suspected of having a KRas-associated cancer. In some embodiments, the subject has a clinical record indicating that the subject has a tumor that has a dysregulation of a KRas gene, a KRas protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a KRas gene, a KRas protein, or expression or activity, or level of any of the same (a KRas-associated cancer).

In some embodiments, the subject has been identified or diagnosed as having a cancer with a dysregulation of a HRas gene, a HRas protein, or expression or activity, or level of any of the same (a HRas-associated cancer) (e.g., as determined using a regulatory agency-approved, e.g., FDA-approved, assay or kit). In some embodiments, the subject has a tumor that is positive for a dysregulation of a HRas gene, a HRas protein, or expression or activity, or level of any of the same (e.g., as determined using a regulatory agency-approved assay or kit). The subject can be a subject with a tumor(s) that is positive for a dysregulation of a HRas gene, a HRas protein, or expression or activity, or level of any of the same (e.g., identified as positive using a regulatory agency-approved, e.g., FDA-approved, assay or kit). The subject can be a subject whose tumors have a dysregulation of a HRas gene, a HRas protein, or expression or activity, or a level of the same (e.g., where the tumor is identified as such using a regulatory agency-approved, e.g., FDA-approved, kit or assay). In some embodiments, the subject is suspected of having a HRas-associated cancer. In some embodiments, the subject has a clinical record indicating that the subject has a tumor that has a dysregulation of a HRas gene, a HRas protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a HRas gene, a HRas protein, or expression or activity, or level of any of the same (a HRas-associated cancer).

In some embodiments, the subject has been identified or diagnosed as having a cancer with a dysregulation of a NRas gene, a NRas protein, or expression or activity, or level of any of the same (a NRas-associated cancer) (e.g., as determined using a regulatory agency-approved, e.g., FDA-approved, assay or kit). In some embodiments, the subject has a tumor that is positive for a dysregulation of a NRas gene, a NRas protein, or expression or activity, or level of any of the same (e.g., as determined using a regulatory agency-approved assay or kit). The subject can be a subject with a tumor(s) that is positive for a dysregulation of a NRas gene, a NRas protein, or expression or activity, or level of any of the same (e.g., identified as positive using a regulatory agency-approved, e.g., FDA-approved, assay or kit). The subject can be a subject whose tumors have a dysregulation of a NRas gene, a NRas protein, or expression or activity, or a level of the same (e.g., where the tumor is identified as such using a regulatory agency-approved, e.g., FDA-approved, kit or assay). In some embodiments, the subject is suspected of having a NRas-associated cancer. In some embodiments, the subject has a clinical record indicating that the subject has a tumor that has a dysregulation of a NRas gene, a NRas protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a NRas gene, a NRas protein, or expression or activity, or level of any of the same (a NRas-associated cancer).

In some embodiments, the subject has been identified or diagnosed as having a cancer with a dysregulation of a SOS1 gene, a SOS1 protein, or expression or activity, or level of any of the same (a SOS1-associated cancer) (e.g., as determined using a regulatory agency-approved, e.g., FDA-approved, assay or kit). In some embodiments, the subject has a tumor that is positive for a dysregulation of a SOS1 gene, a SOS1 protein, or expression or activity, or level of any of the same (e.g., as determined using a regulatory agency-approved assay or kit). The subject can be a subject with a tumor(s) that is positive for a dysregulation of a SOS1 gene, a SOS1 protein, or expression or activity, or level of any of the same (e.g., identified as positive using a regulatory agency-approved, e.g., FDA-approved, assay or kit). The subject can be a subject whose tumors have a dysregulation of a SOS1 gene, a SOS1 protein, or expression or activity, or a level of the same (e.g., where the tumor is identified as such using a regulatory agency-approved, e.g., FDA-approved, kit or assay). In some embodiments, the subject is suspected of having a SOS1-associated cancer. In some embodiments, the subject has a clinical record indicating that the subject has a tumor that has a dysregulation of a SOS1 gene, a SOS1 protein, or expression or activity, or level of any of the same (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to be associated with a dysregulation of a SOS1 gene, a SOS1 protein, or expression or activity, or level of any of the same (a SOS1-associated cancer).

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday).

In certain embodiments, compounds of Formula (I), or a pharmaceutically acceptable salt thereof are useful for preventing diseases and disorders as defined herein (for example, autoimmune diseases, inflammatory diseases, and cancer). The term “preventing” as used herein means the prevention of the onset, recurrence or spread, in whole or in part, of the disease or condition as described herein, or a symptom thereof.

In certain embodiments, compounds of Formula (I), or a pharmaceutically acceptable salt thereof are useful for preventing diseases and disorders as defined herein (for example, Ras pathway-associated diseases or disorders (e.g., autoimmune diseases, inflammatory diseases, and cancer) as described herein. The term “preventing” as used herein means the prevention of the onset, recurrence or spread, in whole or in part, of the disease or condition as described herein, or a symptom thereof.

Aberrant cell growth and proliferation is a hallmark of cancer. One such pathway through which such aberrant cell growth can occur is through Ras family protein signaling. The human Ras proteins (e.g., KRas (V-Ki-Ras2 Kirsten Rat Sarcoma 2 Viral Oncogene Homolog), HRas (V-Ha-Ras Harvey Rat Sarcoma Viral Oncogene Homolog), and/or NRas (Neuroblastoma RAS Viral (V-Ras) Oncogene Homolog); sometimes also called KRAS, HRAS, and NRAS, or KRas H-Ras, and N-Ras, respectively) are membrane-bound guanosine triphosphate (GTP)/guanosine diphosphate (GDP)-binding (G) proteins that are implicated in many oncogenic signaling cascades. Each of these proteins is approximately 21 kD in size. KRas has two common isoforms known as KRas4A and KRas4B.

Mature Ras proteins are typically associated with the cellular membrane via post-translational modification, such as prenylation (e.g., farnesylation of a “CAAX box”, where C represents cysteine, A represents an aliphatic amino acid, and X is methionine, serine, leucine, or glutamine). In the inactive state, Ras proteins are bound to GDP. See, e.g., Adjei, J. Nat'l. Cancer Inst. 93.14 (2001): 1062-1074.

Activation of Ras proteins can be initiated via multiple types of cell-surface receptors including receptor tyrosine kinases (TKIs) (e.g., EGFR, ErbB2, ErbB3, ErbB4, PDGFR-A/B, FGFR1/2/3, IGF1 R, INSR, ALK, ROS, TrkA, TrkB, TrkC, RET, c-MET, VEGFR1/2/3, AXL), T-cell receptors, B-cell receptors, monocyte colony-stimulating factor receptor, G-protein coupled receptors (GPCRs), and integrin family proteins. Activation of one of these types of cell-surface receptors generally leads, directly or indirectly, to activation of one or more guanine nucleotide exchange factors (GEFs), which promote Ras proteins to release GDP, allowing GTP to bind. Non-limiting examples of GEFs include the SOS (Son of Sevenless Homolog) proteins and RASGRF1 (Ras protein specific guanine nucleotide releasing factor 1; also sometimes called Cdc25). For example, upon activation, dimerization, and auto-phosphorylation of EGFR, the receptor can bind to the SH2 domain of the adaptor protein growth-factor-receptor-bound protein 2 (GRB2), which can then bind to a SOS protein (e.g., SOS1 or SOS2, sometimes also called SOS-1 and SOS-2, respectively), thereby co-localizing the SOS protein with the Ras family protein at the cellular membrane. See, e.g., Xuehua et al., Proc. Nat. Acad. Sci. November 2017, 114 (47) E10092-E10101; Vetter and Wittinghofer, Science 294.5545 (2001): 1299-1304; Downward, Nat. Rev. Cancer 3.1 (2003): 11-22; Pierre and Coumoul, Biochem. Pharmacol. 82.9 (2011): 1049-1056. Kortum, et al. Proc. Nat. Acad. Sci. 108.30 (2011): 12407-12412; U.S. Appl. Publ. Nos. 2019/0358230 and 2019/0194192; and PCT Publication Nos. WO 2018/172250 and WO 2019/201848.

Once activated by binding GTP, the Ras proteins can bind to and activate a number of downstream effectors, including the RAF family proteins, phosphatidyl inositol 3-kinases (PI3Ks), and RAL family proteins. See, e.g., Gurung and Bhattacharjee. Oncology & Hematology Review, 2015; 11 (2): 147-52 (2015). For instance, signaling through the Ras-RAF-MAPK pathway has been implicated in many cancers, including, but not limited to, pancreatic cancer, thyroid cancer (e.g., papillary thyroid cancer), colon cancer, lung cancer (e.g., non-small cell lung cancer), melanoma, biliary tract cancer, small intestinal cancer, endometrial cancer, ovarian cancer, cervical cancer, prostate cancer, soft tissue cancers, peritoneal cancer, stomach cancer, liver cancer, urinary tract cancer, breast cancer, and combinations thereof. See, e.g., Kinsey, et al. Nat. Medicine 25.4 (2019): 620-627; Roberts and Der. Oncogene 26.22 (2007): 3291-3310; Santarpia, et al. Expert Opinion on Therapeutic Targets 16.1 (2012): 103-119. As another example, signaling through the Ras-PI3K/AKT/mammalian target of rapamycin (mTOR) pathway has been shown to play a role in many cancers, including, but not limited to, melanoma, ovarian cancer, cervical cancer, endometrial cancer, breast cancer, prostate cancer, brain cancer (e.g., glioblastoma), lung cancer (e.g., non-small cell lung cancer), pancreatic cancer, bladder cancer, colon cancer, head and neck cancer, leukemia, thyroid cancer, lymphoma, bowel cancer, gastric cancer, and combinations thereof. See, e.g., Chappell, et al. Oncotarget 2.3 (2011): 135; Vara, et al. Cancer Treatment Reviews 30.2 (2004): 193-204; Hennessy, et al. Nat. Rev. Drug Disc. 4.12 (2005): 988-1004; Osaki, et al. Apoptosis 9.6 (2004): 667-676; Luo, et al. Cancer Cell 4.4 (2003): 257-262.

Though Ras proteins have intrinsic GTPase activity, it is typically not physiologically relevant. Instead, hydrolysis of the bound GTP is enhanced (e.g., by up to about 5 orders of magnitude) by the binding of a GTPase-activating protein (GAP), such as neurofibromatosis type 1 (NF1) or p120^{GAP}. See, e.g., Adjei, Journal of the National Cancer Institute 93.14 (2001): 1062-1074; Downward, Nature Reviews Cancer 3.1 (2003): 11-22; Scheffzek, et al. Science 277.5324 (1997): 333-339.

Activating mutations (especially, e.g., at residues G12, G13, and/or Q61) in Ras family proteins are estimated to be present in up to about 30% of all human cancers. Commonly, activating mutations in Ras family proteins render the Ras protein insensitive to the activity of GAPs. See, e.g., Santarpia, et al. Expert Opinion on Therapeutic Targets 16.1 (2012): 103-119. Exemplary, non-limiting examples of Ras mutations are presented in Tables 1 (KRas mutations), 2 (HRas mutations), and 3 (NRas mutations).

The term “Ras pathway-associated disease or disorder” as used herein refers to diseases or disorders associated with or having a dysregulation of a gene in a Ras pathway, a protein in a Ras pathway, or the expression or activity or level of any (e.g., one or more) of the same (e.g., any of the types of dysregulation of a gene in a Ras pathway, a protein in a Ras pathway, or the expression or activity or level of any of the same, as described herein). Non-limiting examples of a Ras pathway-associated diseases or disorders include, for example, Neurofibromatosis type 1 (NF1), Noonan Syndrome (NS), LEOPARD syndrome, Capillary Malformation-Arteriovenous Malformation Syndrome (CM-AVM), Costello Syndrome (CS), Cardio-Facio-Cutaneous Syndrome (CFC), Legius Syndrome, Hereditary gingival fibromatosis, and cancers.

In some embodiments, a Ras pathway-associated disease or disorder is a Ras pathway-associated cancer. The term “Ras pathway-associated cancer” as used herein refers to cancers associated with or having a dysregulation of a gene in a Ras pathway, a protein in a Ras pathway, or the expression or activity or level of any (e.g., one or more) of the same (e.g., any of the types of dysregulation of a gene in a Ras pathway, a protein in a Ras pathway, or the expression or activity or level of any of the same, as described herein). Non-limiting examples of a Ras pathway-associated cancer are described herein. In some embodiments, a Ras pathway-associated cancer can be a KRas-associated cancer, a HRas-associated cancer, a NRas-associated cancer, a SOS1-associated cancer, an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof.

The term “Ras-associated cancer” as used herein refers to cancers associated with or having a dysregulation of a Ras gene, a Ras protein, or the expression or activity or level of any (e.g., one or more) of the same (e.g., any of the types of dysregulation of a Ras gene, a Ras protein, or the expression or activity or level of any of the same, as described herein). Non-limiting examples of a Ras-associated cancer are described herein. In some embodiments, a Ras-associated cancer can be a KRas-associated cancer, a HRas-associated cancer, a NRas-associated cancer, or a combination thereof.

The phrase “dysregulation of a Ras gene, a Ras protein, or the expression or activity or level of any of the same” refers to a genetic mutation (e.g., a Ras (e.g., KRas, NRas, or HRas) gene translocation that results in the expression of a fusion protein, a mutation in a Ras gene that results in the expression of a Ras protein that includes a deletion of at least one amino acid as compared to a wild type Ras protein, a mutation in a Ras gene that results in the expression of a Ras protein with one or more point mutations as compared to a wild type Ras protein, a mutation in a Ras gene that results in the expression of a Ras protein with at least one inserted amino acid as compared to a wild type Ras protein, a gene duplication that results in an increased level of Ras protein in a cell, or a mutation in a regulatory sequence (e.g., a promoter and/or enhancer) that results in an increased level of Ras protein in a cell), an alternative spliced version of a Ras mRNA (e.g., that results in a Ras protein having a deletion of at least one amino acid in the Ras protein as compared to the wild type Ras protein or that results in a Ras protein having an insertion of at least one amino acid in the Ras protein as compared to the wild type Ras protein), or increased expression (e.g., increased levels) of a wild type Ras protein in a mammalian cell due to aberrant cell signaling and/or dysregulated autocrine/paracrine signaling (e.g., as compared to a control non-cancerous cell). As another example, a dysregulation of a Ras gene, a Ras protein, or expression or activity, or level of any of the same, can be a mutation in a Ras gene that encodes a Ras protein that is constitutively active or has increased activity as compared to a protein encoded by a Ras gene that does not include the mutation. In some embodiments of any of the methods described herein, a dysregulation of a Ras gene, a Ras protein, or the expression or activity or level of any of the same can be selected from the group consisting of a G12 mutation, a G13 mutation, a Q61 mutation, and a combination thereof.

Table 1 lists some non-limiting exemplary KRas mutations. Table 1A lists a non-limiting exemplary KRas fusion. In some embodiments of any of the methods described herein, a dysregulation of a KRas gene, a KRas protein, or the expression or activity or level of any of the same can be selected from the group consisting of a G12 mutation (e.g., G12I, G12A, G12C, G12D, G12E, G12F, G12L, G12N, G12R, G12S, G12T, G12V, G12W, or G12Y), a G13 mutation (e.g., G13A, G13C, G13D, G13E, G13F, G13I, G13M, G13N, G13P, G13R, G13S, G13V, or G13Y), a Q61 mutation (e.g., Q61D, Q61E, Q61H, Q61K, Q61L, Q61P, Q61R), and a combination thereof.

Table 2 lists some non-limiting exemplary HRas mutations. In some embodiments of any of the methods described herein, a dysregulation of a HRas gene, a HRas protein, or the expression or activity or level of any of the same can be selected from the group consisting of a G12 mutation (e.g., G12A, G12C, G12D, G12R, G12S, G12V), a G13 mutation (e.g., G13A, G13C, G13D, G13R, G13S, G13V), a Q61 mutation (e.g., Q61H, Q61K, Q61L, Q61P, Q61R, Q61*), and a combination thereof.

Table 3 lists some non-limiting exemplary HRas mutations. In some embodiments of any of the methods described herein, a dysregulation of a HRas gene, a HRas protein, or the expression or activity or level of any of the same can be selected from the group consisting of a G12 mutation (e.g., G12A, G12C, G12D, G12R, G12S, G12V, G12W, G12N), a G13 mutation (e.g., G13A, G13C, G13D, G13R, G13S, G13V), a Q61 mutation (e.g., Q61E, Q61H, Q61K, Q61L, Q61P, Q61R, Q61E, Q61N), and a combination thereof.

TABLE 1
KRAS Protein Amino Acid Substitutions/Insertions/Deletions^{A}

| Amino Acid | Non-Limiting | Non-Limiting Exemplary |
|---|---|---|
| Position(s) | Exemplary Mutations | RAS-Associated Cancers |
| 5 | K5E², K5N², K5R¹ | |
| 6 | L6F¹ | |
| 7 | V7E¹, V7M¹ | |
| 8 | V8A¹, V8V^{1†} | |
| 9 | V9I¹, V9V^{1†} | |
| 10 | G10E¹, G10G^{1†}, G10R¹ | |
| 11 | A11A^{1†}, A11P¹, A11T¹, | |
| | A11V² | |
| 12 | G12I¹, G12A², G12C², G12D², | Bile duct carcinoma¹¹, gall bladder |
| | G12E¹, G12F², G12G*^{1†}, | carcinoma¹¹, colorectal cancer¹¹, |
| | G12L², G12N², G12R², G12S², | haematopoietic neoplasm¹¹, lymphoid |
| | G12T¹, G12V², G12W¹, | neoplasm¹¹, lung adenocarcinoma¹¹, |
| | G12Y¹ | NSCLC¹¹, pancreatic ductal carcinoma¹¹, |
| | | prostate cancer¹¹, melanoma¹¹, gastric |
| | | adenocarcinoma¹¹ |
| 13 | G13A², G13C¹, G13D², | colorectal cancer¹¹, haematopoietic |
| | G13E¹, G13F¹, G13G^{1†}, G13I¹, | neoplasm¹¹, lymphoid neoplasm¹¹, gastric |
| | G13M¹, G13N², G13P¹, | adenocarcinoma¹¹ |
| | G13R², G13S², G13V², G13Y¹ | |
| 14 | V14A¹, V14G¹, V14I², V14L¹ | |
| 15 | G15D², G15G^{1†}, G15S¹, | |
| | G15W⁹ | |
| 16 | K16R¹ | |
| 17 | S17G², S17N¹ | |
| 18 | A18D², A18T¹, A18V¹ | |
| 19 | L19F² | |
| 20 | T20A¹ | |
| 20 | T20A¹, T20M¹, T20R², T20S¹, | |
| | T20T^{1†} | |
| 21 | I21R¹ | |
| 22 | Q22*¹, Q22K¹, Q22Q^{1†}, | |
| | Q22R² | |
| 23 | L23I¹, L23R² | |
| 24 | I24F¹, I24N², I24V² | |
| 27 | H27H^{1†}, H27L², H27N² | |
| 28 | F28S¹ | |
| 30 | D30E¹ | |
| 31 | E31K¹, E31Q¹, E37G⁵, E31Q¹ | |
| 33 | D33E¹ | |
| 34 | P34L¹, P34S² | |
| 35 | T35A¹, T35I², T35S⁵, T35T^{1†} | |
| 36 | I36L¹, I36M¹ | |
| 37 | E37G⁵, E37K¹ | |
| 40 | Y40C⁵ | |
| 45 | V45V^{1†} | |
| 49 | E49*¹, E49K¹ | |
| 51 | C51C^{1†} | |
| 52 | L52F¹ | |
| 57 | D57N¹ | |
| 58 | T58I², T58T^{1†} | |
| 59 | A59A^{1†}, A59D⁴, A59del¹, | |
| | A59E², A59G², A59S¹, A59T¹ | |
| 60 | G60A¹, G60D², G60E⁸, | |
| | G60G^{1†}, G60R¹, G60V¹, | |
| | G61H² | |
| 61 | Q61D¹, Q61E², Q61H², | Lung adenocarcinoma¹¹, NSCLC¹¹, colorectal |
| | Q61K², Q61L², Q61P², Q61R¹ | cancer¹¹ |
| 62 | E62D², E62G¹, E62K¹ | |
| 63 | E63del¹, E62E^{1†}, E63K² | |
| 64 | Y64D¹, Y64H¹, Y64N¹ | |
| 65 | S65I¹ | |
| 67 | M67L² | |
| 66 | A66T¹⁰, A66V¹⁰, | |
| 68 | R68G¹, R68M¹, R68S¹ | |
| 69 | D69G¹ | |
| 70 | Q70P¹ | |
| 71 | Y71C¹ | |
| 72 | M72I², M72T¹, M72V¹ | |
| 73 | R73M¹ | |
| 74 | T74P², T74T^{1†} | |
| 75 | G75E¹⁰ | |
| 76 | E76G⁶, E76K⁶, E76Q⁶ | |
| 77 | G77A¹ | |
| 80 | C80S¹, C80Y¹ | |
| 86 | N86K¹ | |
| 88 | K88*¹, K88K^{1†} | |
| 91 | E91K¹ | |
| 92 | D92G¹, D92Y¹ | |
| 95 | H95L¹ | |
| 97 | R97I¹ | |
| 98 | E98*¹ | |
| 102 | R102fs*2¹ | |
| 110 | P110S¹ | |
| 117 | K117E¹, K117N², K117R¹ | |
| 118 | C118S¹ | |
| 120 | L120V⁷ | |
| 121 | P121H¹, P121S¹ | |
| 127 | T127I¹ | |
| 130 | A130V¹ | |
| 134 | A134T¹ | |
| 134 | S136N¹ | |
| 135 | R135T¹ | |
| 138 | G138E¹, G138R¹ | |
| 140 | P140S¹ | |
| 145 | S145T⁷ | |
| 146 | A146A^{1†}, A146G¹, A146P², | |
| | A146T², A146V² | |
| 147 | A147T³, K147N¹ | |
| 153 | D153N¹, D153V¹ | |
| 154 | D154delD¹ | |
| 156 | F156L² | |
| 161 | R161*¹ | |
| 164 | R164L¹, R164Q¹, R164R^{1†} | |
| 173 | D173D¹ | |
| 183 | T183_K184delTK¹ | |
| 185 | C185R¹, C185S¹ | |
| 188 | M188L¹ | |
| Insertions and Deletions | | |
| | A11_G12insGA¹ | |
| | V14_G15insG² | |
| | A66_M67insEEYSA¹ | |
| | D69fs*4¹ | |
| | E3fs*3¹ | |
| | E62 S65 > D² (c.186 | |
| | 194del9) | |
| | G10 A11insG² (c.30 | |
| | 31insGGA) | |
| | G12 G13insA² (c.37 | |
| | 37insGCG) | |
| | G12 G13insG² (c.36 | |
| | 37insGGT) | |
| | G12fs*3¹ | |
| | G12V9F¹ | |
| | G13 V14 > DI¹ | |
| | G13 V14insG² (c.39 | |
| | 40insGGC) | |
| | G60fs*27¹ | |
| | K16_S17insW¹ | |
| | L19_T20 > FA¹ | |
| | M72_R73ins15¹ | |
| | S65_A66ins15¹ | |
| | T183_K184delTK¹ | |
| | T58_A59insVA¹ | |
| | D154delD¹ | |
| | V9 G10insG² (c.27 | |
| | 28insGTA) | |
| | V9 G10insV¹ | |

^{A}The KRAS mutations shown may be activating mutations and/or confer increased resistance of KRAS to a KRAS modulator (e.g., a KRAS inhibitor), e.g., as compared to a wild type KRAS.
^{†}Indicates a synonymous mutation which may affect KRAS protein expression. See, e.g., Waters et al.. PLOS One 2016; 11(9). doi: 10.1371/journal.pone.0163272.
¹U.S. Pat. No. 9,810,690
²U.S. Publication No. 2014/0199405
³P.C.T. Publication No. WO 2012/016050
⁴U.S. Pat. No. 10,238,650
⁵P.C.T. Publication No. WO 2009/052467
⁶U.S. Publication No. 2013/0317037
⁷P.C.T. Publication No. WO 2020/012068
⁸U.S. Publication No. 2017/0130271
⁹U.S. Publication No. 2017/0051356
¹⁰Abe et al. Biochemical and Biophysical Research Communications. 2020: 522(3): P. 360-696.
¹¹Prior et al. Cancer Res. 2012 May 15; 72(10): 2457-2467.

TABLE 1A
Exemplary KRAS Fusion Proteins and Cancers

| Non-limiting Exemplary | Non-limiting Exemplary |
|---|---|
| KRAS Fusions | KRAS-associated Cancer(s) |
| UBE2L3-KRAS¹ | Metastatic Prostate Cancer |

¹Wang et al. Cancer Discovery. 2011; 9(1): 35-43. doi: 10.1158-2159-8274.CD-10-0022.

TABLE 2
HRAS Protein Amino Acid Substitutions/Insertions/Deletions^{A}

| Amino Acid | Non-Limiting | Non-Limiting Exemplary |
|---|---|---|
| Position(s) | Exemplary Mutations | HRAS-Associated Cancers |
| 12 | G12A, G12C, G12D, | Salivary gland adenocarcinoma, |
| | G12R, G12S, G12V ¹ | prostate cancer, melanoma, |
| | | gastric adenocarcinoma ¹, |
| | | Epithelial-Myoepithelial |
| | | Carcinoma ³ |
| 13 | G13A, G13C, G13D, | |
| | G13R, G13S, G13V ¹ | |
| 18 | A18V ² | Oral squamous cell carcinoma ² |
| 61 | Q61H, Q61K, Q61L, Q61P, | Epithelial-Myoepithelial |
| | Q61R, Q61E, Q61* ^{1, 3, 4} | Carcinoma ³ |

^{A}The HRAS mutations shown may be activating mutations and/or confer increased resistance of HRAS to a HRAS modulator (e.g., a HRAS inhibitor), e.g., as compared to a wild type HRAS.
¹ Prior et al. Cancer Res. 2012 May 15; 72(10): 2457-2467.
² Koumaki, Dimitra, et al. Oncology Reports 27 (2012): 1555-1560.
³ Urano, Makoto, et al. The American journal of surgical pathology 43.7 (2019): 984-994.
⁴ U.S. Pat. No. 10,722,484

TABLE 3
NRAS Protein Amino Acid Substitutions/Insertions/Deletions^{A}

| Amino Acid | Non-Limiting | Non-Limiting Exemplary |
|---|---|---|
| Position(s) | Exemplary Mutations | NRAS-Associated Cancers |
| 12 | G12A, G12C, G12D, G12R, | Myeloid leukemia ², |
| | G12S, G12V, G12W, G12N ^{1, 2, 4} | colorectal cancer ⁴ |
| 13 | G13A, G13C, G13D, G13R, | Myeloid leukemia ² |
| | G13S, G13V ^{1, 2} | |
| 15 | G15W ⁶ | Colorectal cancer ⁶ |
| 60 | G60E ^{2, 6} | Myeloid leukemia ² |
| 61 | Q61E, Q61H, Q61K, Q61L, | Myeloid leukemia ², |
| | Q61P, Q61R, Q61E, Q61N ^{1, 2, 4, 5} | colorectal cancer ^{4, 5} |
| 117 | K117N ⁴ | Colorectal cancer ⁴ |
| 146 | A146T, A146P, A146V ^{3, 4} | Colorectal cancer ⁴ |

^{A}The NRAS mutations shown may be activating mutations and/or confer increased resistance of NRAS to a NRAS modulator (e.g., a NRAS inhibitor), e.g., as compared to a wild type NRAS.
¹ Prior et al. Cancer Res. 2012 May 15; 72(10): 2457-2467.
² Tyner, Jeffrey W., et al. Blood, The Journal of the American Society of Hematology 113.8 (2009): 1749-1755.
³ U.S. Pat. No. 10,668,063
⁴ Payandeh, et al. American Journal of Cancer Prevention 3.1 (2015): 19-22.
⁵ Villahermosa, et al. Journal of Clinical Oncology 2014 32: 15_suppl, e22159-e22159
⁶ Shen, et al. PloS One 8.12 (2013): e81628.

However, the Ras proteins have often been considered to be “undruggable”, and no direct Ras inhibitor has been approved by the United States Food and Drug Administration. Accordingly, other targets in Ras signaling pathways have been targeted in order to curb aberrant signaling through these pathways, including targets both upstream and downstream of the Ras family proteins. See, e.g., Cox, et al. Nat. Rev. Drug Disc. 13.11 (2014): 828-851; Khan, et al. Biochimica et Biophysica Acta (BBA)-Molecular Cell Research 1867.2 (2020): 118570; Kessler, et al. Proc. Nat. Acad. Sci. 116.32 (2019): 15823-15829; Dang, et al. Nat. Rev. Cancer 17.8 (2017): 502; Baker and Der, Nature 497.7451 (2013): 577-578.

Guanine nucleotide exchange factors, which promote the exchange of GDP for GTP bound by Ras family proteins, can be suitable targets to reduce signaling through Ras pathways. Inhibition of a GEF may promote the inactive (GDP bound) state of Ras family proteins and therefore decreased signaling through the pathway. See, e.g., Evelyn, et al. Chemistry & Biology 21.12 (2014): 1618-1628; Hillig, et al. Proc. Nat. Acad. Sci. 116.7 (2019): 2551-2560; Patgiri, et al. Nat. Chem. Bio. 7.9 (2011): 585-587: Maurer, et al. Proc. Nat. Acad. Sci. 109.14 (2012): 5299-5304; Winter, et al. J. Med. Chem. 58.5 (2015): 2265-2274. One such GEF is SOS1.

SOS1 has a central “catalytic” core (SOScat) of about 500 residues, which is sufficient for Ras-activating activity. SOS1 has a primary (sometimes also called the “catalytic” site) Ras binding site (e.g., including a Cdc25 homology domain) that can bind to and distort the nucleotide binding site of a Ras protein, thereby promoting the release of the bound nucleotide (e.g., GDP), allowing another nucleotide (e.g., GTP). SOS1 can bind two Ras molecules in a ternary complex, wherein binding of a Ras·GTP complex to a second (sometimes also called the “allosteric” site) site on SOS1, further activating the catalytic activity of SOS1 in a positive feedback-type mechanism. See, e.g., Margarit, et al. Cell 112.5 (2003): 685-695; Freedman, et al. Proc. Nat. Acad. Sci. 103.45 (2006): 16692-16697. Further, it has been shown that small-molecule binders of SOS1 can modulate its GEF activity. See, e.g., Burns, et al. Proc. Nat. Acad. Sci. 111.9 (2014): 3401-3406. In some cases, small-molecule binders of SOS1 can negatively modulate its GEF activity with Ras proteins; such molecules can also be called herein “SOS1 inhibitors” and referred to as inhibiting “SOS1 activity.” Some SOS1 inhibitors have been shown to bind proximal to the primary Ras binding site, for example, causing a movement in the sidechain of Tyr884 and reducing favorable stacking interactions with Arg73 of KRas. Further, antiproliferative activity of some such SOS1 inhibitors has been demonstrated. See, e.g., Hillig, et al., Proc. Nat. Acad. Sci. 116.7 (2019): 2551-2560; U.S. Patent Appl. Publ. Nos. 2019/0358230 and 2019/0194192; and PCT Publication Nos WO 2018/172250 and WO 2019/201848.

The term “SOS1-associated cancer” as used herein refers to cancers associated with or having a dysregulation of a SOS1 gene, a SOS1-GEF (also called herein SOS1 protein), or the expression or activity or level of any (e.g., one or more) of the same (e.g., any of the types of dysregulation of a SOS1 gene, a SOS1 protein, or the expression or activity or level of any of the same, as described herein). Non-limiting examples of a SOS1-associated cancer are described herein.

The phrase “dysregulation of a SOS1 gene, a SOS1 protein, or the expression or activity or level of any of the same” refers to a genetic mutation (e.g., a SOS1 gene translocation that results in the expression of a fusion protein, a mutation in a SOS1 gene that results in the expression of a SOS1 protein that includes a deletion of at least one amino acid as compared to a wild type SOS1 protein, a mutation in a SOS1 gene that results in the expression of a SOS1 protein with one or more point mutations as compared to a wild type SOS1 protein, a mutation in a SOS1 gene that results in the expression of a SOS1 protein with at least one inserted amino acid as compared to a wild type SOS1 protein, a gene duplication that results in an increased level of SOS1 protein in a cell, or a mutation in a regulatory sequence (e.g., a promoter and/or enhancer) that results in an increased level of SOS1 protein in a cell), an alternative spliced version of a SOS1 mRNA (e.g., that results in a SOS1 protein having a deletion of at least one amino acid in the SOS1 protein as compared to the wild type SOS1 protein or that results in a SOS1 protein having an insertion of at least one amino acid in the SOS1 protein as compared to the wild type SOS1 protein), or increased expression (e.g., increased levels) of a wild type SOS1 protein in a mammalian cell due to aberrant cell signaling and/or dysregulated autocrine/paracrine signaling (e.g., as compared to a control non-cancerous cell). As another example, a dysregulation of a SOS1 gene, a SOS1 protein, or expression or activity, or level of any of the same, can be a mutation in a SOS1 gene that encodes a SOS1 protein that is constitutively active or has increased activity as compared to a protein encoded by a SOS1 gene that does not include the mutation. Non-limiting examples of SOS1 protein point mutations/insertions/deletions are described in Table 4. Table 4A lists a non-limiting exemplary SOS1 fusion.

TABLE 4
SOS1 Protein Amino Acid Substitutions/Insertions/Deletions^{A}

| Amino Acid | Non-Limiting | Non-Limiting Exemplary |
|---|---|---|
| Position(s) | Exemplary Mutations | SOS1-Associated Cancers |
| 65 | R65R^{1†} | Colon cancer¹ |
| 102 | P102R³ | Rhabdomyoscarcoma³ |
| 248 | R248H² | T-ALL² |
| 233 | N233Y⁴ | Lung adenocarcinoma⁴ |
| 265 | D265N⁴ | Lung adenocarcinoma⁴ |
| 316 | S316L¹ | Colon cancer¹ |
| 327 | I327T⁴ | Lung adenocarcinoma⁴ |
| 340 | P340S¹ | Colon cancer¹ |
| 378 | T378A³ | Sertoli cell tumor³ |
| 410 | Q410Q^{2†} | Lung adenocarcinoma² |
| 414 | G414G^{1†} | Colon cancer¹ |
| 477 | Q477*¹ | Colon cancer¹ |
| 478 | P478L⁴ | Lung adenocarcinoma⁴ |
| 494 | F494L⁵ | AML⁵ |
| 535 | N535S⁴ | Lung adenocarcinoma⁴ |
| 552 | R552G³ | Granular cell tumors |
| | | of the skin³ |
| 604 | G604V⁴ | Lung adenocarcinoma⁴ |
| 610 | I610T⁵ | AML⁵ |
| 684 | P684S¹ | Colon cancer¹ |
| 688 | R688Q² | Pancreatic cancer² |
| 733 | I733V, I733F⁴ | Lung adenocarcinoma⁴ |
| 806 | G806R¹ | Colon cancer¹ |
| 861 | V861I¹ | Colon cancer¹ |
| 888 | H888Q² | Lung adenocarcinoma² |
| 938 | L938F⁴ | Lung adenocarcinoma⁴ |
| 1019 | R1019Q¹ | Colon cancer¹ |
| 1051 | R1051G⁴ | Lung adenocarcinoma⁴ |
| 1236 | P1236P^{1†} | Colon cancer¹ |
| Insertions, Deletions, and UTRs | | |
| | SOS1-116G > T¹ | Colon cancer¹ |
| | IVS8 + 5G > C¹ | Colon cancer¹ |

^{A}The SOS1 mutations shown may be activating mutations and/or confer increased resistance of SOS1 to a SOS1 modulator (e.g., a SOS1 inhibitor), e.g., as compared to a wild type SOS1.
^{†}Indicates a synonymous mutation, which may or may not affect SOS1 protein expression or other aspects of SOS1 regulation or function.
¹U.S. Patent Application Publication No. 2010/0227778
²Swanson, et al. Genes, Chromosomes and Cancer 47.3 (2008): 253-259. doi: 10.1002/gcc.20527
³Denayer, et al. Genes, Chromosomes and Cancer 49.3 (2010): 242-252. doi: 10.1002/gcc.20735
⁴Cai, et al. Mol. Cancer Res, 17.4 (2019): 1002-1012. doi: 10.1158/1541-7786.MCR-18-0316
⁵Tanizaki, et al. International Journal of Hematology 88.4 (2008): 460-462.

TABLE 4A
Exemplary SOS1 Fusion Proteins and Cancers

| Non-limiting Exemplary | Non-limiting Exemplary |
|---|---|
| SOS1 Fusion Partners | SOS1-associated Cancer(s) |
| ADCY3¹ | Lung adenocarcinoma¹ |

¹P.C.T. Publication No. WO 2013/113942

The term “wild type” describes a nucleic acid (e.g., a SOS1 gene or mRNA) or protein (e.g., a SOS1 protein) that is found in a subject that does not have a disease or disorder associated with that nucleic acid or protein (e.g., a SOS1-related disease or disorder), e.g., a cancer associated with that nucleic acid or protein (and optionally also does not have an increased risk of developing a disease or disorder associated with that nucleic acid or protein and/or is not suspected of having a disease or disorder associated with that nucleic acid or protein), or is found in a cell or tissue from a subject that does not have a disease associated with that nucleic acid or protein, e.g., a cancer associated with that nucleic acid or protein (and optionally also does not have an increased risk of developing a disease or disorder associated with that nucleic acid or protein and/or is not suspected of having a disease or disorder associated with that nucleic acid or protein).

The term “regulatory agency” refers to a country's agency for the approval of the medical use of pharmaceutical agents with the country. For example, a non-limiting example of a regulatory agency is the U.S. Food and Drug Administration (FDA).

### Compounds

Provided herein are compounds of Formula (I):

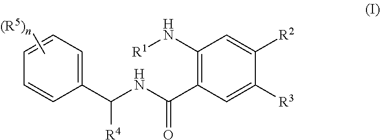

or a pharmaceutically acceptable salt thereof, wherein:
R¹ is a C₁₋₁₂ alkyl, C₃₋₆ cycloalkyl, 3- to 10-membered heterocyclyl or 3- to 10-membered (C₁-C₃ alkylene)-heterocyclyl;
R² is hydrogen, halo, cyano, C₁₋₃ alkyl, C₁₋₃ alkoxy or C₁₋₃ haloalkyl;
R³ is C₁₋₆ alkyl, C₁₋₆ haloalkyl, C₃₋₆ cycloalkyl, 3- to 10-membered heterocyclyl, 3- to 10-membered (—O—) heterocyclyl or 5- to 10-membered heteroaryl, —C(O) R⁷, —C(O)NR⁸R⁹;
R⁴ is hydrogen, C₁₋₃ alkyl or C₁₋₃ haloalkyl;
R⁵ is independently selected from halo, cyano, hydroxy, C₁₋₃ alkyl, C₁₋₃ alkoxy, C₁₋₃ haloalkyl and NH₂;
R⁷ is hydrogen, C₁₋₃ alkyl or C₁₋₃ alkoxy;
R⁸ and R⁹ are independently selected from hydrogen and C₁₋₃ alkyl; or
R⁸ and R⁹ are optionally taken together to form a 5- to 8-membered heterocyclyl; and
n is an integer 0, 1, 2 or 3;
wherein each heterocyclyl and heteroaryl is optionally substituted with one to three substituents each individually selected from halo, OH, C₁₋₃ alkyl, C₁₋₃ alkoxy, and C₁₋₃ haloalkyl, oxo, and —C(O)R⁶, wherein R⁶ is hydrogen or C₁₋₃ alkyl;
wherein each alkyl and cycloalkyl is optionally substituted with one to three substituents each individually selected from halo, OH, amine, cyano, C₁₋₃ alkyl, C₁₋₃ alkoxy, and C₁₋₃ haloalkyl, oxo, and —C(O) R¹⁰, wherein R¹⁰ is hydrogen, OH, C₁₋₃ alkyl or C₁₋₃ alkoxy.

In one embodiment R¹ is a 3- to 10-membered heterocyclyl. In another embodiment R¹ is a 6-membered heterocyclyl, optionally substituted with C₁₋₃ alkyl.

In one embodiment R¹ is azepanyl, 1,3-dioxolanyl, 1,4-dioxolanyl, maleimidyl, succinimidyl, dioxopiperazinyl, hydantoinyl, imidazolinyl, imidazolidinyl, isoxazolinyl, isoxazolidinyl, oxazolinyl, oxazolidinyl, oxazolidinonyl, thiazolinyl, thiazolidinyl, morpholinyl, oxiranyl, piperidinyl N-oxide, piperidinyl, piperazinyl, pyrrolidinyl, pyrrolidonyl, pyrrolidionyl, 4-piperidonyl, pyrazolinyl, pyrazolidinyl, 2-oxopyrrolidinyl, tetrahydropyranyl, quinuclidinyl, 4H-pyran, azetidinyl, oxetanyl, octahydrocyclopenta[c]pyrrole, 2-azaspiro[3.3]heptanyl, 3-oxabicyclo[3.1.0]hexanyl, 3-azabicyclo[3.1.0]hexanyl, 3-azabicyclo[3.1.1]heptanyl, 4-azaspiro[2.5]octanyl, 6-azaspiro[3.5]nonanyl, 2,6-diazaspiro[3.3]heptanyl, 7-azabicyclo[2.2.1]heptanyl, 2-azabicyclo[2.2.1]heptanyl, 2,5-diazabicyclo[2.2.2]octanyl, 2,5-diazabicyclo[2.2.1]heptanyl, 2-oxabicyclo[2.1.1]hexanyl, 3-azabicyclo[3.2.1]octanyl, hexahydro-1H-cyclopenta[c]pyrrolyl, 3-oxa-9-azabicyclo[3.3.1]nonanyl, or hexahydro-1H-pyrrolizinyl, optionally substituted with one or more C₁₋₃ alkyl.

In one embodiment R¹ is tetrahydrofuranyl, tetrahydropyranyl, azetidinyl, pyrrolidinyl, or piperidinyl. In another embodiment R¹ is tetrahydrofuranyl, tetrahydropyranyl, azetidinyl, pyrrolidinyl, or piperidinyl, optionally substituted with one or more C₁₋₃ alkyl.

In one embodiment R¹ is selected from:

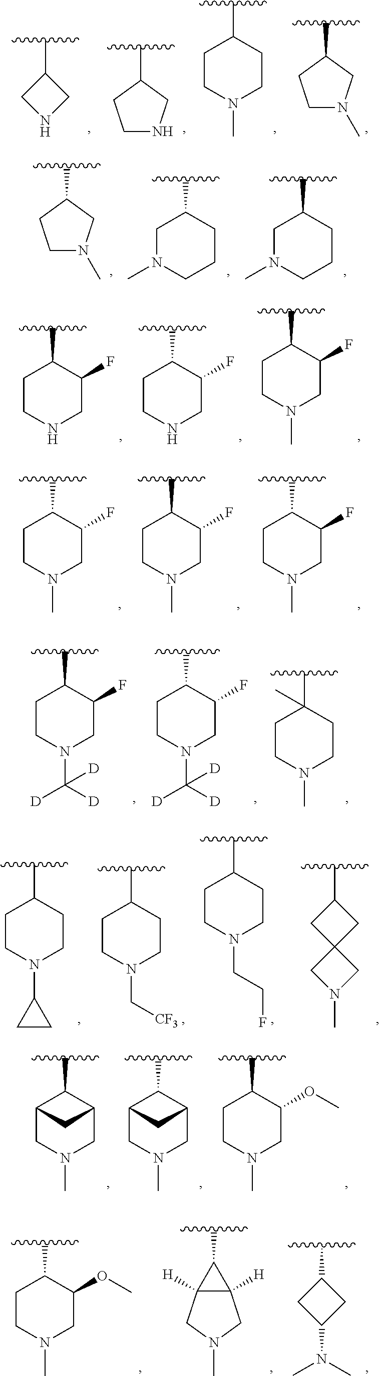

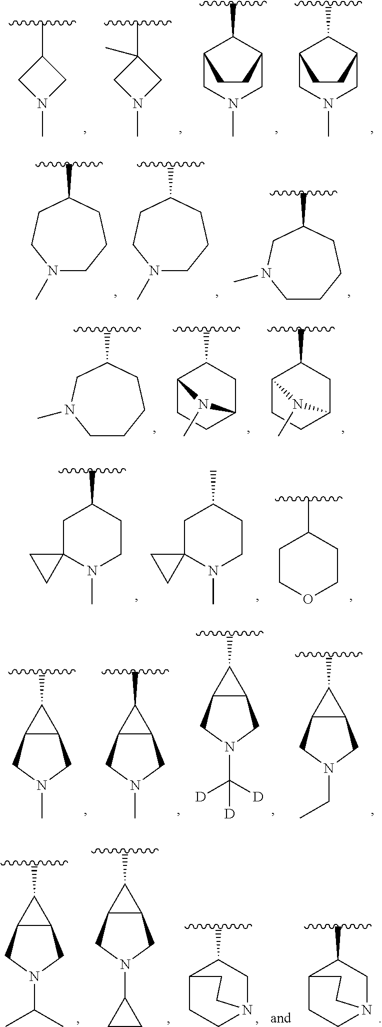

In one embodiment, R¹ is a C₁₋₁₂ alkyl. In one embodiment, the C₁₋₁₂ alkyl is substituted with one to three substituents each individually selected from halo, OH, amine, cyano, C₁₋₃ alkyl, C₁₋₃ alkoxy, and C₁₋₃ haloalkyl, oxo, and —C(O) R¹⁰, where R¹⁰ is hydrogen, OH, C₁₋₃ alkyl or C₁₋₃ alkoxy. In one embodiment, R¹ is an unsubstituted C₁₋₁₂ alkyl.

In one embodiment, R¹ is a C₁₋₆ alkyl. In one embodiment, the C₁₋₆ alkyl is substituted with one to three substituents each individually selected from halo, OH, amine, cyano, C₁₋₃ alkyl, C₁₋₃ alkoxy, and C₁₋₃ haloalkyl, oxo, and —C(O) R″, where R″ is hydrogen, OH, C₁₋₃ alkyl or C₁₋₃ alkoxy.

In one embodiment, R¹ is a C₁₋₄ alkyl substituted with a cyano or an amine. In some embodiments the amine substituent is an alkyl amine (e.g., a dimethyl amine).

In one embodiment, R¹ is a C₃₋₆ cycloalkyl. In one embodiment, the C₃₋₆ cycloalkyl is substituted with one to three substituents each individually selected from halo, OH, amine, cyano, C₁₋₃ alkyl, C₁₋₃ alkoxy, and C₁₋₃ haloalkyl, oxo, and —C(O)R¹⁰, where R¹⁰ is hydrogen, OH, C₁₋₃ alkyl or C₁₋₃ alkoxy. In one embodiment, R¹ is an unsubstituted C₃₋₆ cycloalkyl.

In one embodiment, R¹ is a C₃₋₆ cycloalkyl substituted with an amine. In embodiment, the amine substituent is an alkyl amine (e.g., a dimethyl amine).

In one embodiment R² is hydrogen, halo, cyano or C₁₋₃ haloalkyl.

In one embodiment, R² is a halo.

In one embodiment R³ is a 3- to 10-membered heterocyclyl or a 5- to 10-membered heteroaryl.

In an embodiment R³ is a 4- to 6-membered heterocyclyl. In some embodiments, R³ is a 4- to 6-membered heterocyclyl, optionally substituted with one or more halo, OH or C₁₋₃ alkyl.

In one embodiment R³ is azepanyl, 1,3-dioxolane, 1,4-dioxolanyl, maleimidyl, succinimidyl, dioxopiperazinyl, hydantoinyl, imidazolinyl, imidazolidinyl, isoxazolinyl, isoxazolidinyl, oxazolinyl, oxazolidinyl, oxazolidinonyl, thiazolinyl, thiazolidinyl, morpholinyl, oxiranyl, piperidinyl N-oxide, piperidinyl, piperazinyl, pyrrolidinyl, pyrrolidonyl, pyrrolidionyl, 4-piperidonyl, pyrazolinyl, pyrazolidinyl, 2-oxopyrrolidinyl, tetrahydropyranyl, quinuclidineyl, 4H-pyranyl, azetidinyl, oxetanyl, octahydrocyclopenta[c]pyrrole, 2-azaspiro[3.3]heptanyl, 3-oxabicyclo[3.1.0]hexanyl, 3-azabicyclo[3.1.0]hexanyl, 3-azabicyclo[3.1.1]heptanyl, 4-azaspiro[2.5]octanyl, 6-azaspiro[3.5]nonanyl, 2,6-diazaspiro[3.3]heptanyl, 7-azabicyclo[2.2.1]heptanyl, 2-azabicyclo[2.2.1]heptanyl, 2,5-diazabicyclo[2.2.2]octanyl, 2,5-diazabicyclo[2.2.1]heptanyl, 2-oxabicyclo[2.1.1]hexanyl, 3-azabicyclo[3.2.1]octanyl, hexahydro-1H-cyclopenta[c]pyrrolyl, 3-oxa-9-azabicyclo[3.3.1]nonanyl, 3-oxa-7-azabicyclo[3.3.1]nonanyl, hexahydro-1H-pyrrolizinyl, 2-oxa-7-azaspiro[4.4]nonanyl, and 6-oxa-1-azaspiro[3.4]octanyl, optionally substituted with one or more halo, OH or C₁₋₃ alkyl.

In one embodiment R³ is tetrahydropyranyl, azepanyl, azetidinyl, morpholinyl, pyrrolidinyl, piperidinyl, 4-azaspiro[2.5]octanyl, 7-azabicyclo[2.2.1]heptanyl, 3-azabicyclo[3.2.1]octanyl, 3-azabicyclo[3.1.0]hexanyl, 3-azabicyclo[3.1.1]heptanyl, 2-azaspiro[3.3]heptanyl or hexahydro-1H-cyclopenta[c]pyrrolyl, optionally substituted with one or more one halo, OH or C₁₋₃ alkyl.

In one embodiment R³ is morpholinyl, optionally substituted with one or more halo, OH or C₁₋₃ alkyl.

In one embodiment R³ is piperidinyl, optionally substituted with one or more halo, OH or C₁₋₃ alkyl.

In one embodiment R³ is morpholinyl.

In one embodiment R³ is a 5- to 10-membered heteroaryl.

In one embodiment R³ is oxazolyl, thiazolyl, pyrazolyl, pyridyl, pyridazinyl, pyrimidinyl or pyridone.

In one embodiment R³ is pyridyl, optionally substituted with one or more halo, oxo or C₁₋₃ alkyl.

In one embodiment R³ is C₃₋₆ cycloalkyl.

In one embodiment R³ is —C(O) R⁷ or —C(O) NR⁸R⁹, where R⁷, R⁸ and R⁹ are as described herein including in embodiments.

In one embodiment R⁷ is C₁₋₃ alkoxy.

In one embodiment R⁸ is H or C₁₋₃ alkyl and R⁹ is C₁₋₃ alkyl.

In one embodiment R⁸ and R⁹ are taken together to form a 5- to 8-membered heterocyclyl. In some embodiments, the 5- to 8-membered heterocyclyl is a morpholino or a piperidinyl.

In one embodiment R⁴ is C₁₋₃ alkyl. In another embodiment R⁴ is methyl.

In one embodiment R⁵ is halo, C₁₋₃ alkyl or C₁₋₃ haloalkyl. In another embodiment R⁵ is —CH₃. In another embodiment R⁵ is CN. In another embodiments R⁵ is halogen.

In one embodiment the compound is a compound selected from Examples 1-81.

### Methods of Treatment

Provided herein is a method of treating cancer (e.g., a Ras pathway-associated cancer) in a subject in need of such treatment, the method comprising administering to the subject an effective amount of a compound of Formula (I), or a pharmaceutically acceptable salt thereof or a pharmaceutical composition thereof. In some embodiments, a cancer is a Ras pathway-associated cancer. In some embodiments, a cancer is a Ras-associated cancer. In some embodiments, a cancer is a KRas-associated cancer. In some embodiments, a cancer is a HRas-associated cancer. In some embodiments, a cancer is a NRas-associated cancer. In some embodiments, a cancer is a SOS1-associated cancer.

For example, provided herein are methods for treating a Ras pathway-associated cancer (e.g., a SOS1-associated cancer, a Ras-associated cancer (e.g., a KRas-associated cancer, a HRas-associated cancer, and/or a NRas-associated cancer), an EGFR-associated cancer, an ErbB2-associated cancer, an ErbB3-associated cancer, an ErbB4-associated cancer, a NF1-associated cancer, a PDGFR-A-associated cancer, a PDGFR-B-associated cancer, a FGFR1-associated cancer, FGFR2-associated cancer, FGFR3-associated cancer, a IGF1 R-associated cancer, a INSR-associated cancer, a ALK-associated cancer, a ROS-associated cancer, a TrkA-associated cancer, a TrkB-associated cancer, a TrkC-associated cancer, a RET-associated cancer, a c-MET-associated cancer, a VEGFR1-associated cancer, a VEGFR2-associated cancer, a VEGFR3-associated cancer, an AXL-associated cancer, a SHP2-associated cancer, a RAF-associated cancer (e.g., a BRAF-associated cancer), a PI3K-associated cancer, an AKT-associated cancer, an mTOR-associated cancer, a MEK-associated cancer, an ERK-associated cancer, or a combination thereof) in a subject in need of such treatment, the method comprising a) detecting a dysregulation of a Ras pathway gene, a Ras pathway protein, or the expression or activity or level of any of the same in a sample from the subject; and b) administering a effective amount of a compound of Formula (I), or a pharmaceutically acceptable salt thereof. In some embodiments, the dysregulation of a Ras pathway gene, a Ras pathway protein, or the expression or activity or level of any of the same includes one or more fusion proteins.

For example, provided herein are methods for treating a Ras-associated cancer in a subject in need of such treatment, the method comprising a) detecting a dysregulation of a Ras gene, a Ras protein, or the expression or activity or level of any of the same in a sample from the subject; and b) administering a effective amount of a compound of Formula (I), or a pharmaceutically acceptable salt thereof. In some embodiments, the dysregulation of a Ras gene, a Ras protein, or the expression or activity or level of any of the same includes one or more fusion proteins.

For example, provided herein are methods for treating a KRas-associated cancer in a subject in need of such treatment, the method comprising a) detecting a dysregulation of a KRas gene, a KRas protein, or the expression or activity or level of any of the same in a sample from the subject; and b) administering a effective amount of a compound of Formula (I), or a pharmaceutically acceptable salt thereof. In some embodiments, the dysregulation of a KRas gene, a KRas protein, or the expression or activity or level of any of the same includes one or more fusion proteins.

For example, provided herein are methods for treating a HRas-associated cancer in a subject in need of such treatment, the method comprising a) detecting a dysregulation of a HRas gene, a HRas protein, or the expression or activity or level of any of the same in a sample from the subject; and b) administering a effective amount of a compound of Formula (I), or a pharmaceutically acceptable salt thereof. In some embodiments, the dysregulation of a HRas gene, a HRas protein, or the expression or activity or level of any of the same includes one or more fusion proteins.

For example, provided herein are methods for treating a NRas-associated cancer in a subject in need of such treatment, the method comprising a) detecting a dysregulation of a NRas gene, a NRas protein, or the expression or activity or level of any of the same in a sample from the subject; and b) administering a effective amount of a compound of Formula (I), or a pharmaceutically acceptable salt thereof. In some embodiments, the dysregulation of a NRas gene, a NRas protein, or the expression or activity or level of any of the same includes one or more fusion proteins.

For example, provided herein are methods for treating a SOS1-associated cancer in a subject in need of such treatment, the method comprising a) detecting a dysregulation of a SOS1 gene, a SOS1 protein, or the expression or activity or level of any of the same in a sample from the subject; and b) administering a effective amount of a compound of Formula (I), or a pharmaceutically acceptable salt thereof. In some embodiments, the dysregulation of a SOS1 gene, a SOS1 protein, or the expression or activity or level of any of the same includes one or more fusion proteins.

Also provided are methods for treating cancer in a subject in need thereof, the method comprising: (a) detecting a Ras pathway-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a Ras pathway-associated cancer through the use of a regulatory agency-approved, e.g., FDA-approved test or assay for identifying dysregulation of a Ras pathway gene, a Ras pathway protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a Ras-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a Ras-associated cancer through the use of a regulatory agency-approved, e.g., FDA-approved test or assay for identifying dysregulation of a Ras gene, a Ras protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a KRas-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a KRas-associated cancer through the use of a regulatory agency-approved, e.g., FDA-approved test or assay for identifying dysregulation of a KRas gene, a KRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a HRas-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a HRas-associated cancer through the use of a regulatory agency-approved, e.g., FDA-approved test or assay for identifying dysregulation of a HRas gene, a HRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a NRas-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a NRas-associated cancer through the use of a regulatory agency-approved, e.g., FDA-approved test or assay for identifying dysregulation of a NRas gene, a NRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a SOS1-associated cancer in the subject; and (b) administering to the subject an effective amount of a compound of Formula (I), or a pharmaceutically acceptable salt thereof, or a pharmaceutical composition thereof. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a SOS1-associated cancer through the use of a regulatory agency-approved, e.g., FDA-approved test or assay for identifying dysregulation of a SOS1 gene, a SOS1 protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a Ras pathway gene, a Ras pathway protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a Ras pathway-associated cancer through the use of a regulatory agency-approved, e.g., FDA-approved test or assay for identifying dysregulation of a Ras pathway gene, a Ras pathway protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a Ras gene, a Ras protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a Ras-associated cancer through the use of a regulatory agency-approved, e.g., FDA-approved test or assay for identifying dysregulation of a Ras gene, a Ras protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a KRas gene, a KRas protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a KRas-associated cancer through the use of a regulatory agency-approved, e.g., FDA-approved test or assay for identifying dysregulation of a KRas gene, a KRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a HRas gene, a HRas protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a HRas-associated cancer through the use of a regulatory agency-approved, e.g., FDA-approved test or assay for identifying dysregulation of a HRas gene, a HRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a NRas gene, a NRas protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a NRas-associated cancer through the use of a regulatory agency-approved, e.g., FDA-approved test or assay for identifying dysregulation of a NRas gene, a NRas protein, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: administering an effective amount of a compound of Formula (I), or a pharmaceutically acceptable salt thereof, or a pharmaceutical composition thereof to a subject determined to have a cancer associated with a dysregulation of a SOS1 gene, a SOS1 protein, or expression or activity or level of any of the same. Some embodiments of these methods further include administering to the subject another anticancer agent (e.g., a small molecule or an immunotherapy). In some embodiments, the subject was previously treated with another anticancer treatment, e.g., at least partial resection of the tumor or radiation therapy. In some embodiments, the subject is determined to have a SOS1-associated cancer through the use of a regulatory agency-approved, e.g., FDA-approved test or assay for identifying dysregulation of a SOS1 gene, a SOS1 protein, or expression or activity or level of any of the same, in a subject or a biopsy sample from the subject or by performing any of the non-limiting examples of assays described herein. In some embodiments, the test or assay is provided as a kit.

Also provided are methods of treating a subject that include performing an assay on a sample obtained from the subject to determine whether the subject has a dysregulation of a Ras pathway gene, a Ras pathway protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a Ras pathway gene, a Ras pathway protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a Ras pathway-associated cancer, a subject presenting with one or more symptoms of a Ras pathway-associated cancer, or a subject having an elevated risk of developing a Ras pathway-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a Ras gene, a Ras protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a Ras gene, a Ras protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a Ras-associated cancer, a subject presenting with one or more symptoms of a Ras-associated cancer, or a subject having an elevated risk of developing a Ras-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a KRas gene, a KRas protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a KRas gene, a KRas protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a KRas-associated cancer, a subject presenting with one or more symptoms of a KRas-associated cancer, or a subject having an elevated risk of developing a KRas-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a HRas gene, a HRas protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a HRas gene, a HRas protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a HRas-associated cancer, a subject presenting with one or more symptoms of an HRas-associated cancer, or a subject having an elevated risk of developing a HRas-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a NRas gene, a NRas protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a NRas gene, a NRas protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a NRas-associated cancer, a subject presenting with one or more symptoms of an NRas-associated cancer, or a subject having an elevated risk of developing a NRas-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided are methods of treating a subject that include performing an assay on a sample obtained from the subject to determine whether the subject has a dysregulation of a SOS1 gene, a SOS1 protein, or expression or activity or level of any of the same, and administering (e.g., specifically or selectively administering) an effective amount of a compound of Formula (I), or a pharmaceutically acceptable salt thereof, or a pharmaceutical composition thereof, to the subject determined to have a dysregulation of a SOS1 gene, a SOS1 protein, or expression or activity or level of any of the same. Some embodiments of these methods further include administering to the subject another anticancer agent (e.g., a small molecule or immunotherapy). In some embodiments of these methods, the subject was previously treated with another anticancer treatment, e.g., at least partial resection of a tumor or radiation therapy. In some embodiments, the subject is a subject suspected of having a SOS1-associated cancer, a subject presenting with one or more symptoms of a SOS1-associated cancer, or a subject having an elevated risk of developing a SOS1-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided is a compound of Formula (I), or a pharmaceutically acceptable salt thereof, or a pharmaceutical composition thereof, for use in treating a Ras pathway-associated cancer in a subject identified or diagnosed as having a Ras pathway-associated cancer through a step of performing an assay (e.g., an in vitro assay) on a sample obtained from the subject to determine whether the subject has a dysregulation of a Ras pathway protein, a Ras pathway protein, or expression or activity or level of any of the same, where the presence of a dysregulation of a Ras pathway gene, a Ras pathway protein, or expression or activity or level of any of the same, identifies that the subject has a Ras pathway-associated cancer. Also provided is the use of a compound of Formula (I), or a pharmaceutically acceptable salt thereof, for the manufacture of a medicament for treating a Ras pathway-associated cancer in a subject identified or diagnosed as having a Ras pathway-associated cancer through a step of performing an assay on a sample obtained from the subject to determine whether the subject has a dysregulation of a Ras pathway gene, a Ras pathway protein, or expression or activity or level of any of the same where the presence of dysregulation of a Ras pathway gene, a Ras pathway protein, or expression or activity or level of any of the same, identifies that the subject has a Ras pathway-associated cancer. Some embodiments of any of the methods or uses described herein further include recording in the subject's clinical record (e.g., a computer readable medium) that the subject is determined to have a dysregulation of a Ras pathway gene, a Ras pathway protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a Ras-associated cancer in a subject identified or diagnosed as having a Ras-associated cancer through a step of performing an assay (e.g., an in vitro assay) on a sample obtained from the subject to determine whether the subject has a dysregulation of a Ras gene, a Ras protein, or expression or activity or level of any of the same, where the presence of a dysregulation of a Ras gene, a Ras protein, or expression or activity or level of any of the same, identifies that the subject has a Ras-associated cancer. Also provided is the use of a compound of Formula (I), or a pharmaceutically acceptable salt thereof, for the manufacture of a medicament for treating a Ras-associated cancer in a subject identified or diagnosed as having a Ras-associated cancer through a step of performing an assay on a sample obtained from the subject to determine whether the subject has a dysregulation of a Ras gene, a Ras protein, or expression or activity or level of any of the same where the presence of dysregulation of a Ras gene, a Ras protein, or expression or activity or level of any of the same, identifies that the subject has a Ras-associated cancer. Some embodiments of any of the methods or uses described herein further include recording in the subject's clinical record (e.g., a computer readable medium) that the subject is determined to have a dysregulation of a Ras gene, a Ras protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a KRas-associated cancer in a subject identified or diagnosed as having a KRas-associated cancer through a step of performing an assay (e.g., an in vitro assay) on a sample obtained from the subject to determine whether the subject has a dysregulation of a KRas gene, a KRas protein, or expression or activity or level of any of the same, where the presence of a dysregulation of a KRas gene, a KRas protein, or expression or activity or level of any of the same, identifies that the subject has a KRas-associated cancer. Also provided is the use of a compound of Formula (I), or a pharmaceutically acceptable salt thereof, for the manufacture of a medicament for treating a KRas-associated cancer in a subject identified or diagnosed as having a KRas-associated cancer through a step of performing an assay on a sample obtained from the subject to determine whether the subject has a dysregulation of a KRas gene, a KRas protein, or expression or activity or level of any of the same where the presence of dysregulation of a KRas gene, a KRas protein, or expression or activity or level of any of the same, identifies that the subject has a KRas-associated cancer. Some embodiments of any of the methods or uses described herein further include recording in the subject's clinical record (e.g., a computer readable medium) that the subject is determined to have a dysregulation of a KRas gene, a KRas protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a HRas-associated cancer in a subject identified or diagnosed as having a HRas-associated cancer through a step of performing an assay (e.g., an in vitro assay) on a sample obtained from the subject to determine whether the subject has a dysregulation of a HRas gene, a HRas protein, or expression or activity or level of any of the same, where the presence of a dysregulation of a HRas gene, a HRas protein, or expression or activity or level of any of the same, identifies that the subject has a HRas-associated cancer. Also provided is the use of a compound of Formula (I), or a pharmaceutically acceptable salt thereof, for the manufacture of a medicament for treating a HRas-associated cancer in a subject identified or diagnosed as having a HRas-associated cancer through a step of performing an assay on a sample obtained from the subject to determine whether the subject has a dysregulation of a HRas gene, a HRas protein, or expression or activity or level of any of the same where the presence of dysregulation of a HRas gene, a HRas protein, or expression or activity or level of any of the same, identifies that the subject has a HRas-associated cancer. Some embodiments of any of the methods or uses described herein further include recording in the subject's clinical record (e.g., a computer readable medium) that the subject is determined to have a dysregulation of a HRas gene, a HRas protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a NRas-associated cancer in a subject identified or diagnosed as having a NRas-associated cancer through a step of performing an assay (e.g., an in vitro assay) on a sample obtained from the subject to determine whether the subject has a dysregulation of a NRas gene, a NRas protein, or expression or activity or level of any of the same, where the presence of a dysregulation of a NRas gene, a NRas protein, or expression or activity or level of any of the same, identifies that the subject has a NRas-associated cancer. Also provided is the use of a compound of Formula (I), or a pharmaceutically acceptable salt thereof, for the manufacture of a medicament for treating a NRas-associated cancer in a subject identified or diagnosed as having a NRas-associated cancer through a step of performing an assay on a sample obtained from the subject to determine whether the subject has a dysregulation of a

NRas gene, a NRas protein, or expression or activity or level of any of the same where the presence of dysregulation of a NRas gene, a NRas protein, or expression or activity or level of any of the same, identifies that the subject has a NRas-associated cancer. Some embodiments of any of the methods or uses described herein further include recording in the subject's clinical record (e.g., a computer readable medium) that the subject is determined to have a dysregulation of a NRas gene, a NRas protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, or a pharmaceutical composition thereof, for use in treating a SOS1-associated cancer in a subject identified or diagnosed as having a SOS1-associated cancer through a step of performing an assay (e.g., an in vitro assay) on a sample obtained from the subject to determine whether the subject has a dysregulation of a SOS1 gene, a SOS1 protein, or expression or activity or level of any of the same, where the presence of a dysregulation of a SOS1 gene, a SOS1 protein, or expression or activity or level of any of the same, identifies that the subject has a SOS1-associated cancer. Also provided is the use of a compound of Formula (I), or a pharmaceutically acceptable salt thereof, for the manufacture of a medicament for treating a SOS1-associated cancer in a subject identified or diagnosed as having a SOS1-associated cancer through a step of performing an assay on a sample obtained from the subject to determine whether the subject has a dysregulation of a SOS1 gene, a SOS1 protein, or expression or activity or level of any of the same where the presence of dysregulation of a SOS1 gene, a SOS1 protein, or expression or activity or level of any of the same, identifies that the subject has a SOS1-associated cancer. Some embodiments of any of the methods or uses described herein further include recording in the subject's clinical record (e.g., a computer readable medium) that the subject is determined to have a dysregulation of a SOS1 gene, a SOS1 protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

In some embodiments of any of the methods or uses described herein, the subject has been identified or diagnosed as having a cancer with a dysregulation of a Ras pathway gene, a Ras pathway protein, or expression or activity or level of any of the same. In some embodiments of any of the methods or uses described herein, the subject has a tumor that is positive for a dysregulation of a Ras pathway gene, a Ras pathway protein, or expression or activity or level of any of the same. In some embodiments of any of the methods or uses described herein, the subject can be a subject with a tumor(s) that is positive for a dysregulation of a Ras pathway gene, a Ras pathway protein, or expression or activity or level of any of the same. In some embodiments of any of the methods or uses described herein, the subject can be a subject whose tumors have a dysregulation of a Ras pathway gene, a Ras pathway protein, or expression or activity or level of any of the same. In some embodiments of any of the methods or uses described herein, the subject is suspected of having a Ras pathway-associated cancer. In some embodiments, provided herein are methods for treating a Ras pathway-associated cancer in a subject in need of such treatment, the method comprising a) detecting a dysregulation of a Ras pathway gene, a Ras pathway protein, or the expression or activity or level of any of the same in a sample from the subject; and b) administering an effective amount of a compound of Formula (I), or a pharmaceutically acceptable salt thereof. In some embodiments, the dysregulation of a Ras pathway gene, a Ras pathway protein, or the expression or activity or level of any of the same includes one or more Ras pathway protein point mutations/insertions/deletions.

In some embodiments of any of the methods or uses described herein, the subject has been identified or diagnosed as having a cancer with a dysregulation of a Ras gene, a Ras protein, or expression or activity or level of any of the same. In some embodiments of any of the methods or uses described herein, the subject has a tumor that is positive for a dysregulation of a Ras gene, a Ras protein, or expression or activity or level of any of the same. In some embodiments of any of the methods or uses described herein, the subject can be a subject with a tumor(s) that is positive for a dysregulation of a Ras gene, a Ras protein, or expression or activity or level of any of the same. In some embodiments of any of the methods or uses described herein, the subject can be a subject whose tumors have a dysregulation of a Ras gene, a Ras protein, or expression or activity or level of any of the same. In some embodiments of any of the methods or uses described herein, the subject is suspected of having a Ras-associated cancer. In some embodiments, provided herein are methods for treating a Ras-associated cancer in a subject in need of such treatment, the method comprising a) detecting a dysregulation of a Ras gene, a Ras protein, or the expression or activity or level of any of the same in a sample from the subject; and b) administering an effective amount of a compound of Formula (I), or a pharmaceutically acceptable salt thereof. In some embodiments, the dysregulation of a Ras gene, a Ras protein, or the expression or activity or level of any of the same includes one or more Ras protein point mutations/insertions/deletions.

In some embodiments of any of the methods or uses described herein, the subject has been identified or diagnosed as having a cancer with a dysregulation of a KRas gene, a KRas protein, or expression or activity or level of any of the same. In some embodiments of any of the methods or uses described herein, the subject has a tumor that is positive for a dysregulation of a KRas gene, a KRas protein, or expression or activity or level of any of the same. In some embodiments of any of the methods or uses described herein, the subject can be a subject with a tumor(s) that is positive for a dysregulation of a KRas gene, a KRas protein, or expression or activity or level of any of the same. In some embodiments of any of the methods or uses described herein, the subject can be a subject whose tumors have a dysregulation of a KRas gene, a KRas protein, or expression or activity or level of any of the same. In some embodiments of any of the methods or uses described herein, the subject is suspected of having a KRas-associated cancer. In some embodiments, provided herein are methods for treating a KRas-associated cancer in a subject in need of such treatment, the method comprising a) detecting a dysregulation of a KRas gene, a KRas protein, or the expression or activity or level of any of the same in a sample from the subject; and b) administering an effective amount of a compound of Formula (I), or a pharmaceutically acceptable salt thereof. In some embodiments, the dysregulation of a KRas gene, a KRas protein, or the expression or activity or level of any of the same includes one or more KRas protein point mutations/insertions/deletions. Non-limiting examples of KRas protein point mutations/insertions/deletions are described in Table 1.

In some embodiments of any of the methods or uses described herein, the subject has been identified or diagnosed as having a cancer with a dysregulation of a HRas gene, a HRas protein, or expression or activity or level of any of the same. In some embodiments of any of the methods or uses described herein, the subject has a tumor that is positive for a dysregulation of a HRas gene, a HRas protein, or expression or activity or level of any of the same. In some embodiments of any of the methods or uses described herein, the subject can be a subject with a tumor(s) that is positive for a dysregulation of a HRas gene, a HRas protein, or expression or activity or level of any of the same. In some embodiments of any of the methods or uses described herein, the subject can be a subject whose tumors have a dysregulation of a HRas gene, a HRas protein, or expression or activity or level of any of the same. In some embodiments of any of the methods or uses described herein, the subject is suspected of having a HRas-associated cancer. In some embodiments, provided herein are methods for treating a HRas-associated cancer in a subject in need of such treatment, the method comprising a) detecting a dysregulation of a HRas gene, a HRas protein, or the expression or activity or level of any of the same in a sample from the subject; and b) administering an effective amount of a compound of Formula (I), or a pharmaceutically acceptable salt thereof. In some embodiments, the dysregulation of a HRas gene, a HRas protein, or the expression or activity or level of any of the same includes one or more HRas protein point mutations/insertions/deletions. Non-limiting examples of HRas protein point mutations/insertions/deletions are described in Table 2.

In some embodiments of any of the methods or uses described herein, the subject has been identified or diagnosed as having a cancer with a dysregulation of a NRas gene, a NRas protein, or expression or activity or level of any of the same. In some embodiments of any of the methods or uses described herein, the subject has a tumor that is positive for a dysregulation of a NRas gene, a NRas protein, or expression or activity or level of any of the same. In some embodiments of any of the methods or uses described herein, the subject can be a subject with a tumor(s) that is positive for a dysregulation of a NRas gene, a NRas protein, or expression or activity or level of any of the same. In some embodiments of any of the methods or uses described herein, the subject can be a subject whose tumors have a dysregulation of a NRas gene, a NRas protein, or expression or activity or level of any of the same. In some embodiments of any of the methods or uses described herein, the subject is suspected of having a NRas-associated cancer. In some embodiments, provided herein are methods for treating a NRas-associated cancer in a subject in need of such treatment, the method comprising a) detecting a dysregulation of a NRas gene, a NRas protein, or the expression or activity or level of any of the same in a sample from the subject; and b) administering an effective amount of a compound of Formula (I), or a pharmaceutically acceptable salt thereof. In some embodiments, the dysregulation of a NRas gene, a NRas protein, or the expression or activity or level of any of the same includes one or more NRas protein point mutations/insertions/deletions. Non-limiting examples of NRas protein point mutations/insertions/deletions are described in Table 3.

In some embodiments of any of the methods or uses described herein, the subject has been identified or diagnosed as having a cancer with a dysregulation of a SOS1 gene, a SOS1 protein, or expression or activity or level of any of the same. In some embodiments of any of the methods or uses described herein, the subject has a tumor that is positive for a dysregulation of a SOS1 gene, a SOS1 protein, or expression or activity or level of any of the same. In some embodiments of any of the methods or uses described herein, the subject can be a subject with a tumor(s) that is positive for a dysregulation of a SOS1 gene, a SOS1 protein, or expression or activity or level of any of the same. In some embodiments of any of the methods or uses described herein, the subject can be a subject whose tumors have a dysregulation of a SOS1 gene, a SOS1 protein, or expression or activity or level of any of the same. In some embodiments of any of the methods or uses described herein, the subject is suspected of having a SOS1-associated cancer. In some embodiments, provided herein are methods for treating a SOS1-associated cancer in a subject in need of such treatment, the method comprising a) detecting a dysregulation of a SOS1 gene, a SOS1 protein, or the expression or activity or level of any of the same in a sample from the subject; and b) administering an effective amount of a compound of Formula (I), or a pharmaceutically acceptable salt thereof. In some embodiments, the dysregulation of a SOS1 gene, a SOS1 protein, or the expression or activity or level of any of the same includes one or more SOS1 protein point mutations/insertions/deletions. Non-limiting examples of SOS1 protein point mutations/insertions/deletions are described in Table 4.

In some embodiments, the cancer with a dysregulation of a Ras pathway gene, a Ras pathway protein, or expression or activity or level of any of the same is determined using a regulatory agency-approved, e.g., FDA-approved, assay or kit. In some embodiments, the tumor with a dysregulation of a Ras pathway gene, a Ras pathway protein, or expression or activity or level of any of the same is determined using a regulatory agency-approved, e.g., FDA-approved, assay or kit.

In some embodiments, the cancer with a dysregulation of a Ras gene, a Ras protein, or expression or activity or level of any of the same is determined using a regulatory agency-approved, e.g., FDA-approved, assay or kit. In some embodiments, the tumor with a dysregulation of a Ras gene, a Ras protein, or expression or activity or level of any of the same is determined using a regulatory agency-approved, e.g., FDA-approved, assay or kit.

In some embodiments, the cancer with a dysregulation of a KRas gene, a KRas protein, or expression or activity or level of any of the same is determined using a regulatory agency-approved, e.g., FDA-approved, assay or kit. In some embodiments, the tumor with a dysregulation of a KRas gene, a KRas protein, or expression or activity or level of any of the same is determined using a regulatory agency-approved, e.g., FDA-approved, assay or kit.

In some embodiments, the cancer with a dysregulation of a HRas gene, a HRas protein, or expression or activity or level of any of the same is determined using a regulatory agency-approved, e.g., FDA-approved, assay or kit. In some embodiments, the tumor with a dysregulation of a HRas gene, a HRas protein, or expression or activity or level of any of the same is determined using a regulatory agency-approved, e.g., FDA-approved, assay or kit.

In some embodiments, the cancer with a dysregulation of a NRas gene, a NRas protein, or expression or activity or level of any of the same is determined using a regulatory agency-approved, e.g., FDA-approved, assay or kit. In some embodiments, the tumor with a dysregulation of a NRas gene, a NRas protein, or expression or activity or level of any of the same is determined using a regulatory agency-approved, e.g., FDA-approved, assay or kit.

In some embodiments, the cancer with a dysregulation of a SOS1 gene, a SOS1 protein, or expression or activity or level of any of the same is determined using a regulatory agency-approved, e.g., FDA-approved, assay or kit. In some embodiments, the tumor with a dysregulation of a SOS1 gene, a SOS1 protein, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of a Ras pathway gene, a Ras pathway protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a Ras pathway gene, a Ras pathway protein, or expression or activity or level of any of the same.

In some embodiments of any of the methods or uses described herein, the subject has a clinical record indicating that the subject has a tumor that has a dysregulation of a Ras gene, a Ras protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a Ras gene, a Ras protein, or expression or activity or level of any of the same.

In some embodiments of any of the methods or uses described herein, the subject has a clinical record indicating that the subject has a tumor that has a dysregulation of a KRas gene, a KRas protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a KRas gene, a KRas protein, or expression or activity or level of any of the same.

In some embodiments of any of the methods or uses described herein, the subject has a clinical record indicating that the subject has a tumor that has a dysregulation of a HRas gene, a HRas protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a HRas gene, a HRas protein, or expression or activity or level of any of the same.

In some embodiments of any of the methods or uses described herein, the subject has a clinical record indicating that the subject has a tumor that has a dysregulation of a NRas gene, a NRas protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a NRas gene, a NRas protein, or expression or activity or level of any of the same.

In some embodiments of any of the methods or uses described herein, the subject has a clinical record indicating that the subject has a tumor that has a dysregulation of a SOS1 gene, a SOS1 protein, or expression or activity or level of any of the same. Also provided are methods of treating a subject that include administering an effective amount of a compound of Formula (I), or a pharmaceutically acceptable salt thereof, to a subject having a clinical record that indicates that the subject has a dysregulation of a SOS1 gene, a SOS1 protein, or expression or activity or level of any of the same.

In some embodiments, the methods provided herein include performing an assay on a sample obtained from the subject to determine whether the subject has a dysregulation of a Ras pathway gene, a Ras pathway protein, or expression or level of any of the same. In some such embodiments, the method also includes administering to a subject determined to have a dysregulation of a Ras pathway gene, a Ras pathway protein, or expression or activity, or level of any of the same an effective amount of a compound of Formula (I), or a pharmaceutically acceptable salt thereof. In some embodiments, the method includes determining that a subject has a dysregulation of a Ras pathway gene, a Ras pathway protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a Ras gene, a Ras protein, or expression or level of any of the same. In some such embodiments, the method also includes administering to a subject determined to have a dysregulation of a Ras gene, a Ras protein, or expression or activity, or level of any of the same an effective amount of a compound of Formula (I), or a pharmaceutically acceptable salt thereof. In some embodiments, the method includes determining that a subject has a dysregulation of a Ras gene, a Ras protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a KRas gene, a KRas protein, or expression or level of any of the same. In some such embodiments, the method also includes administering to a subject determined to have a dysregulation of a KRas gene, a KRas protein, or expression or activity, or level of any of the same an effective amount of a compound of Formula (I), or a pharmaceutically acceptable salt thereof. In some embodiments, the method includes determining that a subject has a dysregulation of a KRas gene, a KRas protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a HRas gene, a HRas protein, or expression or level of any of the same. In some such embodiments, the method also includes administering to a subject determined to have a dysregulation of a HRas gene, a HRas protein, or expression or activity, or level of any of the same an effective amount of a compound of Formula (I), or a pharmaceutically acceptable salt thereof. In some embodiments, the method includes determining that a subject has a dysregulation of a HRas gene, a HRas protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a NRas gene, a NRas protein, or expression or level of any of the same. In some such embodiments, the method also includes administering to a subject determined to have a dysregulation of a NRas gene, a NRas protein, or expression or activity, or level of any of the same an effective amount of a compound of Formula (I), or a pharmaceutically acceptable salt thereof. In some embodiments, the method includes determining that a subject has a dysregulation of a NRas gene, a NRas protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments, the methods provided herein include performing an assay on a sample obtained from the subject to determine whether the subject has a dysregulation of a SOS1 gene, a SOS1 protein, or expression or level of any of the same. In some such embodiments, the method also includes administering to a subject determined to have a dysregulation of a SOS1 gene, a SOS1 protein, or expression or activity, or level of any of the same an effective amount of a compound of Formula (I), or a pharmaceutically acceptable salt thereof. In some embodiments, the method includes determining that a subject has a dysregulation of a SOS1 gene, a SOS1 protein, or expression or level of any of the same via an assay performed on a sample obtained from the subject. In such embodiments, the method also includes administering to a subject an effective amount of a compound of Formula (I), or a pharmaceutically acceptable salt thereof.

In some embodiments of any of the methods or uses described herein, the cancer is a hematological cancer. Examples of hematological cancers (e.g., hematological cancers that are Ras pathway-associated cancers) include, for example, leukemias (e.g., acute myeloid leukemia, acute lymphoblastic leukemia, chronic lymphocytic leukemia, chronic myelogenous leukemia, specify juvenile myelomonocytic leukemia (JMML), and hairy cell leukemia) and lymphomas (e.g., non-Hodgkin's lymphoma, Hodgkin's disease cutaneous T-cell lymphoma, and Burkitt lymphoma).

In some embodiments of any of the methods or uses described herein, the cancer is a solid tumor. Examples of solid tumors (e.g., solid tumors that are Ras pathway-associated cancers) include, for example, thyroid cancer (e.g., papillary thyroid carcinoma, medullary thyroid carcinoma), lung cancer (e.g., non-small cell lung cancer, small-cell lung carcinoma, bronchial adenoma, and pleuropulmonary blastoma), pancreatic cancer, pancreatic ductal carcinoma, biliary tract cancer, breast cancer (e.g., invasive ductal carcinoma, invasive lobular carcinoma, ductal carcinoma in situ, and lobular carcinoma in situ), stomach cancer, small intestinal cancer, colon cancer, colorectal cancer, peritoneal cancer, ovarian cancer, uterine cancer, liver cancer, endometrial cancer, prostate cancer (including benign prostatic hyperplasia), testicular cancer, bladder cancer, urinary tract cancer, cervical cancer, head and neck cancer, brain cancer (e.g., glioblastoma, brain stem and hypophtalmic glioma, cerebellar and cerebral astrocytoma, medulloblastoma, and ependymoma), squamous cell carcinoma, and melanoma.

In some embodiments, the subject is a human.

Compounds of Formula (I) and pharmaceutically acceptable salts and solvates thereof are also useful for treating a Ras pathway-associated cancer.

Accordingly, also provided herein is a method for treating a subject diagnosed with or identified as having a Ras pathway-associated cancer, e.g., any of the exemplary Ras pathway-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Accordingly, also provided herein is a method for treating a subject diagnosed with or identified as having a Ras pathway-associated cancer, e.g., any of the exemplary Ras pathway-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Compounds of Formula (I) and pharmaceutically acceptable salts and solvates thereof are also useful for treating a Ras-associated cancer.

Accordingly, also provided herein is a method for treating a subject diagnosed with or identified as having a Ras-associated cancer, e.g., any of the exemplary Ras-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Accordingly, also provided herein is a method for treating a subject diagnosed with or identified as having a Ras-associated cancer, e.g., any of the exemplary Ras-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Compounds of Formula (I) and pharmaceutically acceptable salts and solvates thereof are also useful for treating a KRas-associated cancer.

Accordingly, also provided herein is a method for treating a subject diagnosed with or identified as having a KRas-associated cancer, e.g., any of the exemplary KRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Accordingly, also provided herein is a method for treating a subject diagnosed with or identified as having a KRas-associated cancer, e.g., any of the exemplary KRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Compounds of Formula (I) and pharmaceutically acceptable salts and solvates thereof are also useful for treating a HRas-associated cancer.

Accordingly, also provided herein is a method for treating a subject diagnosed with or identified as having a HRas-associated cancer, e.g., any of the exemplary HRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Accordingly, also provided herein is a method for treating a subject diagnosed with or identified as having a HRas-associated cancer, e.g., any of the exemplary HRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Compounds of Formula (I) and pharmaceutically acceptable salts and solvates thereof are also useful for treating a NRas-associated cancer.

Accordingly, also provided herein is a method for treating a subject diagnosed with or identified as having a NRas-associated cancer, e.g., any of the exemplary NRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Accordingly, also provided herein is a method for treating a subject diagnosed with or identified as having a NRas-associated cancer, e.g., any of the exemplary NRas-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Compounds of Formula (I) and pharmaceutically acceptable salts and solvates thereof are also useful for treating a SOS1-associated cancer.

Accordingly, also provided herein is a method for treating a subject diagnosed with or identified as having a SOS1-associated cancer, e.g., any of the exemplary SOS1-associated cancers disclosed herein, comprising administering to the subject an effective amount of a compound of Formula (I), or a pharmaceutically acceptable salt thereof, or a pharmaceutical composition thereof as defined herein. In some embodiments, the compound of Formula (I) is selected from Examples 1-81, or a pharmaceutically acceptable salt thereof.

Dysregulation of a Ras pathway protein, a Ras pathway gene, or the expression or activity or level of any (e.g., one or more) of the same can contribute to tumorigenesis. For example, a fusion protein can have increased activity as compared to a wild type Ras pathway protein (e.g., for SOS1, increased Ras activation through more advantageous binding and/or increased GEF activity), increased expression (e.g., increased levels) of a wild type Ras pathway protein in a mammalian cell can occur due to aberrant cell signaling and/or dysregulated autocrine/paracrine signaling (e.g., as compared to a control non-cancerous cell), Ras pathway mRNA splice variants may also result in dysregulation of Ras pathway.

In some embodiments, the compounds provided herein exhibit brain and/or central nervous system (CNS) penetrance. Such compounds are capable of crossing the blood brain barrier and inhibiting Ras pathway (e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof)) activity in the brain and/or other CNS structures. In some embodiments, the compounds provided herein are capable of crossing the blood brain barrier in an effective amount. For example, treatment of a subject with cancer (e.g., a Ras pathway-associated cancer such as a Ras pathway-associated brain or CNS cancer) can include administration (e.g., oral administration) of the compound to the subject. In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, the tumors listed in Louis, D. N. et al. Acta Neuropathol 131 (6), 803-820 (June 2016)). In some embodiments, the brain tumor is a primary brain tumor. In some embodiments, the subject has previously been treated with another anticancer agent, e.g., another Ras pathway inhibitor (e.g., a compound that is not a compound of General Formula (I), or an inhibitor of another Ras pathway gene or protein (e.g., Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof), or a combination thereof). In some embodiments, the brain tumor is a metastatic brain tumor. In some embodiments, the subject has previously been treated with another anticancer agent, e.g., another Ras pathway inhibitor (e.g., a compound that is not a compound of Formula (I), or an inhibitor of another Ras pathway gene or protein.

The ability of the compounds described herein, to cross the BBB can be demonstrated by assays known in the art. Such assays include BBB models such as the transwell system, the hollow fiber (dynamic in vitro BBB) model, other microfluidic BBB systems, the BBB spheroid platform, and other cell aggregate-based BBB models. See, e.g., Cho et al. Nat Commun. 2017; 8:15623; Bagchi, et al. Drug Des Devel Ther. 2019; 13:3591-3605; Gastfriend, et al. Curr Opin Biomed Eng. 2018 March; 5:6-12; and Wang et al. Biotechnol Bioeng. 2017 January; 114 (1): 184-194. In some embodiments, the compounds described herein, are fluorescently labeled, and the fluorescent label can be detected using microscopy (e.g., confocal microscopy). In some such embodiments, the ability of the compound to penetrate the surface barrier of the model can be represented by the fluorescence intensity at a given depth below the surface. In some assays, such as a calcein-AM-based assay, the fluorescent label is non-fluorescent until it permeates live cells and is hydrolyzed by intracellular esterases to produce a fluorescent compound that is retained in the cell and can be quantified with a spectrophotometer. Non-limiting examples of fluorescent labels that can be used in the assays described herein include Cy5, rhodamine, infrared IRDye® CW-800 (LICOR #929-71012), far-red IRDye® 650 (LICOR #929-70020), sodium fluorescein (Na—F), lucifer yellow (LY), 5′carboxyfluorescein, and calcein-acetoxymethylester (calcein-AM). In some embodiments, the BBB model (e.g., the tissue or cell aggregate) can be sectioned, and a compound described herein can be detected in one or more sections using mass spectrometry (e.g., MALDI-MSI analyses). In some embodiments, the ability of a compound described herein to cross the BBB through a transcellular transport system, such as receptor-mediated transport (RMT), carrier-mediated transport (CMT), or active efflux transport (AET), can be demonstrated by assays known in the art. See, e.g., Wang, et al. Drug Deliv. 2019; 26 (1): 551-565. In some embodiments, assays to determine if compounds can be effluxed by the P-glycoprotein (Pgp) include monolayer efflux assays in which movement of compounds through Pgp is quantified by measuring movement of digoxin, a model Pgp substrate (see, e.g., Doan et al. 2002. J Pharmacol Exp Ther. 303 (3): 1029-1037). Alternative in vivo assays to identify compounds that pass through the blood-brain barriers include phage-based systems (see, e.g., Peng et al. 2019. ChemRxiv. Preprint doi.org/10.26434/chemrxiv.8242871.v1). In some embodiments, binding of the compounds described herein to brain tissue is quantified. For example, a brain tissue binding assay can be performed using equilibrium dialysis, and the fraction of a compound described herein unbound to brain tissue can be detected using LC-MS/MS (Cyprotex: Brain Tissue Binding Assay www.cyprotex.com/admepk/protein binding/brain-tissue-binding/).

In some embodiments, the subject has been identified or diagnosed as having a cancer with a dysregulation of a Ras pathway gene, a Ras pathway protein, or expression or activity, or level of any of the same (a Ras pathway-associated cancer) (e.g., as determined using a regulatory agency-approved, e.g., FDA-approved, assay or kit). In some embodiments, the subject has a tumor that is positive for a dysregulation of a Ras pathway gene, a Ras pathway protein, or expression or activity, or level of any of the same (e.g., as determined using a regulatory agency-approved assay or kit). The subject can be a subject with a tumor(s) that is positive for a dysregulation of a Ras pathway gene, a Ras pathway protein, or expression or activity, or level of any of the same (e.g., identified as positive using a regulatory agency-approved, e.g., FDA-approved, assay or kit). The subject can be a subject whose tumors have a dysregulation of a Ras pathway gene, a Ras pathway protein, or expression or activity, or a level of the same (e.g., where the tumor is identified as such using a regulatory agency-approved, e.g., FDA-approved, kit or assay). In some embodiments, the subject is suspected of having a Ras pathway-associated cancer. In some embodiments, the subject has a clinical record indicating that the subject has a tumor that has a dysregulation of a Ras pathway gene, a Ras pathway protein, or expression or activity, or level of any of the same (and optionally the clinical record indicates that the subject should be treated with any of the compositions provided herein).

In some embodiments, the subject has been identified or diagnosed as having a cancer with a dysregulation of a Ras gene, a Ras protein, or expression or activity, or level of any of the same (a Ras-associated cancer) (e.g., as determined using a regulatory agency-approved, e.g., FDA-approved, assay or kit). In some embodiments, the subject has a tumor that is positive for a dysregulation of a Ras gene, a Ras protein, or expression or activity, or level of any of the same (e.g., as determined using a regulatory agency-approved assay or kit). The subject can be a subject with a tumor(s) that is positive for a dysregulation of a Ras gene, a Ras protein, or expression or activity, or level of any of the same (e.g., identified as positive using a regulatory agency-approved, e.g., FDA-approved, assay or kit). The subject can be a subject whose tumors have a dysregulation of a Ras gene, a Ras protein, or expression or activity, or a level of the same (e.g., where the tumor is identified as such using a regulatory agency-approved, e.g., FDA-approved, kit or assay). In some embodiments, the subject is suspected of having a Ras-associated cancer. In some embodiments, the subject has a clinical record indicating that the subject has a tumor that has a dysregulation of a Ras gene, a Ras protein, or expression or activity, or level of any of the same (and optionally the clinical record indicates that the subject should be treated with any of the compositions provided herein).

In some embodiments, the subject has been identified or diagnosed as having a cancer with a dysregulation of a KRas gene, a KRas protein, or expression or activity, or level of any of the same (a KRas-associated cancer) (e.g., as determined using a regulatory agency-approved, e.g., FDA-approved, assay or kit). In some embodiments, the subject has a tumor that is positive for a dysregulation of a KRas gene, a KRas protein, or expression or activity, or level of any of the same (e.g., as determined using a regulatory agency-approved assay or kit). For example, the subject has a tumor that is positive for a mutation as described in Table 1. The subject can be a subject with a tumor(s) that is positive for a dysregulation of a KRas gene, a KRas protein, or expression or activity, or level of any of the same (e.g., identified as positive using a regulatory agency-approved, e.g., FDA-approved, assay or kit). The subject can be a subject whose tumors have a dysregulation of a KRas gene, a KRas protein, or expression or activity, or a level of the same (e.g., where the tumor is identified as such using a regulatory agency-approved, e.g., FDA-approved, kit or assay). In some embodiments, the subject is suspected of having a KRas-associated cancer. In some embodiments, the subject has a clinical record indicating that the subject has a tumor that has a dysregulation of a KRas gene, a KRas protein, or expression or activity, or level of any of the same (and optionally the clinical record indicates that the subject should be treated with any of the compositions provided herein).

In some embodiments, the subject has been identified or diagnosed as having a cancer with a dysregulation of a HRas gene, a HRas protein, or expression or activity, or level of any of the same (a HRas-associated cancer) (e.g., as determined using a regulatory agency-approved, e.g., FDA-approved, assay or kit). In some embodiments, the subject has a tumor that is positive for a dysregulation of a HRas gene, a HRas protein, or expression or activity, or level of any of the same (e.g., as determined using a regulatory agency-approved assay or kit). For example, the subject has a tumor that is positive for a mutation as described in Table 2. The subject can be a subject with a tumor(s) that is positive for a dysregulation of a HRas gene, a HRas protein, or expression or activity, or level of any of the same (e.g., identified as positive using a regulatory agency-approved, e.g., FDA-approved, assay or kit). The subject can be a subject whose tumors have a dysregulation of a HRas gene, a HRas protein, or expression or activity, or a level of the same (e.g., where the tumor is identified as such using a regulatory agency-approved, e.g., FDA-approved, kit or assay). In some embodiments, the subject is suspected of having a HRas-associated cancer. In some embodiments, the subject has a clinical record indicating that the subject has a tumor that has a dysregulation of a HRas gene, a HRas protein, or expression or activity, or level of any of the same (and optionally the clinical record indicates that the subject should be treated with any of the compositions provided herein).

In some embodiments, the subject has been identified or diagnosed as having a cancer with a dysregulation of a NRas gene, a NRas protein, or expression or activity, or level of any of the same (a NRas-associated cancer) (e.g., as determined using a regulatory agency-approved, e.g., FDA-approved, assay or kit). In some embodiments, the subject has a tumor that is positive for a dysregulation of a NRas gene, a NRas protein, or expression or activity, or level of any of the same (e.g., as determined using a regulatory agency-approved assay or kit). For example, the subject has a tumor that is positive for a mutation as described in Table 3. The subject can be a subject with a tumor(s) that is positive for a dysregulation of a NRas gene, a NRas protein, or expression or activity, or level of any of the same (e.g., identified as positive using a regulatory agency-approved, e.g., FDA-approved, assay or kit). The subject can be a subject whose tumors have a dysregulation of a NRas gene, a NRas protein, or expression or activity, or a level of the same (e.g., where the tumor is identified as such using a regulatory agency-approved, e.g., FDA-approved, kit or assay). In some embodiments, the subject is suspected of having a NRas-associated cancer. In some embodiments, the subject has a clinical record indicating that the subject has a tumor that has a dysregulation of a NRas gene, a NRas protein, or expression or activity, or level of any of the same (and optionally the clinical record indicates that the subject should be treated with any of the compositions provided herein).

In some embodiments, the subject has been identified or diagnosed as having a cancer with a dysregulation of a SOS1 gene, a SOS1 protein, or expression or activity, or level of any of the same (a SOS1-associated cancer) (e.g., as determined using a regulatory agency-approved, e.g., FDA-approved, assay or kit). In some embodiments, the subject has a tumor that is positive for a dysregulation of a SOS1 gene, a SOS1 protein, or expression or activity, or level of any of the same (e.g., as determined using a regulatory agency-approved assay or kit). For example, the subject has a tumor that is positive for a mutation as described in Table 4. The subject can be a subject with a tumor(s) that is positive for a dysregulation of a SOS1 gene, a SOS1 protein, or expression or activity, or level of any of the same (e.g., identified as positive using a regulatory agency-approved, e.g., FDA-approved, assay or kit). The subject can be a subject whose tumors have a dysregulation of a SOS1 gene, a SOS1 protein, or expression or activity, or a level of the same (e.g., where the tumor is identified as such using a regulatory agency-approved, e.g., FDA-approved, kit or assay). In some embodiments, the subject is suspected of having a SOS1-associated cancer. In some embodiments, the subject has a clinical record indicating that the subject has a tumor that has a dysregulation of a SOS1 gene, a SOS1 protein, or expression or activity, or level of any of the same (and optionally the clinical record indicates that the subject should be treated with any of the compositions provided herein).

In some embodiments of any of the methods or uses described herein, an assay used to determine whether the subject has a dysregulation of a Ras pathway gene, or a Ras pathway protein, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a Ras pathway gene, a Ras pathway protein, or expression or activity or levels of any of the same. In some embodiments, the sample is a biological sample or a biopsy sample (e.g., a paraffin-embedded biopsy sample) from the subject. In some embodiments, the subject is a subject suspected of having a Ras pathway-associated cancer, a subject having one or more symptoms of a Ras pathway-associated cancer, and/or a subject that has an increased risk of developing a Ras pathway-associated cancer).

In some embodiments, dysregulation of a Ras pathway gene, a Ras pathway protein, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3 (3): 36, 2016. Liquid biopsy methods can be used to detect total tumor burden and/or the dysregulation of a Ras pathway gene, a Ras pathway protein, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a Ras pathway gene, a Ras pathway protein, or the expression or activity or level of any of the same. In some embodiments, liquid biopsies can be used to detect the presence of dysregulation of a Ras pathway gene, a Ras pathway protein, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a Ras pathway gene, a Ras pathway protein, or the expression or activity or level of any of the same.

In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a Ras pathway gene, a Ras pathway protein, or the expression or activity or level of any of the same.

In some embodiments, ctDNA derived from a single gene can be detected using a liquid biopsy. In some embodiments, ctDNA derived from a plurality of genes (e.g., 2, 3, 4, 5, 6, 7, 8, 9, 10, 15, 20, 25, 30, 35, 40, 45, 50, 55, 60, 65, 70, 75, 80, 85, 90, 95, 100 or more, or any number of genes in between these numbers) can be detected using a liquid biopsy. In some embodiments, ctDNA derived from a plurality of genes can be detected using any of a variety of commercially-available testing panels (e.g., commercially-available testing panels designed to detect dysregulation of a Ras pathway gene, a Ras pathway protein, or the expression or activity or level of any of the same). Liquid biopsies can be used to detect dysregulation of a Ras pathway gene, a Ras pathway protein, or the expression or activity or level of any of the same including, without limitation, point mutations or single nucleotide variants (SNVs), copy number variants (CNVs), genetic fusions (e.g., translocations or rearrangements), insertions, deletions, or any combination thereof. In some embodiments, a liquid biopsy can be used to detect a germline mutation. In some embodiments, a liquid biopsy can be used to detect a somatic mutation. In some embodiments, a liquid biopsy can be used to detect a primary genetic mutation (e.g., a primary mutation or a primary fusion that is associated with initial development of a disease, e.g., cancer). In some embodiments, a dysregulation of a Ras pathway gene, a Ras pathway protein, or the expression or activity or level of any of the same identified using a liquid biopsy is also present in a cancer cell that is present in the subject (e.g., in a tumor). In some embodiments, any of the types of dysregulation of a Ras pathway gene, a Ras pathway protein, or the expression or activity or level of any of the same described herein can be detected using a liquid biopsy. In some embodiments, a genetic mutation identified via a liquid biopsy can be used to identify the subject as a candidate for a particular treatment. For example, detection of dysregulation of a Ras pathway gene, a Ras pathway protein, or the expression or activity or level of any of the same in the subject can indicate that the subject will be responsive to a treatment that includes administration of a compound of Formula (I), or a pharmaceutically acceptable salt thereof.

Some embodiments of these methods can further include administering to the subject at least one dose of the compound of Formula (I), or a pharmaceutically acceptable salt thereof, between the first and second time points. For example, a reduction (e.g., a 1% to about a 99% reduction, a 1% reduction to about a 50% reduction, a 1% reduction to about a 10% reduction, about a 50% to about a 99% reduction, or about a 75% to about a 95% reduction) in the allele frequency (AF) of the dysregulation of a Ras pathway gene in the cfDNA obtained from the subject at the second time point as compared to the allele frequency (AF) of the dysregulation of a Ras pathway gene in the cfDNA obtained from the subject at the first time point indicates that the compound of Formula (I), or a pharmaceutically acceptable salt thereof, was effective in the subject. In some embodiments, the AF is reduced such that the level is below the detection limit of the instrument. Alternatively, an increase in the allele frequency (AF) of the dysregulation of a Ras pathway gene in the cfDNA obtained from the subject at the second time point as compared to the allele frequency (AF) of the dysregulation of a Ras pathway gene in the cfDNA obtained from the subject at the first time point indicates that the compound of Formula (I), or a pharmaceutically acceptable salt thereof, was not effective in the subject. Some embodiments of these methods can further include, administering additional doses of a compound of Formula (I), or a pharmaceutically acceptable salt thereof, to a subject in which a compound of Formula (I), or a pharmaceutically acceptable salt thereof, was determined to be effective. Some embodiments of these methods can further include, administering a different treatment (e.g., a treatment that does not include the administration of a compound of Formula (I), or a pharmaceutically acceptable salt thereof, as a monotherapy) to a subject in which a compound of Formula (I), or a pharmaceutically acceptable salt thereof, was determined not to be effective.

In some examples of these methods, the time difference between the first and second time points can be about 1 day to about 1 year, about 1 day to about 1 month, about 1 day to about 5 days, about 1 month to about 3 months, about 3 months to about 6 months, or about 7 months to about 9 months. In some embodiments of these methods, the subject can be previously identified as having a cancer having a dysregulated Ras pathway gene (e.g., any of the examples of a dysregulated Ras pathway gene described herein). In some embodiments of these methods, a subject can have been previously diagnosed as having any of the types of cancer described herein. In some embodiments of these methods, the subject can have one or more metastases (e.g., one or more brain metastases).

In some of the above embodiments, the cfDNA comprises ctDNA such as Ras pathway-associated ((e.g., SOS1, Ras (e.g., KRas, HRas, and/or NRas), EGFR, ErbB2, ErbB3, ErbB4, NF1, PDGFR-A, PDGFR-B, FGFR1, FGFR2, FGFR3, IGF1 R, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof)-associated) ctDNA. For example, the cfDNA is ctDNA such as Ras pathway-associated ctDNA. In some embodiments, at least some portion of cfDNA is determined to be Ras pathway-associated ctDNA, for example, a sequenced and/or quantified amount of the total cfDNA is determined to have a Ras pathway fusion and/or overexpression of Ras pathway.

### Combinations

In the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other Ras pathway inhibitors, kinase inhibitors, signal transduction inhibitors, and/or monoclonal antibodies. For example, a surgery may be open surgery or minimally invasive surgery. Compounds of Formula (I), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof for a period of time and then undergo at least partial resection of the tumor. In some embodiments, the treatment with one or more doses of a compound of Formula (I), or a pharmaceutically acceptable salt thereof reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof for a period of time and under one or more rounds of radiation therapy. In some embodiments, the treatment with one or more doses of a compound of Formula (I), or a pharmaceutically acceptable salt thereof reduces the size of the tumor (e.g., the tumor burden) prior to the one or more rounds of radiation therapy.

A “Ras pathway targeted therapeutic agent” as used herein includes any compound exhibiting inactivation activity (e.g., active site (e.g., competitive) inhibition, allosteric inhibition, inhibition of dimerization, inhibition of expression, inhibition of protein-protein interaction, and induction of degradation) of any protein in a Ras pathway. Non-limiting examples of a protein in a Ras pathway include any one of the proteins in the Ras-RAF-MAPK pathway or PI3K/AKT pathway such as Ras (e.g., KRas, HRas, and/or NRas), EGFR, ErbB2, ErbB3, ErbB4, NF1, PDGFR-A, PDGFR-B, FGFR1, FGFR2, FGFR3, IGFIR, INSR, ALK, ROS, TrkA, TrkB, TrkC, RET, c-MET, VEGFR1, VEGFR2, VEGFR3, AXL, SHP2, RAF (e.g., BRAF), PI3K, AKT, mTOR, MEK, ERK, or a combination thereof. In some embodiments, a Ras pathway targeted therapeutic agent can be selective for a protein in a Ras pathway. For example, the Ras pathway targeted therapeutic agent can be selective for a Ras protein (e.g., KRas, HRas, and/or NRas, or mutated forms of any thereof); such an agent can also be called a “Ras modulator”). In some embodiments, a Ras modulator is a covalent inhibitor. In some embodiments, a Ras pathway targeted therapeutic agent can be selective for a particular Ras protein (e.g., KRas, HRas, or NRas), or a mutated form thereof (e.g., a G12 mutant, a G13 mutant, or a Q61 mutant). Non-limiting examples of KRas-targeted therapeutic agents (e.g., KRas inhibitors (such as KRas G12C inhibitors)) include AMG 510, ARS-3248, ARS1620, SML-8-73-1, SML-10-70-1, VSA9, AA12, MRTX-849, MRTX849, LY3499446, JNJ-74699157, ARS853, AZD4785, and JNJ-74699157.

Compounds of Formula (I), or pharmaceutically acceptable salts or thereof, can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof, for a period of time and then undergo at least partial resection of the tumor. In some embodiments, the treatment with one or more doses of a compound of Formula (I), or a pharmaceutically acceptable salt thereof, reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof, for a period of time and under one or more rounds of radiation therapy. In some embodiments, the treatment with one or more doses of a compound of Formula (I), or a pharmaceutically acceptable salt thereof, reduces the size of the tumor (e.g., the tumor burden) prior to the one or more rounds of radiation therapy.

In some embodiments, the one or more additional therapies or therapeutic agents are independently selected from: EGFR inhibitors (e.g., afatinib, erlotinib, gefitinib, lapatinib, cetuximab, panitumumab, osimertinib, and olmutinib), ErbB2/Her2 inhibitors (e.g., afatinib, lapatinib, trastuzumab, and pertuzumab), ALK inhibitors (e.g., crizotinib, alectinib, entrectinib, brigatinib), ROS1 inhibitors (e.g., crizotinib, entrectinib, lorlatinib, ceritinib, and merestinib), MEK inhibitors (e.g., trametinib, cobimetinib, binimetinib, selumetinib, refametinib), RAS (KRas, HRas, and/or NRas) inhibitors (e.g., MRTX849, LY3499446, JNJ-74699157, AMG 510, and AZD4785), Bcr-Abl inhibitors (e.g., imatinib, dasatinib, nilotinib), FGFR1, 2, or 3 inhibitors (e.g., nintedanib), MET inhibitors (e.g., capmatinib), AXL inhibitors (e.g., sitravatinib), RET inhibitors (e.g., sunitinib and selpercatinib), ERK inhibitors (e.g., ulixertinib), Shp2 inhibitors (e.g., RLY-1971, RMC-4630, TNO155, and JAB-3068), Bcl-2 inhibitors (e.g., ABT-263, obatoclax, ABT-737, and navitoclax), mTOR inhibitors (e.g., everolimus and tacrolimus), Trk inhibitors (e.g., larotrectinib and entrectinib), checkpoint inhibitors (e.g., ipilimumab, nivolumab, pembrolizumab, atezolizumab, avelumab, durvalumab, and pidilizumab) or other immunotherapies (e.g., monoclonal antibodies), PARP inhibitors (e.g., olaparib), PI3K inhibitors (e.g., buparlisib), BET inhibitors (e.g., GSK1210151A), Raf inhibitors (e.g., encorafenib), MCL-1 inhibitors (e.g., AZD5991), AKT inhibitors (e.g., miltefosine), PDK1 inhibitors (e.g., GSK 2334470), and other chemotherapeutic agents such as taxanes (e.g., paclitaxel and docetaxel), platinum-based agents (e.g., cisplatin and carboplatin), cytoxic agents (e.g., 5-fluorouracil, capecitabine, floxuridine, cytarabine, and gemcitabine), farnesyltransferase inhibitors, topoisomerase inhibitors (e.g., topotecan and irinotecan), DNA synthesis inhibitors (e.g., capecitabine (Xeloda®) and gemcitabine hydrochloride (Gemzar®)), alkylating agents (e.g., temozolomide (Temodar® and Temodal®), dactinomycin (also known as actinomycin-D, Cosmegen®), carmustine (BiCNU®), bendamustine (Treanda®), and lomustine (CeeNU®)), and cytotoxic agents (e.g., vincristine, cytarabine, and pemetrexed).

Epidermal growth factor receptor (EGFR) inhibitors such as osimertinib (AZD9291, merelectinib, TAGRISSO®), erlotinib (TARCEVA®), gefitinib (IRESSA®), cetuximab (ERBITUX®), necitumumab (PORTRAZZA®, IMC-11F8), neratinib (HKI-272, NERLYNX®), lapatinib (TYKERB®), panitumumab (ABX-EGF, VECTIBIX®), vandetanib (CAPRELSA®), rociletinib (CO-1686), olmutinib (OLITA®, HM61713, BI-1482694), naquotinib (ASP8273), nazartinib (EGF816, NVS-816), PF-06747775, icotinib (BPI-2009H), afatinib (BIBW 2992, GILOTRIFR), dacomitinib (PF-00299804, PF-804, PF-299, PF-299804), avitinib (AC0010), AC0010MA EAI045, matuzumab (EMD-7200), nimotuzumab (h-R³, BIOMAb EGFR®), zalutumab, MDX447, depatuxizumab (humanized mAb 806, ABT-806), depatuxizumab mafodotin (ABT-414), ABT-806, mAb 806, canertinib (CI-1033), shikonin, shikonin derivatives (e.g., deoxyshikonin, isobutyrylshikonin, acetylshikonin, β,β-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, avitinib, GW572016, pyrotinib (SHR1258), SCT200, CPGJ602, Sym004, MAb-425, Modotuximab (TAB-H49), futuximab (992 DS), zalutumumab, KL-140, RO5083945, IMGN289, JNJ-61186372, LY3164530, Sym013, AMG 595, BDTX-189, avatinib, Disruptin, CL-387785, EGFRBi-Armed Autologous T Cells, and EGFR CAR-T Therapy. In some embodiments, the EGFR-targeted therapeutic agent is selected from osimertinib, gefitinib, erlotinib, afatinib, lapatinib, neratinib, AZD-9291, CL-387785, CO-1686, or WZ4002.

Human Epidermal Growth Factor Receptor 2 (HER2 receptor) (also known as Neu, ErbB-2, CD340, or p185) inhibitors such as trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIFR), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

In some embodiments, the FGFR inhibitor is selected from infigratinib, AZD4547, erdafitinib (JNJ-42756493), nintedanib dovitinib, ponatinib, and TAS120.

In some embodiments, the ALK inhibitor is selected from alectinib, crizotinib (XALKORI®), ceritinib, AP26113, ASP3026, TSR-011, PF-06463922, X-396, and CEP-37440.

In some embodiments, the ROS1 inhibitor is selected from crizotinib (XALKORI®), ceritinib, lorlatinib, brigatinib, cabozantinib, and repotrectinib.

In some embodiments, the mTOR inhibitor is selecte from everolimus, tacrolimus rapamycin, perifosine, and temsirolimus.

In some embodiments, the Trk inhibitor is selected from larotrectinib, lestaurtinib, and entrectinib.

In some embodiments, the RET inhibitors is selected from sunitinib (Sutent®), selpercatinib (RETEVMO®), vandetanib (Caprelsa®), motesanib (AMG706), sorafenib, regorafenib, and danusertib.

In some embodiments, the MET inhibitor is selected from capmatinib, tepotinib, savolitinib, crizotinib, cabozantinib, tivantinib, bozitinib, merestinib, glesatinib, sitravatinib, onartuzumab, and emibetuzumab.

In some embodiments, the AXL inhibitor is selected from sitravatinib, bemcentinib, dubermatinib, DS-1205, SLC-391, INCB081776, ONO-7475, and BA3011.

In some embodiments, the Shp2 inhibitor is selected from TNO155, BBP-398, JAB-3068, RMC-4360, and RLY-1971.

In some embodiments, the RAF inhibitor is a BRAF inhibitor, such as vemurafenib (ZELBORAF®), dabrafenib (TAFINLAR®), encorafenib (BRAFTOVI®), BMS-908662, sorafenib, LGX818, PLX3603, RAF265, RO5185426, GSK2118436, ARQ 736, GDC-0879, PLX-4720, AZ304, PLX-8394, HM95573, RO5126766, and LXH254.

In some embodiments, the PI3K inhibitor is selected from buparlisib (BKM120), alpelisib (BYL719), WX-037, copanlisib (ALIQOPAR, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, and GSK2636771.

In some embodiments, the AKT inhibitor is selected from miltefosine (IMPADIVOR), wortmannin, NL-71-101, H-89, GSK690693, CCT128930, AZD5363, ipatasertib (GDC-0068, RG7440), A-674563, A-443654, AT7867, AT13148, uprosertib, afuresertib, DC120, MK-2206, edelfosine, miltefosine, perifosine, erucylphophocholine, erufosine, SR13668, OSU-A9, PH-316, PHT-427, PIT-1, DM-PIT-1, triciribine, API-1, ARQ092, BAY 1125976, 3-oxo-tirucallic acid, lactoquinomycin, GSK2141795, ONC201, tricirbine, A674563, and AT7867.

In some embodiments, the MEK inhibitor is selected from trametinib (MEKINIST®), cobimetinib (COTELLIC®), binimetinib (MEKTOVI®), selumetinib (AZD6244), PD0325901, MSC1936369B, SHR7390, TAK-733, RO5126766, CS3006, WX-554, PD98059, CI1040 (PD184352), and hypothemycin.

In some embodiments, the ERK inhibitor is selected from FRI-20 (ON-01060), VTX-Ile, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, and ONC201.

In some embodiments, the PARP inhibitors include olaparib (LYNPARZA®), talazoparib, rucaparib, niraparib, veliparib, BGB-290 (pamiparib), CEP 9722, E7016, iniparib, IMP4297 November1401, 2X-121, ABT-767, RBN-2397, BMN 673, KU-0059436 (AZD2281), BSI-201, PF-01367338, INO-1001, and JPI-289.

In some embodiments, the RAS inhibitor is MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849.

In some embodiments, the PDK-1 inhibitor is selected from GSK 2334470, JX06, SNS-510, and AR-12.

In some embodiments, the BET inhibitor is selected from GSK1210151A, GSK525762, OTX-015, TEN-010, CPI-203, CPI-0610, olinone, RVX-208, ABBV-744, LY294002, AZD5153, MT-1, and MS645.

In some embodiments, the MCL-1 inhibitor is AZD5991.

In some embodiments, the Bcl-2 protein family inhibitor is selected from ABT-263, Tetrocarcin A, Antimycin, Gossypol ((−) BL-193), obatoclax, HA14-1, oblimersen (Genasense®); (−)-Gossypol acetic acid (AT-101); ABT-737, and navitoclax.

In some embodiments, the Bcr/Abl kinase inhibitor is selected from imatinib (Gleevec®), inilotinib, nilotinib (Tasigna®), dasatinib (BMS-345825), bosutinib (SKI-606), ponatinib (AP24534), bafetinib (INNO406), danusertib (PHA-739358), AT9283, saracatinib (AZD0530), and PF-03814735.

In some embodiments, the checkpoint inhibitor is selected from ipilimumab (YERVOY®), pembrolizumab (KEYTRUDA®), nivolumab (OPDIVO®), cemiplimab (LIBTAYO®), atezolizumab (TECENTRIQ®), avelumab (BAVENCIO®), durvalumab (IMFINZI®), IMP701 (LAG525), CPI-444, MBG453, enoblituzumab, JNJ-61610588, and indoximod. See, e.g., Marin-Acevedo, et. al., J Hematol Oncol. 11:39 (2018).

In some embodiments, the other immunotherapy is an antibody therapy (e.g., a monoclonal antibody). In some embodiments, the antibody therapy is selected from bevacizumab (Mvasti™, Avastin®), trastuzumab (Herceptin®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), necitumumab (Portrazza™), cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab, and amatuximab.

In some embodiments, the other chemotherapeutic agents are selected from an anthracycline, an alkylating agent, a taxane, a platinum-based agent, eribulin (HALAVEN™), a farnesyl transferase inhibitor, a topoisomerase inhibitor, a DNA synthesis inhibitor, and cytotoxic agents.

In some embodiments, the taxane is selected from paclitaxel, docetaxel, cabazitaxel, abraxane, and taxotere.

In some embodiments, the anthracycline is selected from daunorubicin, doxorubicin, epirubicin, idarubicin, and combinations thereof.

In some embodiments, the platinum-based agent is selected from carboplatin, cisplatin, oxaliplatin, nedplatin, triplatin tetranitrate, phenanthriplatin, picoplatin, and satraplatin.

In some embodiments, the farnesyl transferase inhibitor is selected from lonafarnib, tipifarnib, BMS-214662, L778123, L744832, and FTI-277.

In some embodiments, the topoisomerase inhibitor is a topoisomerase I inhibitor (e.g., irinotecan (Camptosar®), topotecan (Hycamtin®), and 7-Ethyl-10-hydroxycampothecin (SN38)) or a topoisomerase II inhibitor (e.g., etoposide (Toposar®, VePesid®, and Etopophos®), teniposide (VM-26, Vumon®), and tafluposide.

In some embodiments, the DNA synthesis inhibitor is selected from capecitabine (Xeloda®), gemcitabine hydrochloride (Gemzar®), nelarabine (Arranon® and Atriance®), and sapacitabine.

In some embodiments, the alkylating agent is selected from temozolomide (Temodar® and Temodal®), dactinomycin (also known as actinomycin-D, Cosmegen®), melphalan (Alkeran®), altretamine (Hexalen®), carmustine (BiCNU®), bendamustine (Treanda®), busulfan (Busulfex® and Myleran®), lomustine (CeeNUR), chlorambucil (Leukeran®), cyclophosphamide (Cytoxan® and Neosar®), dacarbazine (DTIC-Dome®), altretamine (Hexalen®), ifosfamide (Ifex®), prednumustine, procarbazine (Matulane®), mechlorethamine (Mustargen®), streptozocin (Zanosar®), and thiotepa (Thioplex®).

In some embodiments, the cytotoxic agent is selected from bleomycin, cytarabine, dacarbazine, methotrexate, mitomycin C, pemetrexed, and vincristine.

Also provided herein is (i) a pharmaceutical combination for treating a cancer in a subject in need thereof, which comprises (a) a compound of Formula (I), or a pharmaceutically acceptable salt thereof, (b) at least one additional therapeutic agent (e.g., any of the exemplary additional therapeutic agents described herein or known in the art), and (c) optionally at least one pharmaceutically acceptable carrier for simultaneous, separate or sequential use for the treatment of cancer, wherein the amounts of the compound of Formula (I), or pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the cancer is a Ras pathway-associated cancer.

The term “pharmaceutical combination”, as used herein, refers to a pharmaceutical therapy resulting from the mixing or combining of more than one active ingredient and includes both fixed and non-fixed combinations of the active ingredients. The term “fixed combination” means that a compound of Formula (I), or a pharmaceutically acceptable salt thereof, and at least one additional therapeutic agent (e.g., a chemotherapeutic agent), are both administered to a subject simultaneously in the form of a single composition or dosage. The term “non-fixed combination” means that a compound of Formula (I), or a pharmaceutically acceptable salt thereof, and at least one additional therapeutic agent (e.g., chemotherapeutic agent) are formulated as separate compositions or dosages such that they may be administered to a subject in need thereof simultaneously, concurrently or sequentially with variable intervening time limits, wherein such administration provides effective levels of the two or more compounds in the body of the subject. These also apply to cocktail therapies, e.g., the administration of three or more active ingredients.

Accordingly, also provided herein is a method of treating a cancer, comprising administering to a subject in need thereof a pharmaceutical combination for treating cancer which comprises (a) a compound of Formula (I), or pharmaceutically acceptable salt thereof, and (b) an additional therapeutic agent, wherein the compound of Formula (I) and the additional therapeutic agent are administered simultaneously, separately or sequentially, wherein the amounts of the compound of Formula (I), or pharmaceutically acceptable salt thereof, and the additional therapeutic agent are together effective in treating the cancer. In some embodiments, the compound of Formula (I), or pharmaceutically acceptable salt thereof, and the additional therapeutic agent are administered simultaneously as separate dosages. In some embodiments, the compound of Formula (I), or pharmaceutically acceptable salt thereof, and the additional therapeutic agent are administered as separate dosages sequentially in any order, in jointly effective amounts, e.g., in daily or intermittently dosages. In some embodiments, the compound of Formula (I), or pharmaceutically acceptable salt thereof, and the additional therapeutic agent are administered simultaneously as a combined dosage. In some embodiments, the cancer is a Ras pathway-associated cancer.

Accordingly, also provided herein are methods for inhibiting, preventing, aiding in the prevention, or decreasing the symptoms of metastasis of a cancer in a subject in need thereof, the method comprising administering to the subject an effective amount of a compound of Formula (I), or a pharmaceutically acceptable salt thereof or a pharmaceutical composition thereof. Such methods can be used in the treatment of one or more of the cancers described herein. See, e.g., US Publication No. 2013/0029925; International Publication No. WO 2014/083567; and U.S. Pat. No. 8,568,998. See also, e.g., Hezam K et al., Rev Neurosci 2018 Jan. 26; 29:93-98; Gao L, et al., Pancreas 2015 January; 44:134-143; Ding K et al., J Biol Chem 2014 Jun. 6; 289:16057-71; and Amit M et al., Oncogene 2017 Jun. 8; 36:3232-3239. In some embodiments, the cancer is a Ras pathway-associated cancer. In some embodiments, the compound of Formula (I), or a pharmaceutically acceptable salt thereof is used in combination with an additional therapy or another therapeutic agent, such as those described herein.

The term “metastasis” is an art known term and means the formation of an additional tumor (e.g., a solid tumor) at a site distant from a primary tumor in a subject, where the additional tumor includes the same or similar cancer cells as the primary tumor.

Also provided are methods of decreasing the risk of developing a metastasis or an additional metastasis in a subject having a Ras pathway-associated cancer that include: selecting, identifying, or diagnosing a subject as having a Ras pathway-associated cancer, and administering an effective amount of a compound of Formula (I), or a pharmaceutically acceptable salt thereof to the subject selected, identified, or diagnosed as having a Ras pathway-associated cancer. Also provided are methods of decreasing the risk of developing a metastasis or an additional metastasis in a subject having a Ras pathway-associated cancer that includes administering an effective amount of a compound of Formula (I), or a pharmaceutically acceptable salt thereof to a subject having a Ras pathway-associated cancer. The decrease in the risk of developing a metastasis or an additional metastasis in a subject having a Ras pathway-associated cancer can be compared to the risk of developing a metastasis or an additional metastasis in the subject prior to treatment, or as compared to a subject or a population of subjects having a similar or the same Ras pathway-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a Ras-associated cancer that include: selecting, identifying, or diagnosing a subject as having a Ras-associated cancer, and administering an effective amount of a compound of Formula (I), or a pharmaceutically acceptable salt thereof to the subject selected, identified, or diagnosed as having a Ras-associated cancer. Also provided are methods of decreasing the risk of developing a metastasis or an additional metastasis in a subject having a Ras-associated cancer that includes administering an effective amount of a compound of Formula (I), or a pharmaceutically acceptable salt thereof to a subject having a Ras-associated cancer. The decrease in the risk of developing a metastasis or an additional metastasis in a subject having a Ras-associated cancer can be compared to the risk of developing a metastasis or an additional metastasis in the subject prior to treatment, or as compared to a subject or a population of subjects having a similar or the same Ras-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a KRas-associated cancer that include: selecting, identifying, or diagnosing a subject as having a KRas-associated cancer, and administering an effective amount of a compound of Formula (I), or a pharmaceutically acceptable salt thereof to the subject selected, identified, or diagnosed as having a KRas-associated cancer. Also provided are methods of decreasing the risk of developing a metastasis or an additional metastasis in a subject having a KRas-associated cancer that includes administering an effective amount of a compound of Formula (I), or a pharmaceutically acceptable salt thereof to a subject having a KRas-associated cancer. The decrease in the risk of developing a metastasis or an additional metastasis in a subject having a KRas-associated cancer can be compared to the risk of developing a metastasis or an additional metastasis in the subject prior to treatment, or as compared to a subject or a population of subjects having a similar or the same KRas-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a HRas-associated cancer that include: selecting, identifying, or diagnosing a subject as having a HRas-associated cancer, and administering an effective amount of a compound of Formula (I), or a pharmaceutically acceptable salt thereof to the subject selected, identified, or diagnosed as having a HRas-associated cancer. Also provided are methods of decreasing the risk of developing a metastasis or an additional metastasis in a subject having a HRas-associated cancer that includes administering an effective amount of a compound of Formula (I), or a pharmaceutically acceptable salt thereof to a subject having a HRas-associated cancer. The decrease in the risk of developing a metastasis or an additional metastasis in a subject having a HRas-associated cancer can be compared to the risk of developing a metastasis or an additional metastasis in the subject prior to treatment, or as compared to a subject or a population of subjects having a similar or the same HRas-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a NRas-associated cancer that include: selecting, identifying, or diagnosing a subject as having a NRas-associated cancer, and administering an effective amount of a compound of Formula (I), or a pharmaceutically acceptable salt thereof to the subject selected, identified, or diagnosed as having a NRas-associated cancer. Also provided are methods of decreasing the risk of developing a metastasis or an additional metastasis in a subject having a NRas-associated cancer that includes administering an effective amount of a compound of Formula (I), or a pharmaceutically acceptable salt thereof to a subject having a NRas-associated cancer. The decrease in the risk of developing a metastasis or an additional metastasis in a subject having a NRas-associated cancer can be compared to the risk of developing a metastasis or an additional metastasis in the subject prior to treatment, or as compared to a subject or a population of subjects having a similar or the same NRas-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

Also provided are methods of decreasing the risk of developing a metastasis or an additional metastasis in a subject having a SOS1-associated cancer that include: selecting, identifying, or diagnosing a subject as having a SOS1-associated cancer, and administering an effective amount of a compound of Formula (I), or a pharmaceutically acceptable salt thereof to the subject selected, identified, or diagnosed as having a SOS1-associated cancer. Also provided are methods of decreasing the risk of developing a metastasis or an additional metastasis in a subject having a SOS1-associated cancer that includes administering an effective amount of a compound of Formula (I), or a pharmaceutically acceptable salt thereof to a subject having a SOS1-associated cancer. The decrease in the risk of developing a metastasis or an additional metastasis in a subject having a SOS1-associated cancer can be compared to the risk of developing a metastasis or an additional metastasis in the subject prior to treatment, or as compared to a subject or a population of subjects having a similar or the same SOS1-associated cancer that has received no treatment or a different treatment. In some embodiments, the additional therapeutic agent is selected from MRTX849, LY3499446, JNJ-74699157, AMG 510, ARS3248, ARS853, ARS1620, AZD4785, JNJ-74699157, SML-8-73-1, SML-10-70-1, VSA9, AA12, and MRTX-849. In some embodiments, the subject has been administered one or more doses of a compound of Formula (I), or a pharmaceutically acceptable salt thereof, prior to administration of the pharmaceutical composition.

The phrase “risk of developing a metastasis” means the risk that a subject having a primary tumor will develop an additional tumor (e.g., a solid tumor) at a site distant from a primary tumor in a subject over a set period of time, where the additional tumor includes the same or similar cancer cells as the primary tumor. Methods for reducing the risk of developing a metastasis in a subject having a cancer are described herein.

The phrase “risk of developing additional metastases” means the risk that a subject having a primary tumor and one or more additional tumors at sites distant from the primary tumor (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

Treatment of a subject having a cancer with a multi-kinase inhibitor (MKI) or target-specific kinase inhibitor (e.g., a BRAF inhibitor, an EGFR inhibitor, a MEK inhibitor, an ALK inhibitor, a ROS1 inhibitor, a MET inhibitor, an aromatase inhibitor, a RAF inhibitor, a RET inhibitor, or a RAS inhibitor) can result in dysregulation of a Ras pathway gene, a Ras pathway protein, or the expression or activity or level of the same in the cancer. See, e.g., Bhinge et al., Oncotarget 8:27155-27165, 2017; Chang et al., Yonsei Med. J. 58:9-18, 2017; and Lopez-Delisle et al., doi: 10.1038/s41388-017-0039-5, Oncogene 2018.

Treatment of a subject having a cancer with a SOS1 inhibitor in combination with a multi-kinase inhibitor or a target-specific kinase inhibitor (e.g., a BRAF inhibitor, an EGFR inhibitor, a MEK inhibitor, an ALK inhibitor, a ROS1 inhibitor, a MET inhibitor, an aromatase inhibitor, a RAF inhibitor, a RET inhibitor, or a RAS inhibitor) can have increased therapeutic efficacy as compared to treatment of the same subject or a similar subject with the SOS1 inhibitor as a monotherapy, or the multi-kinase inhibitor or the target-specific kinase inhibitor as a monotherapy. See, e.g., Tang et al., doi: 10.1038/modpathol.2017.109, Mod. Pathol. 2017; Andreucci et al., Oncotarget 7:80543-80553, 2017; Nelson-Taylor et al., Mol. Cancer Ther. 16:1623-1633, 2017; and Kato et al., Clin. Cancer Res. 23:1988-1997, 2017.

Provided herein are methods of treating a subject having a cancer (e.g., any of the cancers described herein) and previously administered a multi-kinase inhibitor (MKI) or a target-specific kinase inhibitor (e.g., a Ras inhibitor, a BRAF inhibitor, an EGFR inhibitor, a MEK inhibitor, an ALK inhibitor, a ROS1 inhibitor, a MET inhibitor, an aromatase inhibitor, a RAF inhibitor, a RET inhibitor, or a RAS inhibitor) (e.g., as a monotherapy) that include: administering to the subject (i) an effective dose of a compound of Formula (I), or a pharmaceutically acceptable salt thereof as a monotherapy, or (ii) an effective dose of a compound of Formula (I), or a pharmaceutically acceptable salt thereof, and an effective dose of the previously administered MKI or the previously administered target-specific kinase inhibitor.

Also provided is a method for inhibiting SOS1 activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having SOS1 activity. In some embodiments, the mammalian cell is a mammalian cancer cell. In some embodiments, the mammalian cancer cell is any cancer as described herein. In some embodiments, the mammalian cancer cell is a Ras pathway-associated cancer cell.

Also provided is a method for inhibiting Ras activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having Ras activity. In some embodiments, the mammalian cell is a mammalian cancer cell. In some embodiments, the mammalian cancer cell is any cancer as described herein. In some embodiments, the mammalian cancer cell is a Ras pathway-associated cancer cell.

Also provided is a method for inhibiting a SOS1-Ras (e.g., KRas, HRas, and/or NRas) protein-protein interaction in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having a SOS1-Ras (e.g., KRas, HRAs, and/or NRas) protein-protein interaction. In some embodiments, the mammalian cell is a mammalian cancer cell. In some embodiments, the mammalian cancer cell is any cancer as described herein. In some embodiments, the mammalian cancer cell is a Ras pathway-associated cancer cell.

Also provided is a method for inhibiting Ras pathway activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having Ras pathway activity. In some embodiments, the mammalian cell is a mammalian cancer cell. In some embodiments, the mammalian cancer cell is any cancer as described herein. In some embodiments, the mammalian cancer cell is a Ras pathway-associated cancer cell.

As used herein, the term “contacting” refers to the bringing together of indicated moieties in an in vitro system or an in vivo system. For example, “contacting” a SOS1 protein with a compound provided herein includes the administration of a compound provided herein to a subject, such as a human, having a SOS1 protein, as well as, for example, introducing a compound provided herein into a sample containing a mammalian cellular or purified preparation containing the SOS1 protein.

Also provided herein is a method of inhibiting mammalian cell proliferation, in vitro or in vivo, the method comprising contacting a mammalian cell with an effective amount of a compound of Formula (I), or a pharmaceutically acceptable salt thereof, or a pharmaceutical composition thereof as defined herein.

The phrase “effective amount” means an amount of compound that, when administered to a subject in need of such treatment, is sufficient to (i) treat a Ras pathway-associated disease or disorder (such as a Ras pathway-associated cancer), (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I), or a pharmaceutically acceptable salt thereof that will correspond to such an amount will vary depending upon factors such as the particular compound, disease condition and its severity, the identity (e.g., weight) of the subject in need of treatment, but can nevertheless be routinely determined by one skilled in the art.

### Pharmaceutical Compositions

When employed as pharmaceuticals, compounds of Formula (I), including pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula (I) or pharmaceutically acceptable salt thereof, in combination with one or more pharmaceutically acceptable excipients. For example, a pharmaceutical composition prepared using a compound of Formula (I) or a pharmaceutically acceptable salt thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula (I) or a pharmaceutically acceptable salt thereof with a pharmaceutically acceptable carrier. Pharmaceutical compositions containing a compound of Formula (I) or a pharmaceutically acceptable salt thereof as the active ingredient can be prepared by intimately mixing the compound of Formula (I), or a pharmaceutically acceptable salt thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing the compositions in oral dosage form, any of the usual pharmaceutical media can be employed. Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Solid oral preparations can also be coated with substances such as sugars or be enteric-coated so as to modulate major site of absorption. For parenteral administration, the carrier will usually consist of sterile water and other ingredients can be added to increase solubility or preservation. Injectable suspensions or solutions can also be prepared utilizing aqueous carriers along with appropriate additives. The pharmaceutical compositions herein will contain, per dosage unit, e.g., tablet, capsule, powder, injection, teaspoonful and the like, an amount of the active ingredient necessary to deliver an effective dose as described herein.

The compositions comprising a compound of Formula (I) or a pharmaceutically acceptable salt thereof can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formula (I) or a pharmaceutically acceptable salt thereof) calculated to produce the desired therapeutic effect, in association with a suitable pharmaceutical excipient.

In some embodiments, the compositions provided herein contain from about 5 mg to about 50 mg of the active ingredient.

In some embodiments, the compositions provided herein contain from about 50 mg to about 500 mg of the active ingredient. In some embodiments, the compositions provided herein contain about 10 mg, about 20 mg, about 80 mg, or about 160 mg of the active ingredient.

In some embodiments, the compositions provided herein contain from about 500 mg to about 1,000 mg of the active ingredient.

The daily dosage of the compound of Formula (I) or a pharmaceutically acceptable salt thereof can be varied over a wide range from 1.0 to 10,000 mg per adult human per day, or higher, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 160, 200, 250 and 500 milligrams of the active ingredient for the symptomatic adjustment of the dosage to the subject to be treated. An effective amount of the drug is ordinarily supplied at a dosage level of from about 0.1 mg/kg to about 1000 mg/kg of body weight per day, or any range therein. Preferably, the range is from about 0.5 to about 500 mg/kg of body weight per day, or any range therein. In an example, the range can be from about 0.1 to about 50.0 mg/kg of body weight per day, or any amount or range therein. In another example, the range can be from about 0.1 to about 15.0 mg/kg of body weight per day, or any range therein. In yet another example, the range can be from about 0.5 to about 7.5 mg/kg of body weight per day, or any amount to range therein. Pharmaceutical compositions containing a compound of Formula (I) or a pharmaceutically acceptable salt thereof can be administered on a regimen of 1 to 4 times per day or in a single daily dose.

The active compound may be effective over a wide dosage range and is generally administered in a pharmaceutically effective amount. Optimal dosages to be administered can be readily determined by those skilled in the art. It will be understood, therefore, that the amount of the compound actually administered will usually be determined by a physician, and will vary according to the relevant circumstances, including the mode of administration, the actual compound administered, the strength of the preparation, the condition to be treated, and the advancement of the disease condition. In addition, factors associated with the particular subject being treated, including subject response, age, weight, diet, time of administration and severity of the subject's symptoms, will result in the need to adjust dosages.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. In some embodiments, such administration can be once-daily or twice-daily (BID) administration.

One skilled in the art will recognize that both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy subjects and/or those suffering from a given disorder, can be completed according to methods well known in the clinical and medical arts.

Provided herein are pharmaceutical kits useful, for example, in the treatment of Ras pathway-associated diseases or disorders, such as cancer, which include one or more containers containing a pharmaceutical composition comprising an effective amount of a compound provided herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74 (11), 1297 (1997).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) (“Preparative LC-MS Purification: Improved Compound Specific Method Optimization” K. F. Blom, et al., J. Combi. Chem. 6 (6), 874 (2004), normal phase silica chromatography, and supercritical fluid chromatography (SFC).

All solvents and reagents were obtained from commercial sources and used without further purification unless indicated otherwise. Anhydrous solvents were purchased and used as supplied. Reactions were monitored by thin-layer chromatography (TLC), visualizing with a UV lamp (254 nm) and KMnO4 stain. NMR spectra were obtained on a Bruker Neo 400M spectrometer operating at 400 MHz. Chemical shifts are reported in parts per million (8) from the tetramethysilane resonance in the indicated solvent. LC-Mass spectra were taken with Agilent 1260-6125B single quadrupole mass spectrometer using a Welch Biomate column (C18, 2.7 μm, 4.6*50 mm) or waters H-Class SQD2 system. The detection was by DAD (254 nm and 210 nm and 280 nm). Chiral HPLC was performed on the Waters acquity UPC2 system under base-containing on Daicel chiralpak AD-H (5 μm, 4.6*250 mm), Daicel chiralpak OD-H (5 μm, 4.6*250 mm), Daicel chiralpak IG-3 (3 μm, 4.6*150 mm), Chiral Technologies Europe AD-3 (3 μm, 3.0*150 mm) and Trefoil™ Technology Trefoil™ AMY1 (2.5 μm, 3.0*150 mm). The detection was by DAD (254 nm). Preparative HPLC was performed on GILSON Trilution LC system using a Welch XB—C18 column (5 μm, 21.2*150 mm). Flash chromatography was carried out on Biotage Isolera Prime system using Welch WelFlash flash columns (40-63 μm). The compounds synthesized are all with purity ≥95% unless otherwise specified.

### Abbreviations

° C.=degrees Celsius
¹H NMR=proton nuclear magnetic resonance spectrum
ACN=acetonitrile
AcOH=acetic acid
Boc=tert-butoxycarbonyl
con.=concentrated
d=doublet
DAST=diethylaminosulfur trifluoride
DBU=1,8-diazabicyclo[5.4.0]undec-7-ene
DCM=dichloromethane
DIPEA=N,N-diisopropylethylamine
DMF=N,N-dimethylformamide
DMF-DMA=dimethylformamide dimethyl acetal
DMSO=dimethylsulfoxide
EtOAc and EA=ethyl acetate
EtOH=ethanol
ESI=electrospray ionization
g=gram(s)
hr=hour(s)
HATU=1-[Bis(dimethylamino)methylene]-1H-1,2,3-triazolo[4,5-b]pyridinium 3-oxide hexafluorophosphate
HPLC=high-performance liquid chromatography
IPA=2-propanol
LCMS=liquid chromatograph-mass spectrum
M=mass
m/z=mass-to-charge ratio
MeCN=acetonitrile
MeOH=methanol
MeONa=sodium methoxide
mg=milligram(s)
mL=milliliter
mmol=millimole(s)
mol=mole(s)
MS=mass spectrum
NaBH₃CN=sodium cyanoborohydride
NBS=N-bromosuccinimide
Ni(dtbbpy)Br₂=(2,2′-bipyridine) nickel (II) dibromide
obsd.=observed
Pd(OAc)₂=palladium (II) acetate
Pd(dppf)Cl₂=(1,1′-Bis(diphenylphosphino) ferrocene) palladium (II) dichloride
Pd₂(dba)₃=Tris (dibenzylideneacetone) dipalladium (0)
PE=petroleum ether
ppm=parts per million
rt=room temperature
RuPhos=2-Dicyclohexylphosphino-2′,6′-diisopropoxybiphenyl
s=singlet
S-Phos=Dicyclohexyl(2′,6′-dimethoxy[1,1′-biphenyl]-2-yl)phosphane
Tf=trifluoromethanesulfonate
T3P=Propylphosphonic anhydride
t=triplet
TBAF=tetrabutylammonium fluoride
TEA=triethylamine
TFA=trifluoroacetic acid
THF=tetrahydrofuran
TLC=thin-layer chromatography
XantPhos=(9,9-Dimethyl-9H-xanthene-4,5-diyl)bis(diphenylphosphane)
XantPhos-Pd-G3=[(4,5-Bis(diphenylphosphino)-9,9-dimethylxanthene)-2-(2′-amino-1,1′-biphenyl)]palladium (II) methanesulfonate
XPhos=Dicyclohexyl[2′,4′,6′-tris(propan-2-yl)[1,1′-biphenyl]-2-yl]phosphane
XPhos-Pd-G3=(2-Dicyclohexylphosphino-2′,4′,6′-triisopropyl-1,1′-biphenyl) [2-(2′-amino-1,1′-biphenyl)]palladium (II) methanesulfonate

### EXAMPLES

### Example 1: (R)—N-(1-(3-(difluoromethyl)-2-fluorophenyl)ethyl)-4-fluoro-2-((1-methylpiperidin-4-yl)amino)-5-morpholinobenzamide

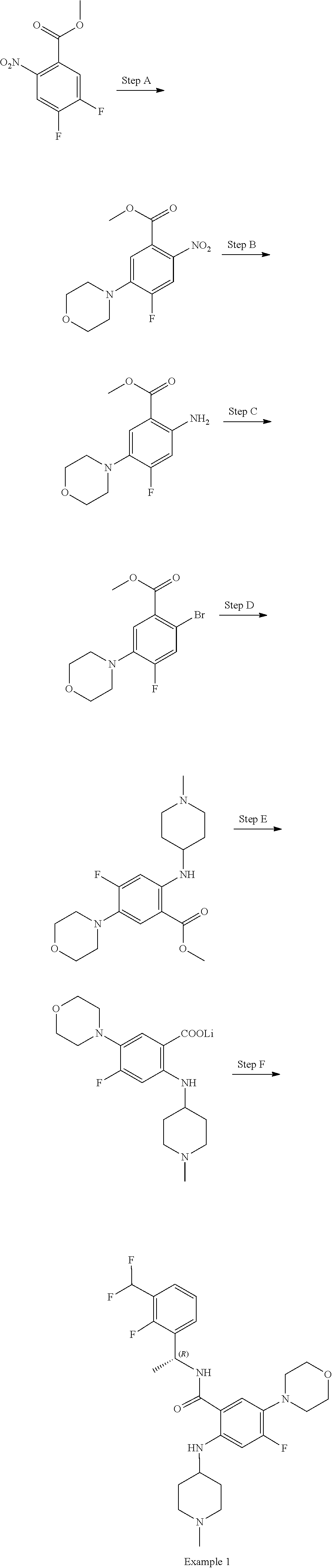

### Step A: methyl 4-fluoro-5-morpholino-2-nitrobenzoate

To a solution of methyl 4,5-difluoro-2-nitro-benzoate (500 mg, 2.30 mmol) and morpholine (301 mg, 3.45 mmol) in THF (10 mL) was added triethylamine (699 mg, 6.91 mmol) at 0° C. The reaction was stirred for 16 h at 0° C. The solvent was removed in vacuo and the residue was purified by flash column chromatography (eluting with 0%-50% EtOAc in PE) to afford the title compound (600 mg, 91% yield). MS obsd. (ESI⁺): 285.2 [(M+H)⁺].

### Step B: methyl 2-amino-4-fluoro-5-morpholinobenzoate

To a solution of methyl 4-fluoro-5-morpholino-2-nitrobenzoate (600 mg, 2.11 mmol) in MeOH (15 mL) was added Pd/C (200 mg, 1.65 mmol, 10 wt %). The reaction was stirred for 16 hr at rt under H₂ atmosphere (1 atm). The mixture was filtered and the filtrate was concentrated in vacuo. The residue was purified by flash column chromatography (eluting with 0%-50% EtOAc in PE) to afford the title compound (530 mg, 87% yield). MS obsd. (ESI⁺): 255.2 [(M+H)⁺].

### Step C: methyl 2-bromo-4-fluoro-5-morpholinobenzoate

To a solution of methyl 2-amino-4-fluoro-5-morpholinobenzoate (200 mg, 0.79 mmol) in acetonitrile (15 mL) was added CuBr (169 mg, 1.18 mmol) and isopentyl nitrite (138 mg, 1.18 mmol). The reaction was stirred for 1 hr at 60° C. The mixture was filtered and the filtrate was concentrated in vacuo. The residue was purified by flash column chromatography (eluting with 0%-50% EtOAc in PE) to afford the title compound (150 mg, 60% yield). MS obsd. (ESI⁺): 79Br/81Br 318.2, 320.2 [(M+H)⁺].

### Step D: methyl 4-fluoro-2-((1-methylpiperidin-4-yl)amino)-5-morpholinobenzoate

To a solution of methyl 2-bromo-4-fluoro-5-morpholinobenzoate (100 mg, 0.31 mmol) in dioxane (0.5 mL) was added 1-methylpiperidin-4-amine (72 mg, 0.63 mmol), XantPhos-Pd-G3 (60 mg, 0.06 mmol) and cesium carbonate (307 mg, 0.94 mmol). The mixture was stirred at 100° C. for 16 hr. The mixture was filtered and the filtrate was concentrated in vacuo. The residue was purified by flash column chromatography (eluting with 0%-5% MeOH in DCM) to afford the title compound (101 mg, 77% yield). MS obsd. (ESI⁺): 352.3 [(M+H)⁺].

### Step E: lithium 4-fluoro-2-((1-methylpiperidin-4-yl)amino)-5-morpholinobenzoate

To a solution of methyl 4-fluoro-2-((1-methylpiperidin-4-yl)amino)-5-morpholinobenzoate (101 mg, 0.29 mmol) in THF (4 mL) and H₂O (1 mL) was added lithium hydroxide (14 mg). The reaction was stirred for 16 hr at 45° C. The solvent was removed in vacuo to afford the title compound (100 mg, crude) as a white solid. The crude product was used for the next step without further purification. MS obsd. (ESI⁺): 338.3 [(M+H)⁺].

### Step F: (R)—N-(1-(3-(difluoromethyl)-2-fluorophenyl)ethyl)-4-fluoro-2-((1-methylpiperidin-4-yl)amino)-5-morpholinobenzamide (example 1)

To a solution of lithium 4-fluoro-2-((1-methylpiperidin-4-yl)amino)-5-morpholinobenzoate (100 mg, crude) in DMF (5 mL) was added HATU (133 mg, 0.35 mmol). The reaction was stirred for 10 mins at rt. Then (1R)-1-[3-(difluoromethyl)-2-fluoro-phenyl]ethanamine; hydrochloride (85 mg, 0.38 mmol) and DIPEA (113 mg, 0.87 mmol) was added and the reaction was stirred for 2 h at rt. The mixture was diluted with water and extracted with (3×20 mL) and the combined organic layers were dried over Na₂SO₄. The solvent was removed in vacuo and the residue was purified by flash column chromatography (eluting with 0%-50% MeOH in DCM) followed by preparative HPLC (ACN/water/0.1% NH₄HCO₃) to afford the title compound (55.5 mg, 36% yield). MS obsd. (ESI⁺): 509.3 [(M+H)⁺]. ¹H NMR (400 MHZ, DMSO-d6) δ ppm 8.68 (1H), 7.69 (1H), 7.63 (1H), 7.52 (1H), 7.41 (1H), 7.38-7.33 (1H), 7.22 (1H), 6.54 (1H), 5.36 (1H), 3.78-3.69 (4H), 3.27-3.21 (1H), 2.97-2.90 (4H), 2.58-2.52 (2H), 2.12 (3H), 2.10-2.00 (2H), 1.85-1.79 (2H), 1.49 (3H), 1.37-1.23 (2H).

### Example 2: 5-((1R,4R)-2-oxa-5-azabicyclo[2.2.1]heptan-5-yl)-N—((R)-1-(3-(difluoromethyl)-2-fluorophenyl)ethyl)-4-fluoro-2-((1-methylpiperidin-4-yl)amino)benzamide

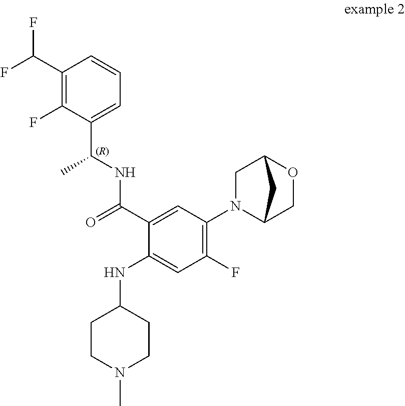

Prepared according to an analogous route as example 1. MS obsd. (ESI⁺): 521.5 [(M+H)⁺]. ¹H NMR (400 MHZ, DMSO-d6) δ: 8.67 (1H), 7.61 (1H), 7.51 (1H), 7.38-7.1 (4H), 6.54 (1H), 5.36 (1H), 4.55 (1H), 4.44 (1H), 3.82 (1H), 3.74 (1H), 3.47 (1H), 3.25-3.17 (1H), 3.08 (1H), 2.58 (1H), 2.14 (3H), 2.06 (2H), 1.85 (4H), 1.48 (3H), 1.35-1.24 (2H).

### Example 3: (R)-4-fluoro-N-(1-(2-methyl-3-(trifluoromethyl)phenyl)ethyl)-2-((1-methylpiperidin-4-yl)amino)-5-morpholinobenzamide

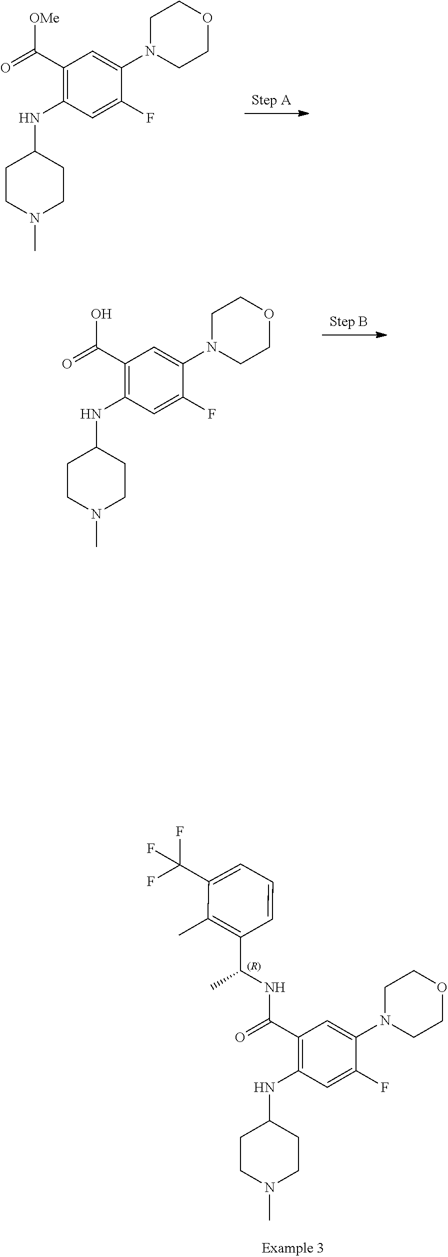

### Step A: 4-fluoro-2-((1-methylpiperidin-4-yl)amino)-5-morpholinobenzoic acid

To a solution of methyl 4-fluoro-2-[(1-methyl-4-piperidyl)amino]-5-morpholino-benzoate (130 mg, 0.37 mmol) in MeOH (1 mL) and THF (1 mL) was added a solution of LiOH (26.5 mg, 1.11 mmol) in H₂O (1 mL) and the mixture was stirred at 40° C. for 12 hr. The mixture was adjusted to pH 5˜6 with diluted hydrochloric acid. The resulting mixture was extracted with EtOAc (3×10 mL). The organic layers were combined, dried over anhydrous sodium sulfate, filtered and concentrated to afford the title compound (100 mg, 80% yield). MS obsd. (ESI⁺): 338.15 [M+H]⁺.

### Step B: (R)-4-fluoro-N-(1-(2-methyl-3-(trifluoromethyl)phenyl)ethyl)-2-((1-methylpiperidin-4-yl)amino)-5-morpholinobenzamide (example 3)

To a solution of 4-fluoro-2-((1-methylpiperidin-4-yl)amino)-5-morpholinobenzoic acid (80 mg, 0.24 mmol) in DMF (2 mL) was added HATU (90.1 mg, 0.24 mmol), DIPEA (76 mg, 0.59 mmol) and (1R)-1-[2-methyl-3-(trifluoromethyl)phenyl]ethanamine (48.2 mg, 0.24 mmol) and the mixture stirred for 2 hr at 20° C. The crude mixture was purified by Preparative HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 50% B to 70% B in 7 min, 70% B) to afford the title compound (21.1 mg, 17% yield). 19F NMR (400 MHZ, DMSO-d6) δ−58.713, −117.110. MS obsd. (ESI): 521.15 [M−H]⁻.

### Example 4. (R)-4-cyano-N-(1-(3-(difluoromethyl)-2-fluorophenylethyl)-2-((1-

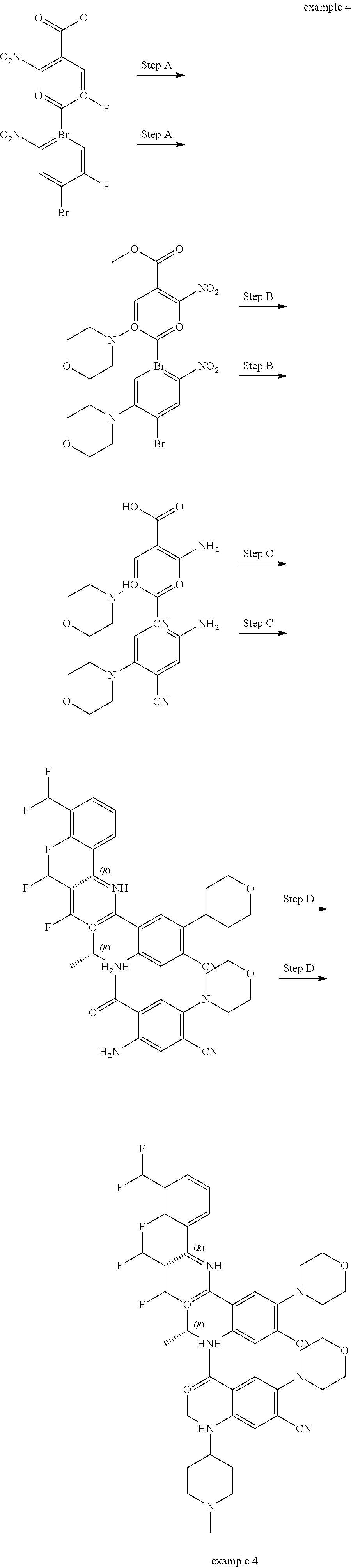

### Step A: methyl 4-bromo-5-morpholino-2-nitrobenzoate

To a mixture of methyl 4-bromo-5-fluoro-2-nitrobenzoate (2.0 g, 7.19 mmol) in CH₃CN (15 mL) was added triethylamine (2.18 g, 21.58 mmol) and morpholine (940 mg, 10.79 mmol). The mixture was stirred at rt for 3 hr. The mixture was diluted with EtOAc (30 mL), and washed with 1 N HCl (15 mL×3) and H₂O (15 mL×3). The organic layer was dried (Na₂SO₄), filtered, and concentrated to provide the title compound (2.2 g, 88% yield), which was used without further purification. MS obsd. (ESI⁺): ⁷⁹Br/⁸¹Br 345.2, 347.2 [(M+H)⁺].

### Step B: 2-amino-4-cyano-5-morpholinobenzoic acid

To a mixture of methyl 4-bromo-5-morpholino-2-nitrobenzoate (1.2 g, 3.48 mmol) in DMF (12 mL) was added zinc cyanide (816 mg, 6.95 mmol), zinc powder (227 mg, 3.48 mmol), triethylamine (1.06 g, 10.43 mmol) and 1,1′-Bis(diphenylphosphino) ferrocene-palladium (II) dichloride dichloromethane complex (564 mg, 0.69 mmol). The mixture was stirred at 120° C. for 16 hr under N₂ atmosphere. The mixture was diluted with MeOH (50 mL) and filtered through a celite pad which was washed with MeOH (20 mL×3). The filtrate was concentrated and the residue was partitioned between EtOAc and H₂O. The aqueous layer was concentrated to afford the title compound (630 mg, crude). This material was used without further purification. MS obsd. (ESI⁺): 248.0 [(M+H)⁺].

### Step C: (R)-2-amino-4-cyano-N-(1-(3-(difluoromethyl)-2-fluorophenyl)ethyl)-5-morpholinobenzamide

To a mixture of 2-amino-4-cyano-5-morpholino-benzoic acid (310 mg, crude) in DMF (15 mL) was added HATU (715 mg, 1.88 mmol), DIPEA (486 mg, 3.76 mmol) and (R)-1-(3-(difluoromethyl)-2-fluorophenyl) ethan-1-amine hydrochloride (339 mg, 1.50 mmol). The mixture was stirred at rt for 3 hr. The mixture was diluted with EtOAc (60 mL) and washed with H₂O (45 mL×3) and brine (45 mL×3). The organic layer was dried (Na₂SO₄), concentrated, and purified by silica gel chromatography column (PE:EtOAc=1:1) to provide the title compound (101 mg). MS obsd. (ESI⁺): 419.0 [(M+H)⁺].

### Step D: (R)-4-cyano-N-(1-(3-(difluoromethyl)-2-fluorophenyl)ethyl)-2-((1-methylpiperidin-4-yl)-amino)-5-morpholinobenzamide (example 4)

To a solution of (R)-2-amino-4-cyano-N-(1-(3-(difluoromethyl)-2-fluorophenyl)ethyl)-5-morpholinobenzamide (91 mg, 0.22 mmol) in acetic acid (6 mL) was added 1-methylpiperidin-4-one (123 mg, 1.09 mmol), and sodium triacetoxyborohydride (230 mg, 1.09 mmol). The mixture was stirred at 25° C. for 3 hr. The mixture was concentrated and the residue was diluted with DCM (20 mL). The mixture was washed with saturated NaHCO₃ followed by H₂O and brine. The organic layer was dried (Na₂SO₄), concentrated, and purified by preparative HPLC (ACN/water/0.1NH₄HCO₃) to provide the title compound (28.3 mg, 25% yield). MS obsd. (ESI⁺): 516.4 [(M+H)⁺]. ¹H NMR (400 MHZ, DMSO-d6) δ 8.98 (1H), 7.64 (1H), 7.53 (1H), 7.45 (s, 1H), 7.39-7.07 (4H), 5.38 (1H), 3.78 (m, 4H), 3.42-3.33 (1H), 3.06-2.97 (4H), 2.55 (2H), 2.13 (3H), 2.07 (2H), 1.83 (2H), 1.50 (3H), 1.36-1.21 (2H).

### Example 5: (R)—N-(1-(3-(difluoromethyl)-2-fluorophenyl)ethyl)-4-fluoro-5-(4-methylpiperazin-1-yl)-2-((1-methylpiperidin-4-yl)amino)benzamide

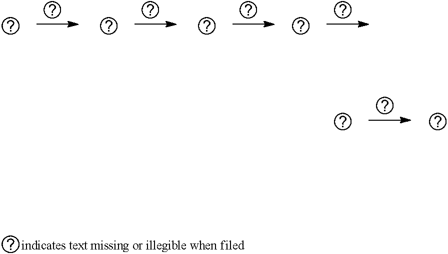

### Step A: Methyl 4-fluoro-5-(4-methylpiperazin-1-yl)-2-nitrobenzoate

To a solution of methyl 4,5-difluoro-2-nitrobenzoate (500 mg, 2.30 mmol) and 1-methylpiperazine (300 mg, 2.3 mmol) in THF (15 mL) was added Et₃N (3.45 mmol, 4.73 mL) at 0° C., then the mixture was stirred for 16 hr at 0° C. The reaction solution was concentrated in vacuo, and the residue was purified by silica gel column chromatography (eluting with 10% MeOH in DCM) to afford the title compound. MS obsd. (ESI⁺): 298.1 [(M+H)]⁺.

### Step B: Methyl 2-amino-4-fluoro-5-(4-methylpiperazin-1-yl)benzoate

To a solution of methyl 4-fluoro-5-(4-methylpiperazin-1-yl)-2-nitrobenzoate (605 mg, 2.04 mmol) in MeOH (12 mL) was added Pd/C (10 wt %, 200 mg), and the mixture was stirred for 1 hr at 40° C. under H₂ (1 atm). The mixture was filtered through a layer of Celite. The filtrate was concentrated in vacuo. Then the residue was purified by silica gel chromatography (eluted with 5% MeOH in DCM) to afford the title compound. MS obsd. (ESI⁺): 268.1 [(M+H)]⁺.

### Step C: 2-amino-4-fluoro-5-(4-methylpiperazin-1-yl)benzoic acid

To a solution of methyl 2-amino-4-fluoro-5-(4-methylpiperazin-1-yl)benzoate (285 mg, 1.07 mmol) in THF (12 mL) and MeOH (3 mL) was added LiOH (1M in water, 7 mL). The reaction was stirred for 2 hr at 60° C. The solvent was removed in vacuo, then acidified with 2 M HCl (3.5 mL) to pH=3. The mixture was concentrated in vacuo to afford the title compound (725 mg, crude), which was used without further purification. MS obsd. (ESI⁺): 254.1 [(M+H)]⁺.

### Step D: (R)-2-amino-N-(1-(3-(difluoromethyl)-2-fluorophenyl)ethyl)-4-fluoro-5-(4-methylpiperazin-1-yl)benzamide

To a solution of methyl 2-amino-4-fluoro-5-(4-methylpiperazin-1-yl)benzoic acid (725 mg, crude) in DMF (6 mL) was added HATU (610 mg, 1.61 mmol), (1R)-1-[3-(difluoromethyl)-2-fluoro-phenyl]ethanamine (202 mg, 1.07 mmol) and DIPEA (415 mg, 3.21 mmol). The reaction was stirred for 2 h at rt. Then the mixture was poured into water (40 mL) and extracted with EtOAc (3×30 mL). The combined organic layers were dried over Na₂SO₄, filtered and concentrated. The residue was purified by silica gel column chromatography (eluting with 3% MeOH in DCM) to afford the title compound (447 mg). MS obsd. (ESI⁺): 425.2 [(M+H)]⁺.

### Step E: (R)—N-(1-(3-(difluoromethyl)-2-fluorophenyl)ethyl)-4-fluoro-5-(4-methylpiperazin-1-yl)-2-((1-methylpiperidin-4-yl)amino)benzamide (example 5)

To a solution of (R)-2-amino-N-(1-(3-(difluoromethyl)-2-fluorophenyl)ethyl)-4-fluoro-5-(4-methylpiperazin-1-yl)benzamide (70 mg, 0.16 mmol) in AcOH (5 mL) was added 1-methylpiperidin-4-one (187 mg, 1.65 mmol) and NaBH₃CN (41 mg, 0.66 mmol). The reaction was stirred for 4 hr at 40° C. The mixture was concentrated in vacuo, then the residue was neutralized with aqueous NaHCO₃ and extracted with DCM (3×10 mL). The combined organic layers were dried over sodium sulfate, filtered and concentrated. The residue was purified by silica gel column chromatography (eluting with 3% MeOH in DCM) followed by reverse phase chromatography (C18, eluting with 30% MeCN in 0.1% NH₄HCO₃—H₂O) to afford the title compound (31 mg, 59.43 μmol, 36% yield). MS obsd. (ESI⁺): 522.3 [(M+H)]⁺. ¹H NMR (400 MHZ, DMSO-d₆) δ: 8.68 (1H), 7.63 (2H), 7.51 (1H), 7.44-7.33 (2H), 7.21 (1H), 6.52 (1H), 5.36 (1H), 3.23 (1H), 2.95 (4H), 2.57 (2H), 2.47 (4H), 2.23 (3H), 2.13 (3H), 2.06 (2H), 1.82 (2H), 1.48 (3H), 1.35-1.23 (2H).

### Example 6:5-(6-oxa-3-azabicyclo[3.1.1]heptan-3-yl)-N—((R)-1-(3-(difluoromethyl)-2-fluorophenyl)ethyl)-4-fluoro-2-((1-methylpiperidin-4-yl)amino)benzamide

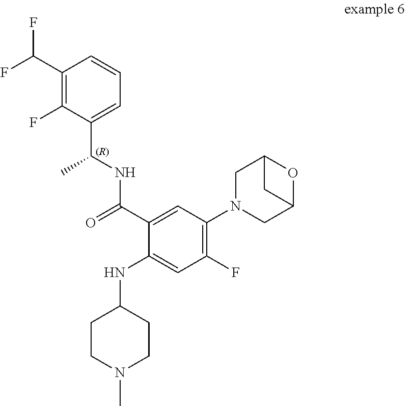

Synthesized according to an analogous route to example 5. MS obsd. (ESI⁺): 521.5 [(M+H)]⁺. ¹H NMR (400 MHZ, DMSO) δ: 8.76 (1H), 7.65 (1H), 7.59-7.46 (3H), 7.39-7.33 (1H), 7.22 (1H), 6.57 (1H), 5.42-5.32 (1H), 4.60 (2H), 3.55-3.42 (4H), 3.30-3.27 (1H), 3.04 (1H), 2.82-2.61 (m, 2H), 2.35-2.08 (6H), 1.94-1.80 (2H), 1.49 (3H), 1.43-1.29 (2H).

### Example 7: (R)—N-(1-(3-(difluoromethyl)-2-fluorophenyl)ethyl)-5-(6,7-dihydropyrazolo[1,5-a]pyrazin-5 (4H)-yl)-4-fluoro-2-((1-methylpiperidin-4-yl)amino)benzamide

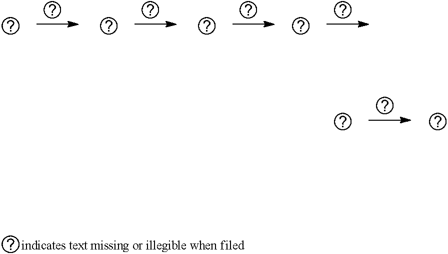

### Step A: Methyl 5-(6,7-dihydropyrazolo[1,5-a]pyrazin-5 (4H)-yl)-4-fluoro-2-nitrobenzoate

To a solution of 4,5,6,7-tetrahydropyrazolo[1,5-a]pyrazine (162 mg, 0.83 mmol) and methyl 4,5-difluoro-2-nitrobenzoate (150 mg, 0.69 mmol) in DMSO (2 mL) was added triethylamine (279 mg, 2.76 mmol). The reaction was stirred at 30° C. for 16 hr. The mixture was diluted with saturated aqueous NaHCO₃ and extracted with EtOAc (2×20 mL). The combined organic layers were washed with brine (2×30 mL), dried over Na₂SO₄, filtered and concentrated. The residue was purified by flash column chromatography (eluting with 50% EtOAc in PE) to afford the target compound (162 mg, 73% yield). MS obsd. (ESI⁺): 321.2, [M+H]⁺.

### Step B: Methyl 2-amino-5-(6,7-dihydropyrazolo[1,5-a]pyrazin-5 (4H)-yl)-4-fluorobenzoate

To a solution of methyl 5-(6,7-dihydropyrazolo[1,5-a]pyrazin-5 (4H)-yl)-4-fluoro-2-nitrobenzoate (162 mg, 505.81 μmol) in acetic acid (4 mL) was added Fe (283 mg, 5.06 mmol). The reaction was stirred at 50° C. for 1 hr. The mixture was filtered and the solvent was removed under vacuum. The residue was dissolved in water and basified with NaHCO₃ (aq.). The mixture was extracted with EtOAc (2×15 mL). The organic layers were washed with brine (2×20 mL), dried over sodium sulfate, and concentrated. The residue was purified by flash column chromatography (eluting with 0%-5% MeOH in DCM) to afford the title compound (154 mg, crude). This material was used without further purification. MS obsd. (ESI⁺): 291.3, [M+H]⁺.

### Step C: Lithium 2-amino-5-(6,7-dihydropyrazolo[1,5-a]pyrazin-5 (4H)-yl)-4-fluorobenzoate

To a solution of methyl 2-amino-5-(6,7-dihydropyrazolo[1,5-a]pyrazin-5 (4H)-yl)-4-fluorobenzoate (154 mg, crude) in MeOH (1.5 mL) and H₂O (0.3 mL) was added lithium hydroxide (89 mg, 2.12 mmol) and the mixture was stirred at 45° C. for 16 hr. The reaction was concentrated under vacuum, to afford the title compound (170 mg, crude), which was used without further purification. MS obsd. (ESI⁺): 277.2, [M+H]⁺ for free acid.

### Step D: (R)-2-amino-N-(1-(3-(difluoromethyl)-2-fluorophenyl)ethyl)-5-(6,7-dihydropyrazolo[1,5-a]pyrazin-5 (4H)-yl)-4-fluorobenzamide

To a solution of lithium 2-amino-5-(6,7-dihydropyrazolo[1,5-a]pyrazin-5 (4H)-yl)-4-fluorobenzoate (170 mg, crude) in DMF (6 mL) was added DIPEA (233 mg, 1.81 mmol) and HATU (274 mg, 0.72 mmol), and the mixture was stirred at rt for 20 min. To the reaction was added 1-(3-(difluoromethyl)-2-fluorophenyl) ethan-1-aminium chloride (135 mg, 0.6 mmol). The reaction was stirred at rt for 3 hr. The reaction was quenched with water and extracted with EtOAc. The organic phase was washed with brine, dried over Na₂SO₄, filtered and concentrated. The crude residue was purified by flash column chromatography (eluting with 0%-5% MeOH in DCM) to afford the impure title compound (334 mg, crude). This material was used without further purification. MS obsd. (ESI⁺): 448.4 [M+H]⁺.

### Step E: (R)-2-amino-N-(1-(3-(difluoromethyl)-2-fluorophenyl)ethyl)-5-(6,7-dihydropyrazolo[1,5-a]pyrazin-5 (4H)-yl)-4-fluorobenzamide (example 7)

To a solution of (R)-2-amino-N-(1-(3-(difluoromethyl)-2-fluorophenyl)ethyl)-5-(6,7-dihydropyrazolo[1,5-a]pyrazin-5 (4H)-yl)-4-fluorobenzamide (334 mg, crude) in acetic acid (6 mL) was added 1-methylpiperidin-4-one (845 mg, 7.47 mmol) at 45° C. After 10 min, sodium cyanoboronhydride (462 mg, 7.47 mmol) was added. The reaction was stirred at 45° C. for 4 hrs. The mixture was adjusted the pH to 8 with aqueous NaHCO₃. The reaction was diluted in water, and extracted with EtOAc (2×30 mL). The combined organic layers were washed with brine (2×30 mL), dried over sodium sulfate, filtered and concentrated. The residue was purified by flash column chromatography (eluting with 50%-60% EtOAc in PE) followed by preparative HPLC (CH₃CN/H₂O/0.1% NH₄HCO₃) to afford the title compound (7.31 mg). MS obsd. (ESI⁺): 545.5, [M+H]⁺.

### Example 8: (R)-5-(2,2-dioxido-2-thia-6-azaspiro[3.3]heptan-6-yl)-4-fluoro-N-(1-(2-methyl-3-(trifluoromethyl)phenyl)ethyl)-2-((1-methylpiperidin-4-yl)amino)benzamide

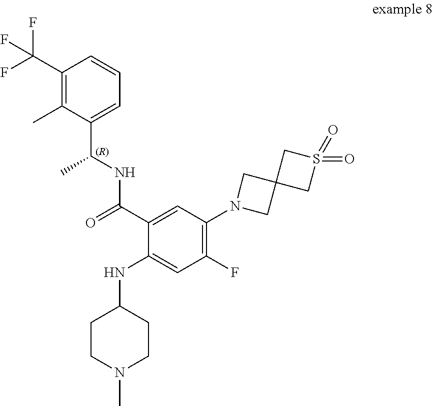

Prepared according to an analogous synthetic route as example 7. MS obsd. (ESI⁺): 583.4, [M+H]⁺. ¹H NMR (400 MHZ, DMSO-d₆) δ:8.67 (1H), 7.72 (1H), 7.57 (1H), 7.48 (1H), 7.40 (1H), 6.96 (1H), 6.52 (1H), 5.37 (1H), 4.46 (4H), 4.01 (4H), 3.22 (1H), 2.57 (2H), 2.47 (3H), 2.14 (3H), 2.07 (2H), 1.82 (2H), 1.44 (3H), 1.27 (2H).

### Example 9: N—((R)-1-(3-(difluoromethyl)-2-fluorophenyl)ethyl)-4-fluoro-2-((1-methylpiperidin-4-yl)amino)-5-(((R)-tetrahydrofuran-3-yl)oxy)benzamide

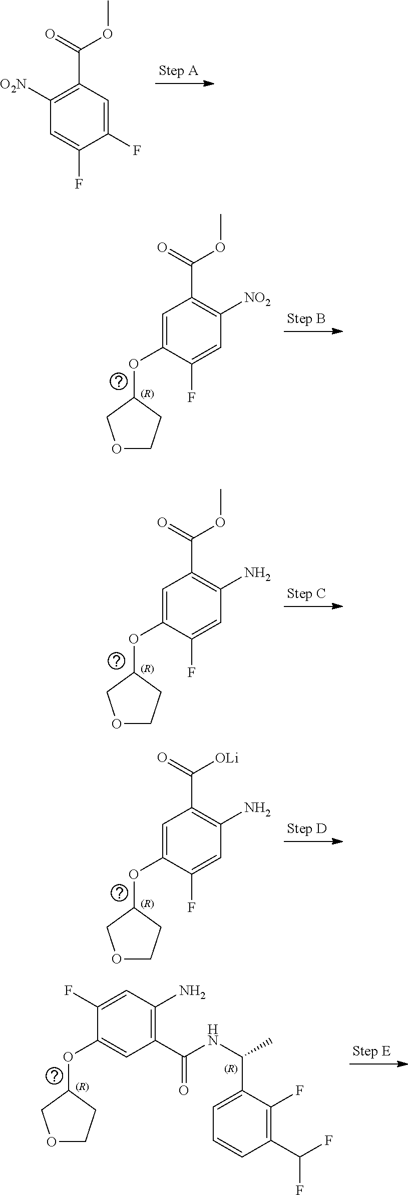

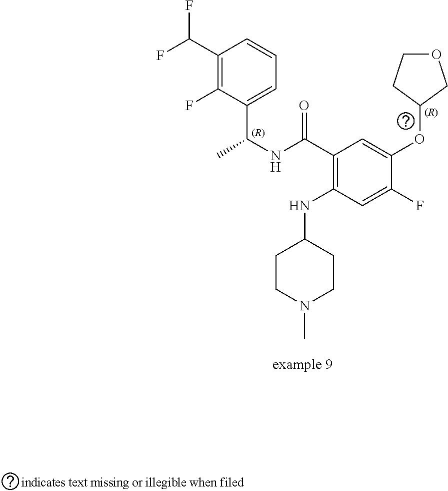

### Step A: Methyl (R)-4-fluoro-2-nitro-5-((tetrahydrofuran-3-yl)oxy)benzoate

To a solution of methyl 4,5-difluoro-2-nitro-benzoate (200 mg, 0.92 mmol) and (3R)-tetrahydrofuran-3-ol (122 mg, 1.38 mmol) in DMSO (6 mL) was added potassium carbonate (255 mg, 1.84 mmol). The reaction was stirred for 16 hr at 30° C. The mixture was diluted with water and extracted with DCM (3×30 mL). The combined organic layers were dried over Na₂SO₄, filtered and concentrated. The residue was purified by flash column chromatography (eluting with 0%-50% EtOAc in PE) to afford the title compound (197 mg, 74% yield). MS obsd. (ESI⁺): 286.2 [(M+H)⁺].

### Steps B-E: N—((R)-1-(3-(difluoromethyl)-2-fluorophenyl)ethyl)-4-fluoro-2-((1-methylpiperidin-4-yl)amino)-5-(((R)-tetrahydrofuran-3-yl)oxy)benzamide (example 9)

Steps B-E were performed according to analogous procedures to example 7. MS obsd. (ESI⁺): 510.5 [(M+H)⁺]. ¹H NMR (400 MHZ, DMSO-d₆) δ:8.67 (1H), 7.76 (1H), 7.63 (1H), 7.58 (1H), 7.52 (1H), 7.38-7.32 (1H), 7.21 (1H), 6.60 (1H), 5.35 (1H), 4.93 (1H), 3.91-3.85 (2H), 3.83-3.74 (2H), 3.24 (1H), 2.61-2.53 (2H), 2.13 (3H), 2.11-1.97 (4H), 1.88-1.77 (2H), 1.48 (3H), 1.36-1.26 (2H).

### Example 10: N—((R)-1-(3-(difluoromethyl)-2-fluorophenyl)ethyl)-4-fluoro-2-((1-methylpiperidin-4-yl)amino)-5-(((S)-tetrahydrofuran-3-yl)oxy)benzamide

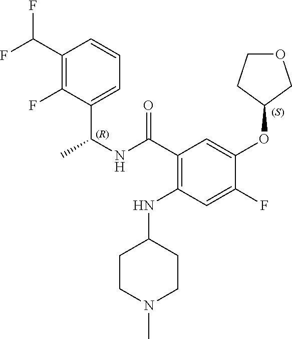

Prepared according to an analogous route to example 9 using (3S)-tetrahydrofuran-3-ol in step A in place of (3R)-tetrahydrofuran-3-ol. MS obsd. (ESI⁺): 510.2 [(M+H)⁺]. ¹H NMR (400 MHZ, DMSO-d₆) δ:8.66 (1H), 7.76 (1H), 7.63 (1H), 7.58 (1H), 7.52 (1H), 7.38-7.32 (1H), 7.21 (1H), 6.60 (1H), 5.35 (1H), 4.93 (1H), 3.91-3.84 (2H), 3.83-3.77 (2H), 3.28-3.19 (1H), 2.57 (2H), 2.13 (3H), 2.11-2.00 (4H), 1.82 (2H), 1.48 (3H), 1.32 (2H).

### Example 11: (R)-4-fluoro-5-(1-methyl-1H-pyrazol-4-yl)-N-(1-(2-methyl-3-(trifluoromethyl)phenyl)ethyl)-2-((1-methylpiperidin-4-yl)amino)benzamide

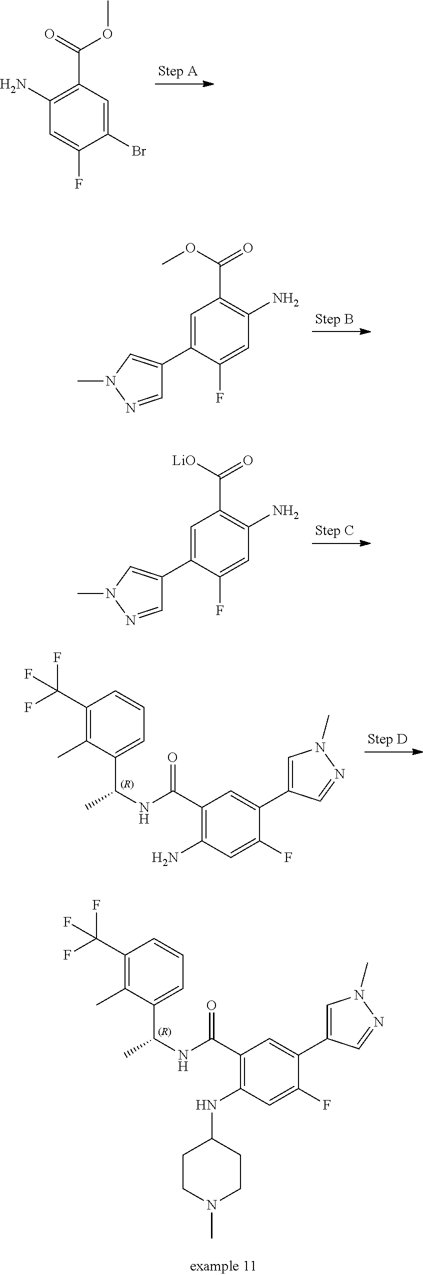

### Step A: Methyl 2-amino-4-fluoro-5-(1-methyl-1H-pyrazol-4-yl)benzoate

To a mixture of methyl 2-amino-5-bromo-4-fluorobenzoate (150 mg, 0.6 mmol) in dioxane (3 mL) and H₂O (0.5 mL) was added 1-methyl-4-(4,4,5,5-tetramethyl-1,3,2-dioxaborolan-2-yl)-1H-pyrazole (151 mg, 0.72 mmol), 1,1′-Bis(diphenylphosphino) ferrocene palladium (II) dichloride (89 mg, 0.12 mmol), and sodium carbonate (192 mg, 1.81 mmol). The mixture was stirred at 110° C. for 3 hr. The mixture was cooled down to rt, diluted with EtOAc (10 mL) and washed with H₂O (10 mL×3) and brine (8 mL×3). The organic layer was dried (Na₂SO₄), concentrated, and purified by silica gel column (PE:EtOAc=3:1) to provide the title compound (131 mg, 86% yield). MS obsd. (ESI⁺): 250.2 [(M+H)⁺].

### Step B: Lithium 2-amino-4-fluoro-5-(1-methyl-1H-pyrazol-4-yl)benzoate

To a solution of methyl 2-amino-4-fluoro-5-(1-methyl-1H-pyrazol-4-yl)benzoate (121 mg, 0.49 mmol) in MeOH (4 mL) at 0° C. was added a solution of lithium hydroxide (2.25 mg, 0.97 mmol) in H₂O (1 mL). The mixture was stirred at 30° C. for 16 hr. The mixture was concentrated to provide the crude title compound (117 mg, crude), which was used without further purification. MS obsd. (ESI⁺): 236.1 [(M+H)⁺] for free acid.

### Step C: (R)-2-amino-4-fluoro-5-(1-methyl-1H-pyrazol-4-yl)-N-(1-(2-methyl-3-(trifluoro-meth-yl)phenyl)ethyl)benzamide

To a mixture of lithium 2-amino-4-fluoro-5-(1-methyl-1H-pyrazol-4-yl)benzoate (117 mg, crude) in DMF (3 mL) was added HATU (277 mg, 0.73 mmol), DIPEA (188 mg, 1.46 mmol) and (R)-1-(2-methyl-3-(trifluoromethyl)phenyl) ethan-1-amine (118 mg, 0.58 mmol). The mixture was stirred at rt for 3 hr. The mixture was diluted with EtOAc (20 mL) and washed with H₂O (15 mL×3) and brine (15 mL×3). The organic layer was dried (Na₂SO₄), concentrated, and purified by preparative HPLC (ACN/water/0.1% NH₄HCO₃) to provide the title compound (160 mg, 78% yield). MS obsd. (ESI⁺): 421.0 [(M+H)⁺].

### Step D: (R)-4-fluoro-5-(1-methyl-1H-pyrazol-4-yl)-N-(1-(2-methyl-3-(trifluoromethyl)phenyl)ethyl)-2-((1-methylpiperidin-4-yl)amino)benzamide (Example 11)

To a mixture of (R)-2-amino-4-fluoro-5-(1-methyl-1H-pyrazol-4-yl)-N-(1-(2-methyl-3-(trifluoromethyl)phenyl)ethyl)benzamide (150 mg, 0.36 mmol) in AcOH (6 mL) was added 1-methylpiperidin-4-one (202 mg, 1.78 mmol) and sodium triacetoxyborohydride (378 mg, 1.78 mmol). The mixture was stirred at 25° C. for 2 hr. The mixture concentrated and the residue was diluted with DCM (20 mL), washed with saturated aq. NaHCO₃ (10 mL×3), H₂O (10 mL×3), and brine (10 mL×3). The organic layer was dried (Na₂SO₄), filtered and concentrated. Purification by preparative HPLC (ACN/water/0.1% NH₄HCO₃) provided the title compound (62 mg, 33% yield). MS obsd. (ESI⁺): 518.5 [(M+H)⁺]. ¹H NMR (400 MHZ, DMSO-d₆) δ:8.78 (1H), 8.00-7.81 (4H), 7.76 (1H), 7.57 (1H), 7.41 (1H), 6.57 (1H), 5.39 (1H), 3.89 (3H), 3.31-3.26 (1H), 2.58 (2H), 2.14 (3H), 2.09 (2H), 1.87 (2H), 1.46 (3H), 1.35 (2H).

### Example 12: (R)-4-fluoro-N-(1-(2-methyl-3-(trifluoromethyl)phenyl)ethyl)-2-((1-methylpiperidin-4-yl)amino)-5-(oxazol-5-yl)benzamide

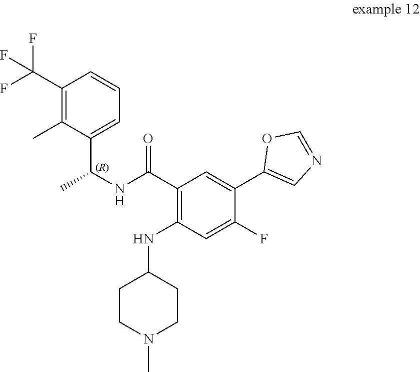

Prepared according to an analogous route as example 11. MS obsd. (ESI⁺): 505.5 [(M+H)⁺]. ¹H NMR (400 MHZ, DMSO) δ 9.07 (1H), 8.49 (1H), 8.24 (1H), 8.15 (1H), 7.76 (1H), 7.58 (1H), 7.42 (1H), 7.32 (1H), 6.79 (1H), 5.39 (1H), 3.55 (1H), 3.13 (2H), 2.79 (2H), 2.59 (3H), 2.49 (3H), 2.12-1.98 (2H), 1.53 (2H), 1.46 (3H).

### Example 13: (R)-4-chloro-N-(1-(2-methyl-3-(trifluoromethyl)phenyl)ethyl)-2-((1-

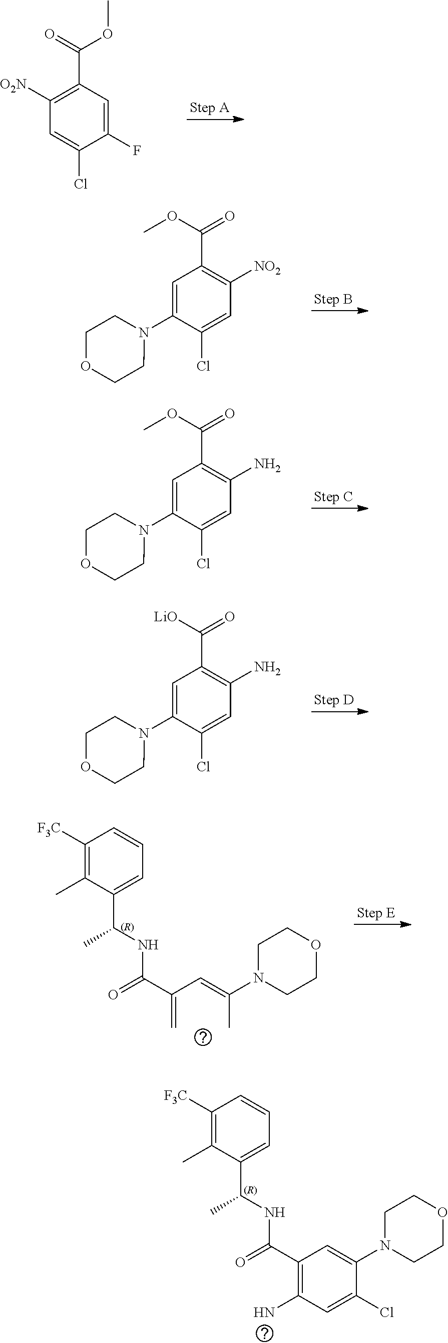

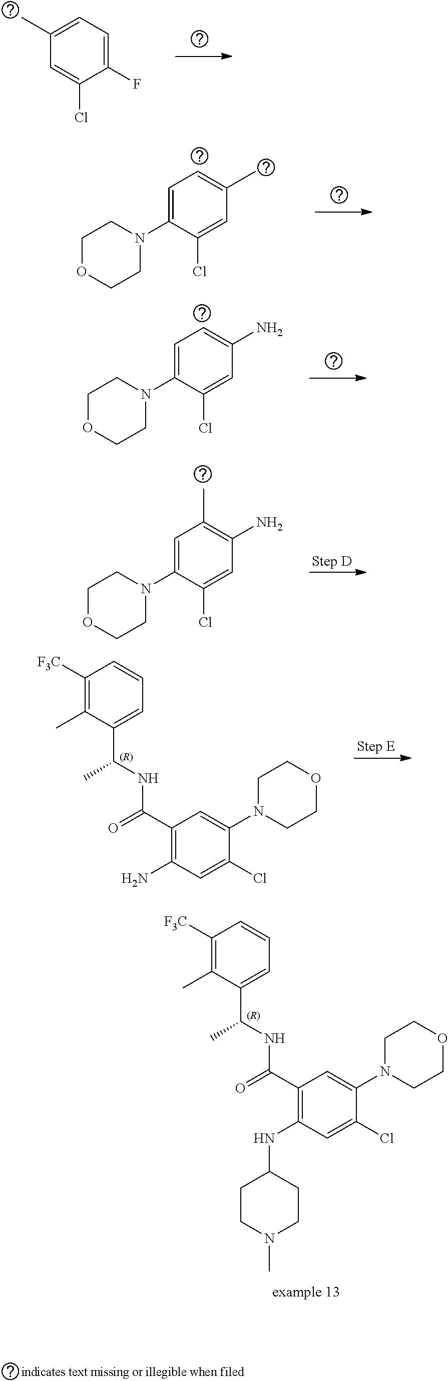

### Step A: Methyl 4-chloro-5-morpholino-2-nitrobenzoate

To a solution of methyl 4-chloro-5-fluoro-2-nitrobenzoate (500 mg, 2.14 mmol) in THF (5.0 mL) was added triethylamine (433 mg, 4.28 mmol) and morpholine (242 mg, 2.78 mmol) at 0° C. The mixture was stirred at 0° C. for 16 hrs. The reaction mixture was quenched with aqueous NH₄Cl (5 mL) and extracted with EtOAc (10 mL×3). The combined organic layers were washed with brine, dried over anhydrous Na₂SO₄, filtered, and concentrated in vacuo. The residue was purified by silica gel chromatography (eluting with 20 to 45% EA in PE) to afford the title compound (500 mg, 77% yield). MS obsd. (ESI⁺): ³⁵C/³⁷Cl 301.1/303.1, [M+H]⁺.

### Step B: Methyl 2-amino-4-chloro-5-morpholinobenzoate

To a solution of methyl 4-chloro-5-morpholino-2-nitrobenzoate (100 mg, 0.33 mmol) in AcOH (6.0 mL) was added Fe powder (186 mg, 3.33 mmol) at 50° C. The reaction mixture was stirred for 1 hr at 50° C. The reaction mixture was concentrated to dryness, the residue was diluted with aqueous NaHCO₃ (15 mL) and extracted with EtOAc (20 mL×3). The combined organic layers were washed with water (15 mL×3), dried over anhydrous Na₂SO₄, filtered and concentrated to afford the title compound (70 mg, 77% yield). MS obsd. (ESI⁺): ³⁵Cl/³⁷Cl 271.0/272.0, [M+H]⁺.

### Step C: Lithium 2-amino-4-chloro-5-morpholinobenzoate

To a solution of methyl 2-amino-4-chloro-5-morpholinobenzoate (60 mg, 0.22 mmol) in water (1.0 mL) and THF (4.0 mL) was added LiOH (16 mg, 0.66 mmol) at rt. The reaction mixture was stirred for 16 hrs at 45° C. The reaction mixture was directly concentrated to afford the crude title compound (60 mg, crude). The crude product was used directly in the next step. MS obsd. (ESI⁺): ³⁵Cl/³⁷Cl 257.1/259.2 [M+H]⁺ for free acid.

### Step D: (R)-2-amino-4-chloro-N-(1-(2-methyl-3-(trifluoromethyl)phenyl)ethyl)-5-morpholinobenzamide

To a solution of lithium 2-amino-4-chloro-5-morpholinobenzoate (60 mg, crude) in DMF (3.0 mL) was added HATU (105 mg, 0.28 mmol, 1.2 eq.), N-ethyl-N-isopropyl-propan-2-amine (89 mg, 0.69 mmol) and (1R)-1-[2-methyl-3-(trifluoromethyl)phenyl]ethanamine (56 mg, 0.28 mmol) at rt. The reaction mixture was stirred for 2 hrs at rt. The reaction mixture was quenched with water (5.0 mL) and extracted with EtOAc (10 mL×3). The combined organic layers were washed with brine, dried over anhydrous Na₂SO₄, filtered, and concentrated in vacuo. The residue was purified by silica gel chromatography (eluting with 0 to 20% EA in PE) to afford the title compound (60 mg, 61% yield). MS obsd. (ESI⁺): ³⁵Cl/37Cl 442.4/444.4 [M+H]⁺

### Step E: (R)-4-chloro-N-(1-(2-methyl-3-(trifluoromethyl)phenyl)ethyl)-2-((1-methylpiperidin-4-yl)amino)-5-morpholinobenzamide (example 13)

To a solution of (R)-2-amino-4-chloro-N-(1-(2-methyl-3-(trifluoromethyl)phenyl)ethyl)-5-morpholinobenzamide (50 mg, 0.11 mmol) in AcOH (4.0 mL) was added 1-methylpiperidin-4-one (128 mg, 1.13 mmol) and sodium triacetoxyborohydride (240 mg, 1.13 mmol). The reaction mixture was stirred for 16 hrs at rt. The solvents were removed under reduced pressure. The residue was diluted with aqueous NaHCO₃ (10 mL) and extracted with EtOAc (10 mL×3). The combined organic layers were washed with brine, dried over anhydrous Na₂SO₄, filtered, and concentrated in vacuo. The residue was purified by preparative HPLC (ACN/water/0.1% NH₄HCO₃) to afford the title compound (22.5 mg, 36% yield). MS obsd. (ESI⁺): ³⁵Cl/³⁷Cl 539.5/541.6 [M+H]⁺. ¹H NMR (400 MHZ, DMSO-d₆) δ: 8.77 (1H), 7.73 (1H), 7.58 (1H), 7.53 (1H), 7.48 (1H), 7.42 (1H), 6.75 (1H), 5.38 (1H), 3.80-3.69 (4H), 3.26 (1H), 2.95-2.89 (4H), 2.61-2.51 (2H), 2.48 (3H), 2.12 (3H), 2.10-1.99 (2H), 1.87-1.76 (2H), 1.46 (3H), 1.35-1.24 (2H).

### Example 14: (R)—N-(1-(3-(difluoromethyl)-2-fluorophenyl)ethyl)-4-fluoro-5-morpholino-

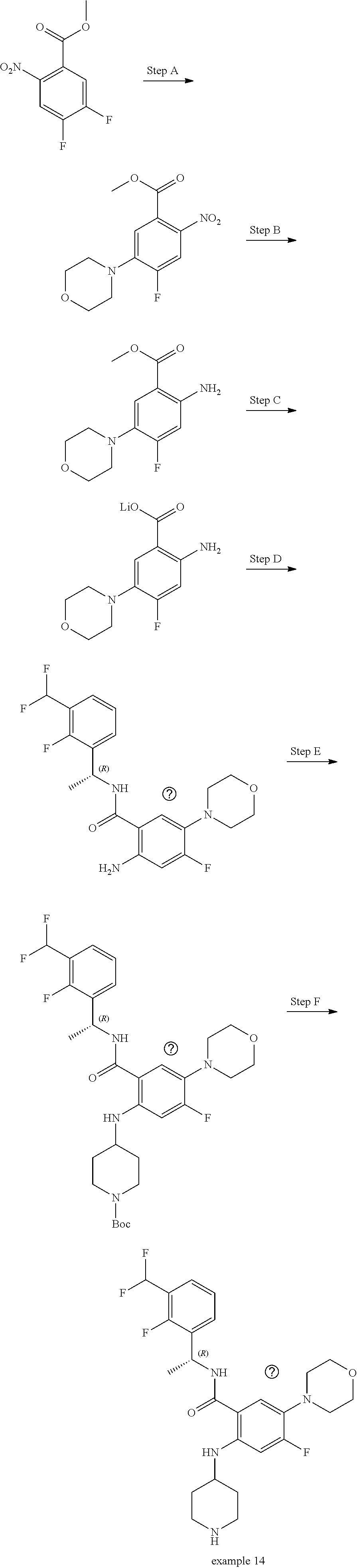

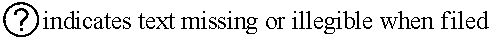

### Step A: Methyl 4-fluoro-5-morpholino-2-nitrobenzoate

To a solution of methyl 4,5-difluoro-2-nitro-benzoate (5 g, 23.03 mmol) in DMSO (40 mL) was added morpholine (2.6 g, 29.94 mmol) and triethylamine (3.5 g, 34.54 mmol). The mixture was stirred at rt for 1 hr. The resulting mixture was diluted with DCM (150 mL) and washed with water (150 mL×3). The organic layer was dried over sodium sulfate, filtered and concentrated. The residue was purified by silica gel column (EtOAc/PE, 10-50%) to afford the title compound (5.5 g, 84% yield). MS obsd. (ESI⁺): 285.2 [M+H]⁺.

### Step B: Methyl 2-amino-4-fluoro-5-morpholinobenzoate

To a solution of methyl 4-fluoro-5-morpholino-2-nitrobenzoate (5.5 g, 19.35 mmol) in acetic acid (50 mL) was added iron powder (10.8 g, 193.50 mmol). The mixture was stirred at 50° C. for 1 hr. The mixture was directly concentrated and the residue was diluted with aqueous NaHCO₃ (100 mL) and DCM (100 mL). The mixture was stirred at rt for 15 min, then was filtered. The filtrate was extracted with DCM (150 mL×3), the combined organic layer was dried over Na₂SO₄, filtered and concentrated to afford the crude title compound (4.8 g, 97% yield), which was used for next step without further purification. MS obsd. (ESI⁺): 255.2 [M+H]⁺.

### Step C: Lithium 2-amino-4-fluoro-5-morpholinobenzoate

To a solution of methyl 2-amino-4-fluoro-5-morpholinobenzoate (4.8 g, 18.88 mmol) in THF (50 mL) was added lithium hydroxide (2.26 g, 94.39 mmol) and water (10 mL). The mixture was stirred at 40° C. for 16 hr, after which the mixture was directly concentrated to afford the crude title compound (4.5 g, crude). This material is used without further purification. MS obsd. (ESI⁺): 241.2 [M+H]⁺ for free acid.

### Step D: (R)-2-amino-N-(1-(3-(difluoromethyl)-2-fluorophenyl)ethyl)-4-fluoro-5-morpholinobenzamide

To a solution of lithium 2-amino-4-fluoro-5-morpholinobenzoate (4.5 g, crude) in DMF (50 mL) was added HATU (9.3 g, 24.35 mmol), triethylamine (5.7 g, 56.20 mmol) and (R)-1-(3-(difluoromethyl)-2-fluorophenyl) ethan-1-amine (5.32 g, 28.10 mmol), and the mixture was stirred at rt for 16 hr. The mixture was poured into water (150 mL) and extracted with DCM (150 mL×3). The combined organic layers were dried over Na₂SO₄, filtered and concentrated. The residue was purified by column chromatography on silica gel (AcOEt/PE, 0-50%) to afford the target compound (6.1 g, 78% yield over 2 steps). MS obsd. (ESI⁺): 412.3 [M+H]⁺

### Step E: Tert-butyl (R)-4-((2-((1-(3-(difluoromethyl)-2-fluorophenyl)ethyl) carbamoyl)-5-fluoro-4-morpholinophenyl)amino) piperidine-1-carboxylate

To a solution of (R)-2-amino-N-(1-(3-(difluoromethyl)-2-fluorophenyl)ethyl)-4-fluoro-5-morpholinobenzamide (200 mg, 0.49 mmol) in acetic acid (10 mL) was added tert-butyl 4-oxopiperidine-1-carboxylate (969 mg, 4.86 mmol). The mixture was stirred at rt for 15 min, then sodium triacetoxyborohydride (1.0 g, 4.86 mmol) was added and the mixture was stirred at rt for 2 hr. The mixture was diluted with DCM (120 mL), the organic layer was washed with water (120 mL×3), dried over Na₂SO₄, filtered and concentrated. The residue was purified by column chromatography on silica gel (AcOEt/DCM, 10-60%) to afford the target compound (243 mg, 0.41 mmol, 84% yield). MS obsd. (ESI⁺): 595.5 [M+H]⁺

### Step F: (R)—N-(1-(3-(difluoromethyl)-2-fluorophenyl)ethyl)-4-fluoro-5-morpholino-2-(piperidin-4-ylamino)benzamide (example 14)

To a solution of tert-butyl (R)-4-((2-((1-(3-(difluoromethyl)-2-fluorophenyl)ethyl) carbamoyl)-5-fluoro-4-morpholinophenyl)amino) piperidine-1-carboxylate (243 mg, 0.49 mmol) in DCM (5 mL) was added TFA (1 mL). The mixture was stirred at rt for 1 hr, after which the mixture was concentrated under vacuum. The residue was neutralized with NH₃ in methanol (2 mL, 7 M), concentrated again, and the residue was purified by preparative HPLC (ACN/water/0.1% HCOOH) to afford the target compound (60.8 mg, 39% yield, 0.7 eq. HCOOH salt). MS obsd. (ESI⁺): 495.4 [M+H]⁺

### Examples 15 and 16: N—((R)-1-(3-(difluoromethyl)-2-fluorophenyl)ethyl)-4-fluoro-5-morpholino-2-(((R)-quinuclidin-3-yl)amino)benzamide (example 15) and N—((R)-1-(3-(difluoromethyl)-2-fluorophenyl)ethyl)-4-fluoro-5-morpholino-2-(((S)-quinuclidin-3-yl)amino)benzamide (example 16)

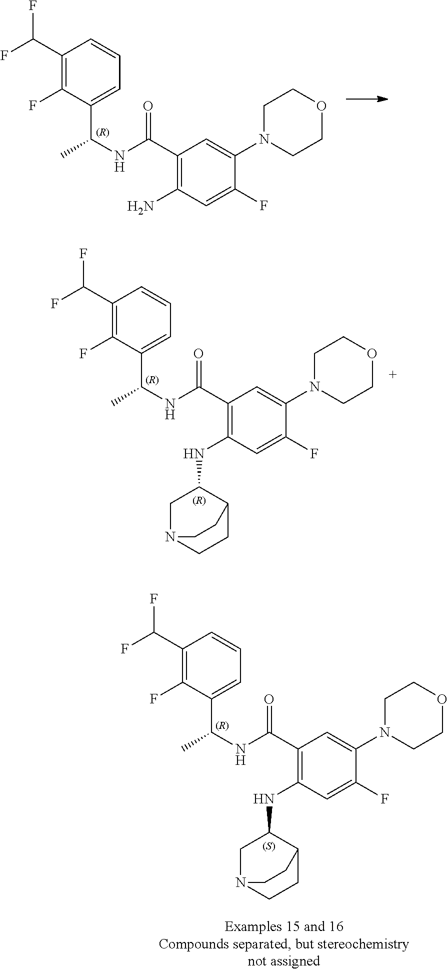

A mixture of (R)-2-amino-N-(1-(3-(difluoromethyl)-2-fluorophenyl)ethyl)-4-fluoro-5-morpholinobenzamide (206 mg, 0.50 mmol), quinuclidin-3-one hydrochloride (809 mg, 5.01 mmol) and NaBH₃CN (158 mg, 2.50 mmol) in AcOH (10 mL) was stirred for 24 hr at 60° C. The reaction solution was concentrated under vacuum. The residue was purified by chromatography column (0-10% MeOH in DCM) to provide N—((R)-1-(3-(difluoromethyl)-2-fluorophenyl)ethyl)-4-fluoro-5-morpholino-2-(quinuclidin-3-ylamino)benzamide as a diastereomeric mixture (90 mg, 0.17 mmol, 34.5% yield). LCMS: m/z, 521.3, [M+H]⁺.

The mixture was purified by SFC (Regis (R,R) Whelk-O1 (25*250.10 μm), CO₂/EtOH [0.5% NH₃ (7M in MeOH)]=65/35)) to afford:

Example 15: (19.1 mg) MS obsd. (ESI⁺): 521.3 [M+H]⁺ Analytical chiral HPLC: (Column: (R,R)-Whelk-O1, 4.6*100 mm 3 μm, Flow rate: 3.0 mL/min, Eluent: EtOH (1% 7M NH3 in MeOH), Temp 400C) Retention time=2.2 min

Example 16: (18.6 mg) MS obsd. (ESI⁺): 521.3 [M+H]⁺ Analytical chiral HPLC: (Column: (R,R)-Whelk-O1, 4.6*100 mm 3 μm, Flow rate: 3.0 mL/min, Eluent: EtOH (1% 7M NH3 in MeOH), Temp 400C) Retention time=1.6 min

### Examples 17 and 18: N—((R)-1-(3-(difluoromethyl)-2-fluorophenyl)ethyl)-4-fluoro-2-(((1R,5S,6s)-3-methyl-3-azabicyclo[3.1.1]heptan-6-yl)amino)-5-morpholinobenzamide and N—((R)-1-(3-(difluoromethyl)-2-fluorophenyl)ethyl)-4-fluoro-2-(((1R,5S,6r)-3-methyl-3-azabicyclo[3.1.1]heptan-6-yl)amino)-5-morpholinobenzamide

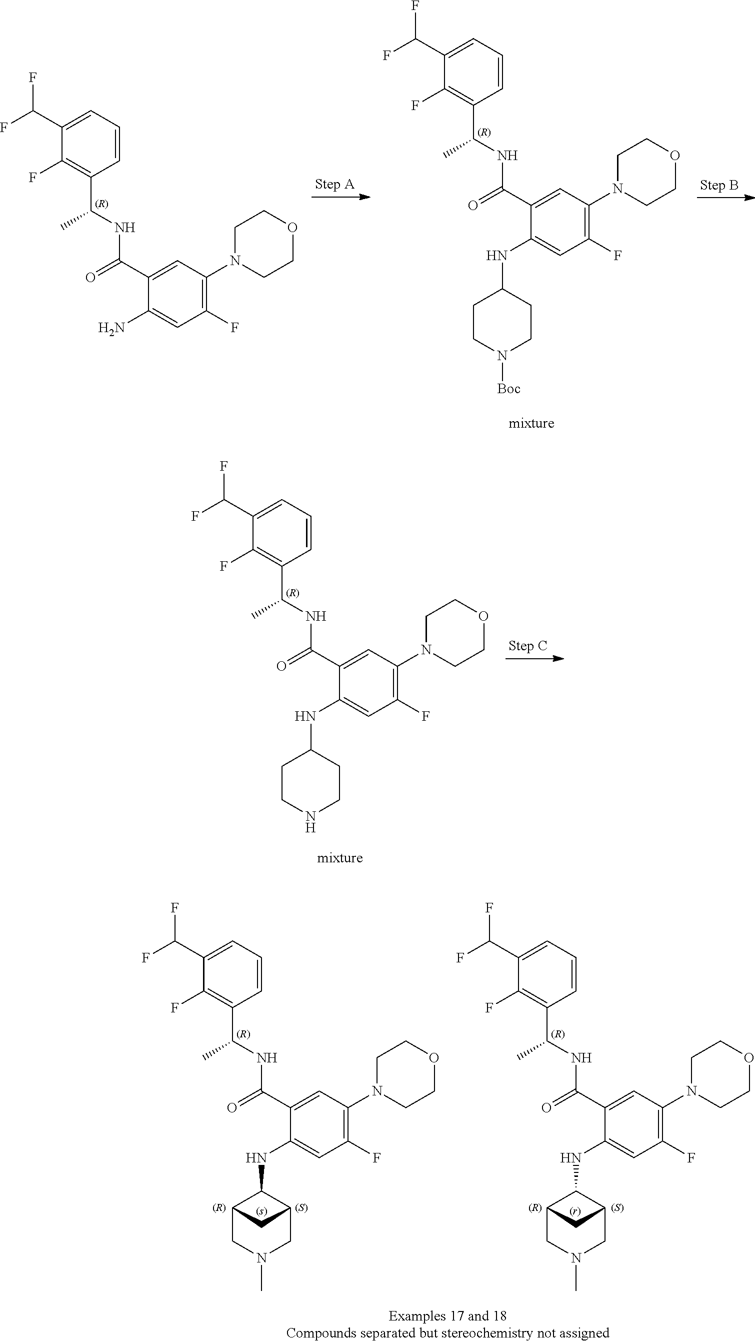

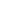

### Step A: tert-butyl 6-((2-(((R)-1-(3-(difluoromethyl)-2-fluorophenyl)ethyl) carbamoyl)-5-fluoro-4-morpholinophenyl)amino)-3-azabicyclo[3.1.1]heptane-3-carboxylate

To a solution of (R)-2-amino-N-(1-(3-(difluoromethyl)-2-fluorophenyl)ethyl)-4-fluoro-5-morpholinobenzamide (200 mg, 0.48 mmol) in AcOH (5 mL) was added tert-butyl 6-oxo-3-azabicyclo[3.1.1]heptane-3-carboxylate (513 mg, 2.43 mmol). The mixture was stirred at room temperature for 1 hr. NaBH (OAc)₃ (410 mg, 1.94 mmol) was added to the mixture and the mixture was stirred at room temperature for another 16 hr. The reaction solution was concentrated. Water (50 mL) was added to the residue, adjusted pH ˜7 with NaHCO₃, and then extracted with EtOAc (30 mL×3). The combined organic layers were dried over sodium sulfate, filtered and concentrated. The residue was purified by column chromatography (0-40% EA in PE) to afford the title compound (200 mg, 68% yield) as a white solid. MS obsd. (ESI⁺): 607.4 [M+H]⁺

### Step B: 2-((3-azabicyclo[3.1.1]heptan-6-yl)amino)-N—((R)-1-(3-(difluoromethyl)-2-fluorophenyl)ethyl)-4-fluoro-5-morpholinobenzamide

Tert-butyl 6-((2-(((R)-1-(3-(difluoromethyl)-2-fluorophenyl)ethyl) carbamoyl)-5-fluoro-4-morpholinophenyl)amino)-3-azabicyclo[3.1.1]heptane-3-carboxylate (180 mg, 0.29 mmol) was dissolved in HCl (4 M solution in 1,4-dioxane, 4 mL). The mixture was stirred at room temperature for 2 hr. The reaction solution was then concentrated. The residue was diluted with water (50 mL) and adjusted to pH˜10 with aqueous NaHCO₃. The mixture was extracted with EtOAc (3×30 mL). The combined organic layers were dried over sodium sulfate, filtered and concentrated to afford the title compound (200 mg, crude) as a diastereomeric mixture. This material was used without further purification. MS obsd. (ESI⁺): 507.4 [M+H]⁺

### Step C: N—((R)-1-(3-(difluoromethyl)-2-fluorophenyl)ethyl)-4-fluoro-2-(((1R,5S,6s)-3-methyl-3-azabicyclo[3.1.1]heptan-6-yl)amino)-5-morpholinobenzamide and N—((R)-1-(3-(difluoromethyl)-2-fluorophenyl)ethyl)-4-fluoro-2-(((1R,5S,6r)-3-methyl-3-azabicyclo[3.1.1]heptan-6-yl)amino)-5-morpholinobenzamide

To a solution of crude 2-((3-azabicyclo[3.1.1]heptan-6-yl)amino)-N—((R)-1-(3-(difluoromethyl)-2-fluorophenyl)ethyl)-4-fluoro-5-morpholinobenzamide (180 mg) in EtOH (3 mL) was added paraformaldehyde (95 mg, 1.07 mmol) and NaBH₃CN (110 mg, 1.78 mmol). The mixture was stirred at room temperature for 16 hr. The reaction solution was poured into water (100 mL). The mixture was extracted with EtOAc (70 mL×3). The combined organic layers were dried over sodium sulfate, filtered and concentrated. The residue was purified by silica gel chromatography (0-10% MeOH in DCM) to afford the title compounds (40 mg) as a diastereomeric mixture. MS obsd. (ESI⁺): 521.3 [M+H]⁺

The aforementioned material (40 mg, 0.076 mmol) was further separated by SFC (Regis (R,R) Whelk-O1 (25*250.10 μm), CO2/MeOH [0.2% NH3 (7M in MeOH)]=65/35) to afford:

Example 17 (10.6 mg). MS obsd. (ESI⁺): 521.4 [M+H]⁺. Analytical chiral HPLC: (Column: (R,R)-Whelk-O1, 4.6*100 mm 3 μm, Flow rate: 3.0 mL/min, Eluent: EtOH (1% 7M NH3 in MeOH), Temp 400C) Retention time=1.26 min.

Example 18 MS obsd. (ESI⁺): 521.4 [M+H]⁺. Analytical chiral HPLC: (Column: (R,R)-Whelk-O1, 4.6*100 mm 3 μm, Flow rate: 3.0 mL/min, Eluent: EtOH (1% 7M NH3 in MeOH), Temp 400C) Retention time=1.59 min.

### Example 19:5-((1S,4S)-2-oxa-5-azabicyclo[2.2.1]heptan-5-yl)-N—((R)-1-(3-(difluoromethyl)-2-fluorophenyl)ethyl)-4-fluoro-2-((1-methylpiperidin-4-yl)amino)benzamide

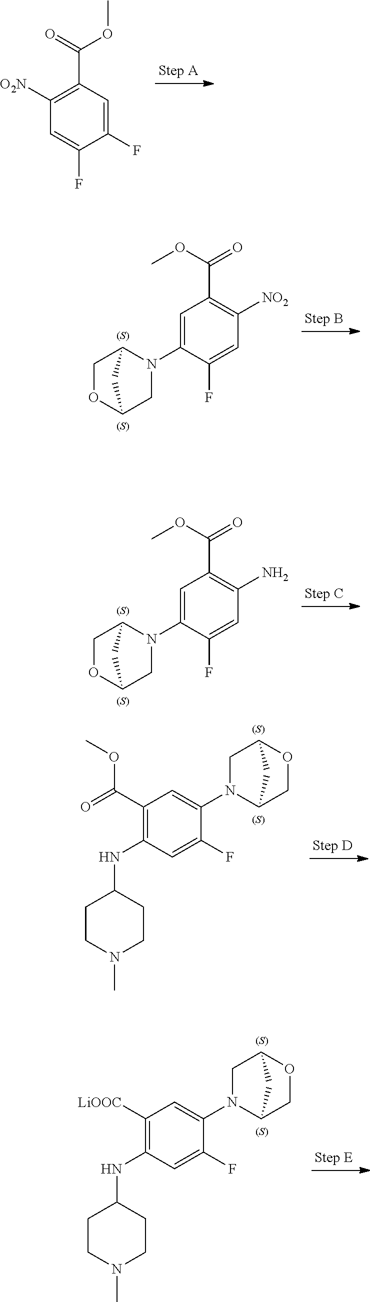

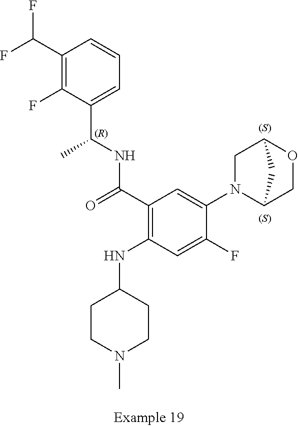

### Step A: methyl 5-((1S,4S)-2-oxa-5-azabicyclo[2.2.1]heptan-5-yl)-4-fluoro-2-nitrobenzoate

To a solution of methyl 4,5-difluoro-2-nitrobenzoate (500 mg, 2.30 mmol) in THF (4.0 mL) was added triethylamine (466 mg, 4.60 mmol) and ((1S,4S)-2-oxa-5-azabicyclo[2.2.1]heptan-5-ium chloride (375 mg, 2.76 mmol) at 0° C. The mixture was stirred at 0° C. for 16 hrs. The reaction mixture was quenched with NH₄Cl (aq. 5.0 mL) and extracted with EtOAc (10 mL×3). The combined organic layers were washed with brine, dried over anhydrous Na₂SO₄, filtered, and concentrated in vacuo. The residue was purified by silica gel chromatography (eluting with 0-30% EtOAc in PE) to afford the title compound (600 mg, 87% yield). MS obsd. (ESI⁺): 297.0 [M+H]⁺.

### Step B: methyl 2-amino-5-((1S,4S)-2-oxa-5-azabicyclo[2.2.1]heptan-5-yl)-4-fluorobenzoate

To a solution of methyl 5-((1S,4S)-2-oxa-5-azabicyclo[2.2.1]heptan-5-yl)-4-fluoro-2-nitrobenzoate (600 mg, 2.03 mmol) in MeOH (10 mL) was added Pd/C (200 mg, 10 wt %) at rt. The reaction mixture was stirred for 16 hrs at rt under H₂ (1 atm). The reaction mixture was filtered and concentrated to afford the title compound (500 mg, crude). The crude product was used directly in the next step. MS obsd. (ESI⁺): 267.2 [M+H]⁺.

### Step C: Methyl-5-((1S,4S)-2-oxa-5-azabicyclo[2.2.1]heptan-5-yl)-4-fluoro-2-((1-methylpiperidin-4-yl)amino)benzoate

To a solution of methyl 2-amino-5-((1S,4S)-2-oxa-5-azabicyclo[2.2.1]heptan-5-yl)-4-fluorobenzoate (100 mg, crude, assumed 0.38 mmol) in AcOH (8 mL) was added 1-methylpiperidin-4-one (425 mg, 3.80 mmol) and sodium triacetoxyborohydride (398 mg, 1.90 mmol) at rt. The reaction mixture was stirred for 16 hrs at rt. The reaction mixture was concentrated to dryness. To the residue was added saturated aqueous NaHCO₃ (15 mL) and the mixture was extracted with EtOAc (10 mL×3). The combined organic layers were washed with brine, dried over anhydrous Na₂SO₄, filtered, and concentrated in vacuo. The residue was purified by silica gel chromatography (eluting with 0 to 30% EtOAc in PE) to afford the title compound (90 mg, 65% yield). MS obsd. (ESI⁺): 364.2 [M+H]⁺.

### Step D: Lithium-5-((1S,4S)-2-oxa-5-azabicyclo[2.2.1]heptan-5-yl)-4-fluoro-2-((1-methylpiperidin-4-yl)amino)benzoate

To a solution of methyl 5-((1S,4S)-2-oxa-5-azabicyclo[2.2.1]heptan-5-yl)-4-fluoro-2-((1-methylpiperidin-4-yl)amino)benzoate (80 mg, 0.22 mmol) in water (0.4 mL) and THF (2.0 mL) at 30° C. was added anhydrous LiOH (16 mg, 0.66 mmol). The reaction mixture was stirred for 16 hrs at 30° C. The reaction mixture was concentrated to afford the crude title compound (80 mg, crude). The crude product was used directly in the next step. MS obsd. (ESI⁺): 350.2 [M+H]⁺ for free acid.

### Step E: 5-((1S,4S)-2-oxa-5-azabicyclo[2.2.1]heptan-5-yl)-N—((R)-1-(3-(difluoromethyl)-2-fluorophenyl)ethyl)-4-fluoro-2-((1-methylpiperidin-4-yl)amino)benzamide (example 19)

To a solution of lithium 5-((1S,4S)-2-oxa-5-azabicyclo[2.2.1]heptan-5-yl)-4-fluoro-2-((1-methylpiperidin-4-yl)amino)benzoate (80 mg, crude) in DMF (5.0 mL) was added HATU (103 mg, 0.27 mmol), N-ethyl-N-isopropyl-propan-2-amine (89 mg, 0.69 mmol) and (R)-1-(3-(difluoromethyl)-2-fluorophenyl) ethan-1-amine (51 mg, 0.27 mmol) at rt. The reaction mixture was stirred for 2 hrs at rt. The reaction mixture was quenched with water (10 mL) and extracted with EtOAc (15 mL×3). The combined organic layers were washed with brine, dried over anhydrous Na₂SO₄, filtered, and concentrated in vacuo. The residue was purified by silica gel chromatography (eluting with 0 to 20% MeOH in DCM) followed by preparative HPLC (ACN/water/0.1% NH₄HCO₃) to afford the title compound (51.5 mg). MS obsd. (ESI⁺): 350.2 [M+H]⁺. ¹H NMR (400 MHZ, DMSO-d6) δ 8.65 (1H), 7.61 (1H), 7.52 (1H), 7.38-7.05 (4H), 6.54 (1H), 5.38-5.30 (1H), 4.55 (1H), 4.43 (1H), 3.82 (1H), 3.73 (1H), 3.45 (1H), 3.20 (1H), 3.10 (1H), 2.56 (2H), 2.13 (3H), 2.10-2.01 (2H), 1.93-1.75 (4H), 1.48 (3H), 1.34-1.23 (2H).

### Example 20: (R)-5-(6-acetyl-2,6-diazaspiro[3.3]heptan-2-yl)-4-fluoro-N-(1-(2-methyl-3-(trifluoromethyl)phenyl)ethyl)-2-((1-methylpiperidin-4-yl)amino)benzamide

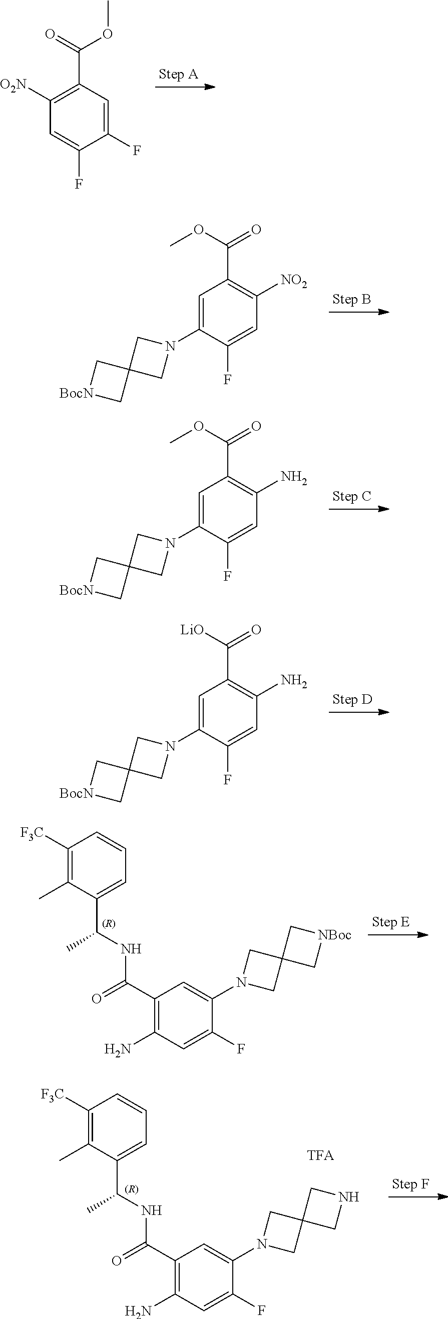

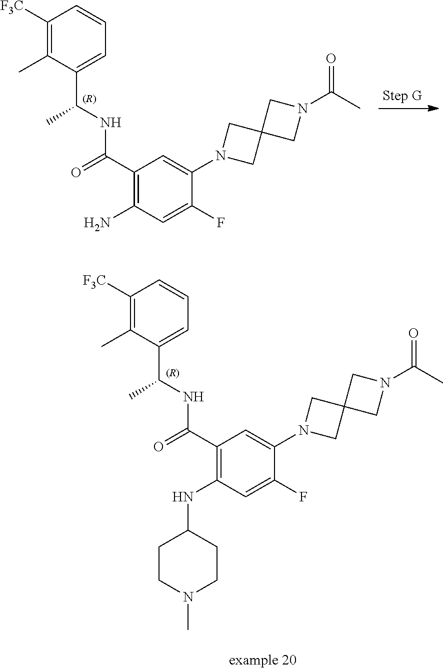

### Step A: Tert-butyl 6-(2-fluoro-5-(methoxycarbonyl)-4-nitrophenyl)-2,6-diazaspiro[3.3]heptane-2-carboxylate

To a mixture of methyl 4,5-difluoro-2-nitrobenzoate (1 g, 4.61 mmol) in DMSO (15 mL) was added tert-butyl 2,6-diazaspiro[3.3]heptane-2-carboxylate oxalate (1.33 g, 4.61 mmol) and triethylamine (1.40 g, 13.82 mmol). The mixture was stirred at 23° C. for 2 hr. The mixture was diluted with EtOAc (60 mL), washed with H₂O (40 mL×3), brine (40 mL×3), dried (Na₂SO₄) and concentrated. The residue was purified by silica gel column chromatography (PE:EtOAc=2:1) to provide the title compound (970 mg, 53% yield). MS obsd. (ESI⁺): 396.4 [(M+H)⁺].

### Step B: Tert-butyl 6-(4-amino-2-fluoro-5-(methoxycarbonyl)phenyl)-2,6-diazaspiro[3.3]heptane-2-carboxylate

To a mixture of tert-butyl 6-(2-fluoro-5-(methoxycarbonyl)-4-nitrophenyl)-2,6-diazaspiro[3.3]heptane-2-carboxylate (300 mg, 0.76 mmol) in acetic acid (10 mL) was added iron powder (424 mg, 7.59 mmol). The mixture was stirred at 50° C. for 2 hr. The mixture was filtered and concentrated, and the residue was diluted with DCM (30 mL). The organic mixture was washed with saturated aqueous NaHCO₃ (15 mL×3), H₂O (15 mL×3) and brine (15 mL×3). The organic layer was dried (Na₂SO₄), filtered and concentrated. The residue was purified by silica gel chromatography (PE:EtOAc=2:1) to provide the title compound (150 mg, 54% yield). MS obsd. (ESI⁺): 366.3 [(M+H)⁺].

### Step C: Lithium 2-amino-5-(6-(tert-butoxycarbonyl)-2,6-diazaspiro[3.3]heptan-2-yl)-4-fluorobenzoate

To a mixture of tert-butyl 6-(4-amino-2-fluoro-5-(methoxycarbonyl)phenyl)-2,6-diazaspiro[3.3]heptane-2-carboxylate (100 mg, 0.27 mmol) in MeOH (3 mL) at 0° C. was added a solution of lithium hydroxide (13 mg, 0.55 mmol) in H₂O (1 mL). The mixture was stirred at 30° C. for 16 hr. The mixture was concentrated to provide the crude title compound (97 mg, crude). This material was used without further purification. MS obsd. (ESI⁺): 352.2 [(M+H)⁺] for free acid.

### Step D: tert-butyl (R)-6-(4-amino-2-fluoro-5-((1-(2-methyl-3-(trifluoromethyl)phenyl)ethyl) carba-moyl)phenyl)-2,6-diazaspiro[3.3]heptane-2-carboxylate

To a mixture of lithium 2-amino-5-(6-(tert-butoxycarbonyl)-2,6-diazaspiro[3.3]heptan-2-yl)-4-fluorobenzoate (97 mg, crude) in DMF (3 mL) was added HATU (155 mg, 0.41 mmol), DIPEA (105 mg, 0.81 mmol) followed by (R)-1-(2-methyl-3-(trifluoromethyl)phenyl) ethan-1-amine (61 mg, 0.3 mmol). The mixture was stirred at rt for 2 hr. The mixture was diluted with EtOAc (20 mL), washed with H₂O (15 mL×3), brine (15 mL×3), dried (Na₂SO₄), filtered and concentrated. The residue was purified by silica gel column (PE:EtOAc=1:1) to provide the title compound (130 mg, 89% yield). MS obsd. (ESI⁺): 537.2 [(M+H)⁺].

### Step E: (R)-2-amino-4-fluoro-N-(1-(2-methyl-3-(trifluoromethyl)phenyl)ethyl)-5-(2,6-diazas-piro[3.3]heptan-2-yl)benzamide 2,2,2-trifluoroacetate salt

To a mixture of tert-butyl (R)-6-(4-amino-2-fluoro-5-((1-(2-methyl-3-(trifluoromethyl)phenyl)ethyl) carbamoyl)phenyl)-2,6-diazaspiro[3.3]heptane-2-carboxylate (115 mg, 0.21 mmol) in DCM (5 mL) was added TFA (2.5 mL). The mixture was stirred at rt for 2 hr. The mixture was concentrated to provide the crude product (117 mg, crude). This material was used without further purification. MS obsd. (ESI⁺): 437.4 [(M+H)⁺] for free amine.

### Step F: (R)-5-(6-acetyl-2,6-diazaspiro[3.3]heptan-2-yl)-2-amino-4-fluoro-N-(1-(2-methyl-3-(trifluoromethyl)phenyl)ethyl)benzamide

To a mixture of acetic acid (13 mg, 0.21 mmol) in DMF (3 mL) was added HATU (121 mg, 0.32 mmol), DIPEA (82 mg, 0.64 mmol) and (R)-2-amino-4-fluoro-N-(1-(2-methyl-3-(trifluoromethyl)phenyl)ethyl)-5-(2,6-diazaspiro[3.3]heptan-2-yl)benzamide 2,2,2-trifluoroacetate salt (117 mg, crude). The mixture was stirred at rt for 2 hr. The mixture was diluted with EtOAc (20 mL). The organic mixture was washed with H₂O (15 mL×3), brine (15 mL×3), dried (Na₂SO₄), filtered and concentrated. The residue was purified by silica gel column (DCM:MeOH=1:1) to provide an impure title compound (85 mg, ˜50% purity, contaminated with some bis-acetylated product). This material is used without further purification. MS obsd. (ESI⁺): 479.2 [(M+H)⁺].

### Step G: (R)-5-(6-acetyl-2,6-diazaspiro[3.3]heptan-2-yl)-4-fluoro-N-(1-(2-methyl-3-(trifluoromethyl)phenyl)ethyl)-2-((1-methylpiperidin-4-yl)amino)benzamide (example 20)

To a mixture of (R)-5-(6-acetyl-2,6-diazaspiro[3.3]heptan-2-yl)-2-amino-4-fluoro-N-(1-(2-methyl-3-(trifluoromethyl)phenyl)ethyl)benzamide (75 mg, crude) in acetic acid (3 mL) was added 1-methylpiperidin-4-one (89 mg, 0.78 mmol) and sodium triacetoxyborohydride (166 mg, 0.78 mmol). The mixture was stirred at 25° C. for 2 hr. The mixture was concentrated, then the residue was diluted in DCM (20 mL). The mixture was washed with saturated NaHCO₃ aqueous (10 mL×3), H₂O (10 mL×3), and brine (10 mL×3). The organic layer was dried (Na₂SO₄), concentrated, and purified by preparative HPLC (ACN/water/0.1% HCOOH) to provide the title compound (13.1 mg, 0.65 eq. FA salt). MS obsd. (ESI⁺): 576.5 [(M+H)⁺]. ¹H NMR (400 MHz, DMSO-d₆) δ:8.68 (1H), 7.72 (1H), 7.57 (1H), 7.40 (1H), 7.34 (1H), 6.91 (1H), 6.51 (1H), 5.36 (1H), 4.28 (2H), 4.00 (2H), 3.93 (4H), 3.22 (1H), 2.61 (2H), 2.47 (3H), 2.16 (3H), 2.14-2.04 (2H), 1.82 (2H), 1.75 (3H), 1.44 (3H), 1.36-1.23 (2H).

### Example 21: (R)-4-fluoro-N-(1-(2-methyl-3-(trifluoromethyl)phenyl)ethyl)-2-((1-methylpiperidin-4-yl)amino)-5-(tetrahydro-2H-pyran-4-yl)benzamide

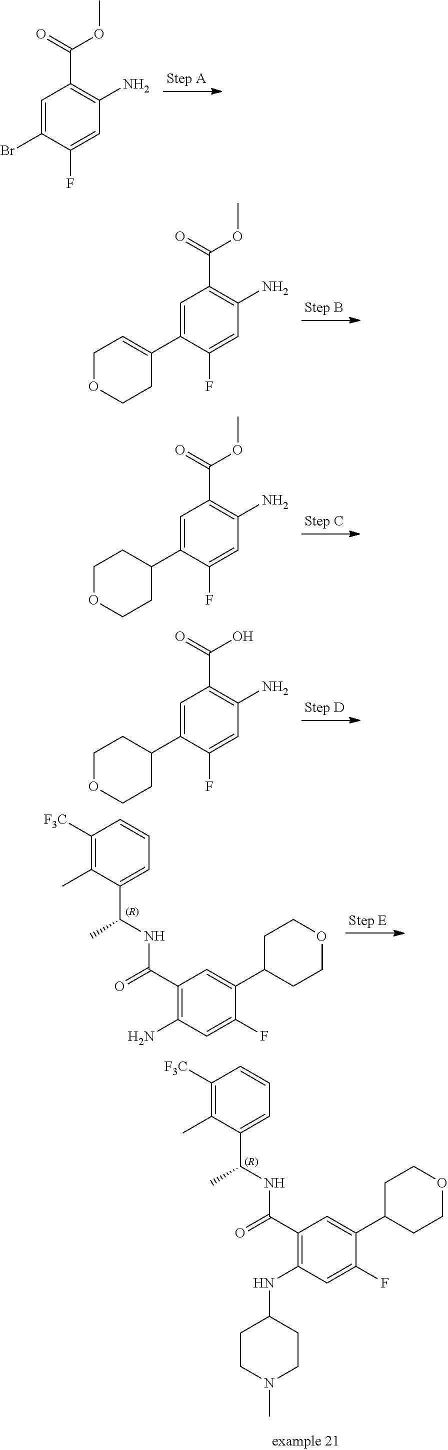

### Step A: Methyl 2-amino-5-(3,6-dihydro-2H-pyran-4-yl)-4-fluorobenzoate

Methyl 2-amino-5-bromo-4-fluoro-benzoate (500 mg, 2.02 mmol), 2-(3,6-dihydro-2H-pyran-4-yl)-4,4,5,5-tetramethyl-1,3,2-dioxaborolane (508 mg, 2.42 mmol), Pd (dppf) Cl₂ (74 mg, 100.79 μmol) and K₂CO₃ (557 mg, 4.03 mmol) were dissolved in 1,4-dioxane (5 mL) and H₂O (1 mL). The reaction mixture was stirred at 110° C. for 8 hr under N₂ atmosphere. The reaction mixture was poured into water (30 mL) and extracted with EtOAc (15 mL×4). The combined organic layers were washed with brine, dried over Na₂SO₄, filtered and concentrated to dryness. The residue was purified by column chromatography (eluted with 0-25% EtOAc in PE) to afford the title compound (263 mg, 51% yield). MS obsd. (ESI⁺): 252.3 [(M+H)⁺].

### Step B: Methyl 1-(1-(difluoromethyl)cyclopropyl)-4-((1-methylpiperidin-4-yl)amino)-6-oxo-1,6-methyl 2-amino-4-fluoro-5-(tetrahydro-2H-pyran-4-yl)benzoate

To a solution of methyl 2-amino-5-(3,6-dihydro-2H-pyran-4-yl)-4-fluoro-benzoate (230 mg, 0.91 mmol) in MeOH (3 mL) and EtOH (10 mL) was added Pd/C (30.0 mg, 0.028 mmol, 10 wt %). The reaction mixture was stirred for 2 hr at rt under H₂ atmosphere (1 atm). Then the reaction mixture was filtered and the filtrate was concentrated to dryness to afford the crude title compound (230 mg, crude). The crude product was used for next step directly without further purification. MS obsd. (ESI⁺): 254.2 [(M+H)⁺].

### Step C: 2-amino-4-fluoro-5-(tetrahydro-2H-pyran-4-yl)benzoic acid

Methyl 2-amino-4-fluoro-5-tetrahydropyran-4-yl-benzoate (220 mg, crude product) was dissolved in MeOH (15 mL) and THF (5 mL). Then LiOH (1 M, 5 mL) was added to the solution. The reaction mixture was stirred at 50° C. for 2 hr. After the reaction was completed, HCl (1 M, 5 mL) was added to the mixture. The mixture was extracted with EtOAc (15 mL×3). The combined organic layers were washed with brine, dried over Na₂SO₄, filtered and concentrated to afford the title compound (205 mg, crude), which was used without further purification. MS obsd. (ESI⁺): 240.3 [(M+H)⁺].

### Step D: (R)-2-amino-4-fluoro-N-(1-(2-methyl-3-(trifluoromethyl)phenyl)ethyl)-5-(tetrahydro-2H-pyran-4-yl)benzamide

2-amino-4-fluoro-5-tetrahydropyran-4-yl-benzoic acid (205 mg, crude product) and HATU (491 mg, 1.29 mmol) were dissolved in DMF (10 mL). The mixture was stirred for 0.5 hr at r.t. Then DIPEA (223 mg, 1.72 mmol) and (R)-1-(2-methyl-3-(trifluoromethyl)phenyl) ethan-1-amine (210 mg, 1.03 mmol) were added to the mixture. The mixture was stirred for 1.5 hr at room temperature. The mixture was poured into water (100 mL) and extracted with EtOAc (15 mL×3). The combined organic layers were washed with brine, dried over Na₂SO₄, filtered and concentrated to dryness. The residue was purified by column chromatography (eluted with 0-25% EtOAc in PE) to afford the title compound (313.5 mg, 85% yield). MS obsd. (ESI⁺): 425.4 [(M+H)⁺].

### Step E: (R)-4-fluoro-N-(1-(2-methyl-3-(trifluoromethyl)phenyl)ethyl)-2-((1-methylpiperidin-4-yl)amino)-5-(tetrahydro-2H-pyran-4-yl)benzamide (example 21)

To a solution of (R)-2-amino-4-fluoro-N-(1-(2-methyl-3-(trifluoromethyl)phenyl)ethyl)-5-(tetrahydro-2H-pyran-4-yl)benzamide (310 mg, 0.73 mmol) in AcOH (10 mL) was added 1-methylpiperidin-4-one (124 mg, 1.10 mmol) at 0° C. Then NaBH (OAc) 3 (310 mg, 1.46 mmol) was added to the mixture. The mixture was stirred at 0° C. for 1 hr. The reaction mixture was then poured into water (50 mL) and the mixture was adjusted to pH=10 with solid Na₂CO₃. The mixture was extracted with EtOAc (15 mL×4). The combined organic layers were washed with brine, dried over Na₂SO₄, filtered and concentrated to dryness. The residue was purified by preparative HPLC (ACN/water/0.1% NH₄HCO₃) to afford the title compound (88.6 mg, 23% yield). MS obsd. (ESI⁺): 522.2 [(M+H)⁺]. ¹H NMR (400 MHZ, DMSO-d₆) δ:8.69 (1H), 7.95 (1H), 7.74 (1H), 7.68 (1H), 7.57 (1H), 7.42 (1H), 6.45 (1H), 5.38 (1H), 3.97 (2H), 3.44 (2H), 3.25 (1H), 2.87 (1H), 2.57 (2H), 2.48 (3H), 2.13 (3H), 2.06 (2H), 1.91-1.80 (4H), 1.60 (2H), 1.45 (3H), 1.39-1.23 (2H).

### Example 22: (R)-5-(4-acetylpiperazin-1-yl)-4-fluoro-N-(1-(2-methyl-3-(trifluoromethyl)phenyl)ethyl)-2-((1-methylpiperidin-4-yl)amino)benzamide

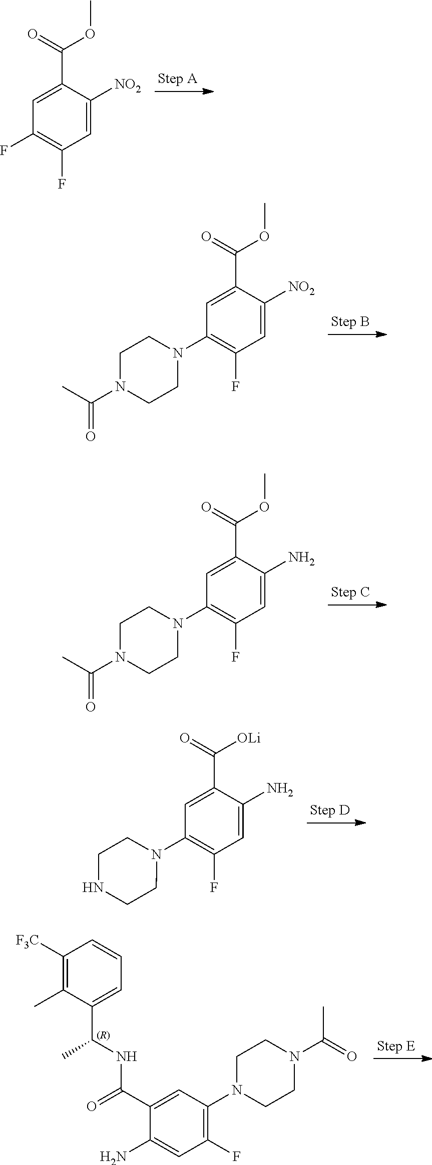

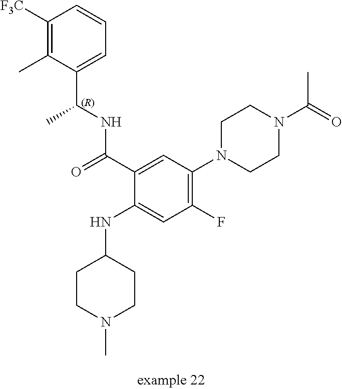

### Step A: Methyl 5-(4-acetylpiperazin-1-yl)-4-fluoro-2-nitrobenzoate

To a solution of methyl 4,5-difluoro-2-nitrobenzoate (300 mg, 1.38 mmol) and 1-(piperazin-1-yl) ethan-1-one (266 mg, 2.07 mmol) in DMSO (2 mL) was added triethylamine (419 mg, 4.15 mmol). The mixture was stirred for 16 hr at 15° C. The mixture was quenched with water and extracted with DCM (3×80 mL). The combined organic layers were washed with water (3×50 mL), dried over Na₂SO₄, filtered and concentrated. Purification by flash column chromatography (eluted with 0˜80% EtOAc in PE) afforded the title compound (405 mg, 90% yield). MS obsd. (ESI⁺): 326.3 [(M+H)⁺].

### Step B: Methyl 5-(4-acetylpiperazin-1-yl)-2-amino-4-fluorobenzoate

To a solution of methyl 5-(4-acetylpiperazin-1-yl)-4-fluoro-2-nitrobenzoate (472 mg, 1.45 mmol) in AcOH (10 mL) was added iron powder (810 mg, 14.51 mmol). The mixture was stirred for 1 hr at 50° C. The mixture was filtered. The filtrate was concentrated in vacuo and to the residue was added saturated NaHCO₃ aqueous solution. Then the mixture was extracted with DCM (3×50 mL). The combined organic layers were dried over sodium sulfate, filtered and concentrated. The residue was purified by silica gel chromatography (eluted with 0-50% EtOAc in PE) to afford the title compound (393 mg, 91% yield). MS obsd. (ESI⁺): 296.3 [(M+H)⁺].

### Step C: Lithium 2-amino-4-fluoro-5-(piperazin-1-yl)benzoate

To a solution of methyl 5-(4-acetylpiperazin-1-yl)-2-amino-4-fluorobenzoate (200 mg, 0.67 mmol) in THF (4 mL) and water (0.5 mL) was added lithium hydroxide (65 mg, 2.71 mmol). The mixture was stirred for 3 hr at 80° C. The solvent was removed in vacuo to afford the crude title compound (166 mg, crude). The crude product was used in the next step directly without further purification. MS obsd. (ESI⁺): 246.3 [(M+H)⁺] for free acid.

### Step D: (R)-5-(4-acetylpiperazin-1-yl)-2-amino-4-fluoro-N-(1-(2-methyl-3-(trifluoromethyl)phenyl)ethyl)benzamide

To a solution of acetic acid (37 mg, 0.62 mmol) in DMF (5 mL) was added HATU (234 mg, 0.62 mmol) and N,N-Diisopropylethylamine (239 mg, 1.85 mmol). The mixture was stirred for 0.5 hr at rt. Then lithium 2-amino-4-fluoro-5-(piperazin-1-yl)benzoate (166 mg, crude) was added into the mixture and stirred for 1 hr at rt. Then HATU (350 mg, 0.92 mmol) was added. The mixture was stirred for 0.5 hr at rt. Then N,N-Diisopropylethylamine (158 mg, 1.23 mmol) and (R)-1-(2-methyl-3-(trifluoromethyl)phenyl) ethan-1-amine (187 mg, 0.92 mmol) were added and the mixture was stirred for 1 hr further. The reaction was quenched with water and extracted with DCM (3×80 mL). The combined organic layers were washed with water (3×50 mL), dried over Na₂SO₄, filtered and concentrated. The residue was purified by flash column chromatography (eluted with 0-5% MeOH in DCM) to afford the title compound (261 mg). MS obsd. (ESI⁺): 467.2 [(M+H)⁺].

### Step E: (R)-5-(4-acetylpiperazin-1-yl)-4-fluoro-N-(1-(2-methyl-3-(trifluoromethyl)phenyl)ethyl)-2-((1-methylpiperidin-4-yl)amino)benzamide (example 22)

To a solution of (R)-5-(4-acetylpiperazin-1-yl)-2-amino-4-fluoro-N-(1-(2-methyl-3-(trifluoromethyl)phenyl)ethyl)benzamide (261 mg, 0.56 mmol) and 1-methylpiperidin-4-one (633 mg, 5.60 mmol) in AcOH (10 mL) was added sodium triacetoxyborohydride (1.2 g, 5.60 mmol). The mixture was stirred for 1 hr at rt. At this time, an equivalent portion of of 1-methylpiperidin-4-one and sodium triacetoxyborohydride were added into the mixture and the mixture stirred for 1 hr further. The reaction mixture was concentrated and diluted with water, stirring for 0.5 hr. Then saturated NaHCO₃ solution was added into the mixture dropwise until the solution was approximately pH 8. The mixture was extracted with DCM (3×50 mL), and the combined organic layers were dried over Na₂SO₄, filtered and concentrated. The residue was purified by flash chromatography (eluted with 0-20% MeOH in DCM) followed by preparative HPLC (ACN/water/0.1% NH₄HCO₃) to afford the title compound (95.8 mg, 30% yield). MS obsd. (ESI⁺): 564.5 [(M+H)⁺]. ¹H NMR (400 MHZ, DMSO-d₆) δ: 8.65 (1H), 7.72 (2H), 7.57 (1H), 7.43-7.39 (2H), 6.54 (1H), 5.37 (1H), 3.62-3.55 (4H), 3.29-3.20 (1H), 2.96-2.84 (4H), 2.59-2.56 (2H), 2.47 (3H), 2.13 (3H), 2.09 (1H), 2.05 (4H), 1.82 (2H), 1.44 (3H), 1.37-1.23 (2H).

### Example 23: (R)—N-(1-(2-methyl-3-(trifluoromethyl)phenyl)ethyl)-2-((1-methylpiperidin-4-yl)amino)-5-morpholinobenzamide

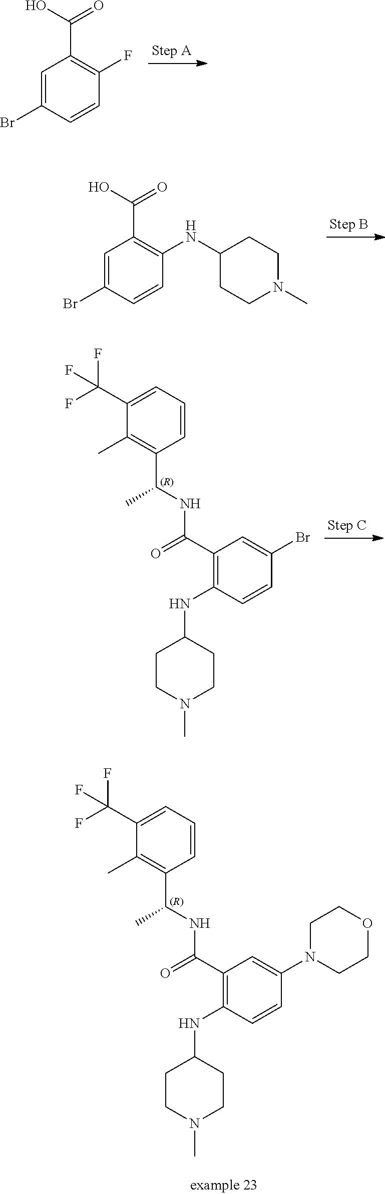

### Step A: 5-bromo-2-((1-methylpiperidin-4-yl)amino)benzoic acid

To a stirred mixture of 5-bromo-2-fluorobenzoic acid (2 g, 9.13 mmol) and 1-methylpiperidin-4-amine (1.15 g, 10.05 mmol) in DMSO (25 mL) was added N-ethyl-N-isopropyl-propan-2-amine (2.35 g, 18.26 mmol) at rt. The resulting mixture was stirred for 48 hr at 100° C. The reaction mixture was quenched by water (50 ml). The resulting mixture was extracted with EtOAc (3×200 mL). The combined organic layers were washed with brine (300 mL), dried over anhydrous sodium sulfate. After filtration, the filtrate was concentrated under reduced pressure. The residue was purified by silica gel column chromatography, eluted with PE/EtOAc (0-60%) to afford the title compound (320 mg, 11% yield). MS obsd. (ESI⁺): 313.25 [M+H]⁺.

### Step B: (R)-5-bromo-N-(1-(2-methyl-3-(trifluoromethyl)phenyl)ethyl)-2-((1-methylpiperidin-4-yl)amino)benzamide

To a stirred mixture of 5-bromo-2-((1-methylpiperidin-4-yl)amino)benzoic acid (320 mg, 1.02 mmol) and (R)-1-(2-methyl-3-(trifluoromethyl)phenyl) ethan-1-amine (207.0 mg, 1.02 mmol) in DMF (4 mL) was added HATU (388.7 mg, 1.02 mmol) and N-ethyl-N-isopropyl-propan-2-amine (259.29 mg, 2.04 mmol). The resulting mixture was stirred at room temperature for 2 hr. The reaction mixture was quenched by water (50 ml). The resulting mixture was extracted with EtOAc (3×100 mL). The combined organic layers were washed with brine (100 mL), dried over anhydrous sodium sulfate. After filtration, the filtrate was concentrated under reduced pressure. The residue was purified by silica gel column chromatography, eluted with PE/EtOAc (0-50%) to afford the title compound (300 mg, 0.60 mmol, 59% yield). MS obsd. (ESI⁺): 498.10 [M+H]⁺.

### Step C: (R)—N-(1-(2-methyl-3-(trifluoromethyl)phenyl)ethyl)-2-((1-methylpiperidin-4-yl)amino)-5-morpholinobenzamide (Example 23)

To a stirred mixture of (R)-5-bromo-N-(1-(2-methyl-3-(trifluoromethyl)phenyl)ethyl)-2-((1-methylpiperidin-4-yl)amino)benzamide (150 mg, 0.30 mmol) and morpholine (131 mg, 1.50 mmol) in dioxane (3 mL) was added Pd₂(dba)₃ (27.5 mg, 0.03 mmol), dicyclohexyl-[2-(2,4,6-triisopropylphenyl)phenyl]phosphane (14.3 mg, 0.03 mmol) and cesium carbonate (196 mg, 0.6 mmol). The resulting mixture was stirred for 3 hr at 100° C. under N₂ atmosphere. The resulting mixture was concentrated under reduced pressure. The residue was purified by Preparative TLC (DCM/MeOH=10/1). Further purification by Preparative HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 35% B to 55% B in 7 min, 55% B). This afforded the title compound (40.2 mg, 26% yield). ¹⁹F NMR (376 MHz, DMSO-d₆) δ−58.706. MS obsd. (ESI⁺): 505.30 [M+H]⁺.

### Examples 24 and 25:4-cyano-N—((R)-1-(3-(difluoromethyl)-2-fluorophenyl)ethyl)-5-morpholino-2-(((R)-quinuclidin-3-yl)amino)benzamide and 4-cyano-N—((R)-1-(3-(difluoromethyl)-2-fluorophenyl)ethyl)-5-morpholino-2-(((S)-quinuclidin-3-yl)amino)benzamide (stereochemistry not assigned)

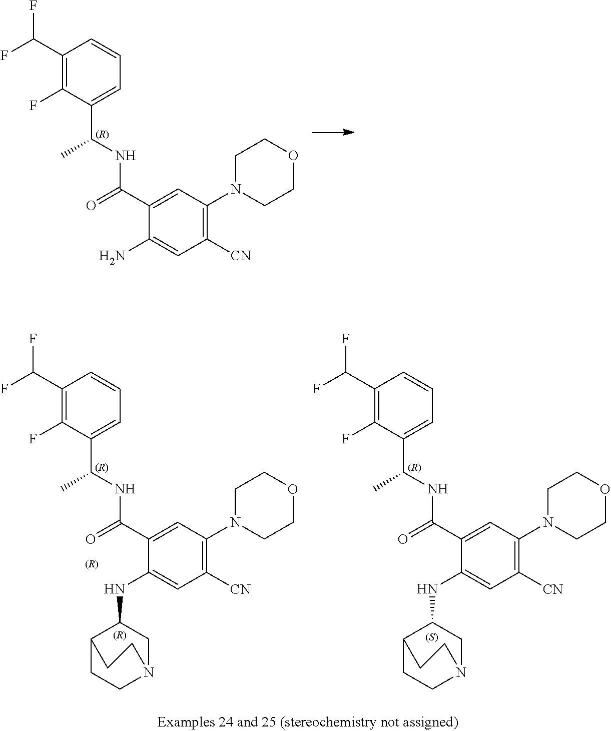

To a solution of (R)-2-amino-4-cyano-N-(1-(3-(difluoromethyl)-2-fluorophenyl)ethyl)-5-morpholinobenzamide (180 mg, 0.43 mmol) in AcOH (5 mL) was added quinuclidin-3-one hydrochloride (695 mg, 4.30 mmol) and sodium cyanoborohydride (135 mg, 2.15 mmol) at 60° C. The reaction mixture was stirred for 4 hrs at 60° C. After cooling to room temperature, the solvent was removed under reduced pressure. The residue was first purified by silica gel chromatography (eluting with 10% to 20% MeOH in DCM) followed by preparative HPLC (ACN/water/0.1% NH₄HCO₃) to afford the title compound as mixture of diastereomers (85 mg, 37% yield).

The mixture was separated by preparative SFC (Regis (R,R) Whelk-O1 (25*250 mm, 10 μm), CO₂/EtOH [0.5% NH₃ (7M in MeOH)]=60/40) to afford the title compounds.

Example 24: First eluting peak. MS obsd. (ESI⁺): 528.8 [M+H]⁺. ¹H NMR (400 MHZ, DMSO-d₆) δ:9.00 (1H), 7.65 (1H), 7.54 (2H), 7.47 (1H), 7.36 (1H), 7.22 (1H), 6.98 (1H), 5.38 (1H), 3.80-3.74 (4H), 3.52 (1H), 3.23 (1H), 3.06-2.98 (4H), 2.72-2.57 (4H), 2.20 (1H), 1.81 (1H), 1.59 (2H), 1.51 (4H), 1.31 (1H).

Example 25: Second eluting peak. MS obsd. (ESI⁺): 528.8 [M+H]⁺. ¹H NMR (400 MHZ, DMSO-d₆) δ: 9.01 (1H), 7.64 (1H), 7.53 (1H), 7.45 (2H), 7.35 (1H), 7.22 (1H), 6.99 (1H), 5.37 (1H), 3.81-3.72 (4H), 3.54 (1H), 3.24 (1H), 3.02 (4H), 2.63 (4H), 2.23 (1H), 1.78 (1H), 1.63-1.54 (2H), 1.51 (3H), 1.43 (1H), 1.25 (1H).

### Examples 26 and 27: N—((R)-1-(3-(difluoromethyl)-2-fluorophenyl)ethyl)-5-(3,3-difluoropyrrolidin-1-yl)-4-fluoro-2-(((S)-quinuclidin-3-yl)amino)benzamide and N—((R)-1-(3-(difluoromethyl)-2-fluorophenyl)ethyl)-5-(3,3-difluoropyrrolidin-1-yl)-4-fluoro-2-(((R)-quinuclidin-3-yl)amino)benzamide (diastereomers not assigned)

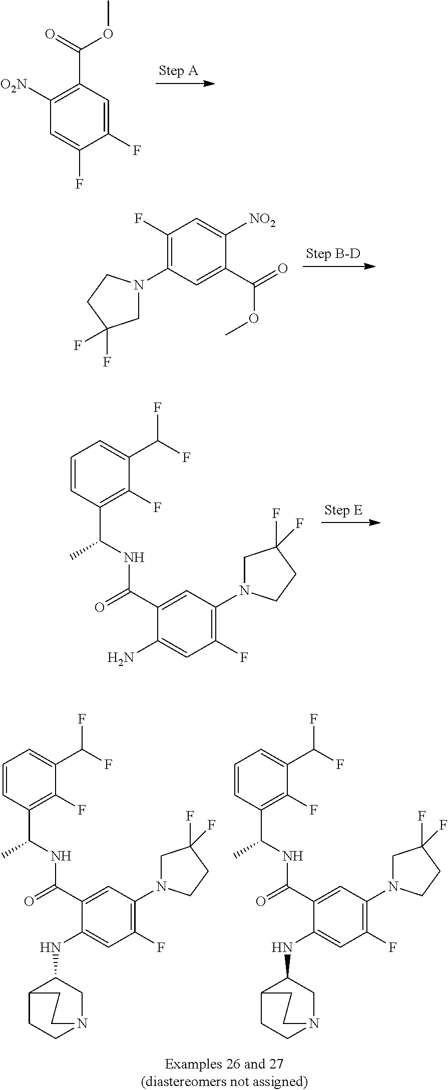

### Step A: Methyl 5-(3,3-difluoropyrrolidin-1-yl)-4-fluoro-2-nitrobenzoate

To a solution of methyl 4,5-difluoro-2-nitro-benzoate (1 g, 4.61 mmol) in DMSO (5 mL) was added 3,3-difluoropyrrolidine hydrochloride (793 mg, 5.53 mmol) and triethylamine (699 mg, 6.91 mmol), and the mixture was stirred at rt for 3 hrs. The mixture was diluted with DCM (150 mL), washed with water (150 mL×2) and the organic layer was dried over Na₂SO₄, filtered and concentrated. The residue was purified by column chromatography on silica gel (EA/PE, 0-50%) to afford the title compound (1.1 g, 78% yield). ¹H NMR (400 MHZ, DMSO-d₆) δ 8.03 (d, J=13.8 Hz, 1H), 6.95 (d, J=8.4 Hz, 1H), 4.03 (td, J=13.0, 2.8 Hz, 2H), 3.84 (s, 3H), 3.80 (m, 2H), 2.59-2.51 (m, 2H).

### Steps B-D: (R)-2-amino-N-(1-(3-(difluoromethyl)-2-fluorophenyl)ethyl)-5-(3,3-difluoropyrrolidin-1-yl)-4-fluorobenzamide

Synthesized according to procedures described for example 7 steps B-D. MS obsd. (ESI⁺): 432.1 [M+H]⁺.

### Step E: N—((R)-1-(3-(difluoromethyl)-2-fluorophenyl)ethyl)-5-(3,3-difluoropyrrolidin-1-yl)-4-fluoro-2-(((R)-quinuclidin-3-yl)amino)benzamide & N—((R)-1-(3-(difluoromethyl)-2-fluorophenyl)ethyl)-5-(3,3-difluoropyrrolidin-1-yl)-4-fluoro-2-(((S)-quinuclidin-3-yl)amino)benzamide (Examples 26 and 27)

To a solution of (R)-2-amino-N-(1-(3-(difluoromethyl)-2-fluorophenyl)ethyl)-5-(3,3-difluoropyrrolidin-1-yl)-4-fluorobenzamide (500 mg, 1.16 mmol) in AcOH (15 mL) was added quinuclidin-3-one hydrochloride (1.9 g, 11.59 mmol), and sodium cyanoborohydride (437 mg, 6.95 mmol), and the mixture was stirred at 60° C. for 16 hrs. The mixture was concentrated under vacuum, and the residue was dissolved in water (50 mL) and neutralized with aqueous sodium carbonate. The mixture was extracted with DCM (80 mL×3). The combined organic layers were dried over Na2SO4, filtered and concentrated. The residue was purified by column chromatography on silica gel (MeOH/DCM, 0-20%) followed by preparative HPLC (0.5% NH₄HCO₃/ACN) to afford the title compounds as a diastereomeric mixture (85 mg, 13% yield). MS obsd. (ESI⁺): 541.0 [M+H]⁺.

The mixture was further separated by chiral SFC (Column Name: Daicel OZ (25*250 mm, 10 μm); CO₂/MeOH [0.2% NH₃ (7M in MeOH)]) to provide the title compounds.

Example 26: First eluting isomer. MS obsd. (ESI⁺): 541.0 [M+H]⁺. ¹H NMR (400 MHZ, DMSO-d₆) δ 8.68 (1H), 7.90 (1H), 7.65 (1H), 7.52 (1H), 7.39-7.08 (3H), 6.48 (1H), 5.39 (1H), 3.59 (2H), 3.41 (3H), 3.23 (1H), 2.66 (4H), 2.44 (2H), 2.24 (1H), 1.82 (1H), 1.63-1.52 (3H), 1.49 (3H), 1.31 (1H).

Example 27: Second eluting isomer. MS obsd. (ESI⁺): 541.0 [M+H]⁺. ¹H NMR (400 MHZ, DMSO-d₆) δ 8.69 (1H), 7.81 (1H), 7.63 (1H), 7.52 (1H), 7.39-7.08 (3H), 6.48 (1H), 5.37 (1H), 3.59 (2H), 3.41 (3H), 3.23 (1H), 2.71-2.57 (4H), 2.49-2.40 (2H), 2.25 (1H), 1.78 (1H), 1.56 (2H), 1.52-1.41 (4H), 1.25 (1H).

### Examples 28 and 29:5-(azetidin-1-yl)-N—((R)-1-(3-(difluoromethyl)-2-fluorophenyl)ethyl)-4-fluoro-2-(((S)-quinuclidin-3-yl)amino)benzamide and 5-(azetidin-1-yl)-N—((R)-1-(3-(difluoromethyl)-2-fluorophenyl)ethyl)-4-fluoro-2-(((R)-quinuclidin-3-yl)amino)benzamide (diastereomers not assigned)

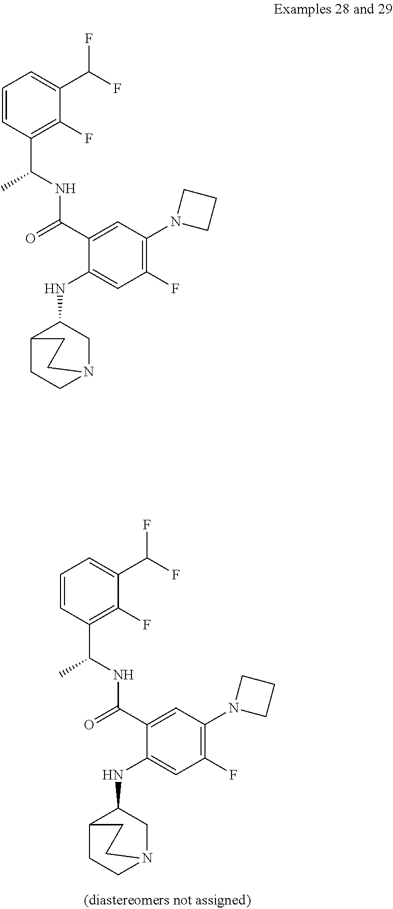 [SMILES: C[C@@H](NC(=O)c1cc(N2CCC2)c(F)cc1N[C@@H]1CN2CCC1CC2)c1cccc(C(F)F)c1F ; C[C@@H](NC(=O)c1cc(N2CCC2)c(F)cc1N[C@H]1CN2CCC1CC2)c1cccc(C(F)F)c1F]

Synthesized according to analogous procedures described for examples 26 and 27, starting with azetidine hydrochloride in step A. The diastereomeric mixture was separated by chiral SFC (column: Daicel OD (25*250 mm, 10 μm)); Mobile phase: CO₂/MeOH [0.2% NH₃ (7M in MeOH)]=80/20) to afford the title compounds.

Example 28: First eluting peak. MS obsd. (ESI⁺): 491.0 [(M+H)⁺]. ¹H NMR (400 MHZ, DMSO-d₆) δ 8.70 (1H), 7.66-7.55 (2H), 7.51 (1H), 7.27 (2H), 6.92 (1H), 6.42 (1H), 5.36 (1H), 3.80 (4H), 3.26-3.21 (2H), 2.72-2.60 (4H), 2.32-2.20 (3H), 1.79 (1H), 1.62-1.55 (2H), 1.52-1.43 (4H), 1.26 (1H).

Example 29: Second eluting peak. MS obsd. (ESI⁺): 491.0 [(M+H)⁺]. ¹H NMR (400 MHZ, DMSO-d₆) δ 8.72 (1H), 7.71 (1H), 7.64 (1H), 7.52 (1H), 7.36 (1H), 7.21 (1H), 6.95 (1H), 6.44 (1H), 5.38 (1H), 3.80 (4H), 3.51 (2H), 2.79 (4H), 2.41 (1H), 2.30-2.20 (2H), 1.92-1.85 (1H), 1.72-1.57 (3H), 1.48 (3H), 1.41 (1H).

### Examples 30 and 31: N—((R)-1-(3-(difluoromethyl)-2-fluorophenyl)ethyl)-4-fluoro-2-((1-methylpiperidin-4-yl)amino)-5-((R)-2-oxa-7-azaspiro[4.4]nonan-7-yl)benzamide and N—((R)-1-(3-(difluoromethyl)-2-fluorophenyl)ethyl)-4-fluoro-2-((1-methylpiperidin-4-yl)amino)-5-((S)-2-oxa-7-azaspiro[4.4]nonan-7-yl)benzamide (diastereomers not assigned)

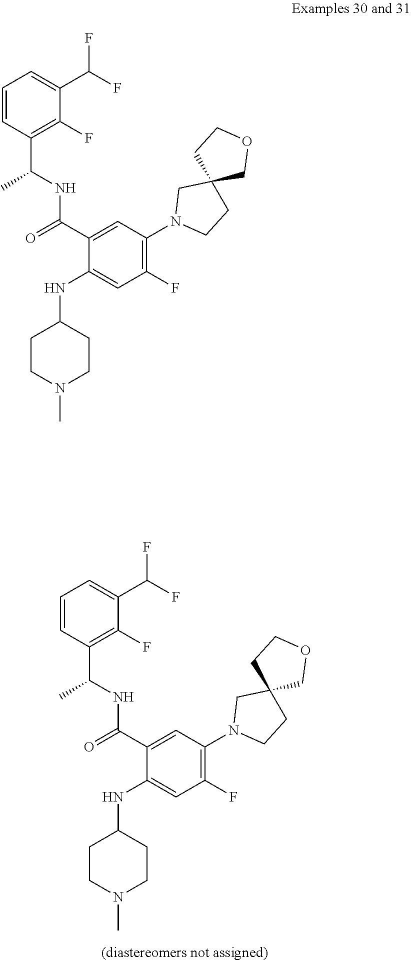 [SMILES: C[C@@H](NC(=O)c1cc(N2CC[C@@]3(CCOC3)C2)c(F)cc1NC1CCN(C)CC1)c1cccc(C(F)F)c1F ; C[C@@H](NC(=O)c1cc(N2CC[C@]3(CCOC3)C2)c(F)cc1NC1CCN(C)CC1)c1cccc(C(F)F)c1F]

Synthesized according to analogous protocols described for Example 5, starting with 2-oxa-7-azaspiro[4.4]nonane. The diastereomeric final mixture was separated via chiral SFC (Daicel AS (4.6*100 mm 3 μm), CO₂/IPA [0.5% NH₃ (7M in MeOH)]=85/15) to give separated enantiomers.

Example 30: First eluting isomer. MS obsd. (ESI⁺): 549.6 [(M+H)⁺]. ¹H NMR (400 MHZ, DMSO-d₆) δ 8.67 (1H), 7.62 (1H), 7.51 (1H), 7.39-6.98 (4H), 6.53 (1H), 5.44-5.26 (1H), 3.80 (2H), 3.60 (2H), 3.30-3.08 (5H), 2.71-2.63 (2H), 2.22-1.72 (11H), 1.48 (3H), 1.38-1.18 (2H).

Example 31: Second eluting isomer. MS obsd. (ESI⁺): 549.6 [(M+H)⁺]. ¹H NMR (400 MHZ, DMSO-d₆) δ 8.67 (1H), 7.63 (1H), 7.51 (1H), 7.38-7.07 (4H), 6.54 (1H), 5.36 (1H), 3.80 (2H), 3.60 (2H), 3.25 (4H), 3.16 (1H), 2.59 (2H), 2.22-2.04 (5H), 2.01-1.77 (6H), 1.48 (3H), 1.39-1.18 (2H).

### Examples 32 and 33: N—((R)-1-(3-(difluoromethyl)-2-fluorophenyl)ethyl)-4-fluoro-5-(pyrrolidin-1-yl)-2-(((S)-quinuclidin-3-yl)amino)benzamide and N—((R)-1-(3-(difluoromethyl)-2-fluorophenyl)ethyl)-4-fluoro-5-(pyrrolidin-1-yl)-2-(((R)-quinuclidin-3-yl)amino)benzamide (diastereomers not assigned)

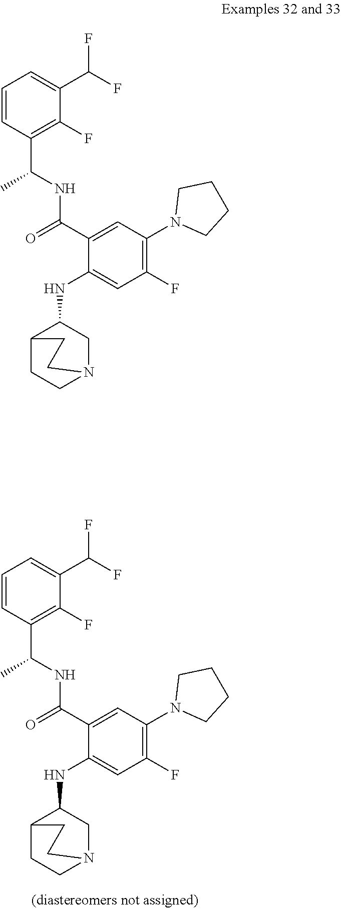 [SMILES: C[C@@H](NC(=O)c1cc(N2CCCC2)c(F)cc1N[C@@H]1CN2CCC1CC2)c1cccc(C(F)F)c1F ; C[C@@H](NC(=O)c1cc(N2CCCC2)c(F)cc1N[C@H]1CN2CCC1CC2)c1cccc(C(F)F)c1F]

Prepared in an analogous fashion to examples 26 and 27. The diastereomeric final mixture was purified by chiral SFC (column: Daicel OZ (25*250 mm, 10 μm)); Mobile phase: CO₂/MeOH [0.2% NH₃ (7M in MeOH)]=60/40) to afford individual diastereomers.

Example 32: First eluting isomer. MS obsd. (ESI⁺): 505.6 [(M+H)⁺]. ¹H NMR (400 MHZ, DMSO-d₆) δ 8.69 (1H), 7.63 (2H), 7.52 (1H), 7.37-7.10 (3H), 6.44 (1H), 5.38 (1H), 3.41 (1H), 3.19 (5H), 2.71-2.58 (4H), 2.22 (1H), 1.88 (4H), 1.80 (1H), 1.55 (3H), 1.48 (3H), 1.28 (1H).

Example 33: Second eluting isomer. MS obsd. (ESI⁺): 505.6 [(M+H)⁺]. ¹H NMR (400 MHZ, DMSO-d₆) δ 8.70 (1H), 7.63 (1H), 7.51 (2H), 7.36-7.09 (3H), 6.44 (1H), 5.37 (1H), 3.42 (1H), 3.25-3.14 (5H), 2.72-2.56 (4H), 2.25 (1H), 1.93-1.82 (4H), 1.77 (1H), 1.55 (2H), 1.45 (4H), 1.22 (1H).

The following examples were synthesized via the below general scheme, steps A-E according to analogous procedures described for example 5 with appropriate reagent substitutions (all reagents used are either commercially available or synthetically reported).

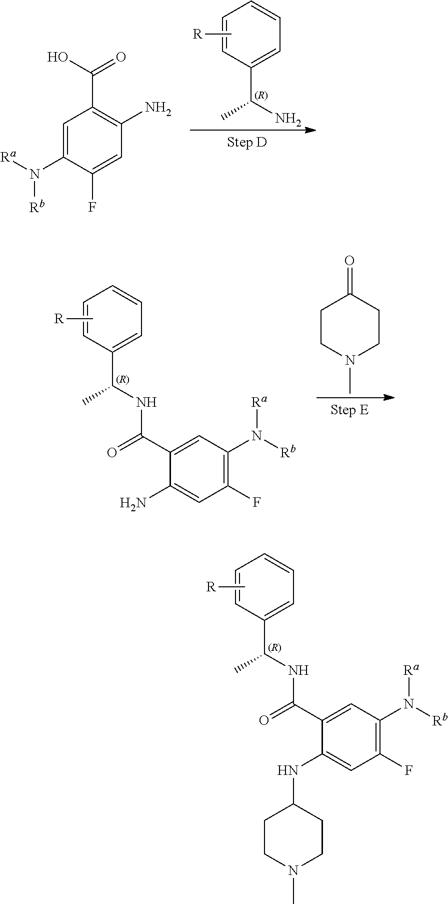

| Example | | | |
|---|---|---|---|
| # | Structure | Compound Name | Analytical Data |
| 34 and 35 | 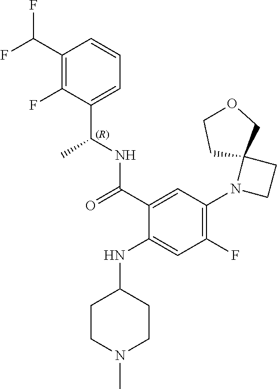 | N-((R)-1-(3- (difluoromethyl)-2- fluorophenyl)ethyl)-4- fluoro-2-((1- methylpiperidin-4- yl)amino)-5-((S)-6-oxa-1- azaspiro[3.4]octan-1- yl)benzamide and N-((R)- 1-(3-(difluoromethyl)-2- fluorophenyl)ethyl)-4- fluoro-2-((1- methylpiperidin-4- yl)amino)-5-((R)-6-oxa-1- azaspiro[3.4]octan-1- yl)benzamide (diastereomers not assigned) | Chiral SFC (Column: YMC Cellulose-SC (20 x 250 mm, 5 um), Mobile phase: CO₂/MeOH [0.2% NH3 (7M in MeOH)] = 65/35) Example 34: First eluting isomer: MS obsd. (ESI⁺): 535.7 [(M + H)⁺]. Example 35: Second eluting isomer: MS obsd. (ESI⁺): 535.7 [(M + H)⁺]. |
| | 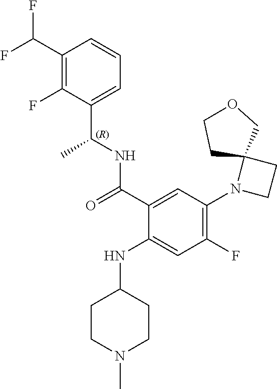 | | |
| 36 and 37 | 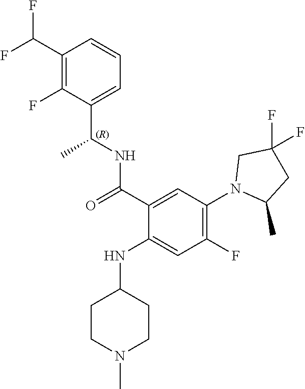 | 5-((R)-4,4-difluoro-2- methylpyrrolidin-1-yl)-N- ((R)-1-(3- (difluoromethyl)-2- fluorophenyl)ethyl)-4- fluoro-2-((1- methylpiperidin-4- yl)amino)benzamide and 5-((S)-4,4-difluoro-2- methylpyrrolidin-1-yl)-N- ((R)-1-(3- (difluoromethyl)-2- fluorophenyl)ethyl)-4- fluoro-2-((1- methylpiperidin-4- yl)amino)benzamide (diastereomers not assigned) | Chiral SFC (Column: YMC Cellulose-SC (25*250 mm, 10 um), Mobile phase: CO₂/MeOH[0.2% NH₃(7M in MeOH)] = 85/15) Example 36: First eluting isomer. MS obsd. (ESI⁺): 543.6 [(M + H)⁺]. Example 37: Second eluting isomer. MS obsd. (ESI⁺): 543.6 [(M + H)⁺]. |
| | 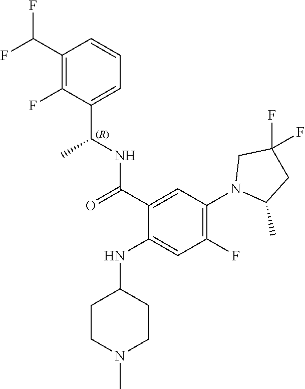 | | |
| 38 | 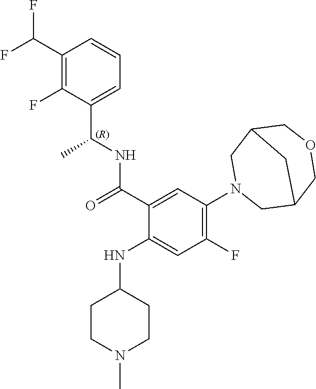 | 5-(3-oxa-7- azabicyclo[3.3.1]nonan-7- yl)-N-((R)-1-(3- (difluoromethyl)-2- fluorophenyl)ethyl)-4- fluoro-2-((1- methylpiperidin-4- yl)amino)benzamide | MS obsd. (ESI⁺): 549.3 [(M + H)⁺]. ¹H NMR (400 MHz, DMSO-d₆) formic acid salt δ 8.69 (1H), 8.18 (1H), 7.63 (1H), 7.58 (1H), 7.52 (1H), 7.29 (3H), 6.53 (1H), 5.37 (1H), 3.84 (2H), 3.74 (2H), 3.32 (1H), 3.02 (2H), 2.72 (2H), 2.31- 2.22 (5H), 1.86 (6H), 1.68 (1H), 1.49 (3H), 1.42-1.28 (2H). |
| 39 | 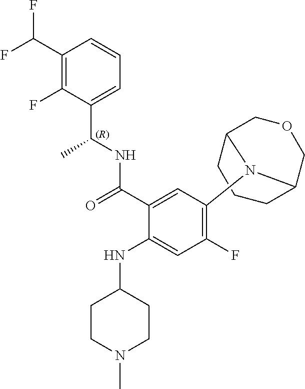 | 5-(3-oxa-9- azabicyclo[3.3.1]nonan-9- yl)-N-((R)-1-(3- (difluoromethyl)-2- fluorophenyl)ethyl)-4- fluoro-2-((1- methylpiperidin-4- yl)amino)benzamide | MS obsd. (ESI⁺): 549.3 [(M + H)⁺]. ¹H NMR (400 MHz, DMSO-d₆) δ 8.70 (1H), 7.61 (1H), 7.52 (1H), 7.36-7.08 (4H), 6.52 (1H), 5.35 (1H), 3.93 (2H), 3.82 (2H), 3.52 (2H), 3.25 (1H), 2.68 (2H), 2.22 (5H), 1.99 (2H), 1.89-1.80 (2H), 1.77-1.68 (2H), 1.60 (1H), 1.48 (3H), 1.31 (2H). |
| 40 | 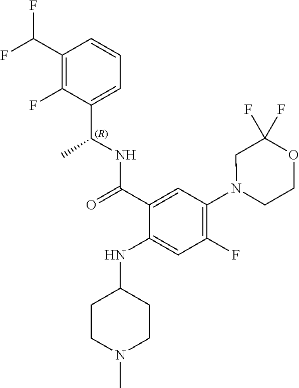 | (R)-N-(1-(3- (difluoromethyl)-2- fluorophenyl)ethyl)-5- (2,2-difluoromorpholino)- 4-fluoro-2-((1- methylpiperidin-4- yl)amino)benzamide | MS obsd. (ESI⁺): 545.6 [(M + H)⁺]. ¹H NMR (400 MHz, DMSO-d₆) δ 8.65 (1H), 7.81 (1H), 7.64 (1H), 7.52 (1H), 7.45 (1H), 7.35 (1H), 7.22 (1H), 6.59 (1H), 5.36 (1H), 4.16 (2H), 3.37 (2H), 3.27 (1H), 3.09 (2H), 2.59 (2H), 2.17- 2.08 (5H), 1.83 (2H), 1.49 (3H), 1.39-1.26 (2H). |
| 41 | 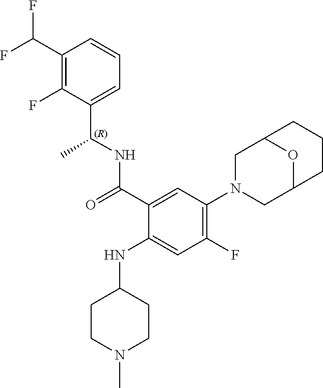 | 5-(9-oxa-3- azabicyclo[3.3.1]nonan-3- yl)-N-((R)-1-(3- (difluoromethyl)-2- fluorophenyl)ethyl)-4- fluoro-2-((1- methylpiperidin-4- yl)amino)benzamide | 9-oxa-3- azabicyclo[3.3.1]nonane hydrochloride (reagent used in step A) was made according to published protocol (RSC Advances 2016, 6, 53955-53957). MS obsd. (ESI⁺): 549.3 [(M + H)⁺]. ¹H NMR (400 MHz, DMSO-d₆): δ 8.67 (1H), 7.64 (2H), 7.52 (1H), 7.36-7.09 (3H), 6.55 (1H), 5.36 (1H), 3.92 (2H), 3.14 (4H), 2.88-2.73 (1H), 2.56 (3H), 2.15 (3H), 2.09 (2H), 1.96 (2H), 1.83 (2H), 1.72 (2H), 1.55 (1H), 1.48 (3H), 1.31 (2H). |

### Examples 42 and 43: N—((R)-1-(3-(1,1-difluoroethyl)-2-fluorophenyl)ethyl)-4-fluoro-5-morpholino-2-(((S)-quinuclidin-3-yl)amino)benzamide and N—((R)-1-(3-(1,1-difluoroethyl)-2-fluorophenyl)ethyl)-4-fluoro-5-morpholino-2-(((R)-quinuclidin-3-yl)amino)benzamide (diastereomers not assigned)

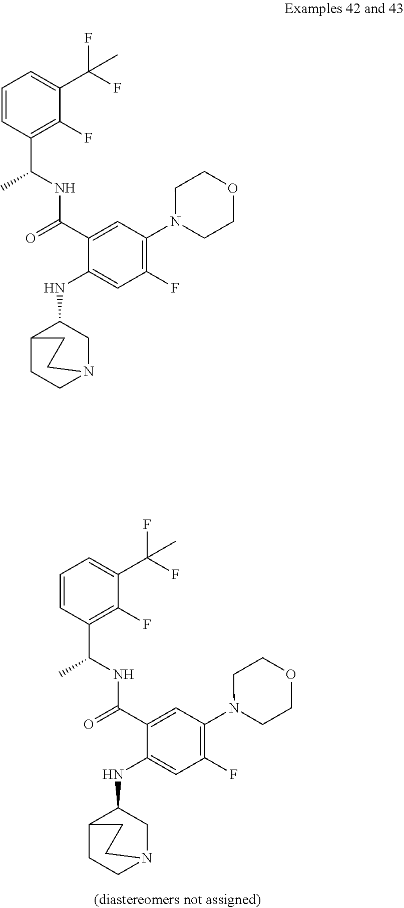 [SMILES: C[C@@H](NC(=O)c1cc(N2CCOCC2)c(F)cc1N[C@@H]1CN2CCC1CC2)c1cccc(C(C)(F)F)c1F ; C[C@@H](NC(=O)c1cc(N2CCOCC2)c(F)cc1N[C@H]1CN2CCC1CC2)c1cccc(C(C)(F)F)c1F]

Synthesized according to analogous procedures described for examples 26 and 27. Diastereomers were separated via chiral SFC: Regis (R,R) Whelk-O1 (25*250 mm, 10 μm); CO₂/IPA [0.5% NH₃ (7M in MeOH)])

Example 42: First eluting peak. MS obsd. (ESI⁺): 535.0 [(M+H)⁺].

Example 43: Second eluting peak. MS obsd. (ESI⁺): 535.0 [(M+H)⁺].

### Examples 44 and 45: N—((R)-1-(3-cyano-2-methylphenyl)ethyl)-4-fluoro-5-morpholino-2-(((S)-quinuclidin-3-yl)amino)benzamide and N—((R)-1-(3-cyano-2-methylphenyl)ethyl)-4-fluoro-5-morpholino-2-(((R)-quinuclidin-3-yl)amino)benzamide (diastereomers not

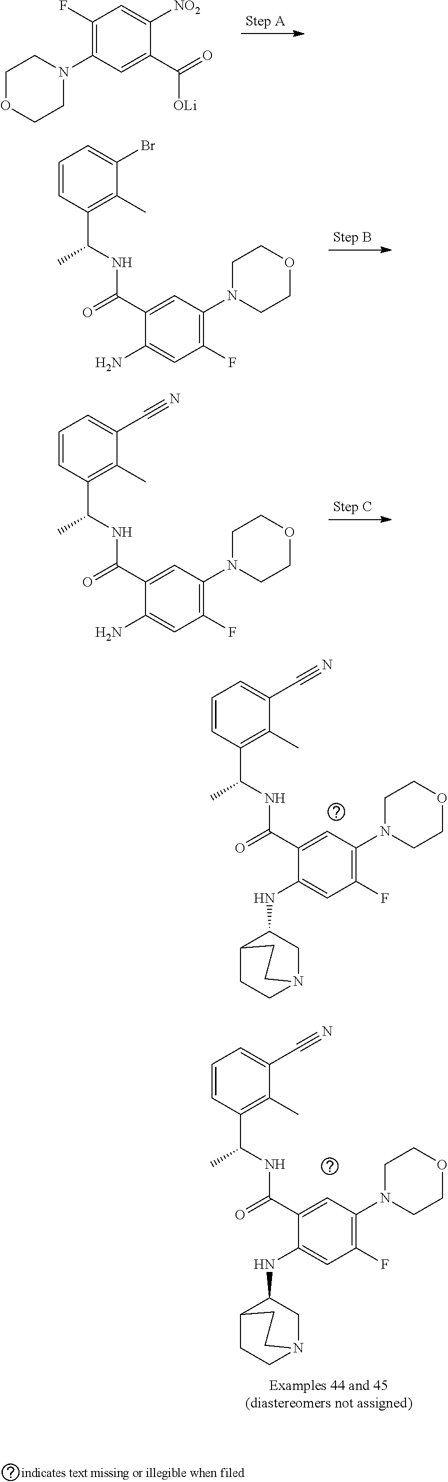

### (Step A: (R)-2-amino-N-(1-(3-bromo-2-methylphenyl)ethyl)-4-fluoro-5-morpholinobenzamide

To a solution of (2-amino-4-fluoro-5-morpholino-benzoyl)oxylithium (210 mg, crude, assumed 0.59 mmol; synthesis described in example 14) in DMF (8 mL) was added HATU (341 mg, 0.90 mmol), DIPEA (386 mg, 2.99 mmol) and (R)-1-(3-bromo-2-methylphenyl) ethan-1-amine hydrochloride (157 mg, 0.60 mmol) at rt. The mixture was stirred at rt for 1 hr. The resulting mixture was diluted with brine and extracted with EA (15 mL×3). The combined organic layers were washed with brine (15 mL×3), dried over anhydrous Na₂SO₄, filtered and concentrated in vacuo. The residue was purified by silica gel chromatography eluting with 0-15% EA in PE to afford the title compound (220 mg, 84% yield). MS obsd. (ESI⁺): 436.5/438.5 [(M+H)⁺].

### Step B: (R)-2-amino-N-(1-(3-cyano-2-methylphenyl)ethyl)-4-fluoro-5-morpholinobenzamide

To a solution of (R)-2-amino-N-(1-(3-bromo-2-methylphenyl)ethyl)-4-fluoro-5-morpholinobenzamide (220 mg, 0.50 mmol) in DMF (8 mL) was added Zn (99 mg, 1.51 mmol), S-Phos (83 mg, 0.2 mmol), Zn (CN) 2 (71 mg, 0.6 mmol) and Pd₂(dba)₃ (92 mg, 0.10 mmol). The resulting mixture was stirred at 140° C. for 4 hrs. The resulting mixture was filtered, and the filtrate was diluted with brine and extracted with EA. The combined organic layers were concentrated in vacuo and the residue was purified by silica gel column chromatography (eluting with 0˜40% EA in PE) to afford the title compound (160 mg, 83% yield). MS obsd. (ESI⁺): 383.7 [(M+H)⁺].

### Step C: N—((R)-1-(3-cyano-2-methylphenyl)ethyl)-4-fluoro-5-morpholino-2-(((R)-quinuclidin-3-yl)amino)benzamide & N—((R)-1-(3-cyano-2-methylphenyl)ethyl)-4-fluoro-5-morpholino-2-(((S)-quinuclidin-3-yl)amino)benzamide (examples 44 and 45)

To a solution of quinuclidin-3-one (327 mg, 2.61 mmol) in AcOH (5.0 mL) was added (R)-2-amino-N-(1-(3-cyano-2-methylphenyl)ethyl)-4-fluoro-5-morpholinobenzamide (100 mg, 0.26 mmol). The mixture was stirred at rt for 1 h. NaBH (OAc) 3 (554 mg, 2.61 mmol) was added and the mixture was stirred at 30° C. for 36 hrs. Quinuclidin-3-one (327 mg, 2.61 mmol) and NaBH (OAc) 3 (554 mg, 2.61 mmol) were added and the mixture was stirred at 30° C. for another 12 hrs. The resulting mixture was concentrated in vacuo. The residue was basified with NaHCO₃ (aq.) and extracted with EA (30 mL×3). The combined organic layers were dried over anhydrous Na₂SO₄ and concentrated in vacuo. The residue (combined with a separate identical reaction performed on 0.16 mmol scale) was purified by silica gel column chromatography (eluting with 0-10% MeOH in DCM) followed by preparative HPLC purification (ACN/H₂O/0.1% FA) to afford the title compounds as a diastereomeric mixture (72 mg, 35% yield). The diastereomers were further separated by chiral SFC (column: Regis (R,R) Whelk-O1 (25×250, 10 μm), Mobile phase: CO₂/EtOH [0.5% NH₃ (7M in MeOH)]=60/40) to afford the title compounds.

Example 44: First eluting isomer. MS obsd. (ESI⁺): 492.7 [(M+H)⁺].

Example 45: Second eluting isomer. MS obsd. (ESI⁺): 492.7 [(M+H)⁺].

### Example 46: (R)—N-(1-(3-cyano-2-methylphenyl)ethyl)-4-fluoro-2-((1-methylpiperidin-4-yl)amino)-5-morpholinobenzamide

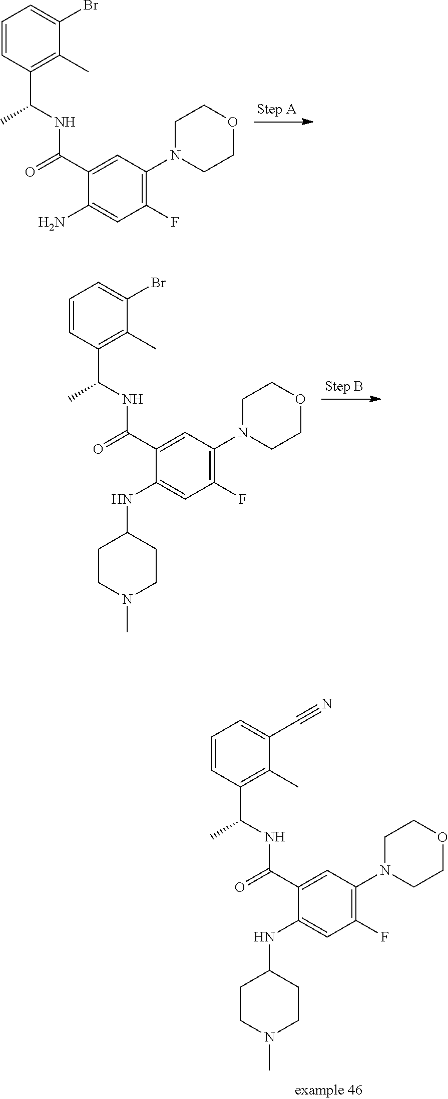 [SMILES: Cc1c(Br)cccc1[C@@H](C)NC(=O)c1cc(N2CCOCC2)c(F)cc1N ; Cc1c(Br)cccc1[C@@H](C)NC(=O)c1cc(N2CCOCC2)c(F)cc1NC1CCN(C)CC1 ; Cc1c(C#N)cccc1[C@@H](C)NC(=O)c1cc(N2CCOCC2)c(F)cc1NC1CCN(C)CC1]

### Step A: (R)—N-(1-(3-bromo-2-methylphenyl)ethyl)-4-fluoro-2-((1-methylpiperidin-4-yl)amino)-5-morpholinobenzamide

To a solution of (R)-2-amino-N-(1-(3-bromo-2-methylphenyl)ethyl)-4-fluoro-5-morpholinobenzamide (300 mg, 0.69 mmol) in AcOH (5 mL) was added 1-methylpiperidin-4-one (1.6 g, 13.75 mmol) and sodium cyanoborohydride (432 mg, 6.88 mmol) at rt. The reaction was stirred at 50° C. for 4 hrs. The rection mixture was concentrated in vacuo. NaHCO₃ (aq, 5 mL) was added and stirred for 5 min. Then the mixture was extracted with DCM (3*15 mL). The organic layers were dried over anhydrous Na₂SO₄ and concentrated in vacuo. The mixture was purified by flash column chromatography (eluting with 0-10% MeOH in DCM) to afford the title compound (278 mg, 75% yield). MS obsd. (ESI⁺): 533.4/535.4 [(M+H)⁺].

### Step B: (R)—N-(1-(3-cyano-2-methylphenyl)ethyl)-4-fluoro-2-((1-methylpiperidin-4-yl)amino)-5-morpholinobenzamide (example 46)

To a solution of (R)—N-(1-(3-bromo-2-methylphenyl)ethyl)-4-fluoro-2-((1-methylpiperidin-4-yl)amino)-5-morpholinobenzamide (100 mg, 0.19 mmol) in N,N-dimethylformamide (2 mL) was added Pd₂(dba)₃ (69 mg, 0.075 mmol), S-Phos (38 mg, 0.093 mmol), zinc powder (61 mg, 0.93 mmol), and zinc cyanide (110 mg, 0.94 mmol) at rt. The reaction was stirred at 150° C. for 5 hrs. The reaction mixture was concentrated in vacuo. The residue was first purified by flash column chromatography (eluting with 0-15% MeOH in DCM) followed by preparative HPLC (ACN/water/0.1% NH₄HCO₃) to afford the title compound (43 mg, 47% yield). MS obsd. (ESI⁺): 480.5 [(M+H)⁺]. ¹H NMR (400 MHZ, DMSO-d₆) δ 8.68 (1H), 7.74 (1H), 7.71-7.64 (2H), 7.41 (2H), 6.54 (1H), 5.29 (1H), 3.73 (4H), 3.27 (1H), 2.94 (4H), 2.58 (5H), 2.16 (3H), 2.11 (2H), 1.84 (2H), 1.44 (3H), 1.31 (2H).

### Example 47: (R)-2-((2-aminoethyl)amino)-N-(1-(3-(difluoromethyl)-2-fluorophenyl)ethyl)-4-fluoro-5-morpholinobenzamide

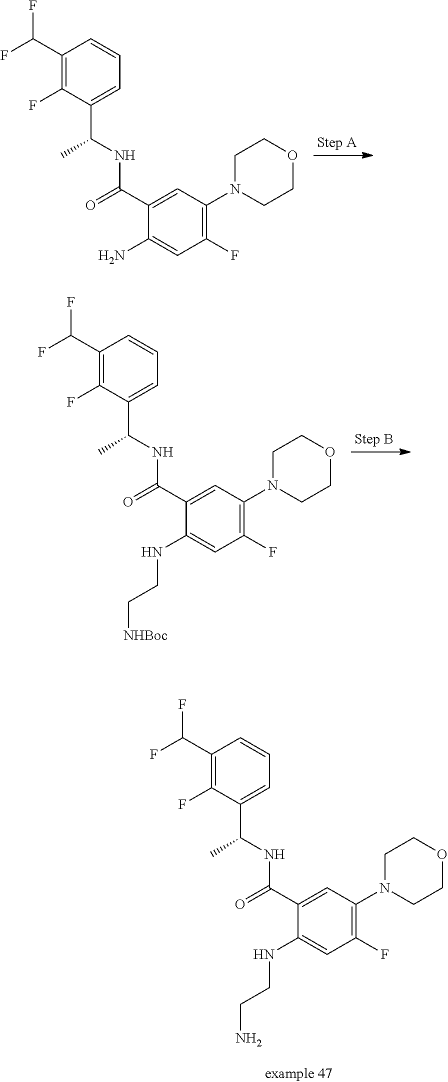 [SMILES: C[C@@H](NC(=O)c1cc(N2CCOCC2)c(F)cc1N)c1cccc(C(F)F)c1F ; C[C@@H](NC(=O)c1cc(N2CCOCC2)c(F)cc1NCCNC(=O)OC(C)(C)C)c1cccc(C(F)F)c1F ; C[C@@H](NC(=O)c1cc(N2CCOCC2)c(F)cc1NCCN)c1cccc(C(F)F)c1F]

### Step A: Tert-butyl (R)-(2-((2-((1-(3-(difluoromethyl)-2-fluorophenyl)ethyl) carbamoyl)-5-fluoro-4-morpholinophenyl)amino)ethyl) carbamate

To a solution of (R)-2-amino-N-(1-(3-(difluoromethyl)-2-fluorophenyl)ethyl)-4-fluoro-5-morpholinobenzamide (100 mg, 0.24 mmol) in ethanol (20 mL) was added tert-butyl N-(2-oxoethyl) carbamate (387 mg, 2.43 mmol) at 80° C. The reaction was stirred for 4 hrs at 80° C. To the reaction mixture was added sodium borohydride (92 mg, 2.43 mmol) and the mixture was stirred for 16 hrs at 80° C. After cooling to room temperature, the solvent was removed under reduced pressure. The residue was purified by silica gel chromatography (eluting with 3% to 5% MeOH in DCM) to afford the title compound (80 mg, 59% yield). MS obsd. (ESI⁺): 555.5 [(M+H)⁺].

### Step B: (R)-2-((2-aminoethyl)amino)-N-(1-(3-(difluoromethyl)-2-fluorophenyl)ethyl)-4-fluoro-5-morpholinobenzamide

To a solution of tert-butyl (R)-(2-((2-((1-(3-(difluoromethyl)-2-fluorophenyl)ethyl) carbamoyl)-5-fluoro-4-morpholinophenyl)amino)ethyl) carbamate (80 mg, 0.144 mmol) in DCM (8 mL) was was added TFA (3 mL) at rt. The mixture was stirred for 1 hr at rt. The reaction was concentrated to dryness and neutralized with 7M NH₃/MeOH and concentrated again. The residue was purified by preparative HPLC (ACN/water/0.1% FA) to afford the title compound (44.1 mg, 62% yield). MS obsd. (ESI⁺): 455.4 [(M+H)⁺]. ¹H NMR (400 MHz, DMSO-d₆) δ: 8.75 (1H), 7.79 (1H), 7.65 (1H), 7.51 (1H), 7.43 (1H), 7.36 (1H), 7.16 (1H), 6.59 (1H), 5.38 (1H), 3.75 (4H), 3.20 (2H), 2.95 (4H), 2.78 (2H), 1.49 (3H).

### Example 48: (R)—N-(1-(3-(difluoromethyl)-2-fluorophenyl)ethyl)-2-((2-(dimethylamino)ethyl)amino)-4-fluoro-5-morpholinobenzamide

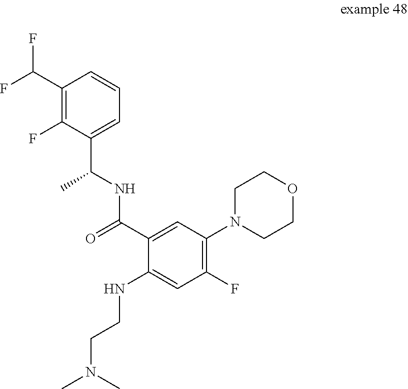 [SMILES: C[C@@H](NC(=O)c1cc(N2CCOCC2)c(F)cc1NCCN(C)C)c1cccc(C(F)F)c1F]

To a solution of (R)-2-((2-aminoethyl)amino)-N-(1-(3-(difluoromethyl)-2-fluorophenyl)ethyl)-4-fluoro-5-morpholinobenzamide (130 mg, crude example 47 prior to HPLC purification) in MeOH (2 mL) was added paraformaldehyde (51 mg) and NaBH₃CN (35 mg, 0.57 mmol). The mixture was stirred at rt for 16 hrs. The reaction solution was concentrated to dryness, and purified by column chromatography (0-20% MeOH in DCM) followed by preparative HPLC [ACN/water/0.1% NH₄HCO₃] to afford the title compound (20.3 mg). MS obsd. (ESI⁺): 483.5 [(M+H)⁺]. ¹H NMR (400 MHZ, DMSO-d₆) δ 8.66 (1H), 7.68-7.61 (2H), 7.52 (1H), 7.40 (1H), 7.35 (1H), 7.22 (1H), 6.49 (1H), 5.36 (1H), 3.74 (4H), 3.08 (2H), 2.94 (4H), 2.43 (2H), 2.15 (6H), 1.48 (3H).

### Examples 49 and 50: N—((R)-1-(3-(difluoromethyl)-2-fluorophenyl)ethyl)-2-(((R)-1-(dimethylamino) propan-2-yl)amino)-4-fluoro-5-morpholinobenzamide and N—((R)-1-(3-(difluoromethyl)-2-fluorophenyl)ethyl)-2-(((S)-1-(dimethylamino) propan-2-yl)amino)-4-fluoro-5-morpholinobenzamide (diastereomers not assigned)

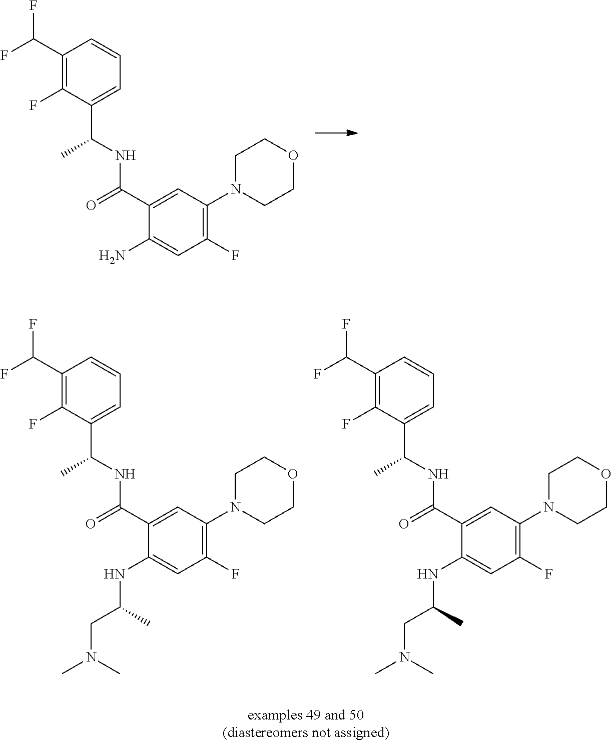 [SMILES: C[C@@H](NC(=O)c1cc(N2CCOCC2)c(F)cc1N)c1cccc(C(F)F)c1F ; C[C@H](CN(C)C)Nc1cc(F)c(N2CCOCC2)cc1C(=O)N[C@H](C)c1cccc(C(F)F)c1F ; C[C@@H](CN(C)C)Nc1cc(F)c(N2CCOCC2)cc1C(=O)N[C@H](C)c1cccc(C(F)F)c1F]

To a solution of (R)-2-amino-N-(1-(3-(difluoromethyl)-2-fluorophenyl)ethyl)-4-fluoro-5-morpholinobenzamide (400 mg, 0.97 mmol) in AcOH (20 mL) was added 1-(dimethylamino) propan-2-one (983 mg, 9.72 mmol) and sodium cyanoborohydride (611 mg, 9.72 mmol). The reaction mixture was stirred for 24 hr at 60° C. After cooling to room temperature, the solvent was removed under reduced pressure. The residue was purified by silica gel chromatography (eluting with 5% to 10% MeOH in DCM) followed by preparative HPLC (ACN/water/0.1% NH₄HCO₃) to afford the target compounds as a diastereomeric mixture (60 mg, 12% yield). The mixture was separated by preparative UPCC (YMC Cellulose-SC (20*250 mm, 5 μm), CO₂/IPA [0.5% NH₃ (7M in MeOH)]=80/20) to afford the title compounds.

Example 49: First eluting peak. MS obsd. (ESI⁺): 497.5 [(M+H)⁺].

Example 50: Second eluting peak. MS obsd. (ESI⁺): 497.5 [(M+H)⁺].

### Example 51: (R)-2-((2-cyanopropan-2-yl)amino)-N-(1-(3-(difluoromethyl)-2-fluorophenyl)ethyl)-4-fluoro-5-morpholinobenzamide

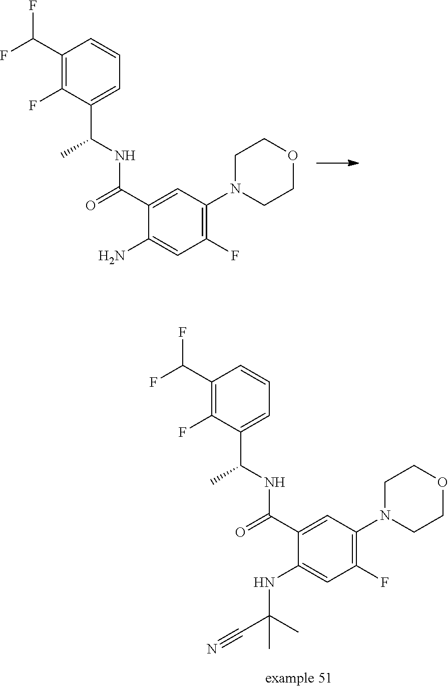

To a solution of (R)-2-amino-N-(1-(3-(difluoromethyl)-2-fluorophenyl)ethyl)-4-fluoro-5-morpholinobenzamide (300 mg, 0.73 mmol) in acetic acid (5 mL) was added acetone (1.3 g, 21.88 mmol, 1.6 mL) at rt. To the reaction mixture was added trimethylsilyl cyanide (217 mg, 2.19 mmol) at 0° C. The reaction mixture was stirred for 16 hrs at rt. To this mixture was added NH₄Cl (5 mL) at 0° C. The mixture was extracted with DCM (30 mL×3), the combined organic layers were washed with brine, dried over anhydrous Na₂SO₄, filtered and concentrated in vacuo. The residue was purified by silica gel chromatography (eluting with 0%-5% MeOH in DCM) followed by preparative HPLC (ACN/water/0.1% NH₄HCO₃) to afford the title compound. MS obsd. (ESI⁺): 479.4 [(M+H)⁺]. ¹H NMR (400 MHZ, DMSO-d₆) δ 8.94 (1H), 8.06 (1H), 7.66 (1H), 7.55-7.49 (2H), 7.39-7.09 (2H), 6.79 (1H), 5.37 (1H), 3.76 (4H), 3.01 (4H), 1.63 (6H), 1.51 (3H).

### Example 52: (R)—N-(1-(3-(difluoromethyl)-2-fluorophenyl)ethyl)-2-((1-(dimethylamino)-2-methylpropan-2-yl)amino)-4-fluoro-5-morpholinobenzamide

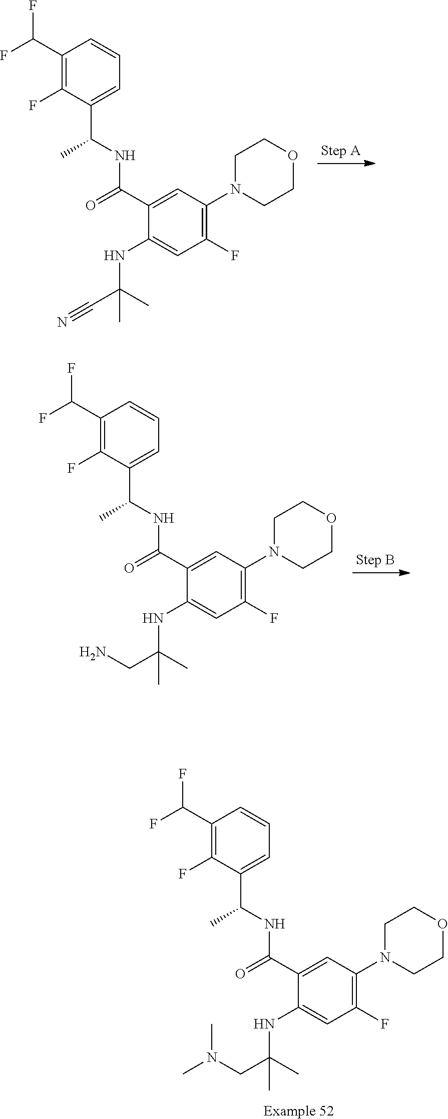

### Step A: (R)-2-((1-amino-2-methylpropan-2-yl)amino)-N-(1-(3-(difluoromethyl)-2-fluorophenyl)ethyl)-4-fluoro-5-morpholinobenzamide

To a solution of (R)-2-((2-cyanopropan-2-yl)amino)-N-(1-(3-(difluoromethyl)-2-fluorophenyl)ethyl)-4-fluoro-5-morpholinobenzamide (150 mg, 0.31 mmol) in 7 M NH₃ in MeOH (5 mL) was added Raney nickel (30 mg, 0.51 mmol). The mixture was stirred for 16 hrs at rt under an atmosphere of hydrogen. The mixture was filtered and the filtrate was concentrated in vacuo and purified by flash column (eluted with 0-10% MeOH in DCM) to afford the title compound (31 mg, 20% yield). MS obsd. (ESI⁺): 483.3 [(M+H)⁺]

### Step B: (R)—N-(1-(3-(difluoromethyl)-2-fluorophenyl)ethyl)-2-((1-(dimethylamino)-2-methylpropan-2-yl)amino)-4-fluoro-5-morpholinobenzamide (example 52)

To a mixture of (R)-2-((1-amino-2-methylpropan-2-yl)amino)-N-(1-(3-(difluoromethyl)-2-fluorophenyl)ethyl)-4-fluoro-5-morpholinobenzamide (31 mg, 0.064 mmol) in EtOH (5 mL) was added paraformaldehyde (19 mg) and NaBH₃CN (32 mg, 0.51 mmol). The mixture was stirred for 16 hrs at rt. The mixture was quenched with water and stirred for 30 min, then extracted with DCM (3×80 mL). The combined organic layers were dried over Na₂SO₄, filtered and concentrated. Purification via silica gel chromatography (eluted with 0-20% MeOH in DCM) followed by preparative HPLC (ACN/water/0.1% NH₄HCO₃) afforded the title compound (3.6 mg, 10% yield). MS obsd. (ESI⁺): 511.5 [(M+H)⁺] ¹H NMR (400 MHZ, DMSO-d₆) δ: 8.70 (1H), 7.63 (1H), 7.52 (2H), 7.38-7.07 (3H), 6.65 (1H), 5.33 (1H), 3.74 (4H), 2.94 (4H), 2.31 (2H), 2.15 (6H), 1.48 (3H), 1.20 (6H).

### Example 53: (R)—N-(1-(3-(difluoromethyl)-2-fluorophenyl)ethyl)-2-((3-(dimethylamino) bicyclo[1.1.1]pentan-1-yl)amino)-4-fluoro-5-morpholinobenzamide

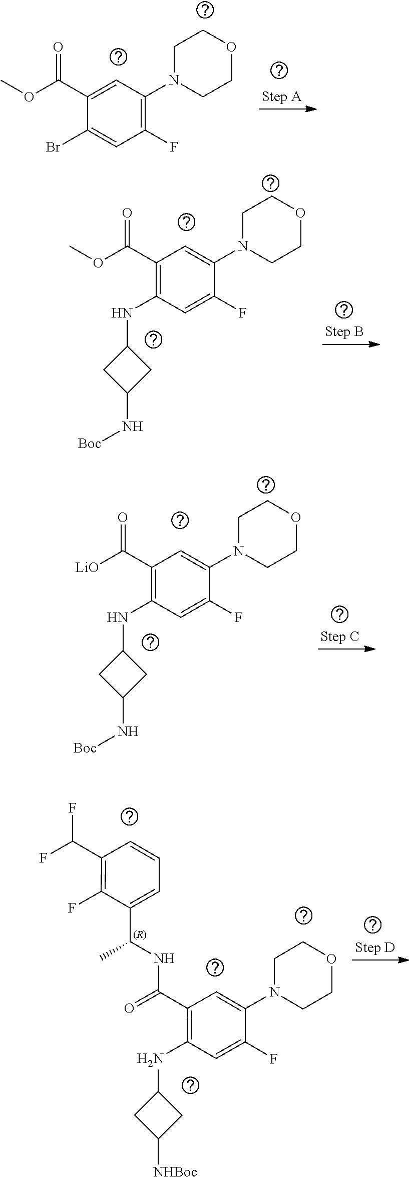

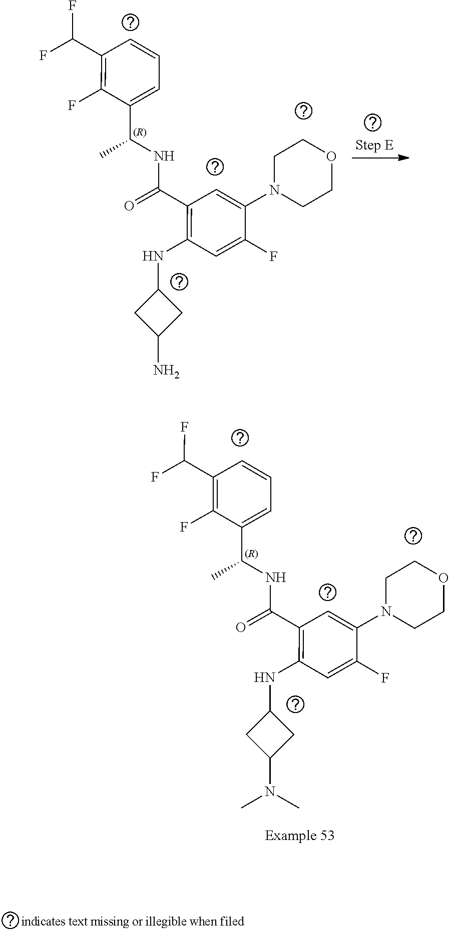

### Step A: Methyl 2-((3-((tert-butoxycarbonyl)amino)bicyclo[1.1.1]pentan-1-yl)amino)-4-fluoro-5-morpholinobenzoate

To a solution of methyl 2-bromo-4-fluoro-5-morpholinobenzoate (200 mg, 0.63 mmol) in 1,4-dioxane (3 mL) was added tert-butyl N-(3-amino-1-bicyclo[1.1.1]pentanyl) carbamate (187 mg, 0.95 mmol), cesium carbonate (614 mg, 1.89 mmol) and Xantphos-Pd-G3 (119 mg, 0.12 mmol). The reaction mixture was heated to 100° C. in a microwave reactor for 16 hrs under N₂. The solvent was removed in vacuo and the residue was purified by flash chromatography (eluting with 0%-50% EA in PE) to afford the title compound (129 mg, 47% yield). MS obsd. (ESI⁺): 436.4 [(M+H)⁺].

### Step B: Lithium 2-((3-((tert-butoxycarbonyl)amino)bicyclo[1.1.1]pentan-1-yl)amino)-4-fluoro-5-morpholinobenzoate

To a solution of methyl 2-((3-((tert-butoxycarbonyl)amino)bicyclo[1.1.1]pentan-1-yl)amino)-4-fluoro-5-morpholinobenzoate (129 mg, 0.30 mmol) in MeOH (6 mL) and H₂O (2 mL) was added LiOH (21 mg, 0.89 mmol). The reaction was stirred for 16 hrs at 50° C. The mixture was cooled to room temperature and the solvent was removed in vacuo to afford the crude title compound (127 mg, crude). The crude product was used for the next step without further purification. MS obsd. (ESI⁺): 422.4 [(M+H)⁺] for free acid.

### Step C: Tert-butyl (R)-(3-((2-((1-(3-(difluoromethyl)-2-fluorophenyl)ethyl) carbamoyl)-5-fluoro-4-morpholinophenyl)amino)bicyclo[1.1.1]pentan-1-yl) carbamate

To a solution of lithium 2-((3-((tert-butoxycarbonyl)amino)bicyclo[1.1.1]pentan-1-yl)amino)-4-fluoro-5-morpholinobenzoate (127 mg, crude) in DMF (4 mL) was added HATU (136 mg, 0.36 mmol). The reaction was stirred for 10 mins at rt. Then (R)-1-(3-(difluoromethyl)-2-fluorophenyl) ethan-1-amine (73 mg, 0.39 mmol) and DIPEA (115 mg, 0.89 mmol) were added. The reaction was stirred for 2 hrs at rt. The mixture was diluted with water and extracted with DCM (3×20 mL). The combined organic layers were dried over Na₂SO₄, filtered and concentrated. The residue was purified by preparative HPLC (ACN/water/0.1% NH₄HCO₃) to afford the title compound (105 mg). MS obsd. (ESI⁺): 593.4 [(M+H)⁺].

### Step D: (R)-2-((3-aminobicyclo[1.1.1]pentan-1-yl)amino)-N-(1-(3-(difluoromethyl)-2-fluorophenyl)ethyl)-4-fluoro-5-morpholinobenzamide

To a solution of tert-butyl (R)-(3-((2-((1-(3-(difluoromethyl)-2-fluorophenyl)ethyl) carbamoyl)-5-fluoro-4-morpholinophenyl)amino)bicyclo[1.1.1]pentan-1-yl) carbamate (100 mg, 0.17 mmol) in DCM (2 mL) was added TFA (0.5 mL). The reaction was stirred for 1 h at rt. The solvent was removed in vacuo and the residue was neutralized with NaHCO₃ (aq) and extracted with DCM (3×20 mL). The combined organic layers were dried over Na₂SO₄, filtered and concentrated to afford the title compound (83 mg, crude). The crude product was used for the next step without further purification. MS obsd. (ESI⁺): 493.4 [(M+H)⁺].

### Step E: (R)—N-(1-(3-(difluoromethyl)-2-fluorophenyl)ethyl)-2-((3-(dimethylamino) bicyclo[1.1.1]pentan-1-yl)amino)-4-fluoro-5-morpholinobenzamide (example 53)

To a solution of (R)-2-((3-aminobicyclo[1.1.1]pentan-1-yl)amino)-N-(1-(3-(difluoromethyl)-2-fluorophenyl)ethyl)-4-fluoro-5-morpholinobenzamide (83 mg, crude, assumed 0.17 mmol) in EtOH (5 mL) was added paraformaldehyde (51 mg) and sodium cyanoborohydride (106 mg, 1.69 mmol). The reaction was stirred for 16 hrs at rt. The reaction was quenched with H₂O (10 mL) and stirred for 0.5 h. The mixture was extracted with DCM (3×20 mL). The combined organic layers were dried over Na₂SO₄, filtered and concentrated. The residue was purified by preparative TLC (DCM/MeOH=7/1) followed by preparative HPLC (ACN/water/0.1% NH₄HCO₃) to afford the title compound (34.2 mg, 38% yield). MS obsd. (ESI⁺): 521.5 [(M+H)⁺]. ¹H NMR (400 MHz, DMSO-d₆) δ8.78 (1H), 8.22 (1H), 7.63 (1H), 7.52 (1H), 7.44 (1H), 7.35 (1H), 7.22 (1H), 6.61 (1H), 5.34 (1H), 3.75 (4H), 2.95 (4H), 2.13 (6H), 1.95 (6H), 1.49 (3H).

### Example 54: (R)—N-(1-(3-(difluoromethyl)-2-fluorophenyl)ethyl)-2-((1,3-dimethylazetidin-3-yl)amino)-4-fluoro-5-morpholinobenzamide

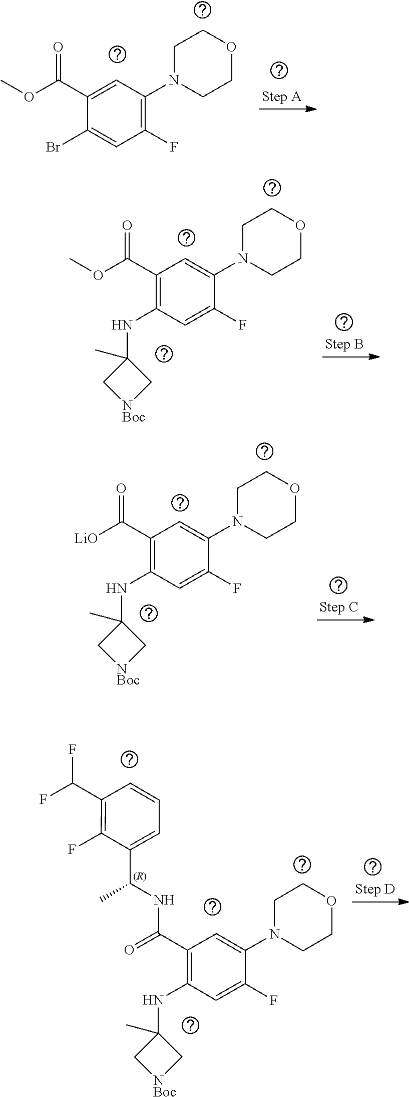

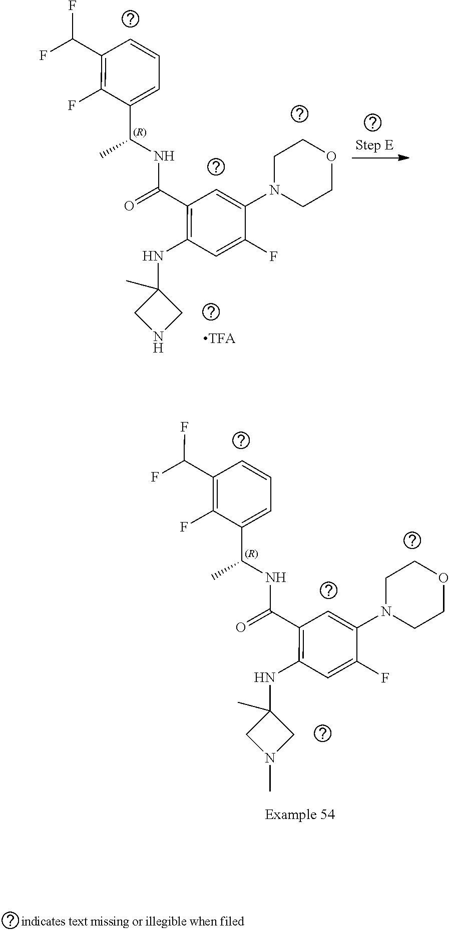

### Step A: Tert-butyl 3-((5-fluoro-2-(methoxycarbonyl)-4-morpholinophenyl)amino)-3-methylazetidine-1-carboxylate

To a solution of methyl 2-bromo-4-fluoro-5-morpholino-benzoate (300 mg, 0.94 mmol) in toluene (4 mL) was added tert-butyl 3-amino-3-methyl-azetidine-1-carboxylate (263 mg, 1.41 mmol), cesium carbonate (922 mg, 2.83 mmol), RuPhos (176 mg, 0.38 mmol) and tris(dibenzylideneacetone) dipalladium (0) (173 mg, 0.19 mmol) at rt. The reaction mixture was heated to 100° C. in a microwave reactor and stirred for 4 hr under N₂. After cooling to room temperature, the solvent was removed in vacuo and the residue was purified by silica gel chromatography (eluting with 40% to 50% EA in PE) to afford the title compound (200 mg, 50% yield). MS obsd. (ESI⁺): 424.7 [(M+H)⁺].

### Step B: Lithium 2-((1-(tert-butoxycarbonyl)-3-methylazetidin-3-yl)amino)-4-fluoro-5-morpholinobenzoate

To a solution of tert-butyl 3-((5-fluoro-2-(methoxycarbonyl)-4-morpholinophenyl)amino)-3-methylazetidine-1-carboxylate (200 mg, 0.47 mmol) in water (1 mL) and THF (4 mL) was added LiOH (45 mg, 1.89 mmol) at rt. The reaction mixture was stirred for 48 hrs at 50° C. The mixture was concentrated to dryness to afford the title compound (190 mg, crude), which was used without further purification. MS obsd. (ESI⁺): 410.7 [(M+H)⁺] for free acid.

### Step C: Tert-butyl (R)-3-((2-((1-(3-(difluoromethyl)-2-fluorophenyl)ethyl) carbamoyl)-5-fluoro-4-morpholinophenyl)amino)-3-methylazetidine-1-carboxylate

To a solution of lithium 2-((1-(tert-butoxycarbonyl)-3-methylazetidin-3-yl)amino)-4-fluoro-5-morpholinobenzoate (180 mg, crude, assumed 0.43 mmol) in DMF (5 mL) was added HATU (214 mg, 0.56 mmol) and DIPEA (168 mg, 1.30 mmol) at rt. The reaction was stirred for 30 min at rt. To the reaction mixture was added (R)-1-(3-(difluoromethyl)-2-fluorophenyl) ethan-1-amine (107 mg, 0.56 mmol) at rt and stirred for 1 hr at rt. The reaction was poured into water (30 mL) and extracted with EA (20 mL×3). The combined organic layers were washed with brine, dried over anhydrous Na₂SO₄, filtered, and concentrated in vacuo. The residue was purified by silica gel chromatography (eluting with 5% to 10% MeOH in DCM) to afford the title compound (210 mg). MS obsd. (ESI⁺): 581.9 [(M+H)⁺].

### Step D: (R)-3-((2-((1-(3-(difluoromethyl)-2-fluorophenyl)ethyl) carbamoyl)-5-fluoro-4-morpholinophenyl)amino)-3-methylazetidin-1-ium 2,2,2-trifluoroacetate

To a solution of tert-butyl (R)-3-((2-((1-(3-(difluoromethyl)-2-fluorophenyl)ethyl) carbamoyl)-5-fluoro-4-morpholinophenyl)amino)-3-methylazetidine-1-carboxylate (210 mg, 0.36 mmol) in DCM (4 mL) was added TFA (2 mL) at rt. The mixture was stirred for 1 hr at rt. The mixture was concentrated to dryness to afford the title compound (200 mg, crude), which was used without further purification. MS obsd. (ESI⁺): 481.7 [(M+H)⁺].

### Step E: (R)—N-(1-(3-(difluoromethyl)-2-fluorophenyl)ethyl)-2-((1,3-dimethylazetidin-3-yl)amino)-4-fluoro-5-morpholinobenzamide (example 54)

To a solution of (R)-3-((2-((1-(3-(difluoromethyl)-2-fluorophenyl)ethyl) carbamoyl)-5-fluoro-4-morpholinophenyl)amino)-3-methylazetidin-1-ium 2,2,2-trifluoroacetate (200 mg, crude) in MeOH (10 mL) was added paraformaldehyde (152 mg) at rt. The reaction was stirred for 30 min at rt. To the reaction mixture was added sodium cyanoborohydride (106 mg, 1.68 mmol) at rt and stirred for 16 hrs at rt. The reaction was quenched with water (30 mL) and stirred for 30 min. The mixture was extracted with DCM (30 mL×3). The combined organic layers were washed with brine, dried over anhydrous Na₂SO₄, filtered, and concentrated in vacuo. The residue was purified by silica gel chromatography (eluting with 15% to 20% MeOH in DCM) followed by preparative HPLC (ACN/water/0.1% NH₄HCO₃) to afford the title compound (60.7 mg). MS obsd. (ESI⁺): 495.5 [(M+H)⁺]. ¹H NMR (400 MHZ, DMSO-d₆) δ:8.73 (1H), 7.99 (1H), 7.64 (1H), 7.52 (1H), 7.46 (1H), 7.36 (1H), 7.22 (1H), 6.03 (1H), 5.35 (1H), 3.74 (4H), 3.37 (2H), 2.94 (4H), 2.90 (2H), 2.22 (3H), 1.49 (3H), 1.44 (3H).

### Example 55: N—((R)-1-(3-(difluoromethyl)-2-fluorophenyl)ethyl)-4-fluoro-2-(((1R,5S,6s)-3-methyl-3-azabicyclo[3.1.0]hexan-6-yl)amino)-5-morpholinobenzamide

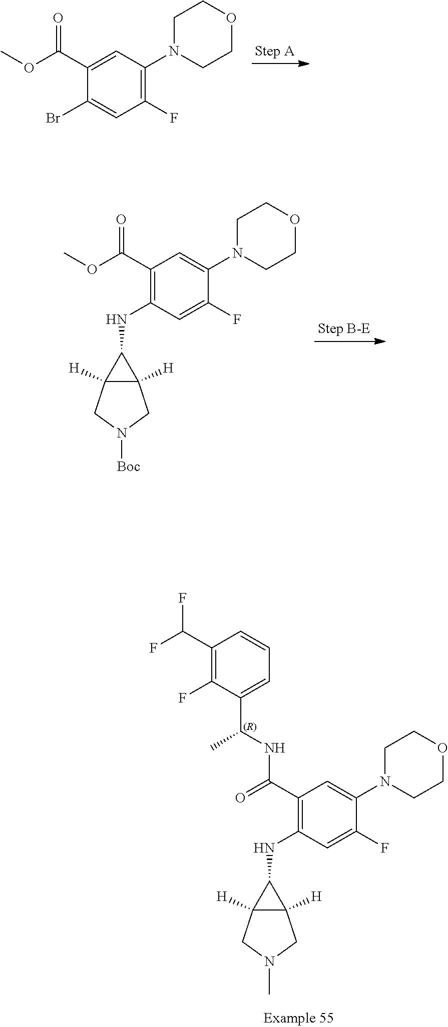

### Step A: Tert-butyl (1R,5S,6s)-6-((5-fluoro-2-(methoxycarbonyl)-4-morpholinophenyl)amino)-3-azabicyclo[3.1.0]hexane-3-carboxylate (VC00847786-2)

A mixture of methyl 2-bromo-4-fluoro-5-morpholinobenzoate (100 mg, 0.31 mmol), tert-butyl (1S,5R)-6-amino-3-azabicyclo[3.1.0]hexane-3-carboxylate (94 mg, 0.47 mmol), XPhos-Pd-G3 (53 mg, 0.063 mmol) and cesium carbonate (205 mg, 0.63 mmol) was dissolved in 1,4-dioxane (1.0 mL). The reaction mixture was stirred at 50° C. for 16 hrs under N₂. The resulting mixture was poured into water (100 mL) and extracted with EtOAc (60 mL×3). The combined organic layers were washed with brine, dried over sodium sulfate, filtered and concentrated. The residue was purified by flash column chromatography (eluting with 0-50% EA in PE) to afford the title compound (90 mg, 65% yield). MS obsd. (ESI⁺): 436.3 [(M+H)⁺].

### Steps B-E: N—((R)-1-(3-(difluoromethyl)-2-fluorophenyl)ethyl)-4-fluoro-2-(((1R,5S,6s)-3-methyl-3-azabicyclo[3.1.0]hexan-6-yl)amino)-5-morpholinobenzamide (example 55)

Steps B-E were performed according to analogous procedures described for example 54, steps B-E. MS obsd. (ESI⁺): 507.5 [(M+H)⁺]. ¹H NMR (400 MHZ, DMSO-d₆) & 8.72 (1H), 7.73 (1H), 7.62 (1H), 7.51 (1H), 7.43 (1H), 7.35 (1H), 7.21 (1H), 6.59 (1H), 5.38-5.26 (1H), 3.81-3.67 (4H), 3.04 (2H), 2.94 (4H), 2.52 (1H), 2.28 (2H), 2.20 (3H), 1.47 (5H)

### Example 56: (R)-4-fluoro-N-(1-(2-methyl-3-(trifluoromethyl)phenyl)ethyl)-2-((1-methylpiperidin-4-yl)amino)-5-(pyridin-4-yl)benzamide

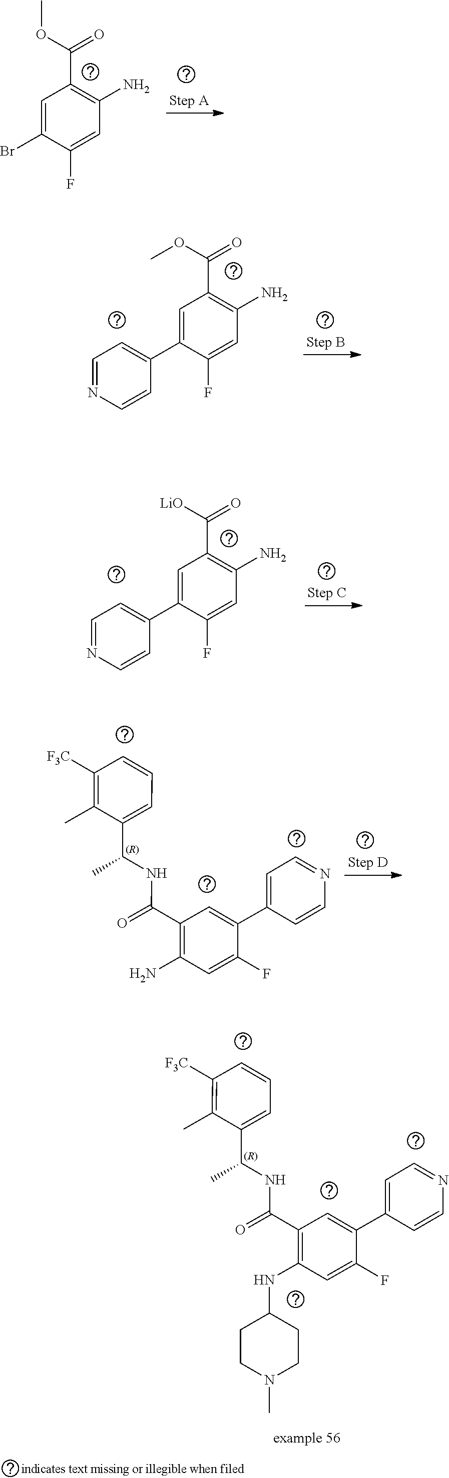

### Step A: Methyl 2-amino-4-fluoro-5-(pyridin-4-yl)benzoate

To a mixture of methyl 2-amino-5-bromo-4-fluoro-benzoate (200 mg, 0.81 mmol) in 1,4-dioxane (4 mL) and H₂O (1 mL) was added 4-(4,4,5,5-tetramethyl-1,3,2-dioxaborolan-2-yl)pyridine (198 mg, 0.97 mmol), 1,1′-Bis(diphenylphosphino) ferrocene palladium (II) dichloride (88 mg, 0.12 mmol), and sodium carbonate (256 mg, 2.42 mmol). The mixture was stirred at 110° C. for 2 h under N₂ atmosphere. The mixture was cooled down to rt, diluted with EA (10 mL), and washed with H₂O (10 mL*3) and brine (8 mL*3). The organic layer was dried (Na₂SO₄), filtered and concentrated. The residue was purified by silica gel column (PE:EA=3:2) to provide the title compound (190 mg, 95% yield). MS obsd. (ESI⁺): 247.0 [(M+H)⁺].

### Step B: Lithium 2-amino-4-fluoro-5-(pyridin-4-yl)benzoate

To a solution of methyl 2-amino-4-fluoro-5-(4-pyridyl)benzoate (90 mg, 0.36 mmol) in water (1 mL) and THF (4 mL) was added LiOH (26 mg, 1.10 mmol) at rt. The reaction mixture was stirred for 16 hr at 45° C. The mixture was cooled to room temperature and the solvent was removed under reduced pressure to afford the crude title compound (90 mg, crude). This material was used without further purification MS obsd. (ESI⁺): 233.0 [(M+H)⁺] for free acid.

### Step C: (R)-2-amino-4-fluoro-N-(1-(2-methyl-3-(trifluoromethyl)phenyl)ethyl)-5-(pyridin-4-yl)benzamide

To a solution of lithium 2-amino-4-fluoro-5-(4-pyridyl)benzoate (90 mg, crude) in DMF (5 mL) was added HATU (215 mg, 0.57 mmol), N,N-diisopropylethylamine (146 mg, 1.13 mmol) and (R)-1-(2-methyl-3-(trifluoromethyl)phenyl) ethan-1-amine (100 mg, 0.49 mmol). The reaction mixture was stirred for 3 hrs at rt. The reaction was quenched with H₂O (10 mL) and stirred for 0.5 hr. The mixture was extracted with DCM (3*20 mL). The combined organic layers were dried over Na₂SO₄, filtered and concentrated. The residue was purified by flash column chromatography (eluting with 0%-30% MeOH in DCM) to afford the title compound (100 mg). MS obsd. (ESI⁺): 418.2 [(M+H)⁺].

### Step D: (R)-4-fluoro-N-(1-(2-methyl-3-(trifluoromethyl)phenyl)ethyl)-2-((1-methylpiperidin-4-yl)amino)-5-(pyridin-4-yl)benzamide (example 56)

To a solution of (R)-2-amino-4-fluoro-N-(1-(2-methyl-3-(trifluoromethyl)phenyl)ethyl)-5-(pyridin-4-yl)benzamide (100 mg, 0.24 mmol) in TFA (4 mL) was added 1-methylpiperidin-4-one (407 mg, 3.59 mmol) and sodium cyanoborohydride (226 mg, 3.59 mmol) at rt. The reaction mixture was stirred for 1 hr at 50° C. The reaction was concentrated to dryness and neutralized with 7M NH₃/MeOH and concentrated again. The residue was first purified by silica gel chromatography (eluting with 10% to 20% MeOH in DCM) followed by preparative HPLC (ACN/water/0.1% NH₄HCO₃) to afford the title compound (34.9 mg, 28% yield). MS obsd. (ESI⁺): 515.5 [(M+H)⁺]. ¹H NMR (400 MHZ, DMSO-d₆) δ:8.90 (1H), 8.62 (2H), 8.27 (1H), 8.02 (1H), 7.74 (1H), 7.61 (2H), 7.57 (1H), 7.40 (1H), 6.67 (1H), 5.39 (1H), 3.40 (1H), 2.58 (2H), 2.49 (3H), 2.15 (3H), 2.06 (2H), 1.88 (2H), 1.45 (3H), 1.36 (2H).

### Example 57: (R)—N-(1-(3-(difluoromethyl)-2-fluorophenyl)ethyl)-4-fluoro-2-((1-methylpiperidin-4-yl)amino)-5-(pyridin-4-yl)benzamide

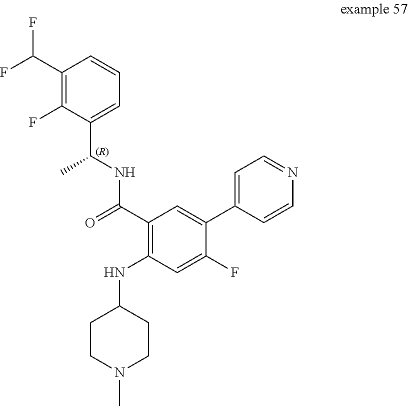

Prepared according to analogous procedures described for example 56. MS obsd. (ESI⁺): 501.3 ([M+H]⁺). ¹H NMR (400 MHZ, DMSO-d₆) δ 8.91 (1H), 8.63 (2H), 8.28 (1H), 8.05 (1H), 7.66 (1H), 7.62 (2H), 7.52 (1H), 7.36 (1H), 7.22 (1H), 6.68 (1H), 5.38 (1H), 3.41 (1H), 2.60 (2H), 2.19-2.05 (5H), 1.88 (2H), 1.49 (3H), 1.47-1.31 (2H).

### Examples 58 and 59: N—((R)-1-(3-(difluoromethyl)-2-fluorophenyl)ethyl)-4-fluoro-5-(pyridin-4-yl)-2-(((R)-quinuclidin-3-yl)amino)benzamide & N—((R)-1-(3-(difluoromethyl)-2-fluorophenyl)ethyl)-4-fluoro-5-(pyridin-4-yl)-2-(((S)-quinuclidin-3-yl)amino)benzamide (diastereomers not assigned)

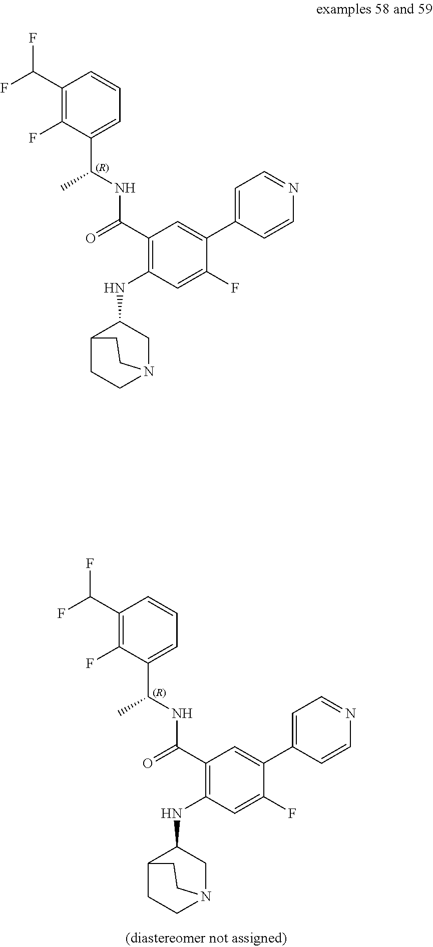

Synthesized according to analogous procedures described for examples 56 and 57. The diastereomeric mixture was separated by chiral SFC (Column Name: Daicel AD (25×250 mm, 10 μm); CO₂/MeOH [0.2% NH₃ (7M in MeOH)] 80/29) to provide the title compounds.

Example 58: First eluting peak. MS obsd. (ESI⁺): 513.2 ([M+H]⁺).

Example 59: Second eluting peak. MS obsd. (ESI⁺): 513.2 ([M+H]⁺).

### Example 60: (R)—N-(1-(3-(difluoromethyl)-2-fluorophenyl)ethyl)-4-fluoro-5-(1-methyl-2-oxo-1,2-dihydropyridin-4-yl)-2-((1-methylpiperidin-4-yl)amino)benzamide

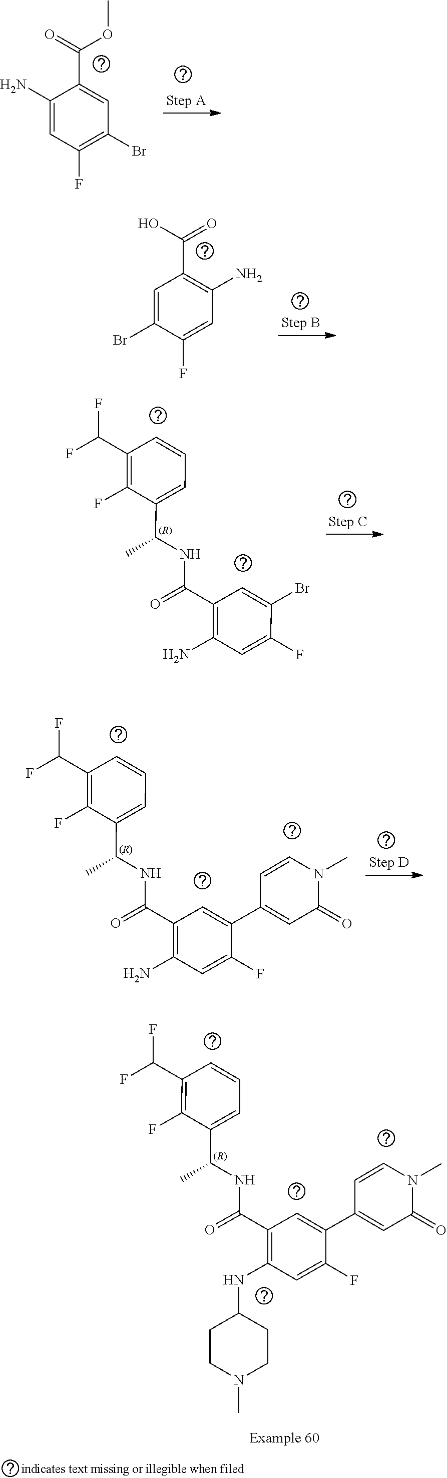

### Step A: 2-amino-5-bromo-4-fluorobenzoic acid

To a solution of methyl 2-amino-5-bromo-4-fluoro-benzoate (1.0 g, 4.03 mmol) in MeOH (4 mL) and THF (20 mL) was added 2M LiOH (2 mL, aqueous). The reaction was stirred for 16 hrs at 45° C. The mixture was cooled to rt, then acidified with 2M HCl (10 mL). The solvent was removed in vacuo to afford the title compound (1.05 g, crude). This material was used without further purification. MS obsd. (ESI⁺): 235.9 ([M+H]⁺).

### Step B: (R)-2-amino-5-bromo-N-(1-(3-(difluoromethyl)-2-fluorophenyl)ethyl)-4-fluorobenzamide

To a solution of 2-amino-5-bromo-4-fluoro-benzoic acid (1.1 g, crude) in DMF (10 mL) was added HATU (2.5 g, 6.67 mmol), DIPEA (1.7 g, 13.35 mmol) and (R)-1-(3-(difluoromethyl)-2-fluorophenyl) ethan-1-amine (926 mg, 4.89 mmol). The reaction was stirred for 2 hrs at rt. The mixture was diluted with water and extracted with EA. The combined organic layers were washed with brine and dried over Na₂SO₄, filtered and concentrated. The residue was purified by silica gel column chromatography (eluting with 30% EA in PE) to afford the title compound (1.27 g). MS obsd. (ESI⁺): 405.3/407.3 ([M+H]⁺).

### Step C: (R)-2-amino-N-(1-(3-(difluoromethyl)-2-fluorophenyl)ethyl)-4-fluoro-5-(1-methyl-2-oxo-1,2-dihydropyridin-4-yl)benzamide

A solution of (R)-2-amino-5-bromo-N-(1-(3-(difluoromethyl)-2-fluorophenyl)ethyl)-4-fluorobenzamide (400 mg, 0.99 mmol), 1-methyl-4-(4,4,5,5-tetramethyl-1,3,2-dioxaborolan-2-yl)pyridin-2-one (278 mg, 1.18 mmol), Pd(dppf)Cl₂ (72 mg, 0.099 mmol) and K₂CO₃ (410 mg, 2.96 mmol) in a mixture of H₂O (0.3 mL) and dioxane (3 mL) was stirred in a sealed vial. The sealed vial was irradiated in a microwave reactor at 110° C. for 3 hours. The mixture was diluted with water and extracted with EA (30 mL×2). The combined organic layers were washed with brine and dried with Na₂SO₄, filtered and concentrated. The residue was purified by silica gel column chromatography (eluting with 2% MeOH in DCM) to afford the title compound (312 mg, 73% yield). MS obsd. (ESI⁺): 434.3 ([M+H]⁺).

### Step D: (R)—N-(1-(3-(difluoromethyl)-2-fluorophenyl)ethyl)-4-fluoro-5-(1-methyl-2-oxo-1,2-dihydropyridin-4-yl)-2-((1-methylpiperidin-4-yl)amino)benzamide (Example 60)

To a solution of (R)-2-amino-N-(1-(3-(difluoromethyl)-2-fluorophenyl)ethyl)-4-fluoro-5-(1-methyl-2-oxo-1,2-dihydropyridin-4-yl)benzamide (250 mg, 0.57 mmol) in TFA (5 mL) was added NaBH₃CN (144 mg, 2.31 mmol). The reaction was stirred for 16 hrs at 60° C. The reaction mixture was concentrated in vacuo. The residue was diluted with aqueous NaHCO₃ and extracted with EA. The combined organic layers were dried over sodium sulfate, filtered and concentrated. The residue was purified by silica gel column chromatography (eluting with 2% MeOH in DCM) followed by preparative HPLC (MeCN in water/0.1% HCOOH) to afford the title compound (181 mg, 59% yield). MS obsd. (ESI⁺): 531.2 ([M+H]⁺). ¹H NMR (400 MHZ, DMSO-d₆) δ 8.99 (1H), 8.32 (1H), 8.01 (1H), 7.73 (1H), 7.65 (1H), 7.52 (1H), 7.36-7.10 (2H), 6.73 (2H), 6.50 (1H), 5.38 (1H), 3.56 (1H), 3.45 (3H), 3.17 (2H), 2.84 (2H), 2.62 (3H), 2.06 (2H), 1.52 (5H).

### Example 61: (R)-4-fluoro-5-(isoxazol-4-yl)-N-(1-(2-methyl-3-(trifluoromethyl)phenyl)ethyl)-2-((1-methylpiperidin-4-yl)amino)benzamide

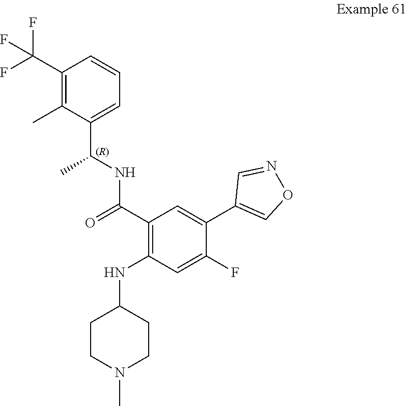

Prepared according to analogous procedures described for example 60 with appropriate reagent substitutions in steps B and C. MS obsd. (ESI⁺): m/z 505.5 (M+H)⁺. ¹H NMR (400 MHZ, DMSO-d₆) δ: 9.18 (1H), 9.08 (1H), 8.76 (1H), 8.13 (1H), 8.07 (1H), 7.77 (1H), 7.58 (1H), 7.41 (1H), 6.67 (1H), 5.39 (1H), 3.38 (1H), 2.60 (2H), 2.49 (3H), 2.16-2.10 (5H), 1.88 (2H), 1.47 (3H), 1.36 (2H).

The following examples can be synthesized according to analogous procedures described for example 60 with appropriate reagent substitution in step C.

| Example | | | |
|---|---|---|---|
| # | Structure | Compound Name | Analytical Data |
| 62 | | (R)-N-(1-(3- | MS obsd. (ESI⁺): m/z |
| | | (difluoromethyl)-2- | 531.2 (M + H)⁺. |
| | | fluorophenyl)ethyl)-4- | ¹H NMR (400 MHz, |
| | | fluoro-5-(1-methyl-6-oxo- | DMSO-d₆) δ: 8.77 |
| | | 1,6-dihydropyridin-3-yl)- | (1H), 8.08 (1H), 7.90 |
| | | 2-((1-methylpiperidin-4- | (1H), 7.84 (1H), 7.65 |
| | | yl)amino)benzamide | (2H), 7.52 (1H), 7.36 |
| | | | (1H), 7.21 (1H), 6.63 |
| | | | (1H), 6.50 (1H), 5.37 |
| | | | (1H), 3.51 (3H), 2.66 |
| | | | (2H), 2.19 (5H), 1.88 |
| | | | (2H), 1.48 (3H), 1.38 |
| | | | (2H). |
| 63 | 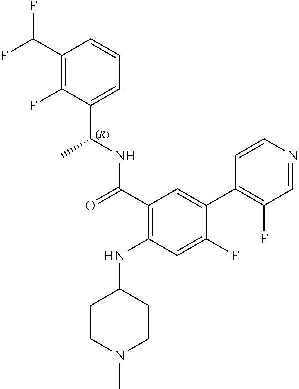 | (R)-N-(1-(3- (difluoromethyl)-2- fluorophenyl)ethyl)-4- fluoro-5-(3-fluoropyridin- 4-yl)-2-((1- methylpiperidin-4- yl)amino)benzamide | MS obsd. (ESI⁺): m/z 519.4 (M + H)⁺. ¹H NMR (400 MHz, DMSO-d₆) δ: 8.82 (1H), 8.65 (1H), 8.54 (1H), 8.34 (1H), 7.98 (1H), 7.63 (2H), 7.52 (1H), 7.37-7.10 (2H), 6.71 (1H), 5.37 (1H), 3.41 (1H), 2.62 (2H), 2.15 (5H), 1.88 (2H), 1.47 (3H), 1.38 (2H). |
| 64 | 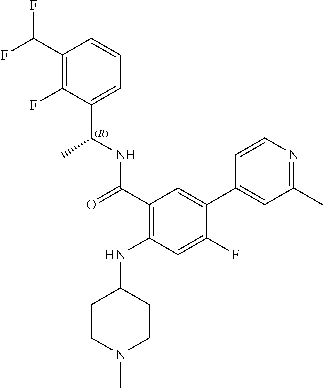 | (R)-N-(1-(3- (difluoromethyl)-2- fluorophenyl)ethyl)-4- fluoro-2-((1- methylpiperidin-4- yl)amino)-5-(2- methylpyridin-4- yl)benzamide | MS obsd. (ESI⁺): m/z 515.3 (M + H)⁺. ¹H NMR (400 MHz, MeOD) δ: 8.39 (1H), 7.93 (1H), 7.58 (2H), 7.48 (2H), 7.28 (1H), 7.01 (1H), 6.60 (1H), 5.45 (1H), 3.48 (1H), 2.76 (2H), 2.58 (3H), 2.41-2.27 (5H), 2.03 (2H), 1.64-1.50 (5H). |
| 65 | 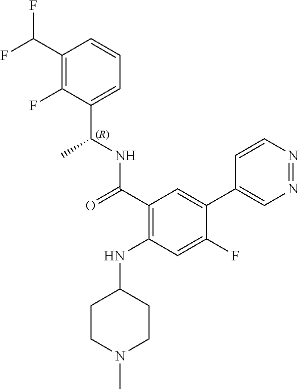 | (R)-N-(1-(3- (difluoromethyl)-2- fluorophenyl)ethyl)-4- fluoro-2-((1- methylpiperidin-4- yl)amino)-5-(pyridazin-4- yl)benzamide | MS obsd. (ESI⁺): m/z 502.4 (M + H)⁺. ¹H NMR (400 MHz, DMSO-d₆) δ 9.57 (1H), 9.24 (1H), 8.91 (1H), 8.45 (1H), 8.19 (1H), 7.89 (1H), 7.69 (1H), 7.53 (1H), 7.36 (1H), 7.22 (1H), 6.75 (1H), 5.39 (1H), 3.48- 3.36 (1H), 2.59 (2H), 2.15 (3H), 2.11 (2H), 1.87 (2H), 1.51 (3H), 1.46-1.32 (2H). |

### Example 66: (R)—N-(1-(3-(difluoromethyl)-2-fluorophenyl)ethyl)-4-fluoro-2-((1-methylpiperidin-4-yl)amino)-5-(pyrimidin-4-yl)benzamide

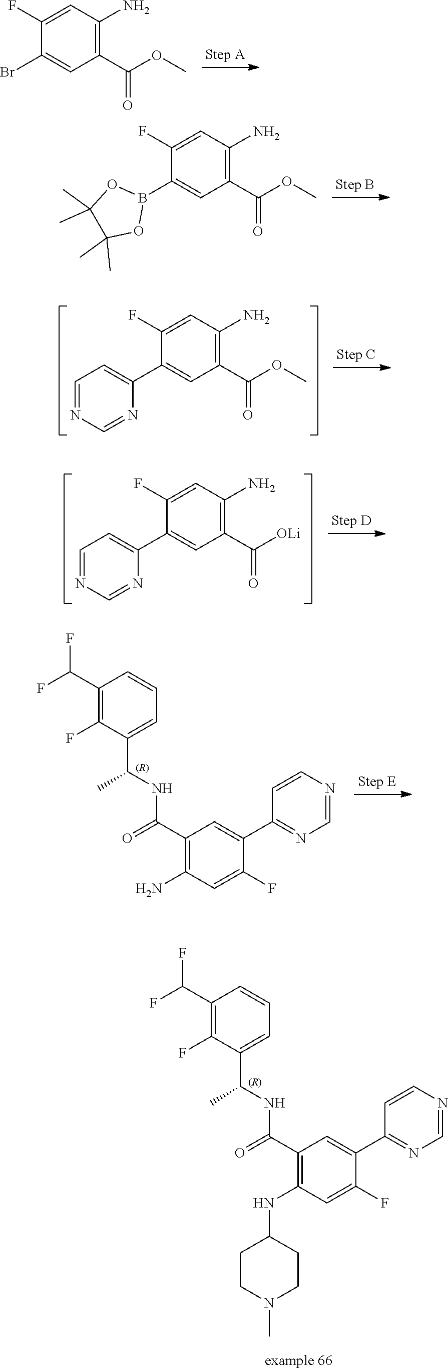

### Step A: Methyl 2-amino-4-fluoro-5-(4,4,5,5-tetramethyl-1,3,2-dioxaborolan-2-yl)benzoate

To a solution of methyl 2-amino-5-bromo-4-fluoro-benzoate (450 mg, 1.81 mmol) and 4,4,5,5-tetramethyl-2-(4,4,5,5-tetramethyl-1,3,2-dioxaborolan-2-yl)-1,3,2-dioxaborolane (553 mg, 2.18 mmol) in 1.4-dioxane (10 mL) was added sodium acetate (446 mg, 5.44 mmol) and Pd(dppf)Cl₂ (265 mg, 0.36 mmol). The reaction was stirred for 16 hrs at 90° C. The mixture was diluted with water and extracted with DCM (3*20 mL). The combined organic layers were dried over Na₂SO₄, filtered and concentrated. The residue was purified by flash column chromatography (eluting with 0%-20% MeOH in DCM) to afford the title compound (230 mg, 42% yield). MS obsd. (ESI⁺): m/z 296.4 (M+H)⁺.

### Step B: Methyl 2-amino-4-fluoro-5-(pyrimidin-4-yl)benzoate

To a solution of methyl 2-amino-4-fluoro-5-(4,4,5,5-tetramethyl-1,3,2-dioxaborolan-2-yl)benzoate (220 mg, 0.74 mmol) and 4-chloropyrimidine (102 mg, 0.89 mmol) in 1,4-dioxane (5 mL) was added Pd(dppf)Cl₂ (109 mg, 0.15 mmol) and Na₂CO₃ (237 mg, 2.24 mmol). The reaction was stirred for 16 hrs at 80° C. The mixture was diluted with water and extracted with DCM (3*20 mL). The combined organic layers were dried over Na₂SO₄, filtered and concentrated to afford the crude title compound (185 mg, crude). The crude product was used for the next step without further purification. MS obsd. (ESI⁺): m/z 248.2 (M+H)⁺.

### Step C: Lithium 2-amino-4-fluoro-5-(pyrimidin-4-yl)benzoate

To a solution of methyl 2-amino-4-fluoro-5-(pyrimidin-4-yl)benzoate (185 mg, crude) in CH₃OH (3 mL) and H₂O (1 mL) was added lithium hydroxide (72 mg, 2.99 mmol), The reaction was stirred for 16 hrs at 50° C. The mixture was extracted with DCM (3*20 mL) The aqueous phase was collected and concentrated in vacuo afford the crude title compound (180 mg, crude). The crude product was used for the next step without further purification. MS obsd. (ESI⁺): m/z 234.2 (M+H)⁺.

### Step D: (R)-2-amino-N-(1-(3-(difluoromethyl)-2-fluorophenyl)ethyl)-4-fluoro-5-(pyrimidin-4-yl)benzamide

A solution of lithium 2-amino-4-fluoro-5-(pyrimidin-4-yl)benzoate (180 mg, crude) and HATU (381 mg, 1.00 mmol) in DMF (5 mL) was stirred 10 min. Then (R)-1-(3-(difluoromethyl)-2-fluorophenyl) ethan-1-amine hydrochloride (226 mg, 1.00 mmol) and DIPEA (299 mg, 2.32 mmol) were added. The reaction was stirred for 2 h at rt. The mixture was diluted with water and extracted with DCM (3*20 mL). The combined organic layers were dried over Na₂SO₄, filtered and concentrated. The residue was purified by flash column chromatography (eluting with 0%-20% MeOH in DCM) to afford the title compound (220 mg). MS obsd. (ESI⁺): m/z 405.3 (M+H)+

### Step E: (R)—N-(1-(3-(difluoromethyl)-2-fluorophenyl)ethyl)-4-fluoro-2-((1-methylpiperidin-4-yl)amino)-5-(pyrimidin-4-yl)benzamide (example 66)

To a solution of (R)-2-amino-N-(1-(3-(difluoromethyl)-2-fluorophenyl)ethyl)-4-fluoro-5-(pyrimidin-4-yl)benzamide (180 mg, 0.44 mmol) and 1-methylpiperidin-4-one (1 g, 8.90 mmol) in TFA (5 mL) was added sodium cyanoborohydride (280 mg, 4.45 mmol). The reaction was stirred for 2 hrs at 50° C. The solvent was removed in vacuo and the residue was purified by flash column chromatography (eluting with 0%-20% MeOH in DCM) followed by preparative HPLC (ACN/water/0.1% CH₃COOH) to afford the title compound (32.5 mg). MS obsd. (ESI⁺): m/z 502.3 (M+H)⁺. ¹H NMR (400 MHZ, MeOD) δ 9.15 (1H), 8.70 (1H), 8.57 (1H), 7.85 (1H), 7.62 (1H), 7.51 (1H), 7.29 (1H), 7.01 (1H), 6.63 (1H), 5.46 (1H), 3.58 (1H), 2.95 (2H), 2.59 (2H), 2.46 (3H), 2.10 (2H), 1.61 (5H).

### Example 67: (R)-5-cyclopropyl-N-(1-(3-(difluoromethyl)-2-fluorophenyl)ethyl)-4-fluoro-2-((1-methylpiperidin-4-yl)amino)benzamide

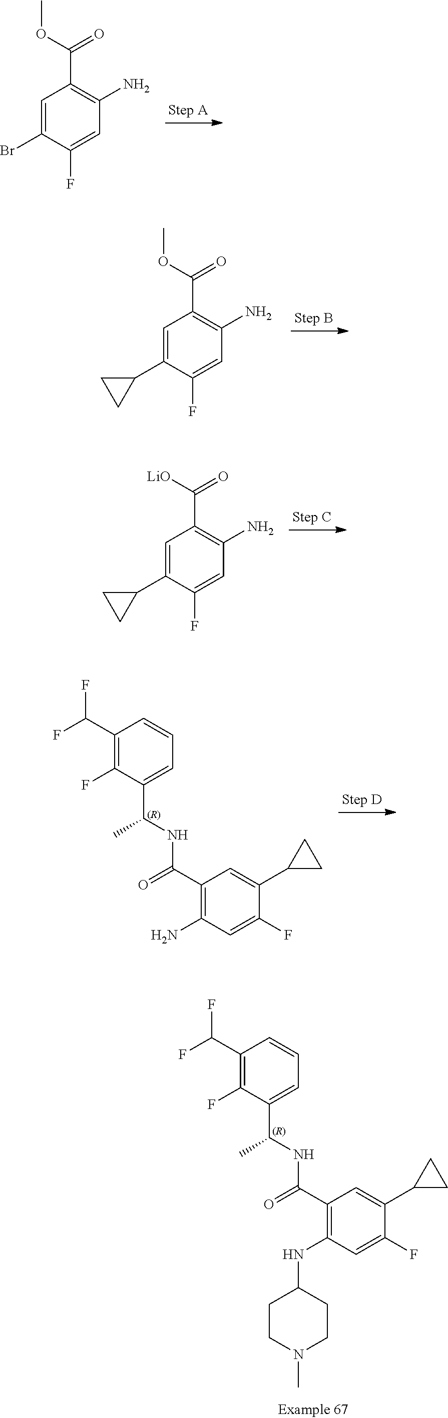

### Step A: methyl 2-amino-5-cyclopropyl-4-fluorobenzoate

A mixture of methyl 2-amino-5-bromo-4-fluoro-benzoate (500 mg, 2.02 mmol), cyclopropylboronic acid (208 mg, 2.42 mmol), PdCl₂ (dppf) (147 mg, 0.20 mmol) and sodium carbonate (641 mg, 6.05 mmol) in 1,4-dioxane (2 mL) and water (0.4 mL) was stirred at 110° C. for 1 hr. The reaction mixture was cooled to room temperature, quenched with ice water and the reaction mixture was extracted with DCM (3*40 mL). The combined organic layers were washed with brine, dried over Na₂SO₄, filtered and concentrated. The residue was purified by flash column chromatography (eluting with 0-40% EA in PE) to afford the title compound (200 mg, 47% yield). MS obsd. (ESI⁺): m/z 210.3 (M+H)⁺.

### Step B: lithium 2-amino-5-cyclopropyl-4-fluorobenzoate

A mixture of methyl 2-amino-5-cyclopropyl-4-fluoro-benzoate (200 mg, 0.95 mmol) and LiOH (229 mg, 9.56 mmol) in THF (5 mL) and H₂O (5 mL) was stirred at 60° C. for 16 hrs. The reaction mixture was cooled to room temperature and concentrated in vacuo to afford the title compound (210 mg, crude). This material was used without further purification. MS obsd. (ESI⁺): m/z 196.3 (M+H)⁺ for free acid.

### Step C: (R)-2-amino-5-cyclopropyl-N-(1-(3-(difluoromethyl)-2-fluorophenyl)ethyl)-4-fluorobenzamide

To a solution of lithium 2-amino-5-cyclopropyl-4-fluorobenzoate (210 mg, crude) in DMF (5 mL) was added HATU (476 mg, 1.25 mmol). The reaction was stirred for 10 mins at rt. Then (R)-1-(3-(difluoromethyl)-2-fluorophenyl) ethan-1-amine (257 mg, 1.36 mmol) and DIPEA (404 mg, 3.13 mmol) were added and the reaction was stirred for 3 hrs at rt. The mixture was diluted with water and extracted with DCM (3*20 mL). The combined organic layers were dried over Na₂SO₄, filtered and concentrated. The residue was purified by flash column chromatography (eluting with 0%-7% MeOH in DCM) to afford the title compound (327 mg). MS obsd. (ESI⁺): m/z 367.8 (M+H)⁺.

### Step D: (R)-5-cyclopropyl-N-(1-(3-(difluoromethyl)-2-fluorophenyl)ethyl)-4-fluoro-2-((1-methylpiperidin-4-yl)amino)benzamide (example 67)

To a solution of (R)-2-amino-5-cyclopropyl-N-(1-(3-(difluoromethyl)-2-fluorophenyl)ethyl)-4-fluorobenzamide (317 mg, 0.86 mmol) in AcOH (10 mL) was added 1-methylpiperidin-4-one (783 mg, 6.92 mmol) and sodium triacetoxyborohydride (1.5 g, 6.92 mmol). The reaction was stirred for 2 hrs at rt. The solvent was removed in vacuo and the residue was redissolved in DCM (20 mL). The mixture was neutralized by NaHCO₃ (aq, 20 mL) and the mixture was extracted with DCM (3*20 mL). The combined organic layers were dried over Na₂SO₄, filtered and concentrated. The residue was purified by flash column chromatography (eluting with 0%-50% MeOH in DCM) followed by preparative HPLC (ACN/water/0.1% NH₄HCO₃) to afford the title compound (195.5 mg, 48% yield) as a white solid. MS obsd. (ESI⁺): m/z 464.0 (M+H)⁺. ¹H NMR (400 MHZ, DMSO-d₆) δ: 8.67 (1H), 7.85 (1H), 7.62 (1H), 7.51 (1H), 7.36 (2H), 7.21 (1H), 6.46 (1H), 5.35 (1H), 3.24 (1H), 2.56 (2H), 2.13 (3H), 2.07 (2H), 1.89-1.78 (3H), 1.47 (3H), 1.31 (2H), 0.86 (2H), 0.73 (2H).

### Example 68: (R)-5-(bicyclo[1.1.1]pentan-1-yl)-N-(1-(3-(difluoromethyl)-2-fluorophenyl)ethyl)-4-fluoro-2-((1-methylpiperidin-4-yl)amino)benzamide

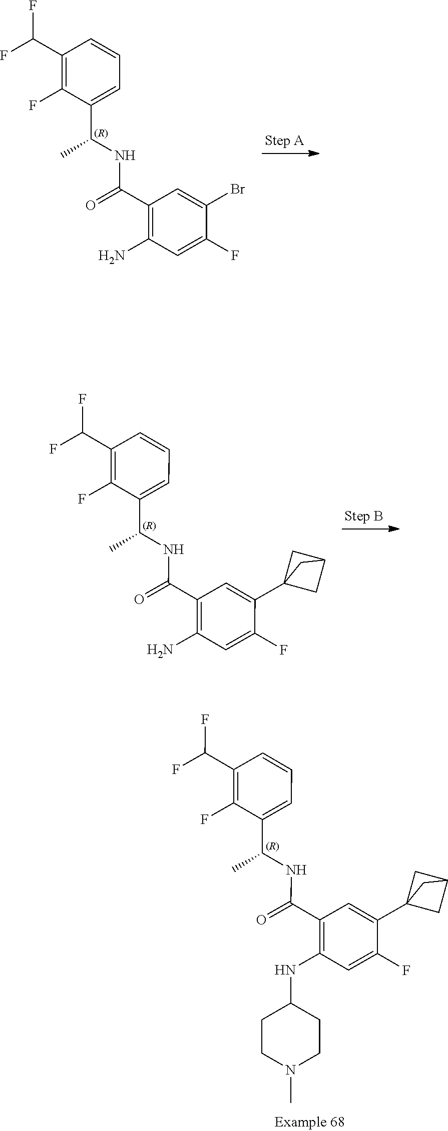

### Step A: (R)-2-amino-5-(bicyclo[1.1.1]pentan-1-yl)-N-(1-(3-(difluoromethyl)-2-fluorophenyl)ethyl)-4-fluorobenzamide

A mixture of (R)-2-amino-5-bromo-N-(1-(3-(difluoromethyl)-2-fluorophenyl)ethyl)-4-fluorobenzamide (100 mg, 0.25 mmol), potassium bicyclo[1.1.1]pentan-1-yltrifluoroborate (64 mg, 0.37 mmol), sodium carbonate (52 mg, 0.49 mmol), Ni (dtbbpy) Br₂ (18 mg, 0.04 mmol) and Ir[dF(CF₃)ppy]₂(bpy)PF₆ (25 mg, 0.024 mmol) in DMA (0.50 mL) was stirred for 12 hrs at rt with blue LED irradiation. The reaction was diluted in EtOAc (100 mL) and washed with water and brine. The organic layer was dried over Na₂SO₄, filtered and concentrated to dryness. The residue was purified by silica gel chromatography (EA/PE=1/4) followed by reverse phase column (ACN/water/0.5% FA=45% to 50%) to afford the title compound (15 mg, 15% yield). MS obsd. (ESI⁺): m/z 393.6 (M+H)⁺.

### Step B: (R)-5-(bicyclo[1.1.1]pentan-1-yl)-N-(1-(3-(difluoromethyl)-2-fluorophenyl)ethyl)-4-fluoro-2-((1-methylpiperidin-4-yl)amino)benzamide (example 68)

To a solution of 1-methylpiperidin-4-one (29 mg, 0.25 mmol) and (R)-5-(bicyclo[1.1.1]pentan-1-yl)-N-(1-(3-(difluoromethyl)-2-fluorophenyl)ethyl)-4-fluoro-2-((1-methylpiperidin-4-yl)amino)benzamide (20 mg, 0.05 mmol) in acetic acid (1 mL) was added sodium cyanoborohydride (16 mg, 0.25 mmol). The reaction was stirred for 16 hr at rt. The reaction was concentrated to dryness and the residue was purified by silica column (MeOH/DCM=1/6) followed by preparative HPLC (MeCN/water/0.5% FA=45%˜50%) to afford the title compound (11.2 mg, 44% yield). MS obsd. (ESI⁺): m/z 490.6 (M+H)⁺. ¹H NMR (400 MHZ, DMSO-d₆) δ 8.74 (1H), 7.89 (1H), 7.63 (1H), 7.59-7.44 (2H), 7.37 (1H), 7.22 (1H), 6.44 (1H), 5.37 (1H), 3.54 (1H), 2.68 (2H), 2.56 (1H), 2.36-2.05 (11H), 1.84 (2H), 1.49 (3H), 1.35 (2H).

### Examples 69 and 70:5-(bicyclo[1.1.1]pentan-1-yl)-N—((R)-1-(3-(difluoromethyl)-2-fluorophenyl)ethyl)-4-fluoro-2-(((R)-quinuclidin-3-yl)amino)benzamide and 5-(bicyclo[1.1.1]pentan-1-yl)-N—((R)-1-(3-(difluoromethyl)-2-fluorophenyl)ethyl)-4-fluoro-2-(((S)-quinuclidin-3-yl)amino)benzamide

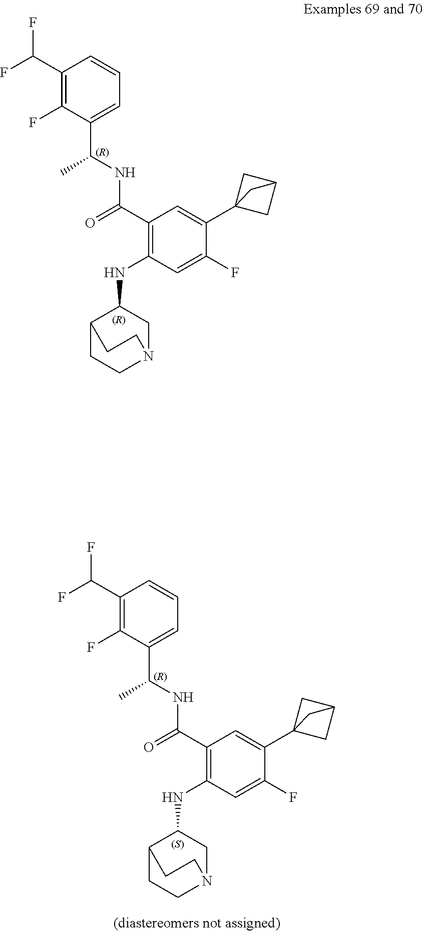

A mixture of quinuclidin-3-one (191 mg, 1.53 mmol) and (R)-2-amino-5-(bicyclo[1.1.1]pentan-1-yl)-N-(1-(3-(difluoromethyl)-2-fluorophenyl)ethyl)-4-fluorobenzamide (60 mg, 0.15 mmol) in titanium (IV) ethoxide (2 mL) was stirred at 60° C. for 16 hrs. The resulting mixture was diluted in EtOH (5 mL) and sodium borohydride (4.5 mg, 0.12 mmol) was added. The reaction was stirred for 1 hr at rt. The reaction was concentrated to dryness and the residue was dissolved in DCM (100 mL) and filtered through celite. The filtrate was collected and concentrated in vacuo. The crude product was purified by preparative HPLC (ACN/water/0.5% FA) to afford the title compounds as a diastereomeric mixture (22 mg, 36% yield). MS obsd. (ESI⁺): m/z 502.7 (M+H)⁺.

Individual diastereomers were separated via chiral SFC: (Column: Daicel AD-3 (4.6×100 mm 3.0 μm), Temperature: 40° C., Mobile phase: CO₂/MeOH [0.2% NH₃ (7M in MeOH)]=80/20, Flow rate: 3 ml/min, Back pressure: 2000 psi) to afford the title compounds

Example 69: First eluting peak. MS obsd. (ESI⁺): m/z 502.8 (M+H)⁺.

Example 70: Second eluting peak. MS obsd. (ESI⁺): m/z 502.8 (M+H)⁺.

### Example 71: methyl (R)-5-((1-(3-(difluoromethyl)-2-fluorophenyl)ethyl) carbamoyl)-2-fluoro-4-((1-methylpiperidin-4-yl)amino)benzoate

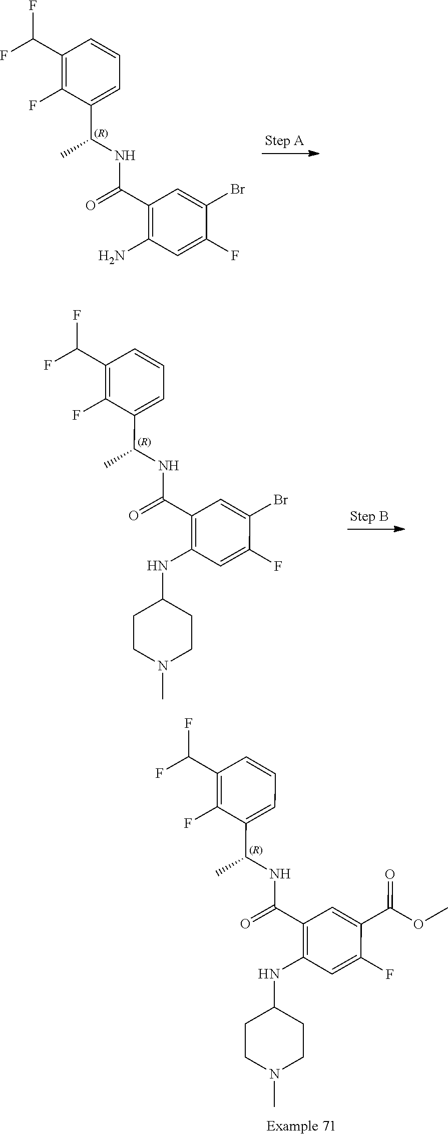

### Step A: (R)-5-bromo-N-(1-(3-(difluoromethyl)-2-fluorophenyl)ethyl)-4-fluoro-2-((1-methylpiperidin-4-yl)amino)benzamide

To a mixture of 1-methylpiperidin-4-one (2.23 g, 19.74 mmol) and (R)-2-amino-5-bromo-N-(1-(3-(difluoromethyl)-2-fluorophenyl)ethyl)-4-fluorobenzamide (800 mg, 1.97 mmol) in TFA (5 mL) was added NaBH₃CN (620 mg, 9.87 mmol). The mixture was stirred for 2 h at 50° C. The solvent was removed in vacuo and the residue was quenched with 50 mL saturated NaHCO₃ aqueous solution and extracted with DCM (3*80 mL). The combined organic layers were dried over Na₂SO₄, filtered and concentrated. The residue was purified by flash column (eluted with 0-10% MeOH in DCM) to afford the title compound (855 mg, 86% yield). MS obsd. (ESI⁺): m/z 502.3/504.3 (M+H)⁺.

### Step B: Methyl (R)-5-((1-(3-(difluoromethyl)-2-fluorophenyl)ethyl) carbamoyl)-2-fluoro-4-((1-methylpiperidin-4-yl)amino)benzoate (example 71)

To a mixture of (R)-5-bromo-N-(1-(3-(difluoromethyl)-2-fluorophenyl)ethyl)-4-fluoro-2-((1-methylpiperidin-4-yl)amino)benzamide (855 mg, 1.70 mmol) and Pd(dppf)Cl₂ (2 g, 2.73 mmol) in MeOH (200 mL) was added TEA (6 mL). The mixture was stirred for 16 h at 100° C. under 10 atm of CO. The solvent was removed in vacuo and the residue was purified by flash column (eluted with 0-5% MeOH in DCM) to afford the title compound (650 mg, 62% yield). MS obsd. (ESI⁺): m/z 482.3 (M+H)⁺. ¹H NMR (400 MHZ, MeOD-d₄) δ: 8.29 (1H), 7.57 (1H), 7.50 (1H), 7.28 (1H), 7.01 (1H), 6.48 (1H), 5.43 (1H), 3.86 (3H), 3.47 (1H), 2.74 (2H), 2.32 (2H), 2.29 (3H), 2.01 (2H), 1.57-1.53 (5H).

### Example 72: (R)—N-(1-(3-(difluoromethyl)-2-fluorophenyl)ethyl)-4-fluoro-2-((1-methylpiperidin-4-yl)amino)-5-(1,3,4-oxadiazol-2-yl)benzamide

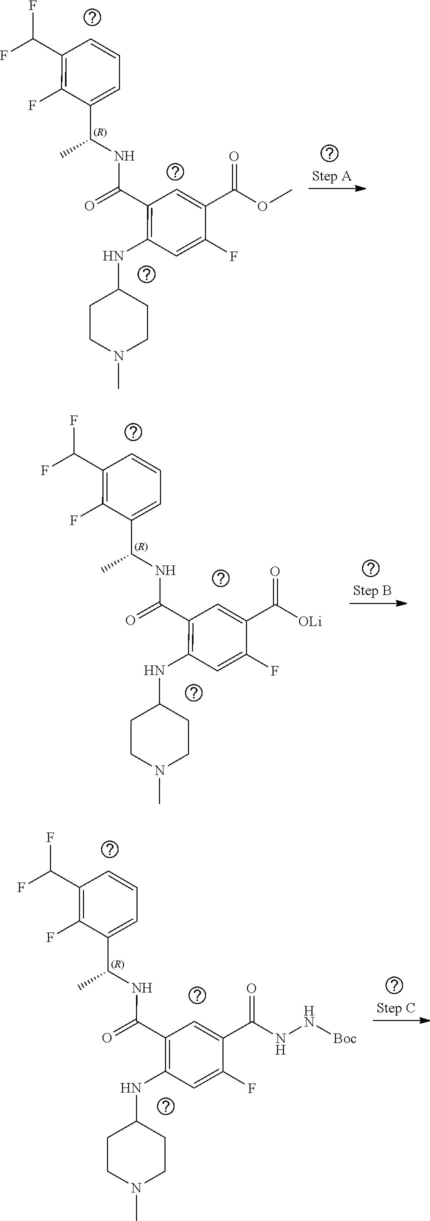

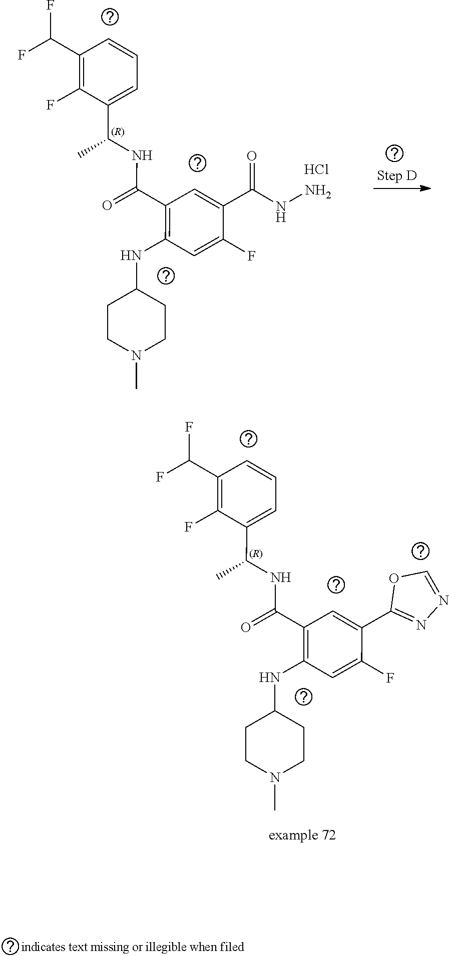

### Step A: Lithium (R)-5-((1-(3-(difluoromethyl)-2-fluorophenyl)ethyl) carbamoyl)-2-fluoro-4-((1-methylpiperidin-4-yl)amino)benzoate

To a mixture of methyl (R)-5-((1-(3-(difluoromethyl)-2-fluorophenyl)ethyl) carbamoyl)-2-fluoro-4-((1-methylpiperidin-4-yl)amino)benzoate (380 mg, 0.79 mmol) in MeOH (4 mL) and water (0.8 mL) was added LiOH (38 mg, 1.58 mmol). The mixture was stirred for 1 h at 50° C. The solvent was removed in vacuo to afford lithium (R)-5-((1-(3-(difluoromethyl)-2-fluorophenyl)ethyl) carbamoyl)-2-fluoro-4-((1-methylpiperidin-4-yl)amino)benzoate (373 mg, crude). This material was used without further purification. MS obsd. (ESI⁺): m/z 468.3 (M+H)⁺ for free acid.

### Step B: Tert-butyl (R)-2-(5-((1-(3-(difluoromethyl)-2-fluorophenyl)ethyl) carbamoyl)-2-fluoro-4-((1-methylpiperidin-4-yl)amino)benzoyl)hydrazine-1-carboxylate

To a mixture of lithium (R)-5-((1-(3-(difluoromethyl)-2-fluorophenyl)ethyl) carbamoyl)-2-fluoro-4-((1-methylpiperidin-4-yl)amino)benzoate (373 mg, crude) in DMF (10 mL) was added HATU (449 mg, 1.18 mmol). The mixture was stirred for 10 min at rt. Then tert-butyl hydrazinecarboxylate (156 mg, 1.18 mmol) and DIPEA (305 mg, 2.36 mmol) were added and stirred for 1 h at rt. The mixture was quenched with 40 mL water and extracted with DCM (3*80 mL). The combined organic layers were dried over Na₂SO₄, filtered and concentrated. The residue was purified by flash column chromatography (eluted with 0˜10% MeOH in DCM) to afford the title compound (137 mg). MS obsd. (ESI⁺): m/z 582.3 (M+H)⁺.

### Step C: (R)—N-(1-(3-(difluoromethyl)-2-fluorophenyl)ethyl)-4-fluoro-5-(hydrazinecarbonyl)-2-((1-methylpiperidin-4-yl)amino)benzamide; hydrochloride

A mixture of tert-butyl (R)-2-(5-((1-(3-(difluoromethyl)-2-fluorophenyl)ethyl) carbamoyl)-2-fluoro-4-((1-methylpiperidin-4-yl)amino)benzoyl)hydrazine-1-carboxylate (75 mg, 0.13 mmol) in 5 mL of 2 M HCl in 1,4-dioxane was stirred for 30 min at rt. The solvent was removed in vacuo to afford the crude title compound (66 mg, crude), which was used without further purification. MS obsd. (ESI⁺): m/z 482.2 (M+H)⁺.

### Step D: (R)—N-(1-(3-(difluoromethyl)-2-fluorophenyl)ethyl)-4-fluoro-2-((1-methylpiperidin-4-yl)amino)-5-(1,3,4-oxadiazol-2-yl)benzamide (example 72)

To a mixture of (R)—N-(1-(3-(difluoromethyl)-2-fluorophenyl)ethyl)-4-fluoro-5-(hydrazinecarbonyl)-2-((1-methylpiperidin-4-yl)amino)benzamide hydrochloride (66 mg, crude) in trimethyl orthoformate (3 mL) was added p-toluenesulfonic acid (0.4 mg). The mixture was stirred for 30 min at 80° C. The mixture was quenched with 20 mL saturated NaHCO₃ solution and extracted with DCM (3*20 mL). The combined organic layers were dried over Na₂SO₄, filtered and concentrated. The residue was purified by flash column (eluted with 0-10% MeOH in DCM) followed by preparative HPLC (ACN/water/0.1% NH₄HCO₃) to afford the title compound (9.8 mg). MS obsd. (ESI⁺): m/z 492.2 (M+H)⁺. ¹H NMR (400 MHZ, DMSO-d6) δ: 9.34 (1H), 9.20 (1H), 8.57 (1H), 8.45 (1H), 7.68 (1H), 7.53 (1H), 7.37 (1H), 7.22 (1H), 6.79 (1H), 5.39 (1H), 3.45 (1H), 2.64 (2H), 2.19 (5H), 1.89 (2H), 1.50 (3H), 1.41 (2H).

### Example 73: (R)—N1-(1-(3-(difluoromethyl)-2-fluorophenyl)ethyl)-4-fluoro-N3-methyl-6-((1-methylpiperidin-4-yl)amino)isophthalamide

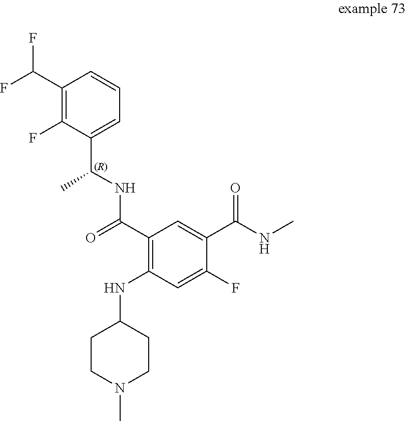

To a mixture of lithium (R)-5-((1-(3-(difluoromethyl)-2-fluorophenyl)ethyl) carbamoyl)-2-fluoro-4-((1-methylpiperidin-4-yl)amino)benzoate (114 mg, crude) in DMF (5 mL) was added HATU (137 mg, 0.36 mmol). The mixture was stirred for 10 min at rt. Then methylamine hydrochloride (24 mg, 0.36 mmol) and DIPEA (93 mg, 0.72 mmol) were added into the mixture and stirred for 1 h at rt. The solvent was removed in vacuo and the residue was diluted with water and extracted with DCM (3*80 mL). The combined organic layers were dried over Na₂SO₄, filtered and concentrated. The residue was purified by flash column (eluted with 0-10% MeOH in DCM) followed by preparative HPLC (ACN/water/0.1% NH₄HCO₃) to afford the title compound (24.0 mg). MS obsd. (ESI⁺): m/z 481.3 (M+H)⁺. ¹H NMR (400 MHZ, DMSO-d6) δ: 9.18 (1H), 8.30 (1H), 8.26 (1H), 8.12 (1H), 7.72 (1H), 7.51 (1H), 7.36 (1H), 7.21 (1H), 6.53 (1H), 5.38 (1H), 3.38 (1H), 2.76 (3H), 2.61 (2H), 2.17 (5H), 1.86 (2H), 1.49 (3H), 1.45-1.31 (2H).

### Example 74: (R)—N-(1-(3-(difluoromethyl)-2-fluorophenyl)ethyl)-5-(4,4-difluoropiperidine-1-carbonyl)-4-fluoro-2-((1-methylpiperidin-4-yl)amino)benzamide

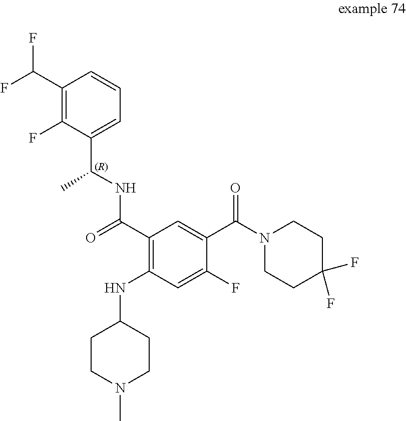

Prepared according to an analogous procedure described for example 73. MS obsd. (ESI⁺): m/z 571.3 (M+H)⁺. ¹H NMR (400 MHZ, DMSO-d₆) δ 8.86 (1H), 8.38 (1H), 7.92 (1H), 7.64 (1H), 7.52 (1H), 7.28 (2H), 6.58 (1H), 5.37 (1H), 3.77 (2H), 2.59 (2H), 2.14 (3H), 2.12-1.94 (6H), 1.86 (2H), 1.47 (3H), 1.43-1.30 (2H).

### Example 75: (R)—N1-(1-(3-(difluoromethyl)-2-fluorophenyl)ethyl)-4-fluoro-N3,N3-dimethyl-6-((1-methylpiperidin-4-yl)amino)isophthalamide

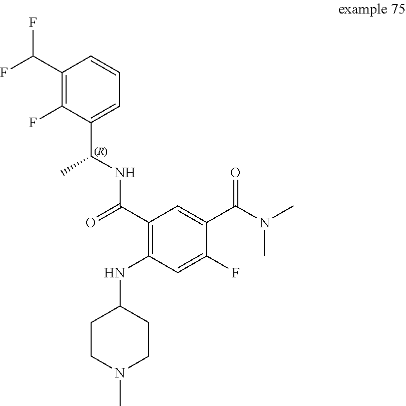

Prepared according to an analogous procedure described for example 73. MS obsd. (ESI⁺): m/z 495.3 (M+H)⁺. ¹H NMR (400 MHZ, DMSO-d6) δ ppm 8.86 (1H), 8.36 (1H), 7.89 (1H), 7.63 (1H), 7.51 (1H), 7.36 (1H), 7.21 (1H), 6.55 (1H), 5.36 (1H), 3.35 (1H), 2.98 (3H), 2.90 (3H), 2.58 (2H), 2.14 (3H), 2.10 (2H), 1.86 (2H), 1.46 (3H), 1.36 (2H).

### Example 76: (R)—N1-(1-(3-(difluoromethyl)-2-fluorophenyl)ethyl)-4-fluoro-N3-isopropyl-6-((1-methylpiperidin-4-yl)amino)isophthalamide

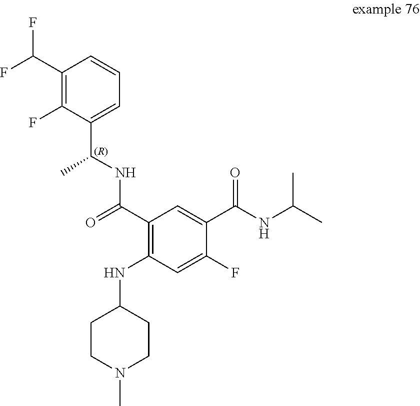

Prepared according to an analogous procedure described for example 73. MS obsd. (ESI⁺): m/z 509.3 (M+H)⁺. ¹H NMR (400 MHZ, DMSO-d₆) δ 8.99 (1H), 8.28 (1H), 8.11 (1H), 7.62 (2H), 7.52 (1H), 7.39-7.06 (2H), 6.52 (1H), 5.37 (1H), 4.06 (1H), 2.19-2.04 (5H), 1.85 (2H), 1.47 (3H), 1.36 (2H), 1.16 (6H).

### Example 77: (R)—N-(1-(3-(difluoromethyl)-2-fluorophenyl)ethyl)-4-fluoro-2-((1-methylpiperidin-4-yl)amino)-5-(morpholine-4-carbonyl)benzamide

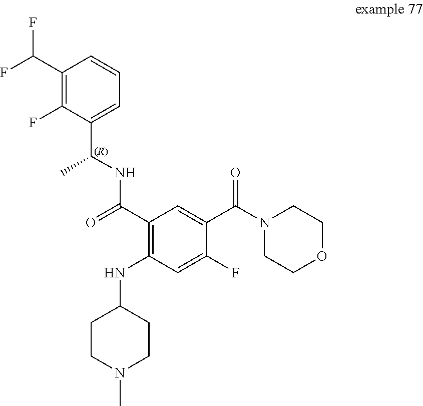

Prepared according to an analogous procedure described for example 73. MS obsd. (ESI⁺): m/z 537.3 (M+H)⁺. ¹H NMR (400 MHZ, MeOD-d₄) δ:7.75 (1H), 7.57 (1H), 7.50 (1H), 7.27 (1H), 7.00 (1H), 6.50 (1H), 5.42 (1H), 3.69 (6H), 3.44 (3H), 2.73 (2H), 2.31-2.28 (5H), 2.00 (2H), 1.60-1.49 (5H).

### Example 78: methyl (R)-3-((1-(3-(difluoromethyl)-2-fluorophenyl)ethyl) carbamoyl)-4-((1-methylpiperidin-4-yl)amino)benzoate

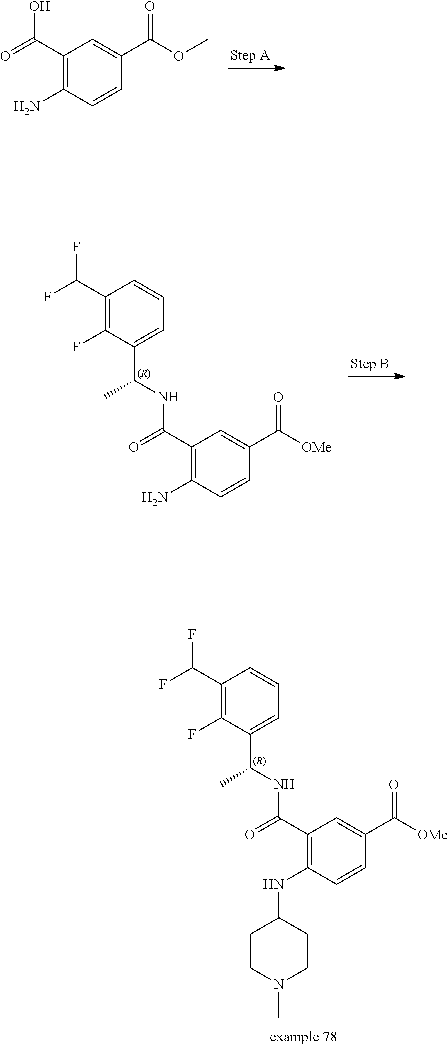

### Step A: methyl (R)-4-amino-3-((1-(3-(difluoromethyl)-2-fluorophenyl)ethyl) carbamoyl)benzoate

To a solution of 2-amino-5-methoxycarbonyl-benzoic acid (50 mg, 0.26 mmol) in DMF (1.5 mL) was added DIPEA (99 mg, 0.77 mmol), (R)-1-(3-(difluoromethyl)-2-fluorophenyl) ethan-1-amine hydrochloride (69 mg, 0.31 mmol) and HATU (146 mg, 0.38 mmol). The mixture was stirred at rt for 2 hr. The reaction mixture was poured into water (40 mL) then extracted with EA (30 mL×3). The combined organic layers were washed with brine, dried over Na₂SO₄, filtered and concentrated under reduced pressure. The crude product was purified by column chromatography (0-40% EA in PE) to afford the title compound (75 mg, 79% yield). MS obsd. (ESI⁺): m/z 367.3 (M+H)⁺.

### Step B: methyl (R)-3-((1-(3-(difluoromethyl)-2-fluorophenyl)ethyl) carbamoyl)-4-((1-methylpiperidin-4-yl)amino)benzoate

To a solution of methyl (R)-4-amino-3-((1-(3-(difluoromethyl)-2-fluorophenyl)ethyl) carbamoyl)benzoate (60 mg, 0.16 mmol) in TFA (2.5 mL) was added 1-methylpiperidin-4-one (185 mg, 1.64 mmol) and NaBH₃CN (51 mg, 0.82 mmol). The mixture was stirred at 50° C. for 2.5 hr. The mixture was concentrated and purified by column chromatography (0-10% MeOH in DCM) followed by preparative HPLC (ACN-H₂O (10 mmol NH₄HCO₃)) to afford the title compound (33.7 mg, 44% yield). MS obsd. (ESI⁺): m/z 464.3 (M+H)⁺. ¹H NMR (400 MHZ, DMSO-d₆) δ 9.10 (1H), 8.36 (2H), 7.82 (1H), 7.66 (1H), 7.52 (1H), 7.38-7.08 (2H), 6.79 (1H), 5.37 (1H), 3.81 (3H), 3.43 (1H), 2.14 (3H), 2.07 (2H), 1.86 (2H), 1.49 (3H), 1.38 (2H).

### Example 79: (R)-4-(difluoromethyl)-N-(1-(3-(difluoromethyl)-2-fluorophenyl)ethyl)-2-((1-methylpiperidin-4-yl)amino)-5-morpholinobenzamide

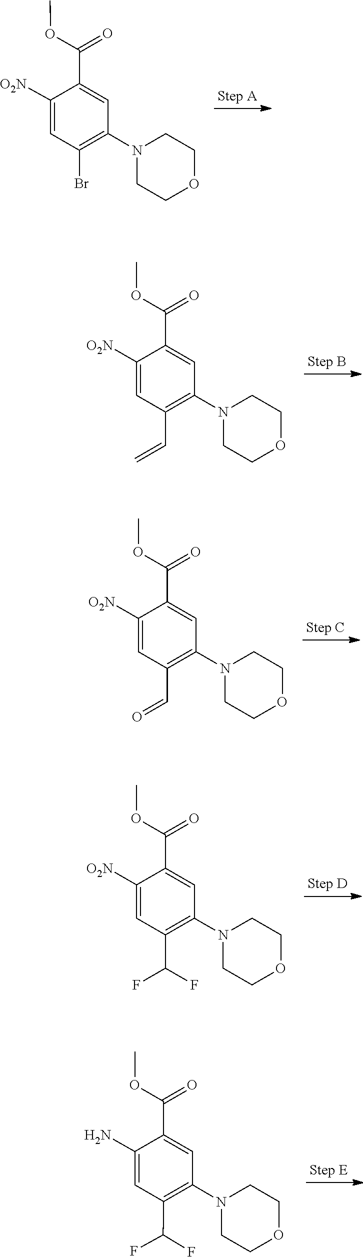 [SMILES: COC(=O)c1cc(N2CCOCC2)c(Br)cc1[N+](=O)[O-] ; C=Cc1cc([N+](=O)[O-])c(C(=O)OC)cc1N1CCOCC1 ; COC(=O)c1cc(N2CCOCC2)c(C=O)cc1[N+](=O)[O-] ; COC(=O)c1cc(N2CCOCC2)c(C(F)F)cc1[N+](=O)[O-] ; COC(=O)c1cc(N2CCOCC2)c(C(F)F)cc1N]

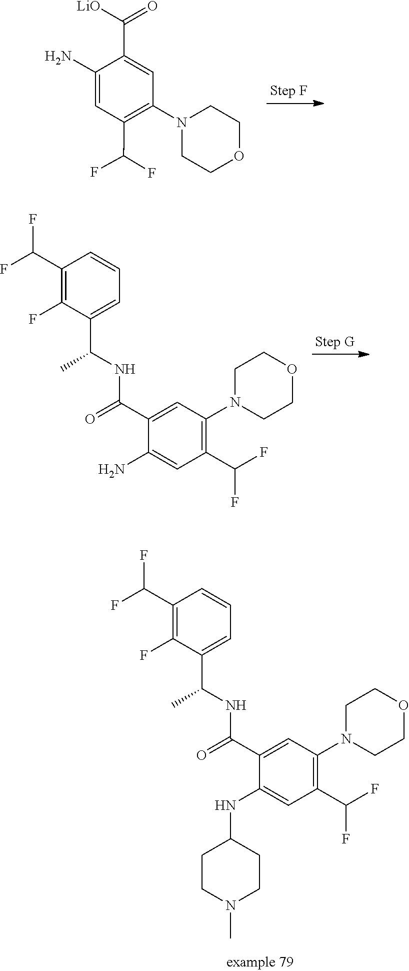

### Step A: Methyl 5-morpholino-2-nitro-4-vinylbenzoate

To a solution of 4,4,5,5-tetramethyl-2-vinyl-1,3,2-dioxaborolane (446 mg, 2.90 mmol) and methyl 4-bromo-5-morpholino-2-nitro-benzoate (1.0 g, 2.90 mmol) in 1,4-dioxane (15 mL) and water (1 mL) was added potassium carbonate (1.2 g, 8.69 mmol) and Pd(dppf)Cl₂ (210 mg, 0.29 mmol). The reaction was heated to 90° C. for 2 hrs. The mixture was cooled to room temperature and concentrated. The residue was dissolved in CH₂Cl₂ (30 mL), and the solution was washed with water (30 mL) twice, dried over Na₂SO₄, and concentrated in vacuo. The resultant crude product was purified by column chromatography to afford the target compound (506 mg, 59% yield). MS obsd. (ESI⁺): m/z 293.4 (M+H)⁺.

### Step B: Methyl 4-formyl-5-morpholino-2-nitrobenzoate

To a solution of methyl 5-morpholino-2-nitro-4-vinyl-benzoate (506 mg, 1.73 mmol) in a mixture of solvent of DCM (15 mL) and water (3 mL) was added (diacetoxyiodo)benzene (1.7 g, 5.19 mmol) and ruthenium (III) chloride (72 mg, 0.35 mmol). The reaction was heated to 30° C. for 1.5 hrs. The reaction was cooled to room temperature and quenched with a saturated aqueous solution of NaS203 and then extracted with CH₂Cl₂ twice (2×40 mL). The combined organic layers were washed with water and brine, dried over Na₂SO₄, filtered and concentrated to afford the crude product, which was purified by flash column chromatograpy (PE-EtOAc, 85:15) to afford the title compound (240 mg, 47% yield). MS obsd. (ESI⁺): m/z 295.2 (M+H)⁺.

### Step C: Methyl 4-(difluoromethyl)-5-morpholino-2-nitrobenzoate

To a solution methyl 4-formyl-5-morpholino-2-nitrobenzoate (240 mg, 0.82 mmol) in DCM (10 mL) was slowly added DAST (394 mg, 2.45 mmol). The reaction was stirred for 16 hrs at 35° C. The mixture was then washed with a saturated solution of sodium bicarbonate (10×3 mL). The organic layer was dried over Na₂SO₄, filtered and concentrated in vacuo. The residue was purified by column chromatography to give the title compound. MS obsd. (ESI⁺): m/z 317.8 (M+H)⁺.

### Step D: Methyl 2-amino-4-(difluoromethyl)-5-morpholinobenzoate

To a solution of methyl 4-(difluoromethyl)-5-morpholino-2-nitro-benzoate (340 mg, 1.08 mmol) in MeOH (20 mL) was added 10% Pd/C (40 mg). The reaction was stirred under 1 atm H₂ for 2 hrs at rt. The mixture was filtered and the filtrate was concentrated in vacuo. The residue was purified by flash column chromatography (eluting with 0%-50% EA in PE) to afford the title compound (305 mg, 99% yield). MS obsd. (ESI⁺): m/z 286.8 (M+H)⁺.

### Step E: Lithium 2-amino-4-(difluoromethyl)-5-morpholinobenzoate

To a solution of methyl 2-amino-4-(difluoromethyl)-5-morpholino-benzoate (120 mg, 0.42 mmol) in MeOH (4 mL) and H₂O (1 mL) was added lithium hydroxide (40 mg, 1.68 mmol). The reaction was stirred for 16 hrs at 50° C. The solvent was removed in vacuo to afford the crude title compound (120 mg, crude). The crude product was used for the next step without further purification. MS obsd. (ESI⁺): m/z 272.8 (M+H)⁺ for free acid.

### Step F: (R)-2-amino-4-(difluoromethyl)-N-(1-(3-(difluoromethyl)-2-fluorophenyl)ethyl)-5-morpholinobenzamide

To a solution of lithium 2-amino-4-(difluoromethyl)-5-morpholinobenzoate (120 mg, crude) in DMF (5 mL) was added HATU (197 mg, 0.52 mmol). The reaction was stirred for 10 mins at rt. Then (R)-1-(3-(difluoromethyl)-2-fluorophenyl) ethan-1-amine (122 mg, 0.65 mmol) and DIPEA (167 mg, 1.29 mmol) were added and the reaction was stirred for 2 hrs at rt. The mixture was diluted with water and extracted with DCM (3*20 mL). The combined organic layers were dried over Na₂SO₄, filtered and concentrated. The residue was purified by flash column chromatography (eluting with 0%-7% MeOH in DCM) to afford the title compound (147 mg, 76% yield). MS obsd. (ESI⁺): m/z 444.8 (M+H)⁺.

### Step G: (R)-4-(difluoromethyl)-N-(1-(3-(difluoromethyl)-2-fluorophenyl)ethyl)-2-((1-methylpiperidin-4-yl)amino)-5-morpholinobenzamide (example 79)

To a solution of (R)-2-amino-4-(difluoromethyl)-N-(1-(3-(difluoromethyl)-2-fluorophenyl)ethyl)-5-morpholinobenzamide (137 mg, 0.31 mmol) in AcOH (5 mL) was added 1-methylpiperidin-4-one (350 mg, 3.09 mmol) and sodium triacetoxyborohydride (655 mg, 3.09 mmol). The reaction was stirred for 2 hrs at rt. The solvent was removed in vacuo and the residue was redissolved by DCM (20 mL). The mixture was neutralized by NaHCO₃ (aq, 20 mL) and the mixture was extracted with DCM (3*20 mL). The combined organic layers were dried over Na₂SO₄ and the solvent was removed in vacuo. The residue was purified by flash column chromatography (eluting with 0%-50% MeOH in DCM) followed by preparative HPLC (ACN/water/0.1% NH₄HCO₃) to afford the title compound (92.7 mg, 52% yield). MS obsd. (ESI⁺): m/z 541.0 (M+H)⁺. ¹H NMR (400 MHZ, DMSO-d₆) δ 8.86 (1H), 7.75 (1H), 7.65 (2H), 7.53 (1H), 7.37 (1H), 7.34-7.03 (2H), 6.78 (1H), 5.39 (1H), 3.74 (4H), 3.35 (1H), 2.85 (4H), 2.55 (2H), 2.13 (3H), 2.08 (2H), 1.84 (2H), 1.51 (3H), 1.42-1.27 (2H).

### Examples 80 and 81:4-chloro-N—((R)-1-(3-(difluoromethyl)-2-fluorophenyl)ethyl)-5-morpholino-2-(((R)-quinuclidin-3-yl)amino)benzamide and 4-chloro-N—((R)-1-(3-(difluoromethyl)-2-fluorophenyl)ethyl)-5-morpholino-2-(((S)-quinuclidin-3-yl)amino)benzamide (Diastereomers unassigned)

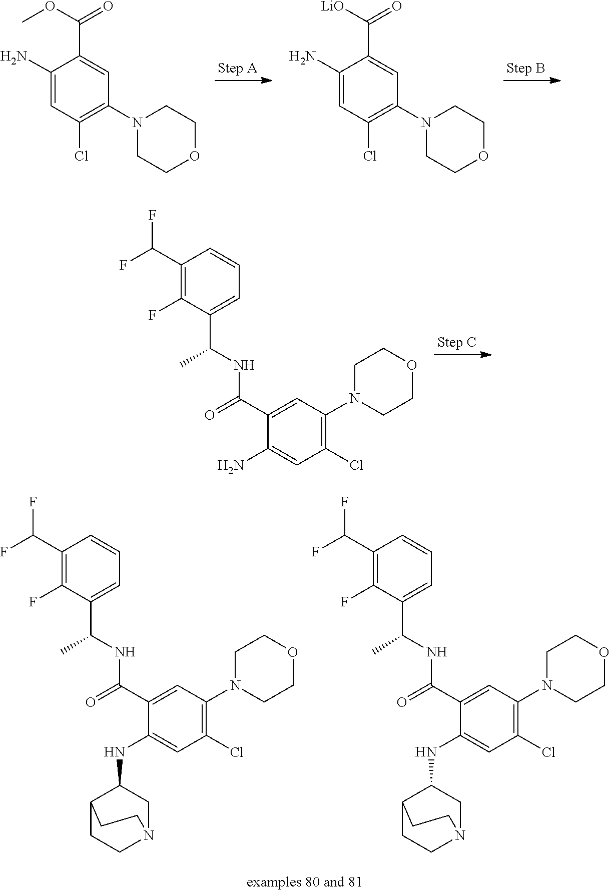

### Step A: Lithium 2-amino-4-chloro-5-morpholinobenzoate

To a solution of methyl 4-chloro-5-morpholino-2-nitro-benzoate (280 mg, 0.93 mmol) in water (2 mL) and THF (8 mL) was added LiOH (67 mg, 2.79 mmol) at rt. The reaction mixture was stirred for 16 hrs at 45° C. The mixture was concentrated in vacuo to afford the title compound (375 mg, crude), which was used without further purification. MS obsd. (ESI⁺): m/z 257.1/259.1 (M+H)⁺.

### Step B: (R)-2-amino-4-chloro-N-(1-(3-(difluoromethyl)-2-fluorophenyl)ethyl)-5-morpholinobenzamide

To a solution of the aforementioned lithium 2-amino-4-chloro-5-morpholino-benzoate (375 mg, crude) in DMF (10 mL) was added HATU (760 mg, 2.00 mmol), DIPEA (517 mg, 4.00 mmol) and (R)-1-(3-(difluoromethyl)-2-fluorophenyl) ethan-1-amine (324 mg, 1.71 mmol) at rt. The reaction mixture was stirred for 2 hrs at rt. To this mixture was added water (20 mL) and the mixture was extracted with DCM (3*20 mL). The combined organic layers were dried over anhydrous Na₂SO₄, filtered and concentrated in vacuo. The residue was purified by flash column chromatography (eluting with 0%-25% EA in PE) to afford the title compound (317 mg, 51% yield). MS obsd. (ESI⁺): m/z 428.1/430.1 (M+H)⁺.

### Step C: 4-chloro-N—((R)-1-(3-(difluoromethyl)-2-fluorophenyl)ethyl)-5-morpholino-2-(((R)-quinuclidin-3-yl)amino)benzamide & 4-chloro-N—((R)-1-(3-(difluoromethyl)-2-fluorophenyl)ethyl)-5-morpholino-2-(((S)-quinuclidin-3-yl)amino)benzamide (Examples 80 and 81)

To a solution of (R)-2-amino-4-chloro-N-(1-(3-(difluoromethyl)-2-fluorophenyl)ethyl)-5-morpholinobenzamide (250 mg, 0.58 mmol) in AcOH (500 uL) was added quinuclidin-3-one; hydrochloride (1.9 g, 11.69 mmol) and sodium cyanoborohydride (367 mg, 5.84 mmol). The reaction was stirred for 4 hrs at 60° C. The reaction was concentrated to dryness and neutralized with 7M NH3/MeOH and concentrated again. The residue was purified by reverse phase column (C18, MeCN/water/0.1% NH4HCO3) to afford the title compounds as a diastereomeric mixture (200 mg, 63% yield).

The mixture of diastereomers was separated by SFC: (Regis (R,R) Whelk-O1 (25*250 mm, 10 μm), CO₂/EtOH [0.5% NH₃ (7M in MeOH)])=65/35 to afford the individual title compounds

Example 80: First eluting isomer. MS obsd. (ESI⁺): 537.0/538.8 [(M+H)⁺].

Example 81: Second eluting isomer. MS obsd. (ESI⁺): 537.0/539.0 [(M+H)⁺].

### Biological Assays

### SOS1-KRas (G12C) FRET Assay

Inhibition of the SOS1: KRAS interaction was measured using purified GST-tagged KRAS (res. 1-169, G12C, purified based on Hillig, et al., Proc Natl Acad Sci USA (2019); 116 (7): 2551-2560) and recombinant His10-SOS1 (res. 564-1049; purified based on Hillig, et al.). The final assay was performed at 20 uL with 0.5 nM SOS1 protein and 2.5 nM KRAS protein in a buffer of PBS, 0.1% BSA, 5 mM MgCl₂, 0.0025% Igepal, 100 mM KF, 5 mM DTT in a white 384 square well OptiPlate (PerkinElmer, Cat. 6007290). A 2×KRAS working solution was prepared in an assay buffer containing 5 nM GST-KRAS G12C and 2 nM anti-GST-Eu (K) (Cisbio, Cat. 61GSTKLA) and pre-incubated for 15 minutes at 25° C. Compounds were serially diluted in 100% DMSO from 2 mM (positive control, compound I-13, PCT Publ. No. WO2018/115380) or 20 mM and then diluted 1:20 in assay buffer before incubation with a solution of SOS1 protein mixed 1:5 with anti-6His-XL665 FRET donor (Cisbio, Cat. 61HISXL) for 15 minutes at 25° C. before addition of 2×KRAS working solution. The final DMSO concentration is 0.5%. Plates were incubated at RT for 2 hrs before the FRET signal was measured using Envision at emission 665 nm and 615 nm. FRET signal was converted to percentage of protein-protein interaction using the following equation:

$$\% Inhibition = 100 \% - ( C - N ) / ( P - N ) * 100 \%$$

C: signal with compound treatment
P: signal for positive control (DMSO)
N: signal for negative control (no SOS1 added)
IC₅₀ and Hill coefficients were obtained using Graph Pad Prism (Graph Pad software, Inc, USA) with non-linear regression analysis.

TABLE A

| Example | SOS1-KRas(G12C) |
|---|---|
| Number | FRET IC₅₀ [nM] |
| 1 | 20.2 |
| 2 | 99.5 |
| 3 | 56.9 |
| 4 | 15.0 |
| 5 | 24.3 |
| 6 | 88.0 |
| 7 | 27.9 |
| 8 | 74.5 |
| 9 | 94.0 |
| 10 | 71.6 |
| 11 | 149.2 |
| 12 | 151.2 |
| 13 | 67.17 |
| 14 | 21.7 |
| 15 | 12.9 |
| 16 | 41.6 |
| 17 | 98.9 |
| 18 | 17.8 |
| 19 | 99.4 |
| 20 | 26.4 |
| 21 | 225.3 |
| 22 | 20.6 |
| 23 | 358.3 |
| 24 | 22.7 |
| 25 | 7.4 |
| 26 | 88.8 |
| 27 | 22.5 |
| 28 | 61.3 |
| 29 | 204.6 |
| 30 | 39.6 |
| 31 | 40.4 |
| 32 | 132.6 |
| 33 | 35.0 |
| 34 | 111.7 |
| 35 | 81.5 |
| 36 | 97.5 |
| 37 | 137.9 |
| 38 | 35.7 |
| 39 | 110.8 |
| 40 | 21.9 |
| 41 | 33.3 |
| 42 | 49.5 |
| 43 | 15.1 |
| 44 | 209.7 |
| 45 | 69.2 |
| 46 | 74.6 |
| 47 | 33.7 |
| 48 | 63.8 |
| 49 | 70.7 |
| 50 | 199.1 |
| 51 | 333.1 |
| 52 | 64.8 |
| 53 | 77.3 |
| 54 | 74.1 |
| 55 | 19.8 |
| 56 | 34.8 |
| 57 | 9.8 |
| 58 | 7.9 |
| 59 | 25.7 |
| 60 | 11.0 |
| 61 | 130.5 |
| 62 | 10.5 |
| 63 | 14.9 |
| 64 | 8.3 |
| 65 | 6.9 |
| 66 | 34.9 |
| 67 | 216.5 |
| 68 | 137.7 |
| 69 | 120.4 |
| 70 | 346.6 |
| 71 | 93.6 |
| 72 | 11.4 |
| 73 | 148.0 |
| 74 | 888.2 |
| 75 | 243.6 |
| 76 | 873.7 |
| 77 | 240.2 |
| 78 | 229.5 |
| 79 | 39.1 |
| 80 | 44.3 |
| 81 | 12.6 |

What is claimed is:

1. A compound of Formula (I): 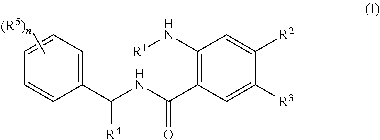
or a pharmaceutically acceptable salt thereof, wherein:
R¹ is a C₁₋₁₂ alkyl, C₃₋₆ cycloalkyl, 3- to 10-membered heterocyclyl or 3- to 10-membered (C₁₋₃ alkylene)-heterocyclyl;
R² is hydrogen, halo, cyano, C₁₋₃ alkyl, C₁₋₃ alkoxy or C₁₋₃ haloalkyl;
R³ is C₁₋₆ alkyl, C₁₋₆ haloalkyl, C₃₋₆ cycloalkyl, 3- to 10-membered heterocyclyl, 3- to 10-membered (—O—) heterocyclyl, 5- to 10-membered heteroaryl, —C(O) R⁷, —C(O) NR⁸R⁹;
R⁴ is hydrogen, C₁₋₃ alkyl or C₁₋₃ haloalkyl;
R⁵ is independently selected from halo, cyano, hydroxy, C₁₋₃ alkyl, C₁₋₃ alkoxy, C₁₋₃ haloalkyl and NH₂;
R⁷ is hydrogen, C₁₋₃ alkyl or C₁₋₃ alkoxy;
R⁸ and R⁹ are independently selected from hydrogen and C₁₋₃ alkyl; or
R⁸ and R⁹ are optionally taken together to form a 5- to 8-membered heterocyclyl; and
n is an integer 0, 1, 2 or 3;
wherein each heterocyclyl and heteroaryl is optionally substituted with one to three substituents each individually selected from halo, OH, C₁₋₃ alkyl, C₁₋₃ alkoxy, and C₁₋₃ haloalkyl, oxo, and —C(O)R⁶, wherein R⁶ is hydrogen or C₁₋₃ alkyl;
wherein each alkyl and cycloalkyl is optionally substituted with one to three substituents each individually selected from halo, OH, amine, cyano, C₁₋₃ alkyl, C₁₋₃ alkoxy, and C₁₋₃ haloalkyl, oxo, and —C(O)R¹⁰, wherein R¹⁰ is hydrogen, OH, C₁₋₃ alkyl or C₁₋₃ alkoxy.

2. The compound of claim 1, wherein R¹ is a 3- to 10-membered heterocyclyl.

3. The compound of claim 2 wherein R¹ is a 6-membered heterocyclyl.

4. The compound of claim 2 wherein R¹ is azepanyl, 1,3-dioxolanyl, 1,4-dioxolanyl, maleimidyl, succinimidyl, dioxopiperazinyl, hydantoinyl, imidazolinyl, imidazolidinyl, isoxazolinyl, isoxazolidinyl, oxazolinyl, oxazolidinyl, oxazolidinonyl, thiazolinyl, thiazolidinyl, morpholinyl, oxiranyl, piperidinyl N-oxide, piperidinyl, piperazinyl, pyrrolidinyl, pyrrolidonyl, pyrrolidionyl, 4-piperidonyl, pyrazolinyl, pyrazolidinyl, 2-oxopyrrolidinyl, tetrahydropyranyl, quinuclidinyl, 4H-pyran, azetidinyl, oxetanyl, octahydrocyclopenta[c]pyrrole, 2-azaspiro[3.3]heptanyl, 3-oxabicyclo[3.1.0]hexanyl, 3-azabicyclo[3.1.0]hexanyl, 3-azabicyclo[3.1.1]heptanyl, 4-azaspiro[2.5]octanyl, 6-azaspiro[3.5]nonanyl, 2,6-diazaspiro[3.3]heptanyl, 7-azabicyclo[2.2.1]heptanyl, 2-azabicyclo[2.2.1]heptanyl, 2,5-diazabicyclo[2.2.2]octanyl, 2,5-diazabicyclo[2.2.1]heptanyl, 2-oxabicyclo[2.1.1]hexanyl, 3-azabicyclo[3.2.1]octanyl, hexahydro-1H-cyclopenta[c]pyrrolyl, 3-oxa-9-azabicyclo[3.3.1]nonanyl, or hexahydro-1H-pyrrolizinyl, optionally substituted with one or more C₁₋₃ alkyl.

5. The compound of claim 4 wherein R¹ is tetrahydrofuranyl, tetrahydropyranyl, azetidinyl, pyrrolidinyl, or piperidinyl.

6. The compound of claim 5 wherein R¹ is piperidinyl, optionally substituted with one or more C₁₋₃ alkyl.

7. The compound of claim 1 wherein R² is hydrogen, halo, cyano or C₁₋₃ haloalkyl.

8. The compound of claim 1 wherein R³ is a 3- to 10-membered heterocyclyl or a 5- to 10-membered heteroaryl.

9. The compound of claim 8 wherein R³ is a 4- to 6-membered heterocyclyl.

10. The compound of claim 8 wherein R³ is azepanyl, 1,3-dioxolane, 1,4-dioxolanyl, maleimidyl, succinimidyl, dioxopiperazinyl, hydantoinyl, imidazolinyl, imidazolidinyl, isoxazolinyl, isoxazolidinyl, oxazolinyl, oxazolidinyl, oxazolidinonyl, thiazolinyl, thiazolidinyl, morpholinyl, oxiranyl, piperidinyl N-oxide, piperidinyl, piperazinyl, pyrrolidinyl, pyrrolidonyl, pyrrolidionyl, 4-piperidonyl, pyrazolinyl, pyrazolidinyl, 2-oxopyrrolidinyl, tetrahydropyranyl, quinuclidineyl, 4H-pyranyl, azetidinyl, oxetanyl, octahydrocyclopenta[c]pyrrole, 2-azaspiro[3.3]heptanyl, 3-oxabicyclo[3.1.0]hexanyl, 3-azabicyclo[3.1.0]hexanyl, 3-azabicyclo[3.1.1]heptanyl, 4-azaspiro[2.5]octanyl, 6-azaspiro[3.5]nonanyl, 2,6-diazaspiro[3.3]heptanyl, 7-azabicyclo[2.2.1]heptanyl, 2-azabicyclo[2.2.1]heptanyl, 2,5-diazabicyclo[2.2.2]octanyl, 2,5-diazabicyclo[2.2.1]heptanyl, 2-oxabicyclo[2.1.1]hexanyl, 3-azabicyclo[3.2.1]octanyl, hexahydro-1H-cyclopenta[c]pyrrolyl, 3-oxa-9-azabicyclo[3.3.1]nonanyl, 1,3-oxa-7-azabicyclo[3.3.1]nonanyl, hexahydro-1H-pyrrolizinyl, 2-oxa-7-azaspiro[4.4]nonanyl, or 6-oxa-1-azaspiro[3.4]octanyl, optionally substituted with one or more halo, OH or C₁₋₃ alkyl.

11. The compound of claim 10 wherein R³ is piperidinyl, substituted with one or more halo, OH or C₁₋₃ alkyl.

12. The compound of claim 10 wherein R³ is morpholinyl, substituted with one or more halo, OH or C₁₋₃ alkyl.

13. The compound of claim 1 wherein R³ is oxazolyl, thiszolyl, pyrazolyl, pyridyl or pyridone.

14. The compound of claim 1, wherein
R³ is-C(O) R⁷; and
R⁷ is C₁₋₃ alkoxy.

15. The compound of claim 1, wherein R³ is —C(O)NR⁸R⁹, and:
i) R⁸ is hydrogen or C₁₋₃ alkyl and R⁹ is C₁₋₃ alkyl; or
ii) R⁸ and R⁹ are optionally taken together to form a 5- to 8-membered heterocycly.

16. The compound of claim 1 wherein R⁴ is C₁₋₃ alkyl.

17. The compound of claim 1 wherein R⁴ is methyl.

18. The compound of claim 1 wherein R⁵ is halo, CN, C₁₋₃ alkyl or C₁₋₃ haloalkyl.

19. A compound selected from the group consisting of:
(R)—N-(1-(3-(difluoromethyl)-2-fluorophenyl)ethyl)-4-fluoro-2-((1-methylpiperidin-4-yl)amino)-5-morpholinobenzamide,
5-((1R,4R)-2-oxa-5-azabicyclo[2.2.1]heptan-5-yl)-N—((R)-1-(3-(difluoromethyl)-2-fluorophenyl)ethyl)-4-fluoro-2-((1-methylpiperidin-4-yl)amino)benzamide,
(R)-4-fluoro-N-(1-(2-methyl-3-(trifluoromethyl)phenyl)ethyl)-2-((1-methylpiperidin-4-yl)amino)-5-morpholinobenzamide,
(R)-4-cyano-N-(1-(3-(difluoromethyl)-2-fluorophenyl)ethyl)-2-((1-methylpiperidin-4-yl)amino)-5-morpholinobenzamide,
(R)—N-(1-(3-(difluoromethyl)-2-fluorophenyl)ethyl)-4-fluoro-5-(4-methylpiperazin-1-yl)-2-((1-methylpiperidin-4-yl)amino)benzamide,
5-(6-oxa-3-azabicyclo[3.1.1]heptan-3-yl)-N—((R)-1-(3-(difluoromethyl)-2-fluorophenyl)ethyl)-4-fluoro-2-((1-methylpiperidin-4-yl)amino)benzamide,
(R)—N-(1-(3-(difluoromethyl)-2-fluorophenyl)ethyl)-5-(6,7-dihydropyrazolo[1,5-a]pyrazin-5 (4H)-yl)-4-fluoro-2-((1-methylpiperidin-4-yl)amino)benzamide,
(R)-5-(2,2-dioxido-2-thia-6-azaspiro[3.3]heptan-6-yl)-4-fluoro-N-(1-(2-methyl-3-(trifluoromethyl)phenyl)ethyl)-2-((1-methylpiperidin-4-yl)amino)benzamide,
N—((R)-1-(3-(difluoromethyl)-2-fluorophenyl)ethyl)-4-fluoro-2-((1-methylpiperidin-4-yl)amino)-5-(((R)-tetrahydrofuran-3-yl)oxy)benzamide,
N—((R)-1-(3-(difluoromethyl)-2-fluorophenyl)ethyl)-4-fluoro-2-((1-methylpiperidin-4-yl)amino)-5-(((S)-tetrahydrofuran-3-yl)oxy)benzamide,
(R)-4-fluoro-5-(1-methyl-1H-pyrazol-4-yl)-N-(1-(2-methyl-3-(trifluoromethyl)phenyl)ethyl)-2-((1-methylpiperidin-4-yl)amino)benzamide,
(R)-4-fluoro-N-(1-(2-methyl-3-(trifluoromethyl)phenyl)ethyl)-2-((1-methylpiperidin-4-yl)amino)-5-(oxazol-5-yl)benzamide,
(R)-4-chloro-N-(1-(2-methyl-3-(trifluoromethyl)phenyl)ethyl)-2-((1-methylpiperidin-4-yl)amino)-5-morpholinobenzamide,
(R)—N-(1-(3-(difluoromethyl)-2-fluorophenyl)ethyl)-4-fluoro-5-morpholino-2-(piperidin-4-ylamino)benzamide,
N—((R)-1-(3-(difluoromethyl)-2-fluorophenyl)ethyl)-4-fluoro-5-morpholino-2-(((R)-quinuclidin-3-yl)amino)benzamide,
N—((R)-1-(3-(difluoromethyl)-2-fluorophenyl)ethyl)-4-fluoro-5-morpholino-2-(((S)-quinuclidin-3-yl)amino)benzamide,
N—((R)-1-(3-(difluoromethyl)-2-fluorophenyl)ethyl)-4-fluoro-2-(((1R,5S,6s)-3-methyl-3-azabicyclo[3.1.1]heptan-6-yl)amino)-5-morpholinobenzamide,
N—((R)-1-(3-(difluoromethyl)-2-fluorophenyl)ethyl)-4-fluoro-2-(((1R,5S,6r)-3-methyl-3-azabicyclo[3.1.1]heptan-6-yl)amino)-5-morpholinobenzamide,
5-((1S,4S)-2-oxa-5-azabicyclo[2.2.1]heptan-5-yl)-N—((R)-1-(3-(difluoromethyl)-2-fluorophenyl)ethyl)-4-fluoro-2-((1-methylpiperidin-4-yl)amino)benzamide,
(R)-5-(6-acetyl-2,6-diazaspiro[3.3]heptan-2-yl)-4-fluoro-N-(1-(2-methyl-3-(trifluoromethyl)phenyl)ethyl)-2-((1-methylpiperidin-4-yl)amino)benzamide,
(R)-4-fluoro-N-(1-(2-methyl-3-(trifluoromethyl)phenyl)ethyl)-2-((1-methylpiperidin-4-yl)amino)-5-(tetrahydro-2H-pyran-4-yl)benzamide,
(R)-5-(4-acetylpiperazin-1-yl)-4-fluoro-N-(1-(2-methyl-3-(trifluoromethyl)phenyl)ethyl)-2-((1-methylpiperidin-4-yl)amino)benzamide,
(R)—N-(1-(2-methyl-3-(trifluoromethyl)phenyl)ethyl)-2-((1-methylpiperidin-4-yl)amino)-5-morpholinobenzamide,
4-cyano-N—((R)-1-(3-(difluoromethyl)-2-fluorophenyl)ethyl)-5-morpholino-2-(((R)-quinuclidin-3-yl)amino)benzamide,
4-cyano-N—((R)-1-(3-(difluoromethyl)-2-fluorophenyl)ethyl)-5-morpholino-2-(((S)-quinuclidin-3-yl)amino)benzamide,
N—((R)-1-(3-(difluoromethyl)-2-fluorophenyl)ethyl)-5-(3,3-difluoropyrrolidin-1-yl)-4-fluoro-2-(((S)-quinuclidin-3-yl)amino)benzamide,
N—((R)-1-(3-(difluoromethyl)-2-fluorophenyl)ethyl)-5-(3,3-difluoropyrrolidin-1-yl)-4-fluoro-2-(((R)-quinuclidin-3-yl)amino)benzamide,
5-(azetidin-1-yl)-N—((R)-1-(3-(difluoromethyl)-2-fluorophenyl)ethyl)-4-fluoro-2-(((S)-quinuclidin-3-yl)amino)benzamide,
5-(azetidin-1-yl)-N—((R)-1-(3-(difluoromethyl)-2-fluorophenyl)ethyl)-4-fluoro-2-(((R)-quinuclidin-3-yl)amino)benzamide,
N—((R)-1-(3-(difluoromethyl)-2-fluorophenyl)ethyl)-4-fluoro-2-((1-methylpiperidin-4-yl)amino)-5-((R)-2-oxa-7-azaspiro[4.4]nonan-7-yl)benzamide,
N—((R)-1-(3-(difluoromethyl)-2-fluorophenyl)ethyl)-4-fluoro-2-((1-methylpiperidin-4-yl)amino)-5-((S)-2-oxa-7-azaspiro[4.4]nonan-7-yl)benzamide,
N—((R)-1-(3-(difluoromethyl)-2-fluorophenyl)ethyl)-4-fluoro-5-(pyrrolidin-1-yl)-2-(((S)-quinuclidin-3-yl)amino)benzamide,
N—((R)-1-(3-(difluoromethyl)-2-fluorophenyl)ethyl)-4-fluoro-5-(pyrrolidin-1-yl)-2-(((R)-quinuclidin-3-yl)amino)benzamide,
N—((R)-1-(3-(difluoromethyl)-2-fluorophenyl)ethyl)-4-fluoro-2-((1-methylpiperidin-4-yl)amino)-5-((S)-6-oxa-1-azaspiro[3.4]octan-1-yl)benzamide;
N—((R)-1-(3-(difluoromethyl)-2-fluorophenyl)ethyl)-4-fluoro-2-((1-methylpiperidin-4-yl)amino)-5-((R)-6-oxa-1-azaspiro[3.4]octan-1-yl)benzamide,
5-((R)-4,4-difluoro-2-methylpyrrolidin-1-yl)-N—((R)-1-(3-(difluoromethyl)-2-fluorophenyl)ethyl)-4-fluoro-2-((1-methylpiperidin-4-yl)amino)benzamide,
5-((S)-4,4-difluoro-2-methylpyrrolidin-1-yl)-N—((R)-1-(3-(difluoromethyl)-2-fluorophenyl)ethyl)-4-fluoro-2-((1-methylpiperidin-4-yl)amino)benzamide,
5-(3-oxa-7-azabicyclo[3.3.1]nonan-7-yl)-N—((R)-1-(3-(difluoromethyl)-2-fluorophenyl)ethyl)-4-fluoro-2-((1-methylpiperidin-4-yl)amino)benzamide,
5-(3-oxa-9-azabicyclo[3.3.1]nonan-9-yl)-N—((R)-1-(3-(difluoromethyl)-2-fluorophenyl)ethyl)-4-fluoro-2-((1-methylpiperidin-4-yl)amino)benzamide,
(R)—N-(1-(3-(difluoromethyl)-2-fluorophenyl)ethyl)-5-(2,2-difluoromorpholino)-4-fluoro-2-((1-methylpiperidin-4-yl)amino)benzamide,
5-(9-oxa-3-azabicyclo[3.3.1]nonan-3-yl)-N—((R)-1-(3-(difluoromethyl)-2-fluorophenyl)ethyl)-4-fluoro-2-((1-methylpiperidin-4-yl)amino)benzamide,
N—((R)-1-(3-(1,1-difluoroethyl)-2-fluorophenyl)ethyl)-4-fluoro-5-morpholino-2-(((S)-quinuclidin-3-yl)amino)benzamide,
N—((R)-1-(3-(1,1-difluoroethyl)-2-fluorophenyl)ethyl)-4-fluoro-5-morpholino-2-(((R)-quinuclidin-3-yl)amino)benzamide,
N—((R)-1-(3-cyano-2-methylphenyl)ethyl)-4-fluoro-5-morpholino-2-(((S)-quinuclidin-3-yl)amino)benzamide,
N—((R)-1-(3-cyano-2-methylphenyl)ethyl)-4-fluoro-5-morpholino-2-(((R)-quinuclidin-3-yl)amino)benzamide,
(R)—N-(1-(3-cyano-2-methylphenyl)ethyl)-4-fluoro-2-((1-methylpiperidin-4-yl)amino)-5-morpholinobenzamide,
(R)-2-((2-aminoethyl)amino)-N-(1-(3-(difluoromethyl)-2-fluorophenyl)ethyl)-4-fluoro-5-morpholinobenzamide,
(R)—N-(1-(3-(difluoromethyl)-2-fluorophenyl)ethyl)-2-((2-(dimethylamino)ethyl)amino)-4-fluoro-5-morpholinobenzamide,
N—((R)-1-(3-(difluoromethyl)-2-fluorophenyl)ethyl)-2-(((R)-1-(dimethylamino) propan-2-yl)amino)-4-fluoro-5-morpholinobenzamide,
N—((R)-1-(3-(difluoromethyl)-2-fluorophenyl)ethyl)-2-(((S)-1-(dimethylamino) propan-2-yl)amino)-4-fluoro-5-morpholinobenzamide,
(R)-2-((2-cyanopropan-2-yl)amino)-N-(1-(3-(difluoromethyl)-2-fluorophenyl)ethyl)-4-fluoro-5-morpholinobenzamide,
(R)—N-(1-(3-(difluoromethyl)-2-fluorophenyl)ethyl)-2-((1-(dimethylamino)-2-methylpropan-2-yl)amino)-4-fluoro-5-morpholinobenzamide,
(R)—N-(1-(3-(difluoromethyl)-2-fluorophenyl)ethyl)-2-((3-(dimethylamino) bicyclo[1.1.1]pentan-1-yl)amino)-4-fluoro-5-morpholinobenzamide,
(R)—N-(1-(3-(difluoromethyl)-2-fluorophenyl)ethyl)-2-((1,3-dimethylazetidin-3-yl)amino)-4-fluoro-5-morpholinobenzamide,
N—((R)-1-(3-(difluoromethyl)-2-fluorophenyl)ethyl)-4-fluoro-2-(((1R,5S,6s)-3-methyl-3-azabicyclo[3.1.0]hexan-6-yl)amino)-5-morpholinobenzamide,
(R)-4-fluoro-N-(1-(2-methyl-3-(trifluoromethyl)phenyl)ethyl)-2-((1-methylpiperidin-4-yl)amino)-5-(pyridin-4-yl)benzamide,
(R)—N-(1-(3-(difluoromethyl)-2-fluorophenyl)ethyl)-4-fluoro-2-((1-methylpiperidin-4-yl)amino)-5-(pyridin-4-yl)benzamide,
N—((R)-1-(3-(difluoromethyl)-2-fluorophenyl)ethyl)-4-fluoro-5-(pyridin-4-yl)-2-(((R)-quinuclidin-3-yl)amino)benzamide,
N—((R)-1-(3-(difluoromethyl)-2-fluorophenyl)ethyl)-4-fluoro-5-(pyridin-4-yl)-2-(((S)-quinuclidin-3-yl)amino)benzamide,
(R)—N-(1-(3-(difluoromethyl)-2-fluorophenyl)ethyl)-4-fluoro-5-(1-methyl-2-oxo-1,2-dihydropyridin-4-yl)-2-((1-methylpiperidin-4-yl)amino)benzamide,
(R)-4-fluoro-5-(isoxazol-4-yl)-N-(1-(2-methyl-3-(trifluoromethyl)phenyl)ethyl)-2-((1-methylpiperidin-4-yl)amino)benzamide,
(R)—N-(1-(3-(difluoromethyl)-2-fluorophenyl)ethyl)-4-fluoro-5-(1-methyl-6-oxo-1,6-dihydropyridin-3-yl)-2-((1-methylpiperidin-4-yl)amino)benzamide,
(R)—N-(1-(3-(difluoromethyl)-2-fluorophenyl)ethyl)-4-fluoro-5-(3-fluoropyridin-4-yl)-2-((1-methylpiperidin-4-yl)amino)benzamide,
(R)—N-(1-(3-(difluoromethyl)-2-fluorophenyl)ethyl)-4-fluoro-2-((1-methylpiperidin-4-yl)amino)-5-(2-methylpyridin-4-yl)benzamide,
(R)—N-(1-(3-(difluoromethyl)-2-fluorophenyl)ethyl)-4-fluoro-2-((1-methylpiperidin-4-yl)amino)-5-(pyridazin-4-yl)benzamide,
(R)—N-(1-(3-(difluoromethyl)-2-fluorophenyl)ethyl)-4-fluoro-2-((1-methylpiperidin-4-yl)amino)-5-(pyrimidin-4-yl)benzamide,
(R)-5-cyclopropyl-N-(1-(3-(difluoromethyl)-2-fluorophenyl)ethyl)-4-fluoro-2-((1-methylpiperidin-4-yl)amino)benzamide,
(R)-5-(bicyclo[1.1.1]pentan-1-yl)-N-(1-(3-(difluoromethyl)-2-fluorophenyl)ethyl)-4-fluoro-2-((1-methylpiperidin-4-yl)amino)benzamide,
5-(bicyclo[1.1.1]pentan-1-yl)-N—((R)-1-(3-(difluoromethyl)-2-fluorophenyl)ethyl)-4-fluoro-2-(((R)-quinuclidin-3-yl)amino)benzamide,
5-(bicyclo[1.1.1]pentan-1-yl)-N—((R)-1-(3-(difluoromethyl)-2-fluorophenyl)ethyl)-4-fluoro-2-(((S)-quinuclidin-3-yl)amino)benzamide,
methyl (R)-5-((1-(3-(difluoromethyl)-2-fluorophenyl)ethyl) carbamoyl)-2-fluoro-4-((1-methylpiperidin-4-yl)amino)benzoate,
(R)—N-(1-(3-(difluoromethyl)-2-fluorophenyl)ethyl)-4-fluoro-2-((1-methylpiperidin-4-yl)amino)-5-(1,3,4-oxadiazol-2-yl)benzamide,
(R)—N1-(1-(3-(difluoromethyl)-2-fluorophenyl)ethyl)-4-fluoro-N3-methyl-6-((1-methylpiperidin-4-yl)amino)isophthalamide,
(R)—N-(1-(3-(difluoromethyl)-2-fluorophenyl)ethyl)-5-(4,4-difluoropiperidine-1-carbonyl)-4-fluoro-2-((1-methylpiperidin-4-yl)amino)benzamide,
(R)—N1-(1-(3-(difluoromethyl)-2-fluorophenyl)ethyl)-4-fluoro-N3,N3-dimethyl-6-((1-methylpiperidin-4-yl)amino)isophthalamide,
(R)—N1-(1-(3-(difluoromethyl)-2-fluorophenyl)ethyl)-4-fluoro-N3-isopropyl-6-((1-methylpiperidin-4-yl)amino)isophthalamide,
(R)—N-(1-(3-(difluoromethyl)-2-fluorophenyl)ethyl)-4-fluoro-2-((1-methylpiperidin-4-yl)amino)-5-(morpholine-4-carbonyl)benzamide,
methyl (R)-3-((1-(3-(difluoromethyl)-2-fluorophenyl)ethyl) carbamoyl)-4-((1-methylpiperidin-4-yl)amino)benzoate,
(R)-4-(difluoromethyl)-N-(1-(3-(difluoromethyl)-2-fluorophenyl)ethyl)-2-((1-methylpiperidin-4-yl)amino)-5-morpholinobenzamide,
4-chloro-N—((R)-1-(3-(difluoromethyl)-2-fluorophenyl)ethyl)-5-morpholino-2-(((R)-quinuclidin-3-yl)amino)benzamide,
4-chloro-N—((R)-1-(3-(difluoromethyl)-2-fluorophenyl)ethyl)-5-morpholino-2-(((S)-quinuclidin-3-yl)amino)benzamide
or a pharmaceutically acceptable salt thereof.

20. A pharmaceutical composition comprising a compound of any one of claims 1-19, or a pharmaceutically acceptable salt thereof, and at least one pharmaceutically acceptable excipient.

21. A method for treating cancer in a subject in need thereof, comprising administering to the subject an effective amount of a compound of any one of claims 1-19.

22. The method according to claim 21, wherein the cancer is a Ras pathway-associated cancer.
